(19) United States
(12) Patent Application Publication
(10) Pub. No.: US 2012/0129854 A1
(43) Pub. Date: May 24, 2012

(54) Pesticidal Arylpyrrolidines

(75) Inventors: Jun Mihara, Oyama-shi (JP); Tetsuya Murata, Oyama-shi (JP); Daiei Yamazaki, Oyama-shi (JP); Yasushi Yoneta, Hanyu-shi (JP); Koichi Araki, Ibaraki (JP); Norio Sasaki, Ibaraki (JP); Kei Domon, Oyama-shi (JP); Mamoru Hatazawa, Ibaraki (JP); Eiichi Shimojo, Kawachinagano-shi (JP); Teruyuki Ichihara, Oyama-shi (JP); Masashi Ataka, Saitama-shi (JP); Katsuhiko Shibuya, Shimotsuke-shi (JP); Ulrich Görgens, Ratingen (DE)

(73) Assignee: Bayer CropScience AG, Monheim (DE)

(21) Appl. No.: 13/318,131

(22) PCT Filed: Apr. 27, 2010
(86) PCT No.: PCT/EP2010/002580

§ 371 Date: Jan. 9, 2012

(30) Foreign Application Priority Data
Apr. 30, 2009 (JP) 2009111390

(51) Int. Cl. A01N 43/84; C07D 417/04; C07D 401/04; C07D 401/14; C07D 413/14; C07D 405/14; C07D 409/14; C07D 403/04; A01N 43/36; A01N 43/78; A01N 43/42; A01N 43/54; A61K 31/402; A61K 31/428; A61K 31/4709; A61K 31/5377; A61K 31/435; A61K 31/517; A01P 7/04; A01P 7/00; A01P 5/00; A01P 9/00; A61P 33/00; A61P 33/02; C07D 207/08

(52) U.S. Cl. 514/234.5; 548/577; 548/162; 546/159; 544/127; 546/112; 544/253; 544/119; 514/429; 514/393; 514/313; 514/235.2; 514/299; 514/258.1

(57) ABSTRACT

The invention is directed to arylpyrrolidines compounds which exhibit excellent insecticidal efficacy and which may be used as in the agrochemical field or in the yield of veterinary medicine. The compounds are represented by formula (I): wherein the respective substituents are defined in the specification.

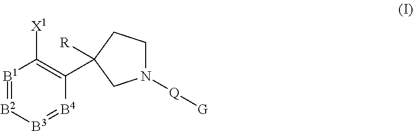

The present invention relates to novel arylpyrrolidines and their use as pesticides in the agrocultural field or as pharmaceutical for treating parasites in or on animals.

It is known that certain compounds containing a 5-membered heterocyclic ring can be used as pesticides. Substituted benzamide compounds containing a 5-membered ring which is isoxazoline, and which compounds are supposed to be useful for controlling noxious animal pests are described in WO2005/085216. Compounds, wherein the 5-membered ring is a pyrrolidine ring and which may be used as an agent for controlling noxious bioorganisms are described in JP2008-110971A and WO2008/128711.

Modern crop protection compositions have to satisfy many demands, for example in relation to efficacy, persistence and spectrum of their action, and possible use. Important questions relate to toxicity, combinability with other active ingredients or formulating assistants, and another is that of the effort and expense of synthesizing an active ingredient. Moreover, resistances can occur. For all these reasons, the search for novel crop protection compositions cannot be considered to be complete, and there is a constant need for novel compounds with improved properties over the known compounds, at least in relation to individual aspects. Thus, the inventors of the present invention intensively studied to develop novel compounds which exhibit a high pesticidal efficacy and have a broad spectrum of use.

As a result, the inventors have found that novel arylpyrrolidines represented by the following formula (I) have high pesticidal activity, a broad spectrum of use, safety, and also have an efficacy against noxious insects that are resistant to an organophosphorus agent or a carbamate agent.

Thus, this invention is directed to arylpyrrolidine compounds of formula (I)

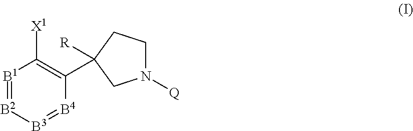

wherein
R represents optionally substituted C₁₋₁₂-alkyl or C₁₋₁₂-haloalkyl;
Q stands for a group selected among the groups Q-1 to Q-12

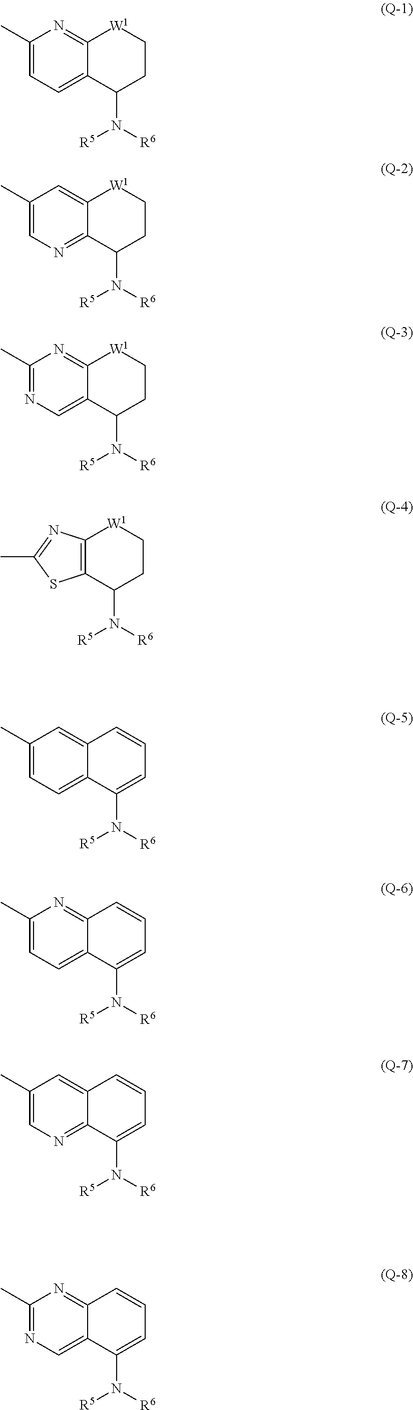

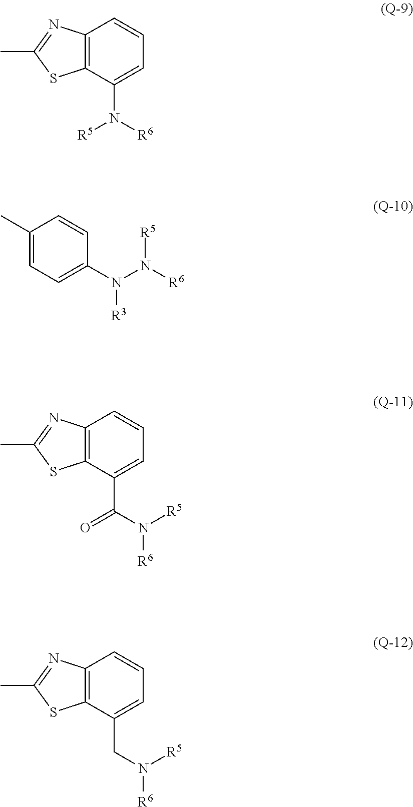

in which
W¹ stands for a single bond or methylene (i.e. a group —CH₂—), and which group may be optionally substituted by at least one substituent Y¹, whereas the substituent is preferably connected to a carbon atom;
B¹, B², B³ and B⁴ each independently represents C—X² or nitrogen, and if either B¹ and B², or B² and B³, or B³ and B⁴ stand for C—X², then the substituents X² may together with the carbon atoms to which they are bound form a 5- or 6-membered saturated or unsaturated hydrocarbon ring or heterocyclic ring:
X¹, X² each independently represents hydrogen, halogen, nitro, cyano, hydroxy, mercapto, amino, alkyl, haloalkyl, alkoxy, haloalkoxy, alkylsulfanyl, alkylsulfinyl, alkylsulfonyl, haloalkylsulfanyl, haloalkylsulfinyl, haloalkylsulfonyl, acylamino, alkoxycarbonylamino, haloalkoxycarbonylamino, alkoxyimino, haloalkoxyimino, alkylsulfonylamino, or pentafluorosulfur;
Y¹ represents hydrogen, halogen, nitro, cyano, hydroxy, mercapto, amino, alkyl, haloalkyl, cycloalkyl, cyclohaloalkyl, alkenyl, alkynyl, alkoxy, haloalkoxy, alkylsulfanyl, alkylsulfinyl, alkylsulfonyl, haloalkylsulfanyl, haloalkylsulfinyl, haloalkylsulfonyl, alkylsulfonyloxy, haloalkylsulfonyloxy, alkylaminosulfonyl, haloalkylaminosulfonyl, dialkylaminosulfonyl, di(haloalkyl)aminosulfonyl, alkylamino, dialkylamino, acylamino, alkoxycarbonylamino, haloalkoxycarbonylamino, alkylsulfonylamino, haloalkylsulfonylamino, trialkylsilyl, alkoxyimino, haloalkoxyimino, alkoxyiminoalkyl, haloalkoxyiminoalkyl, alkylsulfinylimino, alkylsulfinyliminoalkyl, alkylsulfinyliminoalkylcarbonyl, alkylsulfoxyimino, alkylsulfoxyiminoalkyl, alkoxycarbonyl, alkylcarbonyl, aminocarbonyl, alkylaminocarbonyl, aminothiocarbonyl, alkylaminothiocarbonyl, dialkylaminocarbonyl, or dialkylaminothiocarbonyl;
R³ represents hydrogen, cyano, alkyl, alkenyl, alkynyl, cycloalkyl or alkoxycarbonyl;
R⁵ represents hydrogen, amino, hydroxy, cyano, alkyl, haloalkyl, cycloalkyl, alkenyl, alkynyl, alkoxy, aminocarbonylalkyl, iminoalkyl, alkylcarbonyl, alkylcarbonylamino, alkylimino, aryl, aralkyl, a heterocycle, a heterocyclic ring substituted alkyl, R⁷—C(═O)— or R⁷—C(═S)—;
R⁶ represents hydrogen, cyano, carbonyl, thiocarbonyl, alkylcarbonyl, alkylthiocarbonyl, haloalkylcarbonyl, haloalkylthiocarbonyl, alkylaminocarbonyl, alkylaminothiocarbonyl, dialkylaminocarbonyl, dialkylaminothiocarbonyl, alkoxyaminocarbonyl, alkoxyaminothiocarbonyl, alkoxycarbonyl, alkoxythiocarbonyl, alkylsulfanylcarbonyl, alkylsulfanylthiocarbonyl, alkylsulfonyl, haloalkylsulfonyl, cycloalkylcarbonyl, alkenylcarbonyl, alkynylcarbonyl, cycloalkylalkylcarbonyl, alkylsulfanylalkylcarbonyl, alkylsulfinylalkylcarbonyl, alkylsulfonylalkylcarbonyl, alkylcarbonylalkylcarbonyl, cycloalkylaminocarbonyl, alkenylaminocarbonyl, alkynylaminocarbonyl, alkylaminosulfonyl, dialkylaminosulfonyl, alkoxyalkylcarbonyl, alkylcarbonylaminoalkylcarbonyl, haloalkylcarbonylaminoalkylcarbonyl, alkylsulfanylalkylcarbonylaminoalkylcarbonyl, alkylsulfinylalkylcarbonylaminoalkylcarbonyl, alkylsulfonylalkylcarbonylaminoalkylcarbonyl, aralkylcarbonyl, heterocyclic substituted alkylcarbonyl, R⁷—C(═O)— or R⁷—C(═S)—, or
R⁵ and R⁶ may form a 3- to 6-membered heterocyclic ring, together with the nitrogen atom to which they are bound, and said ring may be substituted with keto, thioketo or a nitroimino group; or
R⁷ represents an optionally substituted phenyl or an optionally substituted heterocycle.

Compounds of formula (I) are preferred wherein

R represents C₁₋₆ alkyl, or C₁₋₆ haloalkyl, preferably represents C₁₋₄ alkyl or C₁₋₄ haloalkyl, more preferably represents trifluoromethyl;
Q stands for a group Q-1 to Q-12 as defined above, preferably stands for Q-5 or Q-6:
B¹, B², B³ and B⁴ each independently represents C—X² or nitrogen, and if either B¹ and B², or B² and B³, or B³ and B⁴ stand for C—X², then the substituents X² may together with the carbon atoms to which they are bound form a 5- or 6-membered saturated or unsaturated hydrocarbon ring or heterocyclic ring, preferably B¹, B² and B³ each independently represents C—X² and B⁴ represents nitrogen, or B¹, B³ and B⁴ each independently represents C—X² and B² represents nitrogen;
X¹, X² each independently represents hydrogen, halogen, nitro, cyano, hydroxy, mercapto, amino, C₁₋₆alkyl, C₁₋₆haloalkyl, (particularly CF₃, CF₂H, CFH₂, CH₂CF₃, CF₂CF₃), C₁₋₆alkoxy, C₁₋₆haloalkoxy, C₁₋₆alkylsulfanyl, C₁₋₆alkylsulfinyl, C₁₋₆alkylsulfonyl, C₁₋₆haloalkylsulfanyl, C₁₋₆haloalkylsulfinyl, C₁₋₆haloalkylsulfonyl, (total carbon atom number) C₂₋₇acylamino, (total carbon atom number) C₂₋₇alkoxycarbonylamino, (total carbon atom number) C₂₋₇haloalkoxycarbonylamino, C₁₋₆alkoxyimino, C₁₋₆haloalkoxyimino, C₁₋₆alkylsulfonylamino or pentafluorosulfur; preferably represents hydrogen, halogen, nitro, cyano, hydroxy, mercapto, amino, C₁₋₄alkyl, C₁₋₄haloalkyl, C₁₋₄alkoxy, C₁₋₄haloalkoxy, C₁₋₄alkylsulfanyl, C₁₋₄alkylsulfinyl, C₁₋₄alkylsulfonyl, C₁₋₄haloalkylsulfanyl, C₁₋₄haloalkylsulfinyl, C₁₋₄haloalkylsulfonyl, (total carbon atom number) C₂₋₅acylamino, (total carbon atom number) C₂₋₅alkoxycarbonylamino, (total carbon atom number) C₂₋₅haloalkoxycarbonylamino, C₁₋₄alkoxyimino, C₁₋₄haloalkoxyimino, C₁₋₄alkylsulfonylamino or pentafluorosulfur, more preferably represents hydrogen, fluoro, chloro, bromo, trifluoromethyl;
Y¹ represents hydrogen, halogen, nitro, cyano, hydroxy, mercapto, amino, C₁₋₆alkyl, C₁₋₆haloalkyl, C₃₋₇cycloalkyl, C₃₋₇cycloC₁₋₆haloalkyl, C₂₋₆alkenyl, C₂₋₆alkynyl, C₁₋₆alkoxy, C₁₋₆haloalkoxy, C₁₋₆alkylsulfanyl, C₁₋₆alkylsulfinyl, C₁₋₆alkylsulfonyl, C₁₋₆haloalkylsulfanyl, C₁₋₆haloalkylsulfinyl, C₁₋₆haloalkylsulfonyl, C₁₋₆alkylsulfonyloxy, C₁₋₆haloalkylsulfonyloxy, C₁₋₆alkylaminosulfonyl, C₁₋₆haloalkylaminosulfonyl, (total carbon atom number) C₂₋₁₂dialkylaminosulfonyl, (total carbon atom number) C₂₋₁₂di(haloalkyl)aminosulfonyl, C₁₋₆alkylamino, (total carbon atom number) C₂₋₁₂dialkylamino, (total carbon atom number) C₂₋₇acylamino, (total carbon atom number) C₂₋₇alkoxycarbonylamino, (total carbon atom number) C₂₋₇haloalkoxycarbonylamino, C₁₋₆alkylsulfonylamino, C₁₋₆haloalkylsulfonylamino, (total carbon atom number) C₃₋₁₈trialkylsilyl, C₁₋₆alkoxyimino, C₁₋₆haloalkoxyimino, (total carbon atom number) C₂₋₁₂alkoxyiminoalkyl, (total carbon atom number) C₂₋₁₂haloalkoxyiminoalkyl, C₁₋₆alkylsulfinylimino, (total carbon atom number) C₂₋₁₂alkylsulfinyliminoalkyl, (total carbon atom number) C₃₋₁₃alkylsulfinyliminoalkylcarbonyl, C₁₋₆alkylsulfoxyimino, (total carbon atom number) C₂₋₁₂alkylsulfoxyiminoalkyl, (total carbon atom number) C₂₋₇alkoxycarbonyl, (total carbon atom number) C₂₋₇alkylcarbonyl, aminocarbonyl, (total carbon atom number) C₂₋₇alkylaminocarbonyl, aminothiocarbonyl, (total carbon atom number) C₂₋₇alkylaminothiocarbonyl, (total carbon atom number) C₃₋₁₃dialkylaminocarbonyl or (total carbon atom number) C₃₋₁₃dialkylaminothiocarbonyl, preferably represents hydrogen, halogen, nitro, cyano, hydroxy, mercapto, amino, C₁₋₄alkyl, C₁₋₄haloalkyl, C₃₋₆cycloalkyl, C₃₋₆cyclohaloalkyl, C₂₋₄alkynyl, C₁₋₄alkoxy, C₁₋₄haloalkoxy, C₁₋₄alkylsulfanyl, C₁₋₄alkylsulfinyl, C₁₋₄alkylsulfonyl, C₁₋₄haloalkylsulfanyl, C₁₋₄haloalkylsulfinyl, C₁₋₄haloalkylsulfonyl, C₁₋₄alkylsulfonyloxy, C₁₋₄haloalkylsulfonyloxy, C₁₋₄alkylaminosulfonyl, C₁₋₄haloalkylaminosulfonyl, (total carbon atom number) C₂₋₈dialkylaminosulfonyl, (total carbon atom number) C₂₋₈ di(haloalkyl)aminosulfonyl, C₁₋₄alkylamino, (total carbon atom number) C₂₋₈dialkylamino, (total carbon atom number) C₂₋₅acylamino, (total carbon atom number) C₂₋₅alkoxycarbonylamino, (total carbon atom number) C₂₋₅haloalkoxycarbonylamino, C₁₋₄alkylsulfonylamino, C₁₋₄haloalkylsulfonylamino, (total carbon atom number) C₃₋₁₂trialkylsilyl, C₁₋₄alkoxyimino, C₁₋₄haloalkoxyimino, (total carbon atom number) C₂₋₈alkoxyiminoalkyl, (total carbon atom number) C₂₋₈haloalkoxyiminoalkyl, (total carbon atom number) C₁₋₄alkylsulfinylimino, (total carbon atom number) C₂₋₈alkylsulfinyliminoalkyl, (total carbon atom number) C₃₋₉alkylsulfinyliminoalkylcarbonyl, C₁₋₄alkylsulfoxyimino, (total carbon atom number) C₂₋₈alkylsulfoxyiminoalkyl, (total carbon atom number) C₂₋₅alkoxycarbonyl, (total carbon atom number) C₂₋₅ alkylcarbonyl, aminocarbonyl, (total carbon atom number) C₂₋₅alkylaminocarbonyl, aminothiocarbonyl, (total carbon atom number) C₂₋₅alkylaminothiocarbonyl, (total carbon atom number) C₃₋₁₁dialkylaminocarbonyl or (total carbon atom number) C₃₋₉dialkylaminothiocarbonyl, more preferably represents hydrogen, halogen, methyl, or trifluoromethyl;
R³ represents hydrogen, cyano, C₁₋₆alkyl, C₂₋₆alkenyl, C₂₋₆alkynyl, C₃₋₇cycloalkyl or (total carbon atom number) C₂₋₇alkoxycarbonyl, preferably represents hydrogen, cyano, C₁₋₄ alkyl, C₂₋₄ alkenyl, C₂₋₄ alkynyl, C₃₋₆ cycloalkyl or (total carbon atom number) C₂₋₅ alkoxycarbonyl, more preferably represents hydrogen;
R⁵ represents hydrogen, amino, hydroxy, cyano, C₁₋₆alkyl, C₁₋₆haloalkyl, C₃₋₇cycloalkyl, C₂₋₆alkenyl, C₂₋₆alkynyl, C₂₋₆alkoxy, (total carbon atom number) C₂₋₇aminocarbonylalkyl, C₁₋₆iminoalkyl, (total carbon atom number) C₂₋₇alkylcarbonyl, (total carbon atom number) C₂₋₇alkylcarbonylamino, (total carbon atom number) C₂₋₇alkylimino, (total carbon atom number) C₆₋₁₂aryl, (total carbon atom number) C₇₋₁₆aralkyl, a heterocyclic group, heterocyclic ring substituted C₁₋₆alkyl, R⁷—C(═O)— or R⁷—C(═S)—, preferably represents hydrogen, amino, hydroxy, cyano, C₁₋₄alkyl, C₁₋₄haloalkyl, C₃₋₆cycloalkyl, C₂₋₄alkenyl, C₂₋₄alkynyl, C₁₋₄alkoxy, (total carbon atom number) C₂₋₅aminocarbonylalkyl, C₁₋₄iminoalkyl, (total carbon atom number) C₂₋₅alkylcarbonyl, (total carbon atom number) C₂₋₅alkylcarbonylamino, (total carbon atom number) C₂₋₅alkylimino, (total carbon atom number) C₆₋₁₂aryl, (total carbon atom number) C₇₋₁₀aralkyl, a heterocyclic group, heterocyclic ring substituted C₁₋₆alkyl, R⁷—C(═O)— or R⁷—C(═S)—, more preferably represents hydrogen or methyl;
R⁶ represents hydrogen, cyano, carbonyl, thiocarbonyl, (total carbon atom number) C₂₋₇alkylcarbonyl, (total carbon atom number) C₂₋₇alkylthiocarbonyl, (total carbon atom number) C₂₋₇haloalkylcarbonyl, (total carbon atom number) C₂₋₇haloalkylthiocarbonyl, (total carbon atom number) C₂₋₇alkylaminocarbonyl, (total carbon atom number) C₂₋₇ alkylaminothiocarbonyl, (total carbon atom number) C₃₋₁₃dialkylaminocarbonyl, (total carbon atom number) C₃₋₁₃dialkylaminothiocarbonyl, (total carbon atom number) C₂₋₇ alkoxyaminocarbonyl, (total carbon atom number) C₂₋₇alkoxyaminothiocarbonyl, (total carbon atom number) C₂₋₇alkoxycarbonyl, (total carbon atom number) C₂₋₇alkoxythiocarbonyl, (total carbon atom number) C₂₋₇alkylsulfanylcarbonyl, (total carbon atom number) C₂₋₇alkylsulfanylthiocarbonyl, C₁₋₆alkylsulfonyl, C₁₋₆haloalkylsulfonyl, (total carbon atom number) C₄₋₈ cycloalkylcarbonyl, (total carbon atom number) C₃₋₇alkenylcarbonyl, (total carbon atom number) C₃₋₇alkynylcarbonyl, (total carbon atom number) C₅₋₁₄cycloalkylalkylcarbonyl, (total carbon atom number) C₃₋₁₃alkylsulfanylalkylcarbonyl, (total carbon atom number) C₃₋₁₃alkylsulfinylalkylcarbonyl, (total carbon atom number) C₃₋₁₃alkylsulfonylalkylcarbonyl, (total carbon atom number) C₄₋₁₄alkylcarbonylalkylcarbonyl, (total carbon atom number) C₄₋₈cycloalkylaminocarbonyl, (total carbon atom number) C₃₋₇alkenylaminocarbonyl, (total carbon atom number) C₃₋₇alkynylaminocarbonyl, C₁₋₆alkylaminosulfonyl, (total carbon atom number) C₂₋₁₂dialkylaminosulfonyl, (total carbon atom number) C₃₋₁₃alkoxyalkylcarbonyl, (total carbon atom number) C₄₋₁₄alkylcarbonylaminoalkylcarbonyl, (total carbon atom number) C₄₋₁₄haloalkylcarbonylaminoalkylcarbonyl, (total carbon atom number) C₅₋₂₀alkylsulfanylalkylcarbonylaminoalkylcarbonyl, (total carbon atom number) C₅₋₂₀alkylsulfinylalkylcarbonylaminoalkylcarbonyl, (total carbon atom number) C₅₋₂₀alkylsulfonylalkylcarbonylaminoalkylcarbonyl, (total carbon atom number) C₈₋₁₇aralkylcarbonyl, heterocyclic substituted (total carbon atom number) C₂₋₇alkylcarbonyl, R⁷—C(═O)— or R⁷—C(═S)—, preferably represents hydrogen, cyano, carbonyl, thiocarbonyl, (total carbon atom number) C₂₋₅alkylcarbonyl, (total carbon atom number) C₂₋₅alkylthiocarbonyl, C₂₋₅alkoxycarbonyl, (total carbon atom number) C₂₋₅haloalkylcarbonyl, (total carbon atom number) C₂₋₅haloalkylthiocarbonyl, (total carbon atom number) C₂₋₅alkylaminocarbonyl, (total carbon atom number) C₂₋₅alkylaminothiocarbonyl, (total carbon atom number) C₃₋₉dialkylaminocarbonyl, (total carbon atom number) C₃₋₉ dialkylaminothiocarbonyl, (total carbon atom number) C₂₋₅alkoxyaminocarbonyl, (total carbon atom number) C₂₋₅alkoxyaminothiocarbonyl, (total carbon atom number) C₂₋₅alkoxycarbonyl, (total carbon atom number) C₂₋₅alkoxythiocarbonyl, (total carbon atom number) C₂₋₅alkylsulfanylcarbonyl, (total carbon atom number) C₂₋₅alkylsulfanylthiocarbonyl, C₁₋₄alkylsulfonyl, C₁₋₄haloalkylsulfonyl, (total carbon atom number) C₄₋₇cycloalkylcarbonyl, (total carbon atom number) C₃₋₅alkenylcarbonyl, (total carbon atom number) C₃₋₅alkynylcarbonyl, (total carbon atom number) C₅₋₁₁cycloalkylalkylcarbonyl, (total carbon atom number) C₃₋₁₃alkylsulfanylalkylcarbonyl, (total carbon atom number) C₃₋₉alkylsulfinylalkylcarbonyl, (total carbon atom number) C₃₋₉alkylsulfonylalkylcarbonyl, (total carbon atom number) C₄₋₁₀alkylcarbonylalkylcarbonyl, (total carbon atom number) C₄₋₇cycloalkylaminocarbonyl, (total carbon atom number) C₃₋₅alkenylaminocarbonyl, (total carbon atom number) C₃₋₅alkynylaminocarbonyl, C₁₋₄alkylaminosulfonyl, (total carbon atom number) C₂₋₈dialkylaminosulfonyl, (total carbon atom number) C₃₋₉alkoxyalkylcarbonyl, (total carbon atom number) C₄₋₁₀alkylcarbonylaminoalkylcarbonyl, (total carbon atom number) C₄₋₁₀haloalkylcarbonylaminoalkylcarbonyl, (total carbon atom number) C₅₋₁₄alkylsulfanylalkylcarbonylaminoalkylcarbonyl, (total carbon atom number) C₅₋₁₄alkylsulfinylalkylcarbonylaminoalkylcarbonyl, (total carbon atom number) C₅₋₁₄alkylsulfonylalkylcarbonylaminoalkylcarbonyl, (total carbon atom number) C₈₋₁₁aralkylcarbonyl, heterocyclic substituted (total carbon atom number) C₂₋₅alkylcarbonyl, R⁷—C(═O)— or R⁷—C(═S)—, more preferably represents C₂₋₅alkylcarbonyl particularly acetyl, propionyl, n-propylcarbonyl, iso-propylcarbonyl, n-butylcarbonyl, (total carbon atom number) C₂₋₃haloalkylcarbonyl particularly difluoroacetyl, 3,3,3-trifluoropropanoyl, (total carbon atom number) C₂₋₃alkylaminocarbonyl particularly ethylcarbamoyl and propylcarbamoyl, (total carbon atom number) C₄₋₅cycloalkylcarbonyl particularly cyclopropylcarbonyl and cyclobutylcarbonyl, C₃₋₆cycloalkyl(C₁₋₄)alkylcarbonyl particularly cyclopropylacetyl, (total carbon atom number) C₄₋₇ cycloalkylaminocarbonyl particularly cyclopropylcarbamoyl, cyclobutylcarbamoyl and C₁₋₄alkoxy(C₁₋₄alkylcarbonyl(3-methoxypropanoyl, C₁₋₂alkyl-S(C₁₋₂)alkylcarbonyl, C₂₋₅alkoxycarbonyl, C₁₋₂alkyl-SO(C₁₋₂)alkylcarbonyl, C₁₋₂alkyl-SO₂(C₁₋₂)alkylcarbonyl, particularly R⁶ represents hydrogen, C₁₋₄alkylcarbonyl, C₁₋₄haloalkylcarbonyl, C₃₋₆cycloalkylcarbonyl, C₃₋₆cycloalkyl(C₁₋₄)alkylcarbonyl, C₁₋₄alkoxy(C₁₋₄alkylcarbonyl, C₁₋₄alkyl-S(C₁₋₄)alkylcarbonyl, C₁₋₄alkyl-SO(C₄alkylcarbonyl, C₁₋₄alkyl-SO₂(C₁₋₄)alkylcarbonyl, preferably stands for one of the following chemical groups

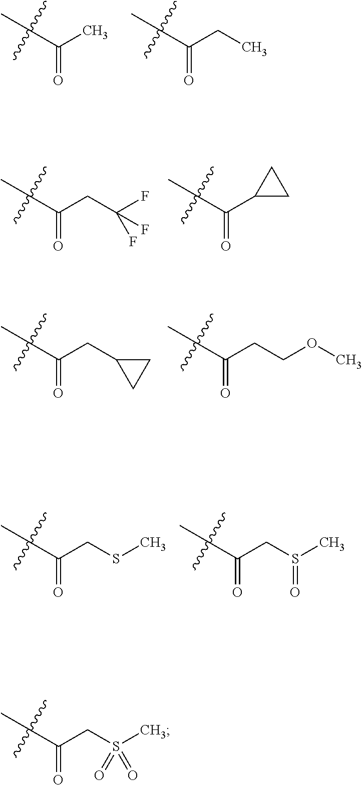

and/or wherein R⁶⁻² stands for hydrogen; or
R⁵ and R⁶ may form a 3- to 6-membered heterocyclic ring, together with the nitrogen atom to which they are bound, and said ring may be substituted with keto, thioketo or a nitroimino group:
R⁷ represents an optionally substituted phenyl or an optionally substituted heterocycle.

Moreover, the invention is directed to a pesticide comprising as an active ingredient a compound according to the invention.

Additionally, the invention is directed to an animal parasite-controlling agent comprising as an active ingredient a compound according to the invention.

Additionally, the invention is directed to a preparation method (a) for the preparation of compounds of formula (I) which comprises

reacting a compound represented by formula (II):

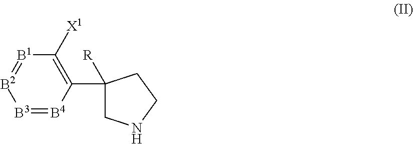

wherein X¹, R and B¹ to B⁴ have the same meaning as defined herein,
with a compound represented by formula (III)

L¹-Q (III)

wherein Q has the same meaning as defined above, and L¹ stands for halogen or a C₁-C₄ haloalkylsulfonyloxy group,
preferably in the presence of a base and, if necessary, in the presence of a metal catalyst.

According to the present invention, the arylpyrrolidines represented by formula (I) of the present invention have a very strong pesticidal efficacy.

In the present specification, the term “alkyl” indicates linear or branched C₁₋₁₂ alkyl, for example, methyl, ethyl, n- or iso-propyl, n-, iso-, sec- or tert-butyl, n-pentyl, n-hexyl, n-heptyl, n-octyl, n-nonyl, n-decyl, n-undecyl, n-dodecyl and the like, preferably C₁₋₆ alkyl, and more preferably C₁₋₄ alkyl.

In addition, for an alkyl moiety that is comprised in other groups as a part of their constitution, those described in the above for the “alkyl” can be exemplified.

The term “haloalkyl” indicates a carbon chain in which at least one hydrogen on linear or branched C₁₋₁₂ alkyl, preferably C₁₋₆ alkyl, and more preferably C₁₋₄ alkyl is substituted with a halogen(s), for example, CH₂F, CHF₂, CF₃, CF₂Cl, CFCl₂, CF₂Br, CF₂CF₃, CFHCF₃, CH₂CF₃, CFClCF₃, CCl₂CF₃, CF₂CH₃, CF₂CH₂F, CF₂CHF₂, CF₂CF₂Cl, CF₂CF₂Br, CFHCH₃, CFHCHF₂, CFHCHF₂, CHFCF₃, CHFCF₂Cl, CHFCF₂Br, CFClCF₃, CCl₂CF₃, CF₂CF₂CF₃, CH₂CF₂CF₃, CF₂CH₂CF₃, CF₂CF₂CH₃, CHFCF₂CF₃, CF₂CHFCF₃, CF₂CF₂CHF₂, CF₂CF₂CH₂F, CF₂CF₂CF₂Cl, and CF₂CF₂CF₂Br. The haloalkyl may be further substituted.

The term “alkoxy” indicates linear or branched C₁₋₁₂, preferably C₁₋₆, and more preferably C₁₋₄ alkoxy, for example, methoxy, ethoxy, n-propoxy, i-propoxy, n-, iso-, sec- or tert-butoxy, pentyloxy, or hexyloxy. The alkoxy may be further substituted.

The term “acylamino” for example indicates alkylcarbonylamino, cyclopropylcarbonylamino and benzoylamino. In addition, for an alkyl moiety that is comprised in these groups as a part of their constitution, those described in the above for the “alkyl” can be exemplified.

The term “halogen” and the halogen moiety that is comprised in halogen-substituted groups represent fluoro, chloro, bromo, or iodo, and preferably fluoro, chloro, or bromo.

The term “cycloalkyl” indicates C₃₋₈ cycloalkyl of cyclopropyl, cyclobutyl, cyclopentyl, cyclohexyl, cycloheptyl, and cyclooctyl, preferably C₃₋₇ cycloalkyl, and more preferably C₃₋₆ cycloalkyl.

The term “alkenyl” indicates C₂₋₆ alkenyl, preferably C₂₋₅ alkenyl, such as vinyl, allyl, 1-propenyl, 1-(or 2-, or 3-) butenyl, 1-pentenyl, and the like, and more preferably C₂₋₄ alkenyl.

The term “alkynyl” indicates C₂₋₆ alkynyl, preferably C₂₋₅ alkynyl such as ethynyl, propargyl, 1-propynyl, butan-3-ynyl, pentan-4-ynyl, and the like, and more preferably C₂₋₄ alkynyl.

The term “aryl” indicates a C₆₋₁₂ aromatic hydrocarbon group, for example, phenyl, naphthyl, and biphenyl, and preferably a C₆₋₁₀ aromatic hydrocarbon group, and more preferably phenyl.

The term “aralkyl” indicates arylalkyl, for example, benzyl and phenethyl.

The term “heterocyclic ring” or “heterocycle” represents a 5- or 6-membered heterocyclic ring wherein at least one heteroatom selected from the group consisting of N, O and S is comprised and also the ring represents a fused heterocyclic ring that can be benzo-fused.

Specific examples of the heterocyclic ring may include furyl, thienyl, pyrrolyl, isoxazolyl, pyrazolyl, oxazolyl, thiazolyl, imidazolyl, triazolyl, oxadiazolyl, thiadiazolyl, tetrazolyl, pyridyl, pyrimidinyl, pyridazinyl, pyrazinyl, triazinyl, indolyl, benzoxazolyl, quinolyl and the like.

All chemical groups, particularly the groups amino, alkyl, haloalkyl, cycloalkyl, cyclohaloalkyl, alkenyl, alkynyl, alkoxy, haloalkoxy, alkylsulfanyl, alkylsulfinyl, alkylsulfonyl, haloalkylsulfanyl, haloalkylsulfinyl, haloalkylsulfonyl, alkylsulfonyloxy, haloalkylsulfonyloxy, alkylaminosulfonyl, haloalkylaminosulfonyl, dialkylaminosulfonyl, di(haloalkyl)aminosulfonyl, alkylamino, dialkylamino, acylamino, alkoxycarbonylamino, haloalkoxycarbonylamino, alkylsulfonylamino, haloalkylsulfonylamino, trialkylsilyl, alkoxyimino, haloalkoxyimino, alkoxyiminoalkyl, haloalkoxyiminoalkyl, alkylsulfinylimino, alkylsulfinyliminoalkyl, alkylsulfinyliminoalkylcarbonyl, alkylsulfoxyimino, alkylsulfoxyiminoalkyl, alkoxycarbonyl, alkylcarbonyl, aminocarbonyl, alkylaminocarbonyl, aminothiocarbonyl, alkylaminothiocarbonyl, dialkylaminocarbonyl, or dialkylaminothiocarbonyl, heterocycle and phenyl may be substituted by at least one substitutent selected among amino, hydroxy, halogen, nitro, cyano, isocyano, mercapto, isothiocyanate, carboxy, carboamide, SF₅, aminosulfonyl, alkyl, cycloalkyl, alkenyl, cycloalkenyl, alkynyl, monoalkylamino, dialkylamino, N-alkylcarbonyl-amino, alkoxy, alkenyloxy, alkynyloxy, cycloalkyloxy, cycloalkenyloxy, alkoxycarbonyl, alkenyloxycarbonyl, alkynyloxycarbonyl, aryloxycarbonyl, alkylcarbonyl, alkenylcarbonyl, alkynylcarbonyl, arylcarbonyl, alkylthio, cycloalkylthio, alkenylthio, cycloalkenylthio, alkynylthio, alkylsulfanyl, alkylsulfinyl, alkylsulfinyl including their isomers, alkylsulfonyl, monoalkylaminosulfonyl, dialkylaminosulfonyl, alkylphosphinyl, alkylphosphonyl, alkylphosphinyl including their isomers, alkylphosphonyl including their isomers, N-alkyl-aminocarbonyl, N,N-dialkyl-aminocarbonyl, N-alkylcarbonyl-aminocarbonyl, N-alkylcarbonyl-N-alkylaminocarbonyl, aryl, aryloxy, benzyl, benzyloxy, benzylthio, arylthio, arylamino, benzylamino, heterocyclic ring, trialkylsilyl, alkoxyalkyl, alkylthioalkyl, alkylthioalkoxy, alkoxyalkoxy, phenethyl, benzyloxy, haloalkyl, haloalkoxy, haloalkylthio, haloalkylcarbonyl, haloalkoxycarbonyl, haloalkoxyalkoxy, haloalkoxyalkylthio, haloalkoxyalkylcarbonyl and haloalkoxyalkyl, preferably, chloro, fluoro, bromo, iodo, amino, nitro, cyano, hydroxy, thio and carboxy.

The phrase “(total carbon atom number)”, which is used in relation to a group with any substituent, indicates the number of carbon atom included in the whole group without such substituent.

In an aspect of the invention compounds of formula (A-I) to (A-III) are preferred having the following formula

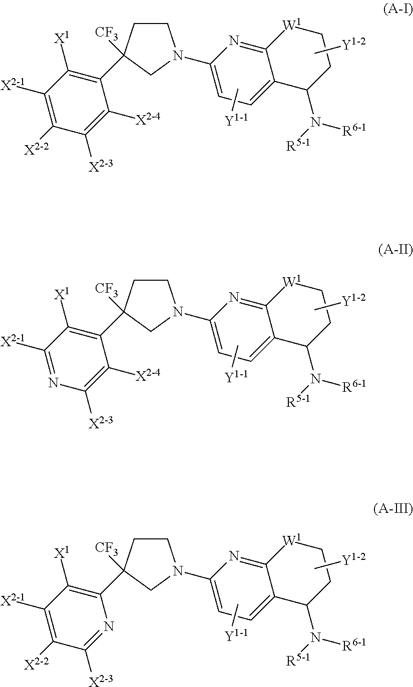

wherein W¹ and X¹ are as defined herein, each X²⁻¹, X²⁻², X²⁻³ and X²⁻⁴ are as defined herein for X², each Y¹⁻¹ and Y¹⁻² are as defined herein for Y¹, and R⁵⁻¹ is defined herein for R⁵, R⁶⁻¹ is defined herein for R⁶.

In another aspect of the invention compounds of formula (B-I) to (B-III) are preferred having the following formula

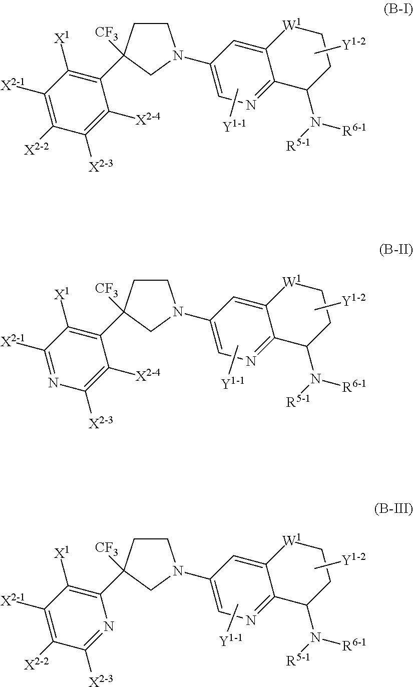

wherein W¹ and X¹ are as defined herein, each X²⁻¹, X²⁻², X²⁻³ and X²⁻⁴ are as defined herein for X², each Y¹⁻¹ and Y¹⁻² are as defined herein for Y¹, R⁵⁻¹ is defined herein for R⁵, and R⁶⁻¹ is defined herein for R⁶.

In another aspect of the invention compounds of formula (C-I) to (C-III) are preferred having the following formula

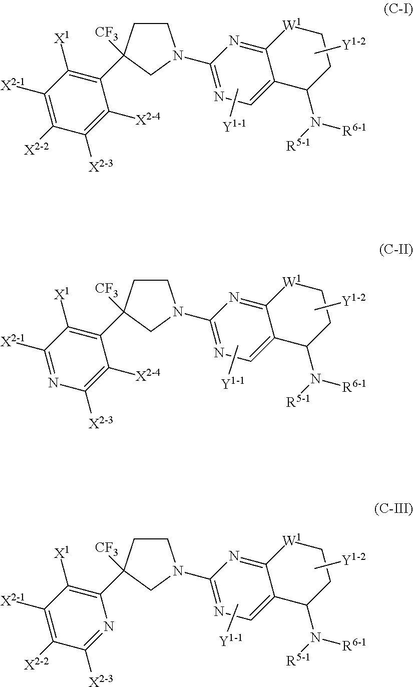

wherein W¹ and X¹ are as defined herein, each X²⁻¹, X²⁻², X²⁻³ and X²⁻⁴ are as defined herein for X², each Y¹⁻¹ and Y¹⁻² are as defined herein for Y¹, R⁵⁻¹ is defined herein for R⁵, and R⁶⁻¹ is defined herein for R⁶.

In another aspect of the invention compounds of formula (D-I) to (D-III) are preferred having the following formula

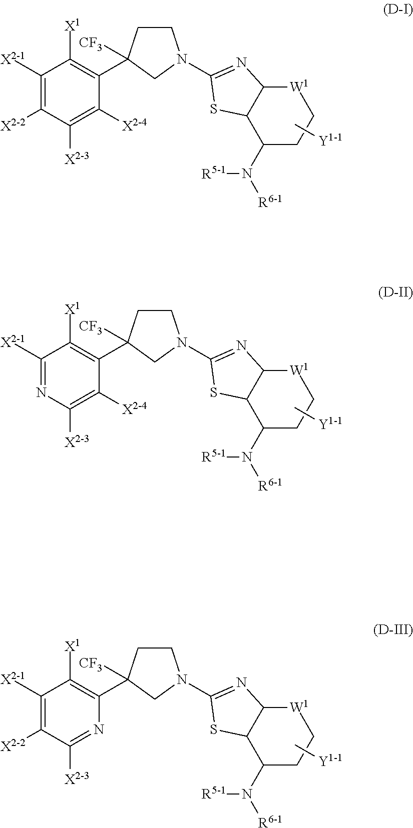

wherein W¹ and X¹ are as defined herein, each X²⁻¹, X²⁻², X²⁻³ and X²⁻⁴ are as defined herein for X², each Y¹⁻¹ is as defined herein for Y¹, R⁵⁻¹ is defined herein for R⁵, and R⁶⁻¹ is defined herein for R⁶.

In another aspect of the invention compounds of formula (E-I) to (E-III) are preferred having the following formula

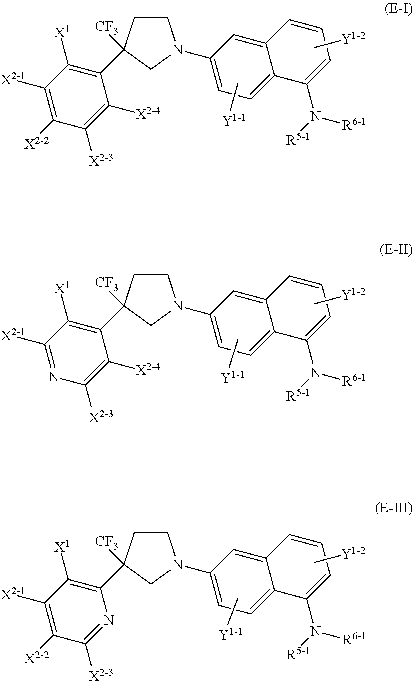

wherein X¹ is as defined herein, each X²⁻¹, X²⁻², X²⁻³ and X²⁻⁴ are as defined herein for X², each Y¹⁻¹ and Y¹⁻² are as defined herein for Y¹, R⁵⁻¹ is defined herein for R⁵ and R⁶⁻¹ is defined herein for R⁶.

In another aspect of the invention compounds of formula (F-I) to (F-III) are preferred having the following formula

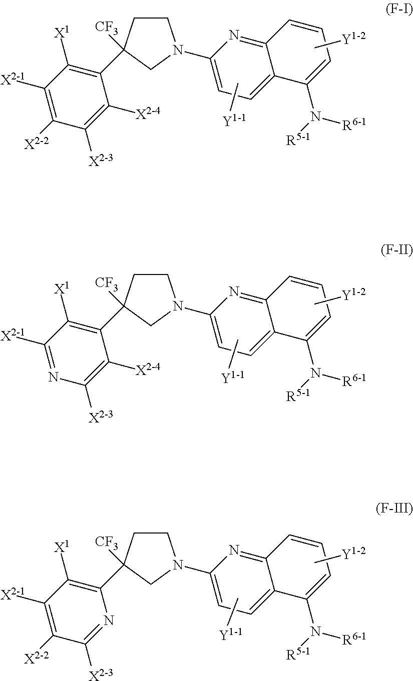

wherein X¹ is as defined herein, each X²⁻¹, X²⁻², X²⁻³ and X²⁻⁴ are as defined herein for X², each Y¹⁻¹ and Y¹⁻² are as defined herein for Y¹, R⁵⁻¹ is as defined herein for R⁵, and R⁶⁻¹ is defined herein for R⁶.

In another aspect of the invention compounds of formula (G-I) to (G-III) are preferred having the following formula

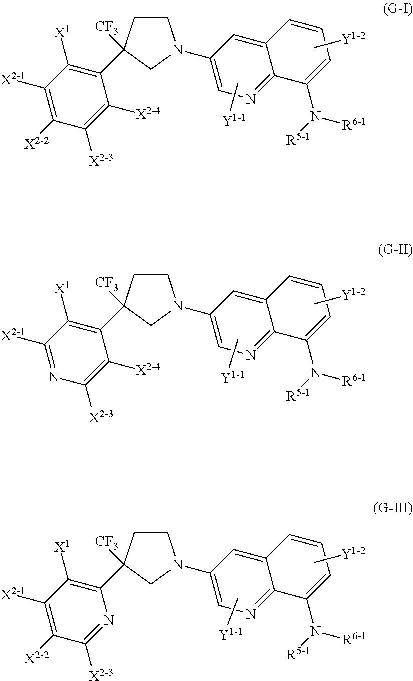

wherein X¹ is as defined herein, each X²⁻¹, X²⁻², X²⁻³ and X²⁻⁴ are as defined herein for X², each Y¹⁻¹ and Y¹⁻² are as defined herein for Y², R⁵⁻¹ is defined herein for R⁵, and R⁶⁻¹ is defined herein for R⁶.

In another aspect of the invention compounds of formula (H-I) to (H-III) are preferred having the following formula

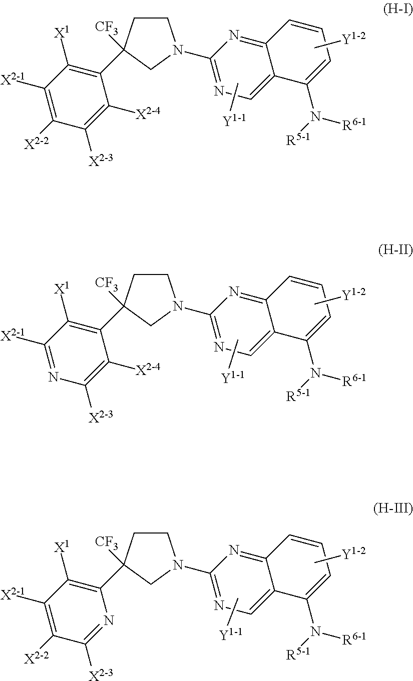

wherein X¹ is as defined herein, each X²⁻¹, X²⁻², X²⁻³ and X²⁻⁴ are as defined herein for X², each Y¹⁻¹ and Y¹⁻² are as defined herein for Y¹, R⁵⁻¹ is defined herein for R⁵, and R⁶⁻¹ is defined herein for R⁶.

In another aspect of the invention compounds of formula (I-I) to (I-III) are preferred having the following formula

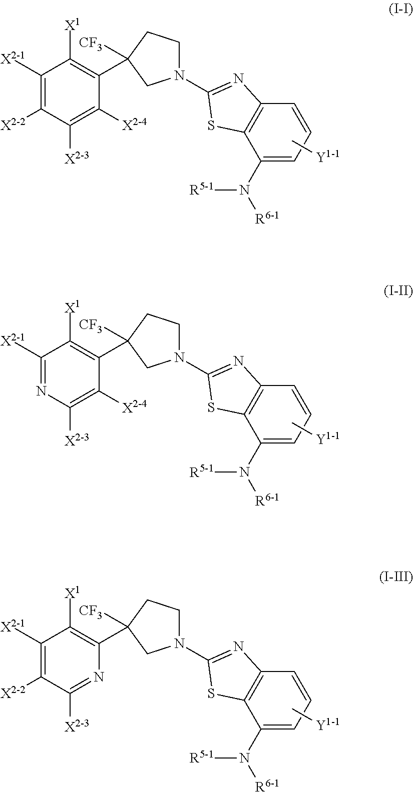

wherein X¹ is as defined herein, each X²⁻¹, X²⁻², X²⁻³ and X²⁻⁴ are as defined herein for X², Y¹⁻¹ is as defined herein for Y¹, R⁵⁻¹ is defined herein for R⁵, and R⁶⁻¹ is defined herein for R⁶.

In another aspect of the invention compounds of formula (J) is preferred having the following formula

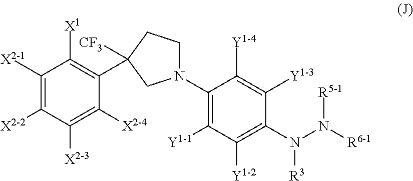

wherein X¹ and R³ are as defined herein, each X²⁻¹, X²⁻², X²⁻³ and X²⁻⁴ are as defined herein for X², each Y¹⁻¹, Y¹⁻², Y¹⁻³ and Y¹⁻⁴ are as defined herein for Y¹, R⁵⁻¹ is defined herein for R⁵, and R⁶⁻¹ is defined herein for R⁶.

In another aspect of the invention compounds of formula (K-I) to (K-III) are preferred having the following formula

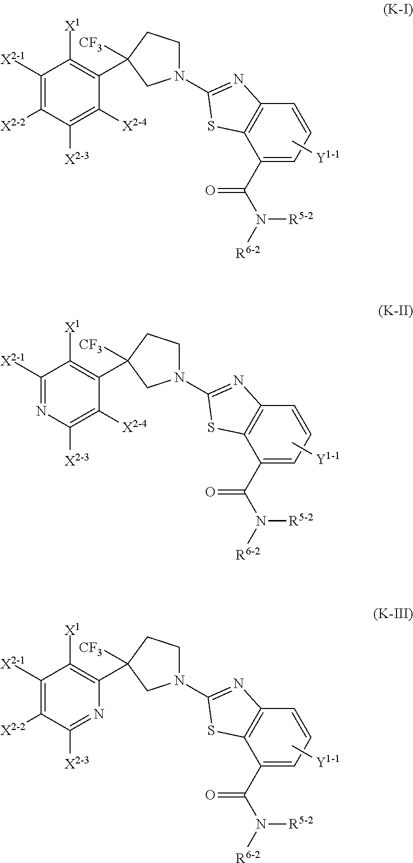

and wherein X¹ is as defined herein, each X²⁻¹, X²⁻², X²⁻³ and X²⁻⁴ are as defined herein for X², R⁵⁻² is defined herein for R⁵, R⁶⁻² is defined herein for R⁶ and each Y¹⁻¹ is as defined herein Y¹;

In another aspect of the invention compounds of formula (L-I) to (L-III) are preferred having the following formula

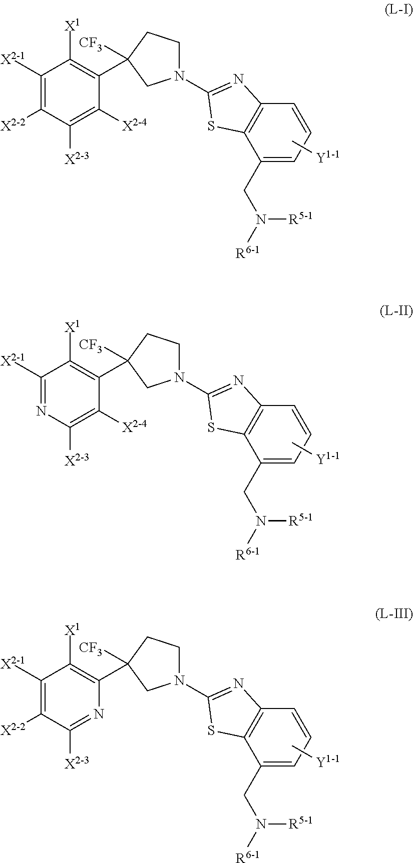

wherein X¹ is as defined herein, each X²⁻¹, X²⁻², X²⁻³ and X²⁻⁴ are as defined herein for X², Y¹⁻¹ is as defined herein for Y¹, R⁵⁻¹ is defined herein for R⁵, and R⁶⁻¹ is defined herein for R⁶.

Further preferred are compounds having one of above mentioned formulae A-I, A-II, A-III, B-I, B-II, B-III, C-I, C-II, C-III, D-I, D-II, D-III, E-I, E-II, E-III, F-I, F-II, F-III, G-I, G-II, G-III, H-I, H-II, H-III, I-I, I-II, I-III, J, K-I, K-II, K-III, L-I, L-II or L-III wherein

X¹, X²⁻¹, X²⁻², X²⁻³ and X²⁻⁴ independently of each other represents hydrogen, halogen (i.e. F, Cl, Br, I) or C₁₋₄-haloalkyl (preferably CF₃, CF₂H, CFH₂, CH₂CF₃, CF₂CF₃) preferably at least one of the groups X¹, X²⁻¹, X²⁻², X²⁻³ and X²⁻⁴ stands for hydrogen, more preferably X¹ and/or X²⁻⁴ and/or X²⁻² stands for hydrogen, most preferably X¹ and X²⁻⁴ stand for hydrogen and X²⁻¹, X²⁻² and X²⁻³ independently of each other stands for CF₃, F, Cl or Br;
and/or wherein Y¹⁻¹, Y¹⁻², Y¹⁻³ and Y¹⁻⁴ independently of each other represents hydrogen, halogen (e.g. F, Cl, Br, I), C₁₋₄-alkyl, C₁₋₄-alkoxy, C₁₋₄-haloalkoxy, C₃₋₆-cycloalkyl, C₁₋₄-haloalkyl (preferably CF₃, CF₂H, CFH₂, CH₂CF₃, CF₂CF₃), C₁₋₄-alkylsulfanyl, or C₁₋₄-haloalkylsulfanyl;
and/or wherein R³ stands for hydrogen;
and/or wherein R⁵⁻¹ stands for hydrogen, C₁₋₄ alkyl or C₁₋₄ haloalkyl, preferably hydrogen,
and/or wherein R⁵⁻² stands for C₁₋₄alkyl, C₁₋₄haloalkyl, C₁₋₄alkoxy(C₁₋₄)alkyl, C₁₋₄alkyl-S(C₁₋₄)alkyl, C₁₋₄alkyl-SO(C₁₋₄alkyl, C₁₋₄ alkyl-SO₂(C₁₋₄)alkyl or 2-pyridyl-C₁₋₄alkyl;
and/or wherein R⁶⁻¹ stands for C₁₋₄alkylcarbonyl, C₁₋₄haloalkylcarbonyl, C₃₋₆cycloalkylcarbonyl, C₃₋₆cycloalkyl(C₁₋₄)alkylcarbonyl, C₁₋₄alkoxy(C₁₋₄)alkylcarbonyl, C₁₋₄alkyl-S(C₁₋₄)alkylcarbonyl, C₁₋₄alkyl-SO(C₁₋₄)alkylcarbonyl, C₁₋₄ alkyl-SO₂(C₁₋₄)alkylcarbonyl, preferably stands for one of the following chemical groups

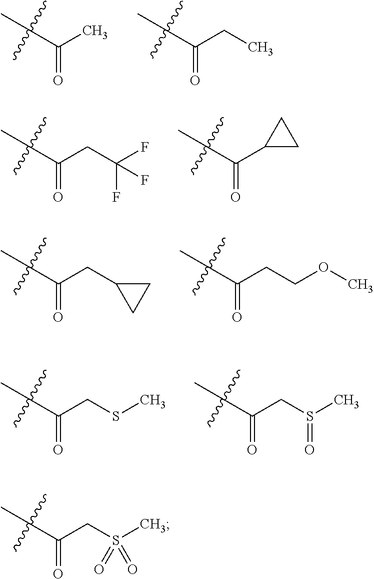

and/or wherein R⁶⁻² stands for hydrogen.

The compounds of formula (I) of the present invention have asymmetric carbons, and thus the compounds encompass optical isomers.

The nitrogen atom on the pyrrolidine backbone of the compounds of formula (I) of the present invention may be substituted with oxygen, alkyl or haloalkyl or may form a salt.

Compounds according to the invention can be prepared analogously to the example described below.

Preparation method (a) is exemplified by the reaction given below in which 3-(3,5-dichlorophenyl)-3-(trifluoromethyl)pyrrolidine and 5-[(tert-butoxycarbonyl)amino]-2-naphthyl trifluoromethanesulfonate are used as starting materials:

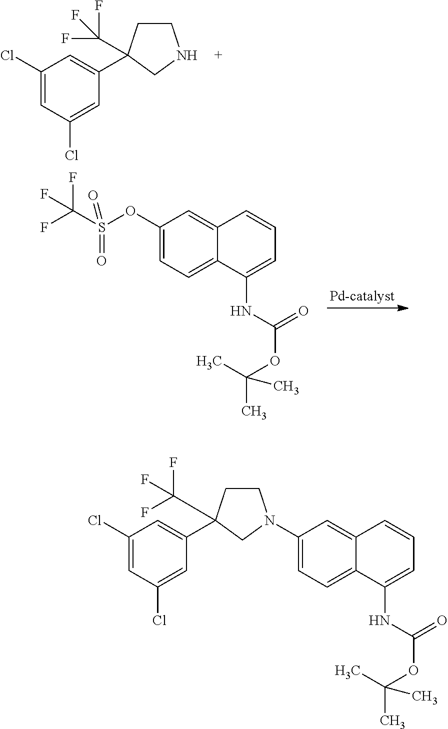

The compound of formula (I) can be prepared analogously to the methods as exemplified in schemes 1 to 5:

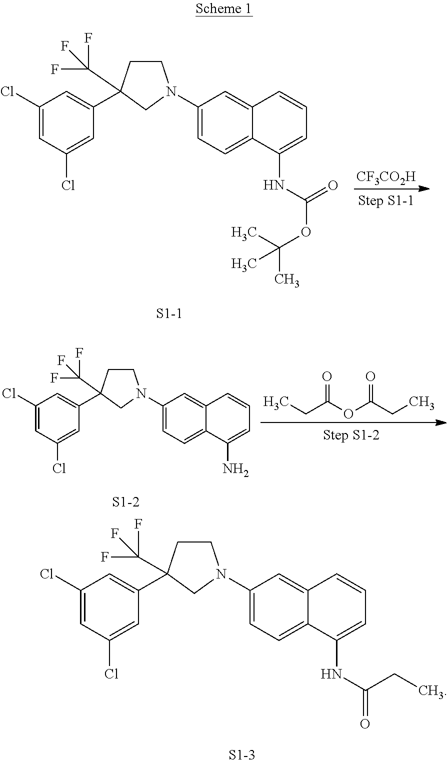

Wherein compound S1-1 is prepared by the preparation method (a) and t-butyl-O—CO group is hydrolyzed by methods known in the art (e.g. acid conditions) to give the compound S1-2. The R⁶ group (here —CO—CH₂CH₃) is introduced by methods known in the art in step S1-2.

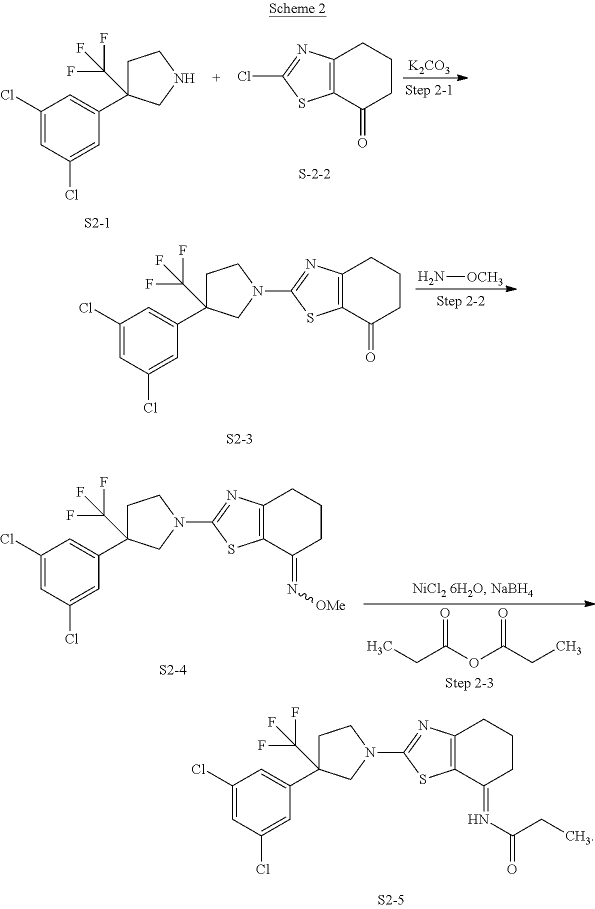

Wherein S2-1 and S2-2 are reacted, if necessary in the presence of a base (e.g. K₂CO₃) to obtain the compound S2-3 (cf: Russian Journal of General Chemistry 2000, 70(5), 784-787) and wherein the compound S2-3 is then converted with methoxyamine to the metoxyimine-derivative S2-4 which is then hydrogenated under standard conditions (e.g. by adding NiCl₂X₆H₂O and NaBH₄) and reacted with propionic acid anhydride to give compound S2-5.

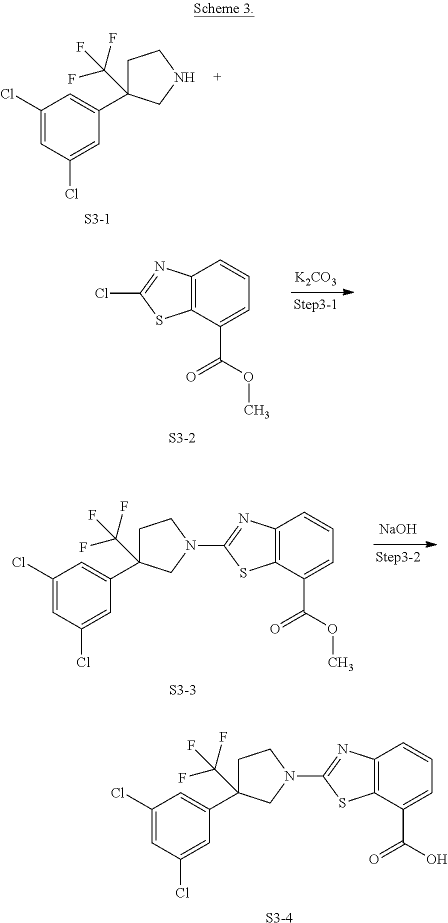

Wherein the compounds S3-1 and S3-2 are reacted under similar basic conditions as described for scheme 2 to obtain the compound S3-3 which is then hydrolyzed by methods known in the art to give S3-4.

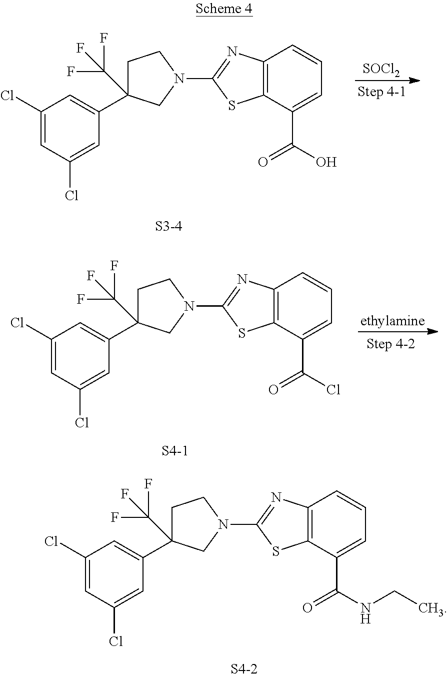

Wherein the compound S3-4 is converted to the compound S4-1 by using thionylchloride and then reacted with ethylamine to obtain S4-2.

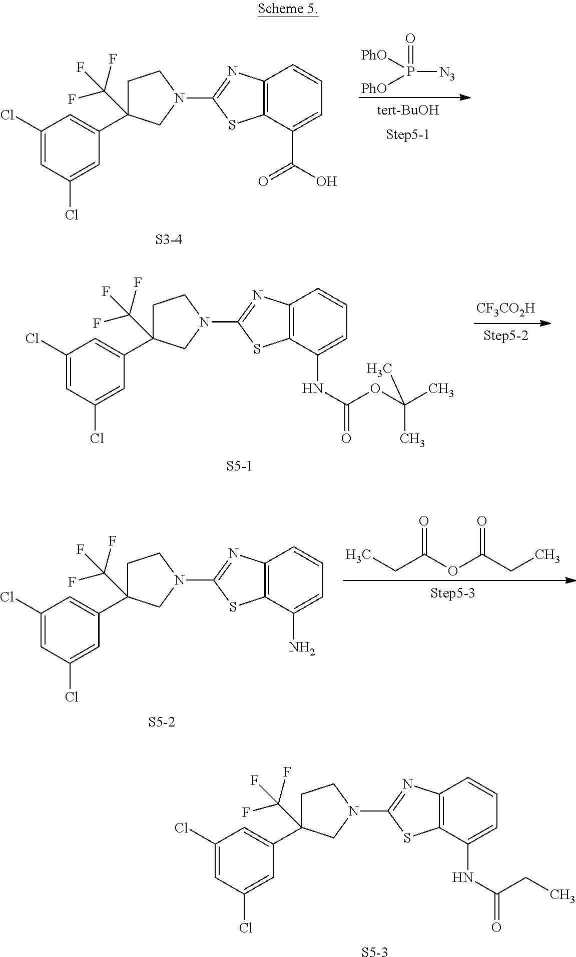

Wherein the compound S3-4 is converted by “Curtius rearrangement” to give the compound S5-1 (Step 5-1, cf.: Tetrahedron, 1974, 30, 2151-2157) which is then hydrolyzed under standard conditions to give the compound S5-2; followed by the reaction with propionic acid anhydride to obtain the compound S5-3.

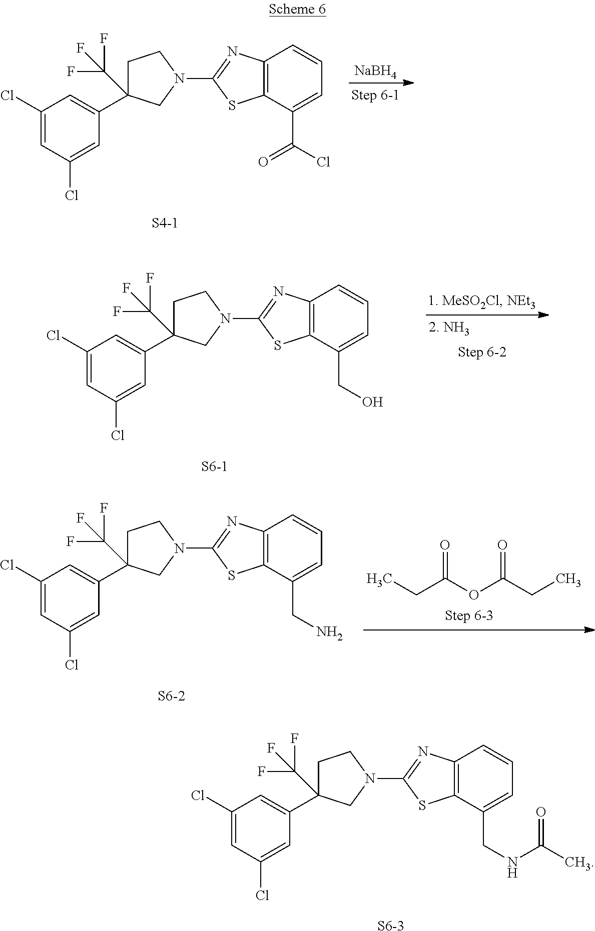

Wherein the compound S4-1 is reduced by standard methods to give the compound S6-1 which is changed to the compound S6-2 in step 6-2 (mesyration and amination) and followed by the reaction with propionic acid anhydride to obtain compound S6-3.

The compounds of formula (II), which are the starting materials in the preparation method (a), can be synthesized by the method described below. Specifically, the compounds represented by the following formula (XV):

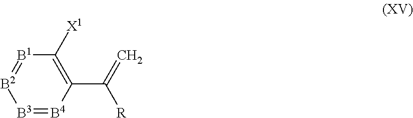

wherein X¹, B¹ to B⁴ and R have the same meaning as defined herein, can be reacted with N-benzyl-1-methoxy-N-[(trimethylsilyl)-methyl]methanamine in the presence of a catalyst (e.g. trifluoroacetic acid, trimethylsilyl trifluoromethane sulfonate, iodotrimethylsilane, cesium fluoride) to give the compounds represented by formula (XVI):

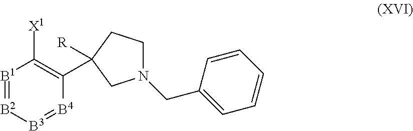

wherein X¹, B¹ to B⁴ and R have the same meaning as defined herein, followed by debenzylation to obtain the compounds represented by the above formula (II).

The reaction which yields compounds of formula (XVI) may be carried out according to the methods described in “Chemistry Letters, 1984, 1117-1120” and “Tetrahedron Letters, 1993, 34, 3279-3282”. The deprotection reaction such as the debenzylation of compounds of formula (XVI) may be carried out according to the methods described in “Journal of the Organic Chemistry, 1984, 49, 2081” and “PROTECTIVE GROUPS in ORGANIC CHEMISTRY THIRD EDITION, JOHN WILEY & SONS, INC”.

The compounds of the above formula (XV) encompass the compounds that are known and described, for example in “The Journal of Organic Chemistry, 1991, vol. 56, pp. 7336-7340”; “The Journal of Organic Chemistry, 1994, vol. 59, pp. 2898-2901”; “Journal of Fluorine Chemistry, 1999, vol. 95, pp. 167-170”; “WO2005/05085216A”. Such compounds may be synthesized by the methods described therein.

Representative examples of the compounds of formula (XV) include [1-(trifluoromethyl)vinyl]benzene, 1-chloro-3-[1-(trifluoromethyl)vinyl]benzene, 1-bromo-3-[1-(trifluoromethyl)vinyl]benzene, 1-nitro-3-[1-(trifluoromethyl)vinyl]benzene, 1-trifluoromethyl-3-[1-(trifluoromethyl)vinyl]benzene, 1,3-difluoro-5-[1-(trifluoromethyl)vinyl]benzene, 1,3-dichloro-5-[1-(trifluoromethyl)vinyl]benzene, 1,3-difluoro-5-[1-(trifluoromethyl)vinyl]benzene, 1-fluoro-2-(trifluoromethyl)-4-[1-(trifluoromethyl)vinyl]benzene, 1,2,3-trichloro-5-[1-(trifluoromethyl)vinyl]benzene, 1,3-dimethyl-2-nitro-5-[1-(trifluoromethyl)vinyl]benzene.

Instead of N-benzyl-1-methoxy-N-[(trimethylsilyl)methyl]methanamine in the above reaction, N-benzyl-1-butoxy-N-[(trimethylsilyl)methyl]methanamine or N-(butoxymethyl)-N-[(trimethyl-Silyl)methyl]cyclohexylamine may be used.

Representative examples of the compounds of formula (II) in the Preparation method (a) include 3-(3,5-dichlorophenyl)-3-(trifluoromethyl)pyrrolidine, 3-[3,5-bis(trifluoromethyl)phenyl]-3-(trifluoromethyl)pyrrolidine, 3-(3,4,5-trichlorophenyl)-3-(trifluoromethyl)pyrrolidine, and 3-(3,5-dimethyl-4-nitrophenyl)-3-(trifluoromethyl)pyrrolidine.

Many of the compounds of formula (III) which are starting materials in the Preparation method (a), are known compounds, and they may be readily synthesized by the methods widely known in organic chemistry.

The specific examples of formula (III) include for example 5-[(tert-butoxycarbonyl)amino]-2-naphthyl trifluoromethanesulfonate, 5-acetamido-2-naphthyl trifluoromethanesulfonate, 5-(propionylamino)-2-naphthyl trifluoromethanesulfonate, 5-[(cyclopropylcarbonyl)amino]-2-naphthyl trifluoromethanesulfonate, tert-butyl (2-chloroquinolin-5-yl)carbamate, N-(2-chloroquinolin-5-yl)propanamide, N-(2-chloroquinolin-5-yl)cyclopropanecarboxamide

Method (a) is used to synthesize starting compounds as given in scheme 2 and 3. The specific examples of the starting compounds include for 2-chloro-5,6-dihydro-1,3-benzothiazol-7(4H)-one (S2-2), methyl 2-chloro-1,3-benzothiazole-7-carboxylate (S3-2) and ethyl 2-chloro-1,3-benzothiazole-7-carboxylate.

The reaction of the preparation method (a) can be carried out in a suitable diluent, and examples thereof include aliphatic hydrocarbons (e.g. hexane, cyclohexane, heptane etc.), aliphatic halogenated hydrocarbons (e.g. dichloromethane, chloroform, carbon tetrachloride, dichloroethane, etc.), aromatic hydrocarbons (e.g. benzene, toluene, xylene, chlorobenzene etc.), ethers (e.g. diethyl ether, dibutyl ether, dimethoxyethane (DME), tetrohydrofuran, dioxane etc.), esters (e.g. ethyl acetate, ethyl propionate etc.), acid amides (e.g. dimethylformamide (DMF), dimethylacetamide (DMA), N-methylpyrrolidone etc.), nitriles (e.g. acetonitrile, propionitrile etc.), dimethylsulfoxide (DMSO), water or mixed solvents thereof and the like.

The reaction of the preparation method (a) can be carried out in the presence of a base, such as alkali metal bases (e.g. lithium hydride, sodium hydride, potassium hydride, butyl lithium, tert-butyl lithium, trimethylsilyl lithium, lithium hexamethyldisilazide, sodium carbonate, potassium carbonate, cesium carbonate, tripotassium phosphate, sodium acetate, potassium acetate, sodium methoxide, sodium ethoxide, sodium-tert-butoxide and potassium-tert-butoxide etc.), organic bases (e.g. triethylamine, diisopropylethylamine, tributylamine, N-methylmorpholine, N,N-dimethylaniline, N,N-diethylaniline, 4-tert-butyl-N,N-dimethylaniline, pyridine, picoline, lutidine, diazabicycloundecene, diazabicyclooctane, imidazole etc.) and optionally in the presence of a metal catalyst such as transition metal catalysts (eg. Pd₂(dba)₃, Pd₂(dba)₃CHCl₃ (dba=dibenzylideneacetone), Pd(OAc)₂, CuI, Cu₂O). The transition metal catalyst can be used in the presence of phosphine ligands such as 2,2′-bis(diphenylphosphino)-1,1′-binaphtalene (BINAP), 4,5-bis(diphenylphosphino)-9,9-dimethylxanthene (Xantphos), tributylphosphine, triphenyl-phosphine, or amine ligands such as 8-quinolinol, proline, N,N-dimethylglycine.

The preparation method (a) can be carried out within a substantially wide temperature range. It may be generally carried out at the temperature between about −78° C. and about 200° C., preferably between −10° C. and about 150° C. Said reaction is desirably carried out at normal pressure although it may be carried out under elevated or reduced pressure. The reaction time is 0.1 to 72 hours, preferably 0.1 to 24 hours.

For carrying out the preparation method (a), for example, 1 mole of the compound of formula (II) can be reacted with 1 to 3 moles of the compound of formula (III) in the presence of 1 to 3 moles of a base (e.g. sodium-tert-butoxide and a catalytic amount (e.g. in the range of about 1 to 10 mol-%) of the metal (preferably transition metal) catalyst (e.g. Pd₂(dba)₃ or Pd₂(dba)₃CHCl₃) and a suitable amount (e.g. in the range of about 3 to 30 mol-%) of phosphine ligand compound (e.g. 4,5-bis(diphenylphosphino)-9,9-dimethylxanthen) in a diluent (e.g. toluene) to obtain the compound of formula (I) of the present invention.

Additionally, the compound of formula (I), wherein R⁵ stands for hydrogen and R⁶ stands for t-Butyl-O—CO is hydrolyzed under acidic condition to synthesize a compound of formula (I), wherein R⁵ and R⁶ are hydrogen. Such a compound of formula (I) is then reacted with a compound having the formula L¹-R⁶, wherein L¹ and R⁶ are as defined herein under the proviso that R⁶ does not stand for hydrogen, if necessary in the presence of base as defined herein and/or a solvent as defined herein, to give a compound of formula (I).

With respect to the above preparation methods for the compounds of formula (I) of the present invention, novel raw materials thereof (i.e., starting materials and intermediates) can be collectively represented by formula (XXIV) and (XXV) described below:

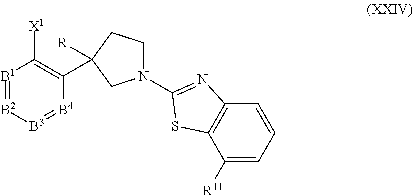

wherein X¹, R, and B¹ to B⁴ have the same meaning as defined above, and R¹¹ represents hydroxycarbonyl or C₁₋₄alkoxycarbonyl,

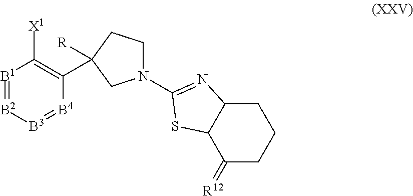

wherein X¹, R, and B¹ to B⁴ have the same meaning as defined above, and R¹² represents oxygen, HO—N or C₁₋₄alkyloxy-N.

The active compounds of the invention, in combination with good plant tolerance and favourable toxicity to warm-blooded animals and being tolerated well by the environment, are suitable for protecting plants and plant organs, for increasing harvest yields, for improving the quality of the harvested material and for controlling animal pests, in particular insects, arachnids, helminths, nematodes and molluscs, which are encountered in agriculture, in horticulture, in animal husbandry, in forests, in gardens and leisure facilities, in the protection of stored products and of materials, and in the hygiene sector. They can be preferably employed as plant protection agents. They are active against normally sensitive and resistant species and against all or some stages of development.

Such umwanted insects which may damage plants and/or technical materials include e.g. beetles (Coleopteran), such as adzuki bean beetle (Callosobruchus Chinensis), maize weevil (Sitophilus zeamais), red flour beetle (Tribolium Castaneum), large twenty-wight-spotted lady beetle (Epilachna vigintioctomaculata), barley wireworm (Agriotes ogurae fuscicollis), soy bean beetle (Anomala rufocuprea), Colorado potato beetle (Leptinotarsa decemlineata), corn root worm (Diabrotica spp.), Matsunomadra long-horned beetle (Monochamus alternatus endai), rice water weevil (Lissorhoptrus oryzophilus), powder-post beetle (Lyctus brunneus); lepidopteran pests, such as gypsy moth (Lymantria dispar), Lackey moth (Malacosoma neustria), small white (Pieris rapae crucivora), cotton leafworm (Spodoptera litura), cabbage moth (Mamestra brassicae), rice stem borer (Chilo suppressalis), European corn borer (Ostrinia nubilalis), dried currant moth (Cadra cautella), chyanokokakumonhamaki (Adoxophyes honmai), codling moth (Cydia pomonella), Turnip Moth (Agrotis segetum), Wax Moth (Galleria mellonella), Diamondback moth (Plutella xylostella), tobacco budworm moth (Heliothis virescens), citrus leaf miner (Phyllocnistis citrella); hemipterous pests, such as green rice leafhopper (Nephotettix cincticeps), brown planthopper (Nilaparvata lugens), comstock mealybug (Pseudococcus comstocki), arrowheat scale (Unaspis yanonensis), Momoaka-aburamusi (Myzus persicas), green apple aphid (Aphis pomi), cotton aphid (Aphis gossypii), turnip aphid (Lipaphis erysimi), Nashi-gunbai (Stephanitis nashi), Nezara (Nezara spp.), greenhouse whitefly (Trialeurodes vaporariorm), Pshylla (Pshylla spp.); thysanoptera pests, such as palm thrips (Thrips palmi), western flower thrips (Franklinella occidentalis); orthopteran pests, such as mole cricket (Gryllotalpa Africana), migratory locust (Locusta migratoria); blattarian pests, such as German cockroach (Blatella germanica), American cockroach (Periplaneta americana), yamato white ant (Reticulitermes speratus), Formosan subterranean termite (Coptotermes formosanus);

dipterous pests, such as housefly (Musca domestica), yellow fever mosquito (Aedes aegypti), Seedcorn maggot (Delia platura), Aka-ie-ka (Culex pipiens pallens), Sina-hamadara-ka (Anopheles sinensis), kodaka-aka-ie-ka (Culex tritaeniorhynchus), serpentine leafminer (Liriomyza trifolii) and the like.

Further, as mites, Carmine spider mite (Tetranychus cinnabarinus), two-spotted spider mite (Tetrahychus urticae), Citrus red mite (Panonychus citri), Pink citrus rust mite (Aculops pelekassi), Tarsonemus (Tarsonemus spp.) and the like can be mentioned.

In addition, as nematodes, sweet potato root-knot nematode (Meloidogyne incognita), pine wood nematode (Bursaphelenchus xylophilus), rice white-tip nematode (Aphelenchoides besseyi), soybean cyst nematode (Heterodera glycines), meadow nematode (Pratylenchus spp.) and the like can be mentioned.

The abovementioned pests include:

From the order of the Anoplura (Phthiraptera), for example, Damalinia spp., Haematopinus spp., Linognathus spp., Pediculus spp., Trichodectes spp.
From the class of the Arachnida, for example, Acarus spp., Aceria sheldoni, Aculops spp., Aculus spp., Amblyomma spp., Amphitetranychus viennensis, Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Halotydeus destructor, Hemitarsonemus spp., Hyalomma spp., Ixodes spp., Latrodectus mactans, Metatetranychus spp., Nuphersa spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.
From the class of the Bivalva, for example, Dreissena spp.
From the order of the Chilopoda, for example, Geophilus spp., Scutigera spp.
From the order of the Coleoptera, for example, Acalymma vittatum, Acanthoscelides obtectus, Adoretus spp., Agelastica alni, Agriotes spp., Amphimallon solstitialis, Anobium punctatum, Anoplophora spp., Anthonomus spp., Anthrenus spp., Apion spp., Apogonia spp., Atomaria spp., Attagenus spp., Bruchidius obtectus, Bruchus spp., Cassida spp., Cerotoma trifurcata, Ceutorrhynchus spp., Chaetocnema spp., Cleonus mendicus, Conoderus spp., Cosmopolites spp., Costelytra zealandica, Ctenicera spp., Curculio spp., Cryptorhynchus lapathi, Cylindrocopturus spp., Dermestes spp., Diabrotica spp., Dichocrocis spp., Diloboderus spp., Epilachna spp., Epitrix spp., Faustinus spp., Gibbium psylloides, Hellula undalis, Heteronychus arator, Heteronyx spp., Hylamorpha elegans, Hylotrupes bajulus, Hypera postica, Hypothenemus spp., Lachnosterna consanguinea, Lema spp., Leptinotarsa decemlineata, Leucoptera spp., Lissorhoptrus oryzophilus, Lixus spp., Luperodes spp., Lyctus spp., Megascelis spp., Melanotus spp., Meligethes aeneus, Melolontha spp., Migdolus spp., Monochamus spp., Naupactus xanthographus, Niptus hololeucus, Oryctes rhinoceros, Oryzaephilus surinamensis, Oryzaphagus oryzae, Otiorrhynchus spp., Oxycetonia jucunda, Phaedon cochleariae, Phyllophaga spp., Phyllotreta spp., Popillia japonica, Premnotrypes spp., Psylliodes spp., Ptinus spp., Rhizobius ventralis, Rhizopertha dominica, Sitophilus spp., Sphenophorus spp., Sternechus spp., Symphyletes spp., Tanymecus spp., Tenebrio molitor, Tribolium spp., Trogoderma spp., Tychius spp., Xylotrechus spp., Zabrus spp.
From the order of the Collembola, for example, Onychiurus armatus.
From the order of the Diplopoda, for example, Blaniulus guttulatus.
From the order of the Diptera, for example, Aedes spp., Agromyza spp., Anastrepha spp., Anopheles spp., Asphondylia spp. Bactrocera spp., Bibio hortulanus, Calliphora erythrocephala, Ceratitis capitata, Chironomus spp., Chrysomyia spp., Cochliomyia spp., Contarinia spp., Cordylobia anthropophaga, Culex spp., Cuterebra spp., Dacus oleae, Dasyneura spp., Delia spp., Dermatobia hominis, Drosophila spp., Echinocnemus spp., Fannia spp., Gastrophilus spp., Hydrellia spp., Hylemyia spp., Hyppobosca spp., Hypoderma spp., Liriomyza spp. Lucilia spp., Musca spp., Nezara spp., Oestrus spp., Oscinella frit, Pegomyia spp., Phorbia spp., Prodiplosis spp., Psila rosae, Rhagoletis spp., Stomoxys spp., Tabanus spp., Tannia spp., Tetanops spp., Tipula spp.
From the class of the Gastropoda, for example, Arion spp., Biomphalaria spp., Bulinus spp., Deroceras spp., Galba spp., Lymnaea spp., Oncomelania spp., Pomacea spp., Succinea spp.
From the class of the Helminthen, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

It is furthermore possible to control protozoa, such as Eimeria.

From the order of the Heteroptera, for example, Anasa tristis, Antestiopsis spp., Blissus spp., Calocoris spp., Campylomma livida, Cavelerius spp., Cimex spp., Collaria spp., Creontiades dilutus, Dasynus piperic, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Monalonion atratum, Nezara spp., Oebalus spp., Pentomidae, Piesma quadrata, Piezodorus spp., Psallus spp., Pseudacysta persea, Rhodnius spp., Sahlbergella singularis, Scaptocoris castanea, Scotinophora spp., Stephanitis nashi, Tibraca spp., Triatoma spp.
From the order of the Homoptera, for example, Acyrthosipon spp., Acrogonia spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp. Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis Tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Drosicha spp., Dysaphis spp., Dysmicoccus spp., Empoasca spp., Eriosoma spp., Erythroneura spp., Euscelis bilobatus, Ferrisia spp., Geococcus coffeae, Hieroglyphus spp., Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva spp., Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes spp., Trioza spp., Typhlocyba spp., Unaspis spp., Viteus vitifolii, Zygina spp.
From the order of the Hymenoptera, for example, Athalia spp., Diprion spp., Hoplocampa spp., Lasius spp., Monomorium pharaonis, Vespa spp.
From the order of the Isopoda, for example, Armadillidium vulgare, Oniscus asellus, Porcellio scaber.
From the order of the Isoptera, for example, Acromyrmex spp., Atta spp., Cornitermes cumulans, Microtermes obeli, Odontotermes spp., Reticulitermes spp.
From the order of the Lepidoptera, for example, Acronicta major, Adoxophyes spp., Aedia leucomelas, Agrotis spp., Alabama spp., Amyelois transitella, Anarsia spp., Anticarsia spp., Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Chematobia brumata, Chilo spp., Choristoneura spp., Clysia ambiguella, Cnaphalocerus spp., Cnephasia spp., Conopomorpha spp., Conotrachelus spp., Copitarsia spp., Cydia spp., Dalaca noctuides, Diaphania spp., Diatraea saccharalis, Earias spp., Ecdytolopha aurantium, Elasmopalpus lignosellus, Eldana saccharina, Ephestia kuehniella, Epinotia spp., Epiphyas postvittana, Etiella spp., Eulia spp., Eupoecilia ambiguella, Euproctis spp., Euxoa spp., Feltia spp., Galleria mellonella, Gracillaria spp. Grapholitha spp., Hedylepta spp., Helicoverpa spp., Heliothis spp., Hofmannophila pseudospretella, Homoeosoma spp., Homona spp., Hyponomeuta padella, Kakivoria flavofasciata, Laphygma spp., Laspeyresia molesta, Leucinodes orbonalis, Leucoptera spp., Lithocolletis spp., Lithophane antennata, Lobesia spp., Loxagrotis albicosta, Lymantria spp., Lyonetia spp., Malacosoma neustria, Maruca testulalis, Mamestra brassicae, Mocis spp., Mythimna separata, Nymphula spp., Oiketicus spp., Oria spp., Orthaga spp., Ostrinia spp., Oulema oryzae, Panolis flammea, Parnara spp., Pectinophora spp., Perileucoptera spp., Phthorimaea spp., Phyllocnistis citrella, Phyllonorycter spp., Pieris spp., Platynota stultana, Plusia spp., Plutella xylostella, Prays spp., Prodenia spp., Protoparce spp., Pseudaletia spp., Pseudoplusia includens, Pyrausta nubilalis, Rachiplusia nu, Schoenobius spp., Scirpophaga spp., Scotia segetum, Sesamia spp., Sparganothis spp., Spodoptera spp., Stathmopoda spp., Stomopteryx subsecivella, Synanthedon spp., Tecia solanivora, Thermesia gemmatalis, Tinea pellionella, Tineola bisselliella, Tortrix spp., Trichoplusia spp., Tuta absoluta, Virachola spp.
From the order of the Orthoptera, for example, Acheta domesticus, Blatta orientalis, Blattella germanica, Dichroplus spp., Gryllotalpa spp., Leucophaea maderae, Locusta spp., Melanoplus spp., Periplaneta americana, Schistocerca gregaria.
From the order of the Siphonaptera, for example, Ceratophyllus spp., Xenopsylla cheopis.
From the order of the Symphyla, for example, Scutigerella spp.
From the order of the Thysanoptera, for example, Anaphothrips obscurus, Baliothrips biformis, Drepanothris reuteri, Enneothrips flavens, Frankliniella spp., Heliothrips spp., Hercinothrips femoralis, Rhipiphorothrips cruentatus, Scirtothrips spp., Taeniothrips cardamoni, Thrips spp.
From the order of the Thysanura, for example, Lepisma saccharina.
The phytoparasitic nematodes include, for example, Aphelenchoides spp., Bursaphelenchus spp., Ditylenchus spp., Globodera spp., Heterodera spp., Longidorus spp., Meloidogyne spp., Pratylenchus spp., Radopholus similis, Trichodorus spp., Tylenchulus semipenetrans, Xiphinema spp.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes.

The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on their surroundings, habitat or storage space by the customary treatment methods, for example by immersion, spraying, evaporation, fogging, scattering, painting on, injecting and, in the case of propagation material, in particular in the case of seed, also by applying one or more coats.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetically Modified Organisms), and parts thereof, are treated. The terms “parts”, “parts of plants” and “plant parts” have been explained above.

Particularly preferably, plants of the plant cultivars which are in each case commercially available or in use are treated according to the invention. Plant cultivars are understood as meaning plants having novel properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive “synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, sugar beet, tomatoes, peas and other vegetable varieties, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized in particular are the increased defence of the plants against insects, arachnids, nematodes and slugs and snails by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to hereinbelow as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD gARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya beans), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plant cultivars will be developed and/or marketed in the future.

The plants listed can be treated according to the invention in a particularly advantageous manner with the compounds according to the invention at a suitable concentration.

In the veterinary sector, that is, in veterinary medicine, the active compounds according to the present invention are effective in controlling animal parasites, specifically endoparasites or ectoparasites. The term “endoparasite” includes, specifically, helminths (tapeworms, nematodes, trematodes, and the like) and protozoa (coccidium and the like).

Ectoparasites include, typically and preferably, arthropods, specifically, insects (flies (stinging and licking), parasitic fly larvae, sucking lice, crab lice, biting lice, fleas, and the like), acaridae (ticks and the like, for example, hard ticks or soft ticks), or acarina (itchmites, trombiculid mites, bird mites, and the like).

These parasites are as follows:

from Anoplurida, for example, Haematopinus spp., Linognathus spp., Pediculus spp., Phtirus spp., Solenopotes spp.; particularly, for representative examples, Linognathus setosus, Linognathus vituli, Linognathus ovillus, Linognathus oviformis, Linognathus pedalis, Linognathus stenopsis, Haematopinus asini macrocephalus, Haematopinus eurysternus, Haematopinus suis, Pediculus humanus capitis, Pediculus humanus corporis, Phylloera vastatrix, Phthirus pubis, Solenopotes capillatus;
from Mallophagida, Amblycerina, and Ischnocerina, for example, Trimenopon spp., Menopon spp., Trinoton spp., Bovicola spp., Werneckiella spp., Lepikentron spp., Damalina spp., Trichodectes spp., Felicola spp.; particularly, for representative examples, Bovicola bovis, Bovicola ovis, Bovicola limbata, Damalina bovis, Trichodectes canis, Felicola subrostratus, Bovicola caprae, Lepikentron ovis, Werneckiella equi;
from Diptera, Nematocerina, and Brachycerina, for example, Aedes spp., Anopheles ssp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; particularly, for representative examples, Aedes aegypti, Aedes albopictus, Aedes taeniorhynchus, Anopheles gambiae, Anopheles maculipennis, Calliphora erythrocephala, Chrysozona pluvialis, Culex quinquefasciatus, Culex pipiens, Culex tarsalis, Fannia canicularis, Sarcophaga carnaria, Stomoxys calcitrans, Tipula paludosa, Lucilia cuprina, Lucilia sericata, Simulium reptans, Phlebotomus papatasi, Phlebotomus longipalpis, Odagmia ornata, Wilhelmia equina, Boophthora erythrocephala, Tabanus bromius, Tabanus spodopterus, Tabanus atratus, Tabanus sudeticus, Hybomitra ciurea, Chrysops caecutiens, Chrysops relictus, Haematopota pluvialis, Haematopota italica, Musca autumnalis, Musca domestica, Haematobia irritans irritans, Haematobia irritans exigua, Haematobia stimulans, Hydrotaea irritans, Hydrotaea albipuncta, Chrysomya chloropyga, Chrysomya bezziana, Oestrus ovis, Hypoderma bovis, Hypoderma lineatum, Przhevalskiana silenus, Dermatobia hominis, Melophagus ovinus, Lipoptena capreoli, Lipoptena cervi, Hippobosca variegata, Hippobosca equina, Gasterophilus intestinalis, Gasterophilus haemorroidalis, Gasterophilus interrnis, Gasterophilus nasalis, Gasterophilus nigricornis, Gasterophilus pecorum, Braula coeca;
from Siphonapterida, for example, Pulex spp., Ctenocephalides spp., Tunga spp., Xenopsylla spp. Ceratophyllus spp.; particularly, for representative examples, Ctenocephalides canis, Ctenocephalides felis, Pulex irritans, Tunga penetrans, Xenopsylla cheopis;
from Heteropterida, for example, Cimex spp., Triatoma spp., Rhodnius spp., Panstrongylus spp.;
from Blattarida, for example, Blatta orientalis, Periplaneta americana, Blattela germanica, Supella spp. (for example, Suppella longipalpa);
from Acari (Acarina), Metastigmata, and Mesostigmata, for example, Argas spp., Ornithodorus spp., Otobius spp., Ixodes spp., Amblyomma spp., Rhipicephalus (Boophilus) spp., Dermacentor spp., Haemophysalis spp., Hyalomma spp., Dermanyssus spp., Rhipicephalus spp. (original genus of heteroxenous mites), Ornithonyssus spp., Pneumonyssus spp., Raillietia spp., Pneumonyssus spp., Sternostoma spp., Varroa spp., Acarapis spp.); particularly, for representative examples, Argas persicus, Argas reflexus, Ornithodorus moubata, Otobius megnini, Rhipicephalus (Boophilus) microplus, Rhipicephalus (Boophilus) decoloratus, Rhipicephalus (Boophilus) annulatus, Rhipicephalus (Boophilus) calceratus, Hyalomma anatolicum, Hyalomma aegypticum, Hyalomma marginatum, Hyalomma transiens, Rhipicephalus evertsi, Ixodes ricinus, Ixodes hexagonus, Ixodes canisuga, Ixodes pilosus, Ixodes rubicundus, Ixodes scapularis, Ixodes holocyclus, Haemaphysalis concinna, Haemaphysalis punctata, Haemaphysalis cinnabarina, Haemaphysalis otophila, Haemaphysalis leachi, Haemaphysalis longicomi, Dermacentor marginatus, Dermacentor reticulatus, Dermacentor pictus, Dermacentor albipictus, Dermacentor andersoni, Dermacentor variabilis, Hyalomma mauritanicum, Rhipicephalus sanguineus, Rhipicephalus bursa, Rhipicephalus appendiculatus, Rhipicephalus capensis, Rhipicephalus turanicus, Rhipicephalus zambeziensis, Amblyomma americanum, Amblyomma variegatum, Amblyomma maculatum, Amblyomma hebraeum, Amblyomma cajennense, Dermanyssus gallinae, Ornithonyssus bursa, Ornithonyssus sylviarum, Varroa jacobsconi;
from Actinedida (Prostigmata), and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.; particularly, for example, Cheyletiella yasguri, Cheyletiella blakei, Demodex canis, Demodex bovis, Demodex ovis, Demodex caprae, Demodex equi, Demodex caballi, Demodex suis, Neotrombicula autumnalis, Neotrombicula desaleli, Neoschonegastia xerothermobia, Trombicula akamushi, Otodectes cynotis, Notoedres cati, Sarcoptis canis, Sarcoptes bovis, Sarcoptes ovis, Sarcoptes rupicaprae (=S. caprae), Sarcoptes equi, Sarcoptes suis, Psoroptes ovis, Psoroptes cuniculi, Psoroptes equi, Chorioptes bovis, Psoergates ovis, Pneumonyssoidic mange, Pneumonyssoides caninum, Acarapis woodi.

In addition, the compounds according to the invention can be used for controlling pathogenic endoparasites which occur in humans and in animal breeding and animal husbandry in livestock, breeding, zoo, laboratory, experimental and domestic animals. In this case they are effective against all or specific stages of development of the endoparasites and also against resistant and normally sensitive species. By controlling the pathogenic endoparasites, it is intended to reduce illness, deaths and performance reductions (e.g. in the production of meat, milk, wool, hides, eggs, honey and the like), so that more economical and simpler animal keeping is made possible by the use of the active compounds. Pathogenic endoparasites include cestodes, trematodes, nematodes, acanthocephales.

The active compounds according to the present invention are suitable for the control of arthropods, helminths, and protozoa that attack animals. The animals include, for example, agricultural animals such as cattle, sheep, goats, horses, pigs, donkeys, camels, buffalo, rabbits, chickens, turkeys, ducks, geese, hatchery fish, and bees. In addition, the animals include pet animals (also known as companion animals), such as dogs, cats, cage birds, and aquarium fish, and experimental animals (for example, hamsters, guinea pigs, rats and mice).

Death rates of host animals will be reduced and productivity (in the cases of meat, milk, wool, hides, eggs, honey, and the like) and animal health will be improved by the control of these arthropods, helminths, and/or protozoa using the active compounds according to the present invention, and as a result, a more economic and simpler animal husbandry can be achieved.

For example, it is preferable that the acceptance of blood from a host by parasites should be prevented or inhibited (where applicable). The control of parasites can be helpful for prevention of infections by infectious organisms.

The term “control” used herein in the veterinary field means that the active compounds are effective in reducing each parasite incidence in animals infected by these parasites to harmless levels. More specifically, “controlling” used herein means that the active compounds are effective in killing, inhibiting the growth of, or inhibiting the proliferation of each parasite.

In the present invention, substances having insecticidal efficacies against noxious insects encompassing all of such pests are referred to as insecticides.

In the case of the active compounds of the present invention used as insecticides, they may be formed in general formulation forms. Such formulation forms may include, for example, solutions, emulsions, wettable powders, wettable granules, suspensions, powders, foams, pastes, tablets, granules, aerosols, natural products and synthetic products impregnated with the active compounds, microcapsules, coating agents for seeds, formulations with combustion device (e.g. the combustion devices include fumigation or fume cartridges, cans, coils and the like), UVL (cold mist, warm mist) and the like.

These formulations may be prepared by a method known per se. For example, they can be prepared by mixing the active compounds together with spreading agents, i.e. liquid diluents or carriers; liquefied gas diluents or carriers; solid diluents or carriers, and, optionally, with surfactants i.e. emulsifiers and/or dispersants and/or foam-forming agents.

When water is used as a spreading agent, for example, organic solvents may be used as an auxiliary solvent.

Liquid diluents or carriers may include, for example, aromatic hydrocarbons (e.g. xylene, toluene, alkylnaphthalene etc.), chlorinated aromatic or chlorinated aliphatic hydrocarbons (e.g. chlorobenzenes, ethylene chlorides, methylene chlorides etc.), aliphatic hydrocarbons (e.g. cyclohexanes or paraffins (e.g. mineral oil fractions)), alcohols (e.g. butanol, glycol and ethers or esters thereof, etc.), ketones (e.g. acetone, methyl ethyl ketone, methyl isobutyl ketone, cyclohexanone etc.), strong polar solvents (e.g. dimethylformamide, dimethylsulfoxide etc.), water and the like.

Liquefied gas diluents or carriers may include substances which exist as gas at ambient temperature and normal pressure, for example aerosol propellants such as bulan, propane, nitrogen gas, carbon dioxide and halogenated hydrocarbons.

Solid diluents may include, for example, crushed natural minerals (e.g. kaolin, clay, talc, chalk, quartz, attapulgite, montmorillonite or diatomaceous earth etc.), crushed synthetic minerals (e.g. highly-dispersive silic acid, alumina, silicate etc.) and the like.

Solid carriers for granules may include, for example, crushed and fractionated rocks (e.g. calcite, marble, pumice stone, sepiolite, dolomite etc.), synthetic granules of inorganic or organic powders, fine particles of organic materials (e.g. sawdust, coconut shells, maize cobs, tobacco stalks, etc.).

Emulsifiers and/or foam-forming agents may include, for example, nonionic or anionic emulsifiers (e.g. polyoxyethylene fatty acid esters, polyoxyethylene fatty alcohol ethers (e.g. alkylarylpolyglycol ether), alkyl sulfonates, alkyl sulfates, aryl sulfonates etc.), albumin hydrolysates and the like.

Examples of dispersants include, for example, lignin sulfite waste liquor, methylcellulose and the like.

Adhesive agents may also be used for the formulations (powders, granules, emulsions), such as carboxymethylcellulose, natural or synthetic polymers (e.g. gum Arabic, polyvinyl alcohols, polyvinyl acetates etc.) and the like.

Colorants may be used, such as inorganic pigments (e.g. iron oxide, titanium oxide, Prussian blue etc.), organic pigments (e.g. alizarin dyes, azo dyes or metallophthalocyanine dyes etc.) as well as trace elements (e.g. salts of iron, manganese, boron, copper, cobalt, molybdenum or zinc etc.).

Generally such formulations may contain the active compounds described above in a range from 0.1 to 95% by weight, preferably 0.5 to 90% by weight.

The active compounds of formula (I) of the present invention may be present in the commercially useful formulations and usage forms prepared from their formulations, as mixed formulation forms with other active compounds such as insecticides, poison baits, bactericides, acaricides, nematocides, fungicides, growth regulating agents, herbicides and the like. Above mentioned insecticides may include, for example, organophosphorus agents, carbamate agents, carboxylate chemical agents, chlorohydrocarbon type chemical agents, neonicotinoid type insecticides, insecticidal substances produced by microorganisms and the like.

In addition, the active compounds of formula (I) of the present invention may be present as mixed formulations with synergists and such formulations and usage forms may include commercially useful formulations and forms. Such synergists, which are not necessarily active per se, are the compounds that are capable of enhancing the activity of the active compounds.

The content of the active compounds of formula (I) of the present invention in a commercially useful usage form may vary over a wide range.

The practical usage concentration of the active compounds of formula (I) of the present invention may be within a range of 0.0000001 to 100% by weight, preferably 0.00001 to 1% by weight.

The compounds of formula (I) of the present invention may be used in general manners suitable for their usage forms.

The active compounds of the present invention, when used against hygienic insects and stored grain pests, have effective stability against alkaline substances present in lime materials. In addition, they have excellent residual efficacies in woods and soils.

In general, for the treatment of animals, the active compounds of the present invention may be directly applied to animals. Preferably, they are applied as pharmaceutical compositions which may contain either one or both of pharmaceutically acceptable excipients and adjuvants widely known in the art.

The active compounds in the veterinary field and animal husbandry are applied (administered) by known means, such as by enteral administration in the form of, for example, tablets, capsules, drinks, orally-taken medicines, granulates, pastes, boluses, the feed-through process, and suppositories; by parenteral administration, for example, by injection (intramuscular, subcutaneous, intravenous, intraperitoneal, and the like), by implants, by intranasal application, by dermal use in the form of, for example, dipping or immersing, spraying, pour-on and spot-on, washing, and powdering, and with the help of appliances containing the active compound, such as collars, ear markers, tail markers, limb bands, halters, marking devices, and the like. The active compounds may be formulated in a suitable form such as shampoo, aerosol, or non-pressurized spray, for example, pump spray and nebulizer.

When used in live stock, poultry, and pet animals, the active compounds according to the present invention may be applied in any formulation (for example, powder, wettable powder (WP), emulsion, emulsion concentrate (EC), flowable agent, homogenous solution, and suspension concentrate (SC)) containing the active compounds in an amount of 1 to 80% by weight, either directly or after dilution (for example, 10- to 10000-fold dilution), or by other methods such as chemical baths.

When used in the veterinary field, the active compounds according to the present invention may be used in combination with a suitable synergist or another active compound, such as a tickicide, insecticide, anthelmintic, and antiprotozoal agent.

Furthermore, the active compounds of the present invention have low toxicity and can be safely used for warm-blooded animals.

When used in the animal health field the active compounds according to the invention may be used in combination with suitable synergists or other active compounds, such as for example, acaricides, insecticides, anthelmintics, growth regulators (e.g. juvenile hormone analogues, chitin synthesis inhibitors) and anti-protozoal drugs.

An active compound of the present invention can be prepared in conventional formulation forms, when used as an insecticide. Examples of the formulation forms include solutions, emulsions, wettable powders, water dispersible granules, suspensions, powders, foams, pastes, tablets, granules, aerosols, active compound-infiltrated natural and synthetic materials, microcapsules, seed coating agents, formulations used with a combustion apparatus (for example, fumigation and smoking cartridges, cans, coils or the like as the combustion apparatus), ULV (cold mist, warm mist), and the like.

These formulations can be produced by methods that are known per se. For example, a formulation can be produced by mixing the active compound with a developer, that is, a liquid diluent or carrier; a liquefied gas diluent or carrier; a solid diluent or carrier, and optionally with a surfactant, that is, an emulsifier and/or dispersant and/or foaming agent.

In the case where water is used as the developer, for example, an organic solvent can also be used as an auxiliary solvent.

Examples of the liquid diluent or carrier include aromatic hydrocarbons (for example, xylene, toluene, alkylnaphthalene and the like), chlorinated aromatic or chlorinated aliphatic hydrocarbons (for example, chlorobenzenes, ethylene chlorides, methylene chlorides), aliphatic hydrocarbons (for example, cyclohexanes), paraffins (for example, mineral oil fractions), alcohols (for example, butanol, glycols and their ethers, esters and the like), ketones (for example, acetone, methyl ethyl ketone, methyl isobutyl ketone, cyclohexanone and the like), strongly polar solvents (for example, dimethylformamide, dimethylsulfoxide and the like), water and the like.

The liquefied gas diluent or carrier may be those which are gaseous at normal temperature and normal pressure, for example, aerosol propellants such as butane, propane, nitrogen gas, carbon dioxide and halogenated hydrocarbons.

Examples of the solid diluent include pulverized natural minerals (for example, kaolin, clay, talc, chalk, quartz, attapulgite, montmorillonite, diatomaceous earth, and the like), pulverized synthetic minerals (for example, highly dispersed silicic acid, alumina, silicates and the like), and the like.

Examples of the solid carrier for granules include pulverized and screened rocks (for example, calcite, marble, pumice, sepiolite, dolomite and the like), synthetic granules of inorganic and organic powder, fine particles of organic materials (for example, sawdust, coconut shells, maize cobs, tobacco stalk and the like), and the like.

Examples of the emulsifier and/or foaming agent include nonionic and anionic emulsifiers [for example, polyoxyethylene fatty acid esters, polyoxyethylene fatty acid alcohol ethers (for example, alkylaryl polyglycol ether), alkylsulfonates, alkylsulfates, arylsulfonates and the like], albumin hydrolyzate, and the like.

Examples of the dispersant include lignin sulfite waste liquor and methylcellulose.

Fixing agents can also be used in the formulations (powders, granules, emulsions), and examples of the fixing agent include carboxymethylcellulose, natural and synthetic polymers (for example, gum arabic, polyvinyl alcohol, polyvinyl acetate, and the like) and the like.

Colorants can also be used, and examples of the colorants include inorganic pigments (for example, iron oxide, titanium oxide, Prussian Blue and the like), organic dyes such as alizarin dyes, azo dyes or metal phthalocyanine dyes, and in addition, trace elements such as the salts of iron, manganese, boron, copper, cobalt, molybdenum and zinc.

The formulations in general can contain the active ingredient in an amount ranging from 0.1 to 95% by weight, and preferably 0.5 to 90% by weight.

The compound according to the present invention can also exist as an admixture with other active compounds, for example, insecticides, poisonous baits, bactericides, miticides, nematicides, fungicides, growth regulators, herbicides and the like, in the form of their commercially useful formulation forms and in the application forms prepared from those formulations.

The content of the compound according to the present invention in a commercially useful application form can be varied within a wide range.

The concentration of the active compound according to the present invention in actual usage can be, for example, in the range of 0.0000001 to 100% by weight, and preferably 0.00001 to 1% by weight.

The compounds according to the present invention can be used through conventional methods that are appropriate for the usage form.

The active compound of the present invention have, when used against hygiene pests and pests associated with stored products, stability effective against alkali on lime materials, and also shows excellent residual effectiveness on wood and soil.

Next, the present invention is exemplified by way of the following examples, but the invention is not intended to be limited thereto.

### SYNTHETIC EXAMPLE 1

### Synthesis of N-{6-[3-(3,5-dichlorophenyl)-3-(trifluoromethyl)-pyrrolidin-1-yl]-1-naphthyl}propanamide (No. 12-34)

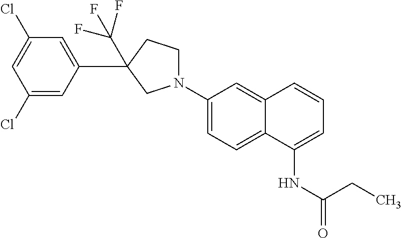

### Step 1-1. Synthesis of t-butyl (6-hydroxy-1-naphthyl)carbamate (cf.: Jacobsen, Eric Jon; US2003/236270 A1)

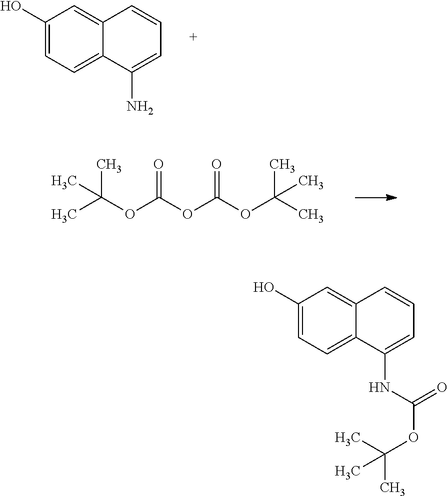

5-Amino-2-naphthol (6.65 g) and di-t-butyl dicarbonate (9.6 ml) were added to tetrahydrofuran (270 ml) and stirred at 70° C. for 16 hours. Di-t-butyl dicarbonate (2 ml) was added to the solution and further stirred at 70° C. for 8 hours. After the solvent was evaporated under reduced pressure, the residue was separated and purified by a column chromatography to obtain t-butyl (6-hydroxy-1-naphthyl)carbamate (10.47 g).

¹H-NMR (CDCl₃) δ: 1.56 (9H, s), 6.41-6.42 (1H, m), 6.77 (1H, br s), 7.02-7.04 (2H, m), 7.33-7.36 (2H, m), 7.59 (1H, d), 7.70 (1H, d).

### Step 1-2. Synthesis of 5-[(t-butoxycarbonyl)amino]-2-naphthyl trifluoromethanesulfonate (See Jacobsen, Eric Jon; US2003/236270 A1)

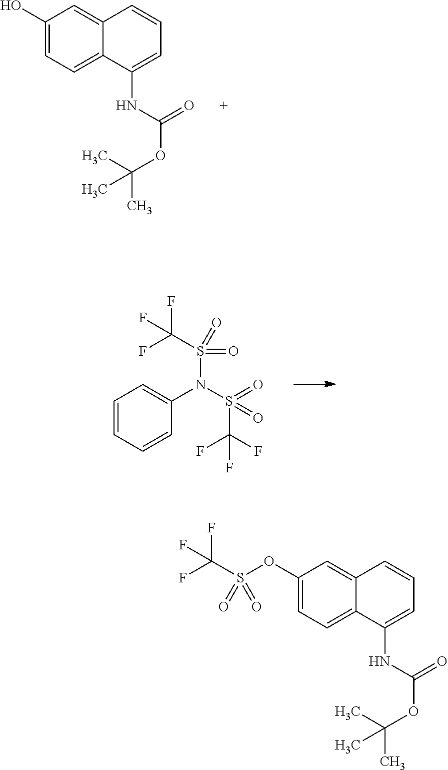

t-Butyl (6-hydroxy-1-naphthyl)carbamate (5.23 g) was dissolved in dichloromethane (100 ml), and triethylamine (2.8 ml) followed by N-phenyl-bis(trifluoromethanesulfonimide) (7.35 g) were added thereto. Then, the mixture was stirred at room temperature under argon atmosphere for 20 hours. The reaction liquid was poured in ethyl acetate and washed three times with brine. The organic layer was dried over anhydrous magnesium sulfate, and after the solvent was evaporated, the residue was purified by a silica gel chromatography to obtain 5-[(t-butoxycarbonyl)amino]-2-naphthyl trifluoromethanesulfonate (7.9 g).

¹H-NMR (CDCl₃) δ: 1.53 (9H, s), 6.77 (1H, br s), 7.39-7.42 (1H, m), 7.55-7.58 (1H, m), 7.67 (1H, d), 7.76 (1H, d), 7.92 (1H, d), 8.00 (1H, d).

### Step 1-3. Synthesis of t-butyl {6-[3-(3,5-dichlorophenyl)-3-(trifluoromethyl)-pyrrolidin-1-yl]-1-naphthyl}carbamate

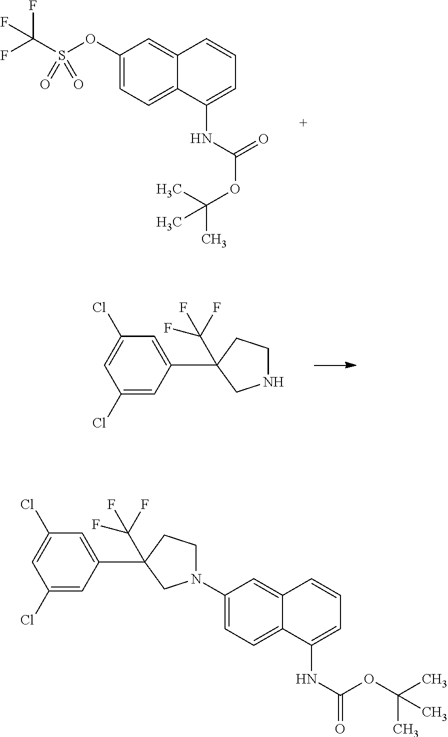

5-[(t-Butoxycarbonyl)amino]-2-naphthyl trifluoromethanesulfonate (1.56 g) and 3-(3,5-dichlorophenyl)-3-(trifluoromethyl)pyrrolidine (1.13 g) were dissolved in toluene (20 ml), and sodium t-butoxide (770 mg), tris(dibenzylideneacetone)-dipalladium (0) chloroform adduct (83 mg) and 4,5-bis(diphenylphosphino)-9,9-dimethylxanthene (143 mg) were added to the solution. Then, the mixture was heated at 100° C. under argon atmosphere for 3 hours. After the reaction mixture was cooled, the mixture was poured into water and extracted with ethyl acetate. The organic layer was washed with brine and dried over anhydrous magnesium sulfate. After the solvent was evaporated under reduced pressure, the residue was purified by a silica gel chromatography to obtain t-butyl {6-[3-(3,5-dichlorophenyl)-3-(trifluoromethyl)-pyrrolidin-1-yl]-1-naphthyl}carbamate (582 mg).

¹H-NMR (CDCl₂) δ: 1.58 (9H, s), 2.56-2.60 (1H, m), 2.85-2.91 (1H, m), 3.64-3.67 (2H, m), 3.90 (1H, d), 4.16 (1H, d), 6.74 (1H, br s), 6.83 (1H, d), 7.01 (1H, d), 7.15-7.61 (6H, m), 7.79 (1H, d).

### Step 1-4. Synthesis of 6-[3-(3,5-dichlorophenyl)-3-(trifluoromethyl)-pyrrolidin-1-yl]naphthalen-1-amine

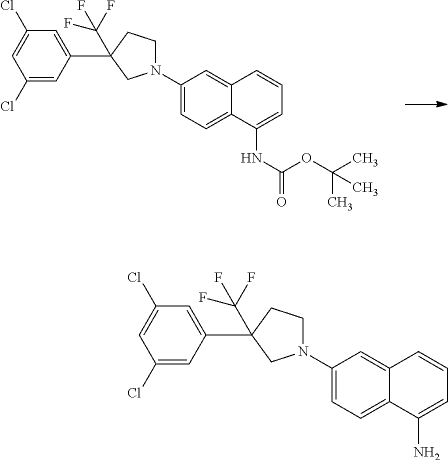

A mixture comprising t-butyl {6-[3-(3,5-dichlorophenyl)-3-(trifluoromethyl)-pyrrolidin-1-yl]-1-naphthyl}carbamate (582 mg), conc. hydrochloric acid (2 ml), and ethanol (10 ml) was stirred at 50° C. for 4 hours. After cooling the mixture to room temperature, the reaction liquid was diluted by adding water (150 mL) and ethyl acetate (150 mL). The solution was neutralized by adding sodium carbonate in small portions. After the extraction using ethyl acetate, the organic layer was washed with brine, and dried over anhydrous magnesium sulfate. The solvent was evaporated under reduced pressure to obtain 6-[3-(3,5-dichlorophenyl)-3-(trifluoromethyl)pyrrolidin-1-yl]naphthalen-1-amine (380 mg).

### Step 1-5. Synthesis of N-{6-[3-(3,5-dichlorophenyl)-3-(trifluoromethyl)-pyrrolidin-1-yl]-1-naphthyl}propanamide

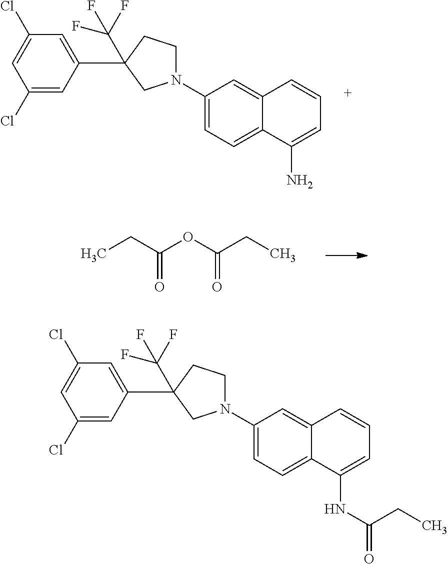

6-[3-(3,5-Dichlorophenyl)-3-(trifluoromethyl)pyrrolidin-1-yl]naphthalen-1-amine (95 mg) was dissolved in tetrahydrofuran (10 ml). Triethylamine (0.5 ml) and propionic acid anhydride (0.5 ml) were added to the solution, and the mixture was heated and refluxed for 2 hours. After cooling the mixture, the solvent was evaporated and the residue was purified by a silica gel chromatography to obtain N-{6-[3-(3,5-dichlorophenyl)-3-(trifluoromethyl)pyrrolidin-1-yl]-1-naphthyl}propanamide (24 mg).

¹H-NMR (CDCl₃) δ: 1.11-1.16 (3H, m), 2.33-2.58 (3H, m), 2.85-2.90 (1H, m), 3.61-3.64 (2H, m), 3.89 (1H, d), 4.16 (1H, d), 6.83 (1H, s), 7.00-7.03 (1H, m), 7.33-7.73 (8H, m).

### SYNTHESIS EXAMPLE 2

### Synthesis of N-{2-[3-(3,5-dichlorophenyl)-3-(trifluoromethyl)-pyrrolidin-1-yl]-4,5,6,7-tetrahydro-1,3-benzothiazol-7-yl}propanamide (No. 11-437)

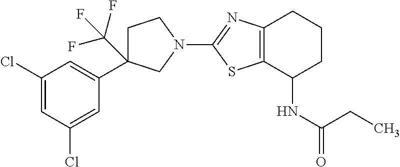

### Step 2-1. Synthesis of 2-[3-(3,5-dichlorophenyl)-3-(trifluoromethyl)pyrrolidin-1-yl]-5,6-dihydro-1,3-benzothiazol-7(4H)-one (Compound No. 21-1)

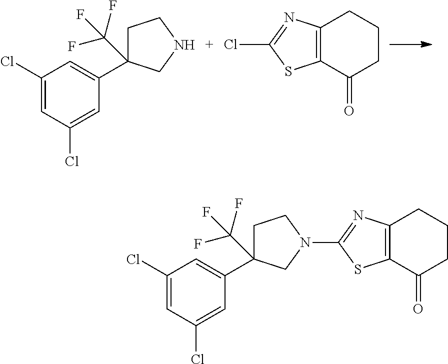

Mixture of 3-(3,5-dichlorophenyl)-3-(trifluoromethyl)pyrrolidine (1.0 g, 5.33 mmol), 2-chloro-5,6-dihydro-1,3-benzothiazol-7(4H)-one (1.51 g, 5.33 mmol), potassium carbonate (1.1 g, 8 mmol) and N,N-dimethylformamide (10 ml) was stirred at 80° C. for 4 hours. The reaction mixture was poured into water, extracted twice with ethyl acetate. The combined organic phase was washed with water and then brine, then dried over anhydrous magnesium sulfate and evaporated. The crude material was chromatographed on silica gel using hexane/ethyl acetate (50/50), to obtain 2-[3-(3,5-dichlorophenyl)-3-(trifluoromethyl)pyrrolidin-1-yl]-5,6-dihydro-1,3-benzothiazol-7(4H)-one (2.0 g; 86.2%).

¹H NMR: see the table below.

### Step 2-2. Synthesis of 2-[3-(3,5-dichlorophenyl)-3-(trifluoromethyl)pyrrolidin-1-yl]-N-methoxy-5,6-dihydro-1,3-benzothiazol-7(4H)-imine (Compound No. 21-3)

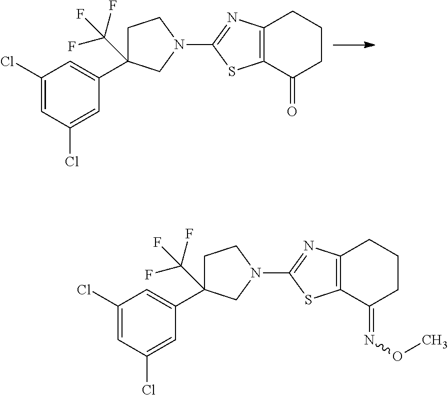

2-[3-(3,5-Dichlorophenyl)-3-(trifluoromethyl)pyrrolidin-1-yl]-5,6-dihydro-1,3-benzothiazol-7(4H)-one (200 mg, 0.46 mmol), methoxyamine hydrochloride (77 mg, 0.92 mmol) and ethanol (10 ml) refluxed for 1 hour. After completion of the reaction, solvent was removed under reduced pressure. Residue was solved in ethyl acetate and washed (water then brine). Dried over anhydrous magnesium sulfate and evaporated to obtain 2-[3-(3,5-dichlorophenyl)-3-(trifluoromethyl)pyrrolidin-1-yl]-N-methoxy-5,6-dihydro-1,3-benzothiazol-7(4H)-imine (200 mg, 94%).

¹H NMR: see the table below.

### Step 2-3. synthesis of N-{2-[3-(3,5-dichlorophenyl)-3-(trifluoromethyl)pyrrolidin-1-yl]-4,5,6,7-tetrahydro-1,3-benzothiazol-7-yl}propanamide (Compound No. 11-437).

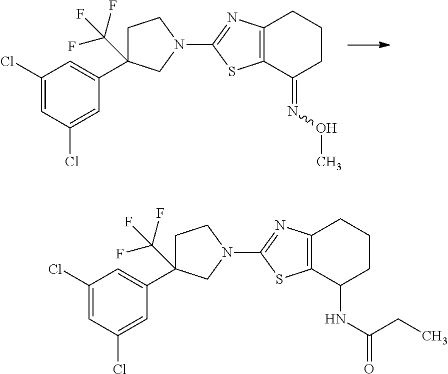

2-[3-(3,5-Dichlorophenyl)-3-(trifluoromethyl)pyrrolidin-1-yl]-N-methoxy-5,6-dihydro-1,3-benzothiazol-7(4H)-imine (160 mg, 0.345 mmol), Propionic acid anhydride (90 mg, 0.69 mmol), nickel(II) chloride hexahydrate (82 mg, 0.345 mmol), methanol (5 ml) and 1,4-dioxane (10 ml) were mixed and stirred over a ice-water bath. Sodiumborohydride (123 mg, 3.45 mmol) was added portionwise, then stirred 0.5 hour. Diethylenetriamine (710 mg, 6.9 mmol) was added and then stirred for 0.5 hour at room temperature. Water and t-BuOMe was added, then separated, organic phase is washed (water then brine), dried over anhydrous magnesium sulfate, and the solvent was removed under reduced pressure. The crude material was chromatographed on silica gel using Hexane/Ethyl acetate (70/30->50/50), to obtain N-{2-[3-(3,5-dichlorophenyl)-3-(trifluoromethyl)pyrrolidin-1-yl]-4,5,6,7-tetrahydro-1,3-benzothiazol-7-yl}propanamide (25 mg, 15%).

¹H NMR: see the table below.

### SYNTHETIC EXAMPLE 3

### Synthesis of N-(2-{3-[3,5-bis(trifluoromethyl)phenyl]-3-(trifluoromethyl)pyrrolidin-1-yl}quinolin-5-yl)propanamide (No. 13-293)

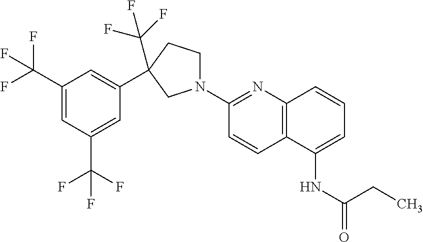

### Step 3-1. Synthesis of tert-butyl quinolin-5-ylcarbamate

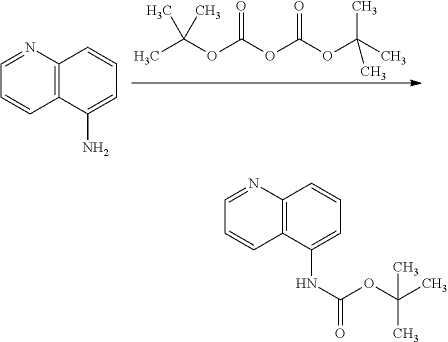

5-Aminoquinoline (5.4 g) was dissolved in THF (150 ml). To the solution were added triethylamine (10 ml) and di-tert-butyldicarbonate (10 ml). The reaction mixture was stirred for 14 hours at 70° C. After cooling, the reaction mixture was concentrated in vacuo to give crude material that was purified by silica gel column chromatography to give tert-butyl quinolin-5-ylcarbamate (6.4 g, 70% yield).

¹H-NMR (CDCl3) δ: 1.31 (9H, s), 7.40-7.46 (2H, m), 7.70-7.72 (1H, m), 8.09-8.19 (2H, m), 8.94-8.96 (1H, m)

### Step 3-2. Synthesis of tert-butyl (1-oxidoquinolin-5-yl)carbamate

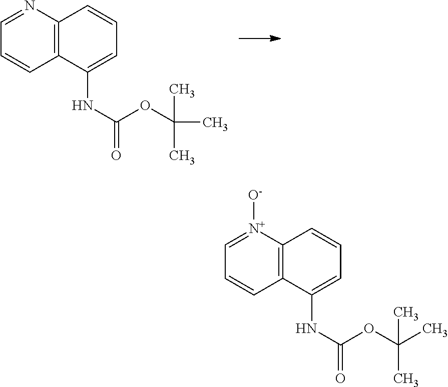

Tert-butyl-quinolin-5-ylcarbamate (2.4 g) was dissolved in dichloromethane (100 ml). The solution was treated with sodium hydrogen carbonate (4.2 g) followed by 3-chloroperbenzoic acid (1.7 g). The reaction mixture was stirred for 17 hours at room temperature. The solution was washed with water, saturated sodium hydrogen carbonate water and then brine. The organic phase was dried over anhydrous magnesium sulfate. After filtration, the filtrate was concentrated in vacuo to give tert-butyl (1-oxidoquinolin-5-yl)carbamate (2.4 g, 93% yield).

¹H-NMR (CDCl3) δ: 1.32 (9H, s), 7.33-7.36 (1H, m), 7.48-7.50 (1H, m), 7.70-7.78 (2H, m), 8.55 (1H, d), 8.77 (1H, d)

### Step 3-3. Synthesis of 2-chloroquinolin-5-amine

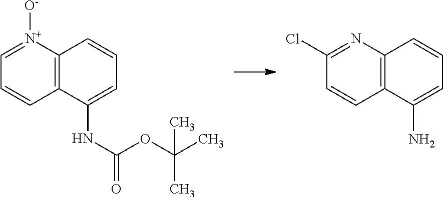

Tert-butyl-(1-oxidoquinolin-5-yl)carbamate (2.3 g) was dissolved in phosphorus oxide chloride (20 ml). The reaction mixture was refluxed for 2.5 hours. After cooling, the reaction mixture was concentrated in vacuo to give crude material. The crude material was treated with ethyl acetate and saturated sodium hydrogen carbonate water. The organic phase was separated. The water phase was extracted with ethyl acetate. The combined extracts were dried over anhydrous magnesium sulfate. After filtered off, the filtrate was concentrated in vacuo to give 2-chloroquinolin-5-amine (0.18 g, 11% yield).

¹H-NMR (CDCl3) δ: 4.21 (21-1, br s), 6.82 (1H, dd,), 7.31 (1H, d), 7.45-7.55 (2H, m), 8.11 (1H, dd)

### Step 3-4. Synthesis of N-(2-chloroquinolin-5-yl)propanamide

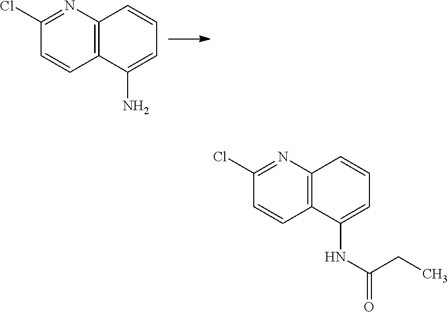

2-Chloroquinolin-5-amine (0.18 g) and triethylamine (0.16 ml) were dissolved in THF (15 ml). To the solution was added propionic acid anhydride (0.16 ml). The reaction mixture was stirred for 3 days at room temperature. The reaction mixture was concentrated in vacuo to give crude material that was purified by silica gel column chromatography to give N-(2-chloroquinolin-5-yl)propanamide (0.19 g, 79% yield).

¹H-NMR (CDCl3) δ: 1.26-1.34 (3H, m), 2.53-2.56 (2H, m), 7.40-7.43 (2H, m), 7.70-7.93 (3H, m), 8.12 (1H, d)

### Step 3-5

### Synthesis of N-(2-{3-[3,5-bis(trifluoromethyl)phenyl]-3-(trifluoromethyl)pyrrolidin-1-yl}-quinolin-5-yl)propanamide (No. 13-293)

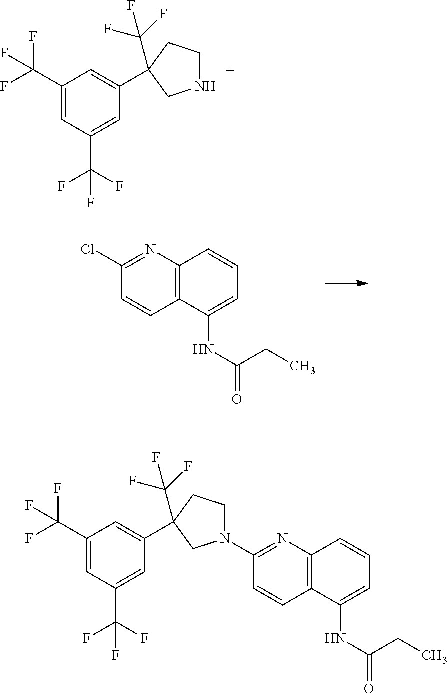

N-(2-chloroquinolin-5-yl)propanamide (0.09 g) and 3-[3,5-bis(trifluoromethyl)phenyl]-3-(trifluoromethyl)pyrrolidine (0.17 g) were dissolved in 1-methyl-2-pyrrolidone (6 ml). To the solution was added potassium carbonate (0.07 g). The reaction mixture was stirred for 2 hours at 180° C. by microwave reactor Initiator™ Biotage. After cooling, the reaction mixture was diluted with brine and extracted with ethyl acetate. The organic phase was dried over anhydrous magnesium silfate. After filtered off, the filtrate was concentrated in vacuo to give crude material that was purified by silica gel column chromatography to give N-(2-{3-[3,5-bis(trifluoromethyl)phenyl]-3-(trifluoromethyl)pyrrolidin-1-yl}quinolin-5-yl)propan amide (0.08 g, 37% yield).

¹H NMR: see the table below.

### SYNTHETIC EXAMPLE 4

### Synthesis of 2-[3-(3,5-dichlorophenyl)-3-(trifluoromethyl)pyrrolidin-1-yl]-N-ethyl-1,3-benzothiazole-7-carboxamide (compound No. 18-2)

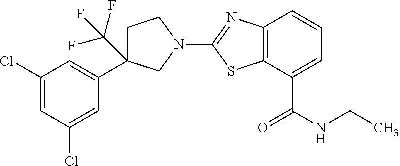

### Step 4-1. Synthesis of methyl 2-[3-(3,5-dichlorophenyl)-3-(trifluoromethyl)pyrrolidin-1-yl]-1,3-benzothiazole-7-carboxylate (Compound No. 20-1)

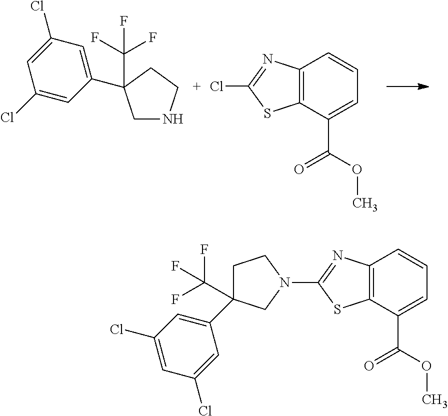

Methyl 2-chloro-1,3-benzothiazole-7-carboxylate (0.20 g) and 3-(3,5-dichlorophenyl)-3-(trifluoromethyl)pyrrolidine (0.25 g) were dissolved in N,N-dimethylformamide (50 ml). Potassium carbonate (0.20 g) was added to the solution. The reaction mixture was stirred for 5 hours at 100° C. After cooling, the reaction mixture was diluted with brine and extracted with ethyl acetate. The organic layer was washed with brine and dried over anhydrous magnesium sulfate. After the solvent was evaporated under reduced pressure, the residue was purified by a column chromatography to obtain methyl 2-[3-(3,5-dichlorophenyl)-3-(trifluoromethyl)pyrrolidin-1-yl]-1,3-benzothiazole-7-carboxylate (0.25 g).

¹H NMR: see the table below.

### Step 4-2. Synthesis of 2-[3-(3,5-dichlorophenyl)-3-(trifluoromethyl)pyrrolidin-1-yl]-1,3-benzothiazole-7-carboxylic acid

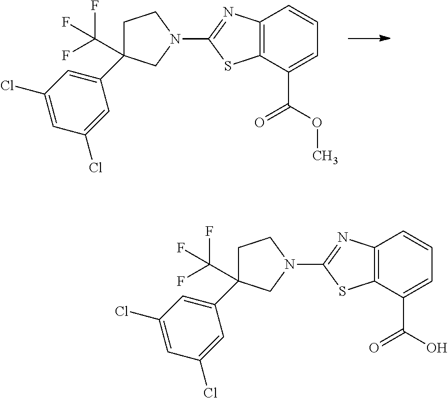

Methyl 2-[3-(3,5-dichlorophenyl)-3-(trifluoromethyl)pyrrolidin-1-yl]-1,3-benzothiazole-7-carboxylate (1.60 g) was dissolved in 1,4-dioxane (25 ml). Sodium hydroxide (2.00 g) in water (40 ml) was added to the solution. The reaction mixture was stirred for 10 hours at 80° C. After cooling, the reaction mixture was diluted with water and acidified with conc. hydrochloric acid. After the extraction using ethyl acetate, the organic layer was washed with brine and dried over anhydrous magnesium sulfate. The solvent was evaporated under reduced pressure to obtain 2-[3-(3,5-dichlorophenyl)-3-(trifluoromethyl)pyrrolidin-1-yl]-1,3-benzothiazole-7-carboxylic acid (1.55 g) as a crude product that was used for next step without further purification.

### Step 4-3. Synthesis of 2-[3-(3,5-dichlorophenyl)-3-(trifluoromethyl)pyrrolidin-1-yl]-1,3-benzothiazole-7-carbonyl chloride

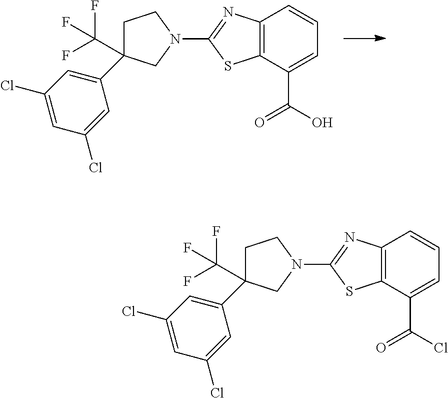

2-[3-(3,5-dichlorophenyl)-3-(trifluoromethyl)pyrrolidin-1-yl]-1,3-benzothiazole-7-carboxylic acid (1.55 g) and thionyl chloride (1.00a) was added to 1,2-dichloroethane (50 ml). The reaction mixture was refluxed for 5 hours. After cooling, the solvent was evaporated under reduced pressure to obtain 2-[3-(3,5-dichlorophenyl)-3-(trifluoromethyl)pyrrolidin-1-yl]-1,3-benzothiazole-7-carbonyl chloride (1.60 g) as a crude product that was used for the next step without further purification.

### Step 4-4. Synthesis of 2-[3-(3,5-dichlorophenyl)-3-(trifluoromethyl)pyrrolidin-1-yl]-N-ethyl-1,3-benzothiazole-7-carboxamide (Compound No. 18-2).

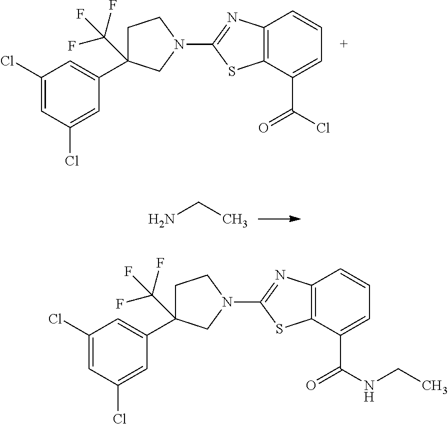

70% Ethylamine solution was added to acetonitrile (20 ml). 2-[3-(3,5-dichlorophenyl)-3-(trifluoromethyl)pyrrolidin-1-yl]-1,3-benzothiazole-7-carbonyl chloride (0.15 g) in tetrahydrofuran (5 ml) was added to the solution. The reaction mixture was stirred for 1 hour at room temperature. After the solvent was evaporated under reduced pressure, the residue was purified by a column chromatography to obtain 2-[3-(3,5-dichlorophenyl)-3-(trifluoromethyl)pyrrolidin-1-yl]-N-ethyl-1,3-benzothiazole-7-carboxamide (0.10 g).

¹H NMR: see the table below.

### SYNTHETIC EXAMPLE 5

### Synthesis of N-{2-[3-(3,5-dichlorophenyl)-3-(trifluoromethyl)pyrrolidin-1-yl]-1,3-benzothiazol-7-yl}propanamide (Compound No. 16-34)

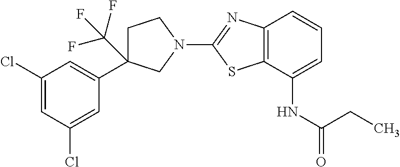

### Step 5-1. Synthesis of tert-butyl {2-[3-(3,5-dichlorophenyl)-3-(trifluoromethyl)pyrrolidin-1-yl]-1,3-benzothiazol-7-yl}carbamate (Compound No. 16-156)

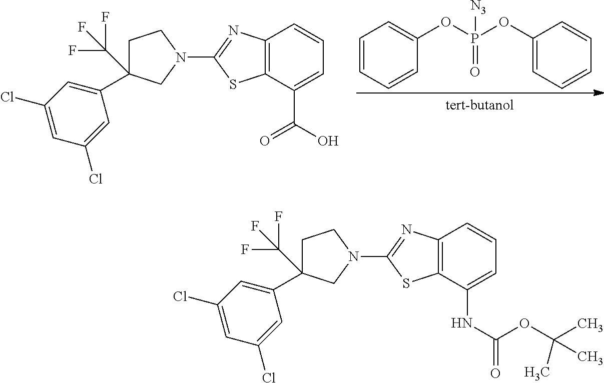

2-[3-(3,5-dichlorophenyl)-3-(trifluoromethyl)pyrrolidin-1-yl]-1,3-benzothiazole-7-carboxylic acid (0.75 g) and diphenyl phosphorazidate (0.50 g) and t-butanol (1.00 g) were dissolved in toluene (50 ml). Triethylamine (0.20 g) in toluene (10 ml) was added to the solution at 0° C. The reaction mixture was refluxed for 6 hours. After cooling, the reaction mixture was poured into saturated sodium hydrogen carbonate water and extracted with ethyl acetate. The organic layer was washed with brine and dried over anhydrous magnesium sulfate. After the solvent was evaporated under reduced pressure, the residue was purified by a column chromatography to obtain {2-[3-(3,5-dichlorophenyl)-3-(trifluoromethyl)pyrrolidin-1-yl]-1,3-benzothiazol-7-yl}carbamate (0.70 g).

¹H NMR: see the table below.

### Step 5-2. Synthesis of 2-[3-(3,5-dichlorophenyl)-3-(trifluoromethyl)pyrrolidin-1-yl]-1,3-benzothiazol-7-amine

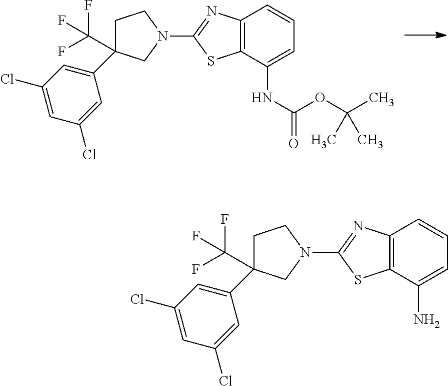

{2-[3-(3,5-dichlorophenyl)-3-(trifluoromethyl)pyrrolidin-1-yl]-1,3-benzothiazol-7-yl}carbamate (0.60 g) was dissolved in dichloromethane (30 ml). Trifluoroacetic acid (3.00 g) in dichloromethane (10 ml) was added to the solution. The reaction mixture was stirred for 10 hours at room temperature. The reaction mixture was washed with saturated sodium hydrogen carbonate water and dried over anhydrous magnesium sulfate. The solvent was evaporated under reduced pressure to obtain 2-[3-(3,5-dichlorophenyl)-3-(trifluoromethyl)pyrrolidin-1-yl]-1,3-benzothiazol-7-amine (0.45 g) as a crude product that was used for the next step without further purification.

### Step 5-3. Synthesis of N-{2-[3-(3,5-dichlorophenyl)-3-(trifluoromethyl)pyrrolidin-1-yl]-1,3-benzothiazol-7-yl}propanamide (Compound No. 16-34)

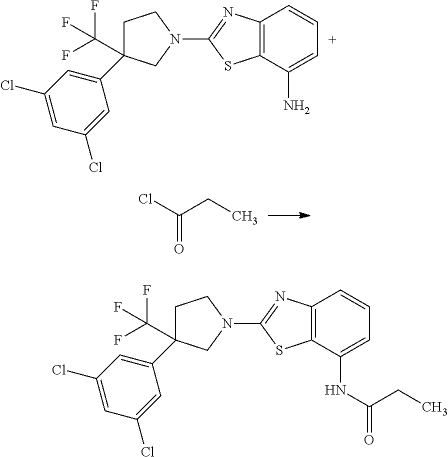

2-[3-(3,5-dichlorophenyl)-3-(trifluoromethyl)pyrrolidin-1-yl]-1,3-benzothiazol-7-amine (0.15 g) and triethylamine (0.10 g) was dissolved in dichloromethane (20 ml). Propionyl chloride (0.05 g) in dichloromethane (10 ml) was added to the solution. The reaction mixture was stirred for 1 hour at room temperature. After the solvent was evaporated under reduced pressure, the residue was purified by a column chromatography to obtain N-{2-[3-(3,5-dichlorophenyl)-3-(trifluoromethyl)pyrrolidin-1-yl]-1,3-benzothiazol-7-yl}propanamide (0.15 g).

¹H NMR: see the table below.

### SYNTHETIC EXAMPLE 6

### Synthesis of N-({2-[3-(3,5-dichlorophenyl)-3-(trifluoromethyl)pyrrolidin-1-yl]-1,3-benzothiazol-7-yl}methyl)propanamide (Compound No. 19-3)

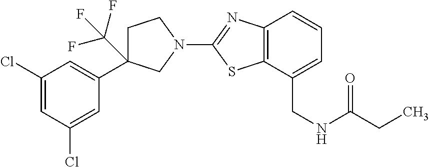

### Step 6-1. Synthesis of {2-[3-(3,5-dichlorophenyl)-3-(trifluoromethyl)pyrrolidin-1-yl]-1,3-benzothiazol-7-yl}methanol

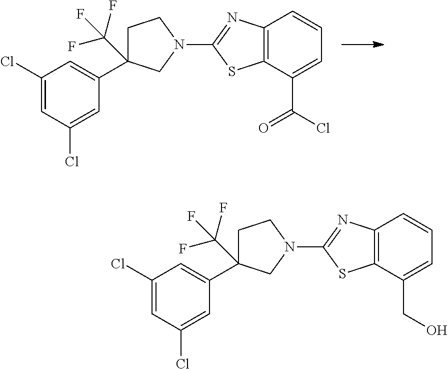

Sodium borohydride (0.30 g) and ice (30 g) was added to 1,4-dioxane (20 ml). 2-[3-(3,5-dichlorophenyl)-3-(trifluoromethyl)pyrrolidin-1-yl]-1,3-benzothiazole-7-carbonyl chloride (0.15 g) in 1,4-dioxane (20 ml) was added to the solution at 0° C. The reaction mixture was refluxed for 1 hour. After cooling, the reaction mixture was poured into water and extracted with ethyl acetate. The organic layer was washed with brine and dried over anhydrous magnesium sulfate. After the solvent was evaporated under reduced pressure, the residue was purified by a column chromatography to obtain {2-[3-(3,5-dichlorophenyl)-3-(trifluoromethyl)pyrrolidin-1-yl]-1,3-benzothiazol-7-yl}methanol (0.50 g).

### Step 6-2. Synthesis of 1-{2-[3-(3,5-dichlorophenyl)-3-(trifluoromethyl)pyrrolidin-1-yl]-1,3-benzothiazol-7-yl}methanamine

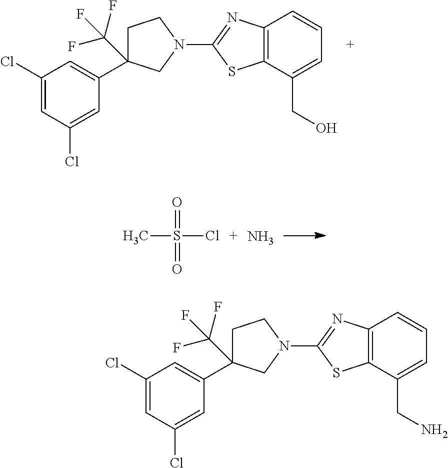

{2-[3-(3,5-dichlorophenyl)-3-(trifluoromethyl)pyrrolidin-1-yl]-1,3-benzothiazol-7-yl}methanol (0.45 g) and triethylamine (0.15 g) was dissolved in acetonitrile (50 ml). Methanesulfonyl chloride (0.15 g) in acetonitrile (10 ml) was added to the solution at 0° C. The reaction mixture was stirred for 1 hour at room temperature. The reaction mixture was added to the solution of 30% ammonia water (50 ml) and acetonitrile (100 ml) at 0° C. The reaction mixture was stirred for 10 hours at room temperature. The organic solvent was evaporated under reduced pressure. The residue was diluted with water and extracted with ethyl acetate. The organic layer was washed with brine and dried over anhydrous magnesium sulfate. The solvent was evaporated under reduced pressure to obtain 1-{2-[3-(3,5-dichlorophenyl)-3-(trifluoromethyl)pyrrolidin-1-yl]-1,3-benzothiazol-7-yl}methanamine (0.40 g) as a crude product that was used for the next step without further purification.

### Step 6-3. Synthesis of N-({2-[3-(3,5-dichlorophenyl)-3-(trifluoromethyl)pyrrolidin-1-yl]-1,3-benzothiazol-7-yl}methyl)propanamide (Compound No. 19-3)

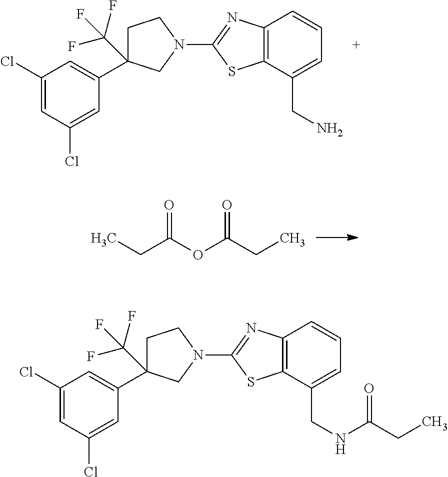

1-{2-[3-(3,5-dichlorophenyl)-3-(trifluoromethyl)pyrrolidin-1-yl]-1,3-benzothiazol-7-yl}methanamine (0.20 g) and propionic anhydride (0.10 g) were dissolved in dichloromethane (20 ml). Triethylamine (0.10 g) in dichloromethane (10 ml) was added to the solution. The reaction mixture was stirred for 1 hour at room temperature. After the solvent was evaporated under reduced pressure, the residue was purified by a column chromatography to obtain N-({2-[3-(3,5-dichlorophenyl)-3-(trifluoromethyl)pyrrolidin-1-yl]-1,3-benzothiazol-7-yl}methyl)propanamide (0.15 g).

¹H NMR: see the table below.

The compounds according to the present invention as well as useful intermediates for the manufacturing of the compounds are described in the following tables.

In tables, abbreviations are as follows: Me: methyl, Et: ethyl, Pr: propyl, Bu: butyl, Allyl: allyl, Pen: pentyl, Hex: hexyl, Ph: phenyl, cyclo-: cyclic, py: pyridyl, pyrro or pyrrolidine: pyrrolidinyl, pipe or piperidine: piperidinyl, morph or morpholine: morpholinyl, thiomorph or thiomorpholine: thiomorpholinyl. In the tables, cyclic groups to which a carbonyl group is attached to,

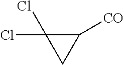

are bound to the appropriate position in the general formula through the carbonyl carbon atom as follows:

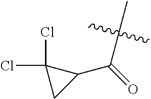

TABLE 8

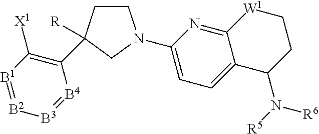

| No. | B¹ | B² | B³ | B⁴ | X¹ | R | W¹ | R⁵ | R⁶ |
|---|---|---|---|---|---|---|---|---|---|
| 8-1 | C—Cl | C—H | C—H | C—H | H | CF3 | — | H | CH3CO |
| 8-2 | C—Cl | C—H | C—H | C—H | H | CF3 | — | H | CH3CH2CO |
| 8-3 | C—Cl | C—H | C—H | C—H | H | CF3 | — | H | n-PrCO |
| 8-4 | C—Cl | C—H | C—H | C—H | H | CF3 | — | H | cyclo-PrCO |
| 8-5 | C—Cl | C—H | C—H | C—H | H | CF3 | — | H | cyclo-PrCH2CO |
| 8-6 | C—Cl | C—H | C—H | C—H | H | CF3 | — | H | CF3CH2CO |
| 8-7 | C—Cl | C—H | C—H | C—H | H | CF3 | — | H | CH3SCH2CO |
| 8-8 | C—Cl | C—H | C—H | C—H | H | CF3 | — | H | CH3S(O)CH2CO |
| 8-9 | C—Cl | C—H | C—H | C—H | H | CF3 | — | H | CH3SO2CH2CO |
| 8-10 | C—Cl | C—H | C—H | C—H | H | CF3 | — | H | CH3CH2NHCO |
| 8-11 | C—Cl | C—H | C—H | C—H | F | CF3 | — | H | CH3CO |
| 8-12 | C—Cl | C—H | C—H | C—H | F | CF3 | — | H | CH3CH2CO |
| 8-13 | C—Cl | C—H | C—H | C—H | F | CF3 | — | H | n-PrCO |
| 8-14 | C—Cl | C—H | C—H | C—H | F | CF3 | — | H | cyclo-PrCO |
| 8-15 | C—Cl | C—H | C—H | C—H | F | CF3 | — | H | cyclo-PrCH2CO |
| 8-16 | C—Cl | C—H | C—H | C—H | F | CF3 | — | H | CF3CH2CO |
| 8-17 | C—Cl | C—H | C—H | C—H | F | CF3 | — | H | CH3SCH2CO |
| 8-18 | C—Cl | C—H | C—H | C—H | F | CF3 | — | H | CH3S(O)CH2CO |
| 8-19 | C—Cl | C—H | C—H | C—H | F | CF3 | — | H | CH3SO2CH2CO |
| 8-20 | C—Cl | C—H | C—H | C—H | F | CF3 | — | H | CH3CH2NHCO |
| 8-21 | C—H | C—H | C—Cl | C—H | F | CF3 | — | H | CH3CO |
| 8-22 | C—H | C—H | C—Cl | C—H | F | CF3 | — | H | CH3CH2CO |
| 8-23 | C—H | C—H | C—Cl | C—H | F | CF3 | — | H | n-PrCO |
| 8-24 | C—H | C—H | C—Cl | C—H | F | CF3 | — | H | cyclo-PrCO |
| 8-25 | C—H | C—H | C—Cl | C—H | F | CF3 | — | H | cyclo-PrCH2CO |
| 8-26 | C—H | C—H | C—Cl | C—H | F | CF3 | — | H | CF3CH2CO |
| 8-27 | C—H | C—H | C—Cl | C—H | F | CF3 | — | H | CH3SCH2CO |
| 8-28 | C—H | C—H | C—Cl | C—H | F | CF3 | — | H | CH3S(O)CH2CO |
| 8-29 | C—H | C—H | C—Cl | C—H | F | CF3 | — | H | CH3SO2CH2CO |
| 8-30 | C—H | C—H | C—Cl | C—H | F | CF3 | — | H | CH3CH2NHCO |
| 8-31 | C—Cl | C—H | C—Cl | C—H | H | CF3 | — | H | H |
| 8-32 | C—Cl | C—H | C—Cl | C—H | H | CF3 | — | H | HCO |
| 8-33 | C—Cl | C—H | C—Cl | C—H | H | CF3 | — | H | CH3CO |
| 8-34 | C—Cl | C—H | C—Cl | C—H | H | CF3 | — | H | CH3CH2CO |
| 8-35 | C—Cl | C—H | C—Cl | C—H | H | CF3 | — | H | n-PrCO |
| 8-36 | C—Cl | C—H | C—Cl | C—H | H | CF3 | — | H | iso-PrCO |
| 8-37 | C—Cl | C—H | C—Cl | C—H | H | CF3 | — | H | cyclo-PrCO |
| 8-38 | C—Cl | C—H | C—Cl | C—H | H | CF3 | — | H | n-BuCO |
| 8-39 | C—Cl | C—H | C—Cl | C—H | H | CF3 | — | H | tert-BuCO |
| 8-40 | C—Cl | C—H | C—Cl | C—H | H | CF3 | — | H | iso-BuCO |
| 8-41 | C—Cl | C—H | C—Cl | C—H | H | CF3 | — | H | cyclo-BuCO |
| 8-42 | C—Cl | C—H | C—Cl | C—H | H | CF3 | — | H | (CH3)3CCH2CO |
| 8-43 | C—Cl | C—H | C—Cl | C—H | H | CF3 | — | H | cyclo-PenCO |
| 8-44 | C—Cl | C—H | C—Cl | C—H | H | CF3 | — | H | cyclo-HexCO |
| 8-45 | C—Cl | C—H | C—Cl | C—H | H | CF3 | — | H | cyclo-PrCH2CO |
| 8-46 | C—Cl | C—H | C—Cl | C—H | H | CF3 | — | H | CF3CO |
| 8-47 | C—Cl | C—H | C—Cl | C—H | H | CF3 | — | H | CCl3CO |
| 8-48 | C—Cl | C—H | C—Cl | C—H | H | CF3 | — | H | ClCH2CO |
| 8-49 | C—Cl | C—H | C—Cl | C—H | H | CF3 | — | H | CF2ClCO |
| 8-50 | C—Cl | C—H | C—Cl | C—H | H | CF3 | — | H | ClCH2CH2CO |
| 8-51 | C—Cl | C—H | C—Cl | C—H | H | CF3 | — | H | HCF2CF2CO |
| 8-52 | C—Cl | C—H | C—Cl | C—H | H | CF3 | — | H | CF3CH2CO |
| 8-54 | C—Cl | C—H | C—Cl | C—H | H | CF3 | — | H | HCF2CO |
| 8-55 | C—Cl | C—H | C—Cl | C—H | H | CF3 | — | H | ClCH2CO |
| 8-56 | C—Cl | C—H | C—Cl | C—H | H | CF3 | — | H | 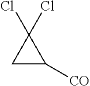 |
| 8-57 | C—Cl | C—H | C—Cl | C—H | H | CF3 | — | H | 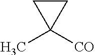 |
| 8-58 | C—Cl | C—H | C—Cl | C—H | H | CF3 | — | H | 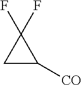 |
| 8-59 | C—Cl | C—H | C—Cl | C—H | H | CF3 | — | H | 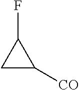 |
| 8-60 | C—Cl | C—H | C—Cl | C—H | H | CF3 | — | H | 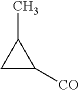 |
| 8-61 | C—Cl | C—H | C—Cl | C—H | H | CF3 | — | H | 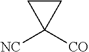 |
| 8-62 | C—Cl | C—H | C—Cl | C—H | H | CF3 | — | H | CH2═CH—CO |
| 8-63 | C—Cl | C—H | C—Cl | C—H | H | CF3 | — | H | CH3CH═CH—CO |
| 8-64 | C—Cl | C—H | C—Cl | C—H | H | CF3 | — | H | CH2═C(CH3)—CO |
| 8-65 | C—Cl | C—H | C—Cl | C—H | H | CF3 | — | H | CH3CH═C(CH3)—CO |
| 8-66 | C—Cl | C—H | C—Cl | C—H | H | CF3 | — | H | (CH3)2C═CH—CO |
| 8-67 | C—Cl | C—H | C—Cl | C—H | H | CF3 | — | H | CH2═C(Cl)—CO |
| 8-68 | C—Cl | C—H | C—Cl | C—H | H | CF3 | — | H | ClCH═C(CH3)—CO |
| 8-69 | C—Cl | C—H | C—Cl | C—H | H | CF3 | — | H | CH2═CHCH2—CO |
| 8-70 | C—Cl | C—H | C—Cl | C—H | H | CF3 | — | H | HCC—CO |
| 8-71 | C—Cl | C—H | C—Cl | C—H | H | CF3 | — | H | CH3CC—CO |
| 8-72 | C—Cl | C—H | C—Cl | C—H | H | CF3 | — | H | NCCH2—CO |
| 8-73 | C—Cl | C—H | C—Cl | C—H | H | CF3 | — | H | CH3OCH2—CO |
| 8-74 | C—Cl | C—H | C—Cl | C—H | H | CF3 | — | H | C2H5OCH2—CO |
| 8-75 | C—Cl | C—H | C—Cl | C—H | H | CF3 | — | H | CH3OCH2CH2—CO |
| 8-76 | C—Cl | C—H | C—Cl | C—H | H | CF3 | — | H | CH3CH2OCH2CH2—CO |
| 8-77 | C—Cl | C—H | C—Cl | C—H | H | CF3 | — | H | CH3SCH2CO |
| 8-78 | C—Cl | C—H | C—Cl | C—H | H | CF3 | — | H | CH3S(O)CH2CO |
| 8-79 | C—Cl | C—H | C—Cl | C—H | H | CF3 | — | H | CH3SO2CH2CO |
| 8-80 | C—Cl | C—H | C—Cl | C—H | H | CF3 | — | H | CH3O(CO)CH2—CO |
| 8-81 | C—Cl | C—H | C—Cl | C—H | H | CF3 | — | H | CH3(CO)OCH2—CO |
| 8-82 | C—Cl | C—H | C—Cl | C—H | H | CF3 | — | H | CH3O(CO)—CO |
| 8-83 | C—Cl | C—H | C—Cl | C—H | H | CF3 | — | H | CF3O(CO)—CO |
| 8-84 | C—Cl | C—H | C—Cl | C—H | H | CF3 | — | H | CH3(CO)CH2—CO |
| 8-85 | C—Cl | C—H | C—Cl | C—H | H | CF3 | — | H | (CH3)2NCH2—CO |
| 8-86 | C—Cl | C—H | C—Cl | C—H | H | CF3 | — | H | PhCO |
| 8-87 | C—Cl | C—H | C—Cl | C—H | H | CF3 | — | H | PhCH2CO |
| 8-88 | C—Cl | C—H | C—Cl | C—H | H | CF3 | — | H | 2-py—CO |
| 8-89 | C—Cl | C—H | C—Cl | C—H | H | CF3 | — | H | 3-py—CO |
| 8-90 | C—Cl | C—H | C—Cl | C—H | H | CF3 | — | H | 4-py—CO |
| 8-91 | C—Cl | C—H | C—Cl | C—H | H | CF3 | — | H | 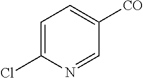 |
| 8-92 | C—Cl | C—H | C—Cl | C—H | H | CF3 | — | H | 2-F—PhCO |
| 8-93 | C—Cl | C—H | C—Cl | C—H | H | CF3 | — | H | 3-F—PhCO |
| 8-94 | C—Cl | C—H | C—Cl | C—H | H | CF3 | — | H | 4-F—PhCO |
| 8-95 | C—Cl | C—H | C—Cl | C—H | H | CF3 | — | H | 2-Cl—PhCO |
| 8-96 | C—Cl | C—H | C—Cl | C—H | H | CF3 | — | H | 3-Cl—PhCO |
| 8-97 | C—Cl | C—H | C—Cl | C—H | H | CF3 | — | H | 4-Cl—PhCO |
| 8-98 | C—Cl | C—H | C—Cl | C—H | H | CF3 | — | H | 2-Br—PhCO |
| 8-99 | C—Cl | C—H | C—Cl | C—H | H | CF3 | — | H | 2-CF3—PhCO |
| 8-100 | C—Cl | C—H | C—Cl | C—H | H | CF3 | — | H | 2-CH3—PhCO |
| 8-101 | C—Cl | C—H | C—Cl | C—H | H | CF3 | — | H | 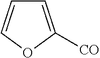 |
| 8-102 | C—Cl | C—H | C—Cl | C—H | H | CF3 | — | H | 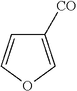 |
| 8-103 | C—Cl | C—H | C—Cl | C—H | H | CF3 | — | H | 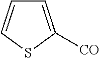 |
| 8-104 | C—Cl | C—H | C—Cl | C—H | H | CF3 | — | H | 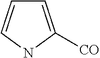 |
| 8-105 | C—Cl | C—H | C—Cl | C—H | H | CF3 | — | H | 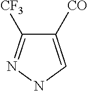 |
| 8-106 | C—Cl | C—H | C—Cl | C—H | H | CF3 | — | H | 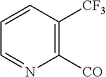 |
| 8-107 | C—Cl | C—H | C—Cl | C—H | H | CF3 | — | H | 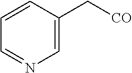 |
| 8-108 | C—Cl | C—H | C—Cl | C—H | H | CF3 | — | H | 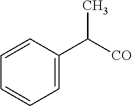 |
| 8-109 | C—Cl | C—H | C—Cl | C—H | H | CF3 | — | H | CH3NHCO |
| 8-110 | C—Cl | C—H | C—Cl | C—H | H | CF3 | — | H | CH3CH2NHCO |
| 8-111 | C—Cl | C—H | C—Cl | C—H | H | CF3 | — | H | CF3CH2NHCO |
| 8-112 | C—Cl | C—H | C—Cl | C—H | H | CF3 | — | H | n-PrNHCO |
| 8-113 | C—Cl | C—H | C—Cl | C—H | H | CF3 | — | H | iso-PrNHCO |
| 8-114 | C—Cl | C—H | C—Cl | C—H | H | CF3 | — | H | cyclo-PrNHCO |
| 8-115 | C—Cl | C—H | C—Cl | C—H | H | CF3 | — | H | n-BuNHCO |
| 8-116 | C—Cl | C—H | C—Cl | C—H | H | CF3 | — | H | tert-BuNHCO |
| 8-117 | C—Cl | C—H | C—Cl | C—H | H | CF3 | — | H | cyclo-BuNHCO |
| 8-118 | C—Cl | C—H | C—Cl | C—H | H | CF3 | — | H | cyclo-PenNHCO |
| 8-119 | C—Cl | C—H | C—Cl | C—H | H | CF3 | — | H | cyclo-HexNHCO |
| 8-120 | C—Cl | C—H | C—Cl | C—H | H | CF3 | — | H | CH2═CHCH2NHCO |
| 8-121 | C—Cl | C—H | C—Cl | C—H | H | CF3 | — | H | HCCCH2NHCO |
| 8-122 | C—Cl | C—H | C—Cl | C—H | H | CF3 | — | H | cyclo-PrCH2NHCO |
| 8-123 | C—Cl | C—H | C—Cl | C—H | H | CF3 | — | H | NCCH2NHCO |
| 8-124 | C—Cl | C—H | C—Cl | C—H | H | CF3 | — | H | CF3(CH3)CHNHCO |
| 8-125 | C—Cl | C—H | C—Cl | C—H | H | CF3 | — | H | CF3(CH3)2CNCO |
| 8-126 | C—Cl | C—H | C—Cl | C—H | H | CF3 | — | H | NCCH2OCO |
| 8-127 | C—Cl | C—H | C—Cl | C—H | H | CF3 | — | H | FCH2CH2NHCO |
| 8-128 | C—Cl | C—H | C—Cl | C—H | H | CF3 | — | H | CF3CH2CH2NHCO |
| 8-129 | C—Cl | C—H | C—Cl | C—H | H | CF3 | — | H | HCF2CF2CH2NHCO |
| 8-130 | C—Cl | C—H | C—Cl | C—H | H | CF3 | — | H | 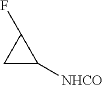 |
| 8-131 | C—Cl | C—H | C—Cl | C—H | H | CF3 | — | H | 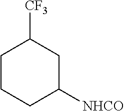 |
| 8-132 | C—Cl | C—H | C—Cl | C—H | H | CF3 | — | H | EtO2CCH2NHCO |
| 8-133 | C—Cl | C—H | C—Cl | C—H | H | CF3 | — | H | 2-CF3—PhNHCO |
| 8-134 | C—Cl | C—H | C—Cl | C—H | H | CF3 | — | H | 3-CF3—PhNHCO |
| 8-135 | C—Cl | C—H | C—Cl | C—H | H | CF3 | — | H | 4-CF3—PhNHCO |
| 8-136 | C—Cl | C—H | C—Cl | C—H | H | CF3 | — | H | 2-py—NHCO |
| 8-137 | C—Cl | C—H | C—Cl | C—H | H | CF3 | — | H | 3-py—NHCO |
| 8-138 | C—Cl | C—H | C—Cl | C—H | H | CF3 | — | H | 4-py—NHCO |
| 8-139 | C—Cl | C—H | C—Cl | C—H | H | CF3 | — | H | 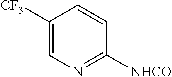 |
| 8-140 | C—Cl | C—H | C—Cl | C—H | H | CF3 | — | H | 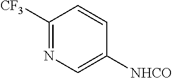 |
| 8-141 | C—Cl | C—H | C—Cl | C—H | H | CF3 | — | H | PhCH2HNCO |
| 8-142 | C—Cl | C—H | C—Cl | C—H | H | CF3 | — | H | 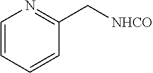 |
| 8-143 | C—Cl | C—H | C—Cl | C—H | H | CF3 | — | H | (CH3)2NCO |
| 8-144 | C—Cl | C—H | C—Cl | C—H | H | CF3 | — | H | pyrrolidineCO |
| 8-145 | C—Cl | C—H | C—Cl | C—H | H | CF3 | — | H | piperidineCO |
| 8-146 | C—Cl | C—H | C—Cl | C—H | H | CF3 | — | H | morpholineCO |
| 8-147 | C—Cl | C—H | C—Cl | C—H | H | CF3 | — | H | CH3OCONH |
| 8-148 | C—Cl | C—H | C—Cl | C—H | H | CF3 | — | H | CH3CH2OCO |
| 8-149 | C—Cl | C—H | C—Cl | C—H | H | CF3 | — | H | n-PrOCO |
| 8-150 | C—Cl | C—H | C—Cl | C—H | H | CF3 | — | H | cyclo-PrOCO |
| 8-151 | C—Cl | C—H | C—Cl | C—H | H | CF3 | — | H | CF3CH2OCO |
| 8-152 | C—Cl | C—H | C—Cl | C—H | H | CF3 | — | H | CH2═CHCH2OCO |
| 8-153 | C—Cl | C—H | C—Cl | C—H | H | CF3 | — | H | HCCCH2OCO |
| 8-154 | C—Cl | C—H | C—Cl | C—H | H | CF3 | — | H | PhOCO |
| 8-155 | C—Cl | C—H | C—Cl | C—H | H | CF3 | — | H | PhCH2OCO |
| 8-156 | C—Cl | C—H | C—Cl | C—H | H | CF3 | — | H | tert-BuOCO |
| 8-157 | C—Cl | C—H | C—Cl | C—H | H | CF3 | — | H | CH3SO2 |
| 8-158 | C—Cl | C—H | C—Cl | C—H | H | CF3 | — | H | CH3CH2SO2 |
| 8-159 | C—Cl | C—H | C—Cl | C—H | H | CF3 | — | H | CF3SO2 |
| 8-160 | C—Cl | C—H | C—Cl | C—H | H | CF3 | — | H | CF3CH2SO2 |
| 8-161 | C—Cl | C—H | C—Cl | C—H | H | CF3 | — | H | PhSO2 |
| 8-162 | C—Cl | C—H | C—Cl | C—H | H | CF3 | — | H | CH3NHCONHSO2 |
| 8-163 | C—Cl | C—H | C—Cl | C—H | H | CF3 | — | H | (CH3)2NCONHSO2 |
| 8-164 | C—Cl | C—H | C—Cl | C—H | H | CF3 | — | H | CH3CS |
| 8-165 | C—Cl | C—H | C—Cl | C—H | H | CF3 | — | H | CH3CH2CS |
| 8-166 | C—Cl | C—H | C—Cl | C—H | H | CF3 | — | H | cyclo-PrCS |
| 8-167 | C—Cl | C—H | C—Cl | C—H | H | CF3 | — | H | CF3CH2CS |
| 8-168 | C—Cl | C—H | C—Br | C—H | H | CF3 | — | H | CH3CO |
| 8-169 | C—Cl | C—H | C—Br | C—H | H | CF3 | — | H | CH3CH2CO |
| 8-170 | C—Cl | C—H | C—Br | C—H | H | CF3 | — | H | n-PrCO |
| 8-171 | C—Cl | C—H | C—Br | C—H | H | CF3 | — | H | cyclo-PrCO |
| 8-172 | C—Cl | C—H | C—Br | C—H | H | CF3 | — | H | cyclo-PrCH2CO |
| 8-173 | C—Cl | C—H | C—Br | C—H | H | CF3 | — | H | CF3CH2CO |
| 8-175 | C—Cl | C—H | C—Br | C—H | H | CF3 | — | H | CH3S(O)CH2CO |
| 8-176 | C—Cl | C—H | C—Br | C—H | H | CF3 | — | H | CH3SO2CH2CO |
| 8-177 | C—Cl | C—H | C—Br | C—H | H | CF3 | — | H | CH3CH2NHCO |
| 8-178 | C—Br | C—H | C—Br | C—H | H | CF3 | — | H | CH3CO |
| 8-179 | C—Br | C—H | C—Br | C—H | H | CF3 | — | H | CH3CH2CO |
| 8-180 | C—Br | C—H | C—Br | C—H | H | CF3 | — | H | n-PrCO |
| 8-181 | C—Br | C—H | C—Br | C—H | H | CF3 | — | H | cyclo-PrCO |
| 8-182 | C—Br | C—H | C—Br | C—H | H | CF3 | — | H | cyclo-PrCH2CO |
| 8-183 | C—Br | C—H | C—Br | C—H | H | CF3 | — | H | CF3CH2CO |
| 8-185 | C—Br | C—H | C—Br | C—H | H | CF3 | — | H | CH3S(O)CH2CO |
| 8-186 | C—Br | C—H | C—Br | C—H | H | CF3 | — | H | CH3SO2CH2CO |
| 8-187 | C—Br | C—H | C—Br | C—H | H | CF3 | — | H | CH3CH2NHCO |
| 8-188 | C—Cl | C—F | C—Cl | C—H | H | CF3 | — | H | CH3CO |
| 8-189 | C—Cl | C—F | C—Cl | C—H | H | CF3 | — | H | CH3CH2CO |
| 8-190 | C—Cl | C—F | C—Cl | C—H | H | CF3 | — | H | n-PrCO |
| 8-191 | C—Cl | C—F | C—Cl | C—H | H | CF3 | — | H | cyclo-PrCO |
| 8-192 | C—Cl | C—F | C—Cl | C—H | H | CF3 | — | H | cyclo-PrCH2CO |
| 8-193 | C—Cl | C—F | C—Cl | C—H | H | CF3 | — | H | CF3CH2CO |
| 8-195 | C—Cl | C—F | C—Cl | C—H | H | CF3 | — | H | CH3S(O)CH2CO |
| 8-196 | C—Cl | C—F | C—Cl | C—H | H | CF3 | — | H | CH3SO2CH2CO |
| 8-197 | C—Cl | C—F | C—Cl | C—H | H | CF3 | — | H | CH3CH2NHCO |
| 8-198 | C—Cl | C—Cl | C—Cl | C—H | H | CF3 | — | H | CH3CO |
| 8-199 | C—Cl | C—Cl | C—Cl | C—H | H | CF3 | — | H | CH3CH2CO |
| 8-200 | C—Cl | C—Cl | C—Cl | C—H | H | CF3 | — | H | n-PrCO |
| 8-201 | C—Cl | C—Cl | C—Cl | C—H | H | CF3 | — | H | cyclo-PrCO |
| 8-202 | C—Cl | C—Cl | C—Cl | C—H | H | CF3 | — | H | cyclo-PrCH2CO |
| 8-203 | C—Cl | C—Cl | C—Cl | C—H | H | CF3 | — | H | CF3CH2CO |
| 8-205 | C—Cl | C—Cl | C—Cl | C—H | H | CF3 | — | H | CH3CH2OCH2CH2—CO |
| 8-206 | C—Cl | C—Cl | C—Cl | C—H | H | CF3 | — | H | CH3SCH2CO |
| 8-207 | C—Cl | C—Cl | C—Cl | C—H | H | CF3 | — | H | CH3S(O)CH2CO |
| 8-208 | C—Cl | C—Cl | C—Cl | C—H | H | CF3 | — | H | CH3SO2CH2CO |
| 8-209 | C—Cl | C—Cl | C—Cl | C—H | H | CF3 | — | H | CH3CH2NHCO |
| 8-210 | C—Cl | C—Br | C—Cl | C—H | H | CF3 | — | H | CH3CO |
| 8-211 | C—Cl | C—Br | C—Cl | C—H | H | CF3 | — | H | CH3CH2CO |
| 8-212 | C—Cl | C—Br | C—Cl | C—H | H | CF3 | — | H | n-PrCO |
| 8-213 | C—Cl | C—Br | C—Cl | C—H | H | CF3 | — | H | cyclo-PrCO |
| 8-214 | C—Cl | C—Br | C—Cl | C—H | H | CF3 | — | H | cyclo-PrCH2CO |
| 8-215 | C—Cl | C—Br | C—Cl | C—H | H | CF3 | — | H | CF3CH2CO |
| 8-217 | C—Cl | C—Br | C—Cl | C—H | H | CF3 | — | H | CH3S(O)CH2CO |
| 8-218 | C—Cl | C—Br | C—Cl | C—H | H | CF3 | — | H | CH3SO2CH2CO |
| 8-219 | C—Cl | C—Br | C—Cl | C—H | H | CF3 | — | H | CH3CH2NHCO |
| 8-220 | C—Cl | C—Cl | C—Br | C—H | H | CF3 | — | H | CH3CO |
| 8-221 | C—Cl | C—Cl | C—Br | C—H | H | CF3 | — | H | CH3CH2CO |
| 8-222 | C—Cl | C—Cl | C—Br | C—H | H | CF3 | — | H | n-PrCO |
| 8-223 | C—Cl | C—Cl | C—Br | C—H | H | CF3 | — | H | cyclo-PrCO |
| 8-224 | C—Cl | C—Cl | C—Br | C—H | H | CF3 | — | H | cyclo-PrCH2CO |
| 8-225 | C—Cl | C—Cl | C—Br | C—H | H | CF3 | — | H | CF3CH2CO |
| 8-227 | C—Cl | C—Cl | C—Br | C—H | H | CF3 | — | H | CH3S(O)CH2CO |
| 8-228 | C—Cl | C—Cl | C—Br | C—H | H | CF3 | — | H | CH3SO2CH2CO |
| 8-229 | C—Cl | C—Cl | C—Br | C—H | H | CF3 | — | H | CH3CH2NHCO |
| 8-230 | C—Cl | C—F | C—Cl | C—H | F | CF3 | — | H | CH3CO |
| 8-231 | C—Cl | C—F | C—Cl | C—H | F | CF3 | — | H | CH3CH2CO |
| 8-232 | C—Cl | C—F | C—Cl | C—H | F | CF3 | — | H | n-PrCO |
| 8-233 | C—Cl | C—F | C—Cl | C—H | F | CF3 | — | H | cyclo-PrCO |
| 8-234 | C—Cl | C—F | C—Cl | C—H | F | CF3 | — | H | cyclo-PrCH2CO |
| 8-235 | C—Cl | C—F | C—Cl | C—H | F | CF3 | — | H | CF3CH2CO |
| 8-237 | C—Cl | C—F | C—Cl | C—H | F | CF3 | — | H | CH3S(O)CH2CO |
| 8-238 | C—Cl | C—F | C—Cl | C—H | F | CF3 | — | H | CH3SO2CH2CO |
| 8-239 | C—Cl | C—F | C—Cl | C—H | F | CF3 | — | H | CH3CH2NHCO |
| 8-240 | C—CF3 | C—H | C—H | C—H | H | CF3 | — | H | CH3CO |
| 8-241 | C—CF3 | C—H | C—H | C—H | H | CF3 | — | H | CH3CH2CO |
| 8-242 | C—CF3 | C—H | C—H | C—H | H | CF3 | — | H | n-PrCO |
| 8-243 | C—CF3 | C—H | C—H | C—H | H | CF3 | — | H | cyclo-PrCO |
| 8-244 | C—CF3 | C—H | C—H | C—H | H | CF3 | — | H | cyclo-PrCH2CO |
| 8-245 | C—CF3 | C—H | C—H | C—H | H | CF3 | — | H | CF3CH2CO |
| 8-247 | C—CF3 | C—H | C—H | C—H | H | CF3 | — | H | CH3S(O)CH2CO |
| 8-248 | C—CF3 | C—H | C—H | C—H | H | CF3 | — | H | CH3SO2CH2CO |
| 8-249 | C—CF3 | C—H | C—H | C—H | H | CF3 | — | H | CH3CH2NHCO |
| 8-250 | C—CF3 | C—F | C—H | C—H | H | CF3 | — | H | CH3CO |
| 8-251 | C—CF3 | C—F | C—H | C—H | H | CF3 | — | H | CH3CH2CO |
| 8-252 | C—CF3 | C—F | C—H | C—H | H | CF3 | — | H | n-PrCO |
| 8-253 | C—CF3 | C—F | C—H | C—H | H | CF3 | — | H | cyclo-PrCO |
| 8-254 | C—CF3 | C—F | C—H | C—H | H | CF3 | — | H | cyclo-PrCH2CO |
| 8-255 | C—CF3 | C—F | C—H | C—H | H | CF3 | — | H | CF3CH2CO |
| 8-257 | C—CF3 | C—F | C—H | C—H | H | CF3 | — | H | CH3S(O)CH2CO |
| 8-258 | C—CF3 | C—F | C—H | C—H | H | CF3 | — | H | CH3SO2CH2CO |
| 8-259 | C—CF3 | C—F | C—H | C—H | H | CF3 | — | H | CH3CH2NHCO |
| 8-260 | C—CF3 | C—H | C—H | C—H | F | CF3 | — | H | CH3CO |
| 8-261 | C—CF3 | C—H | C—H | C—H | F | CF3 | — | H | CH3CH2CO |
| 8-262 | C—CF3 | C—H | C—H | C—H | F | CF3 | — | H | n-PrCO |
| 8-263 | C—CF3 | C—H | C—H | C—H | F | CF3 | — | H | cyclo-PrCO |
| 8-264 | C—CF3 | C—H | C—H | C—H | F | CF3 | — | H | cyclo-PrCH2CO |
| 8-265 | C—CF3 | C—H | C—H | C—H | F | CF3 | — | H | CF3CH2CO |
| 8-267 | C—CF3 | C—H | C—H | C—H | F | CF3 | — | H | CH3S(O)CH2CO |
| 8-268 | C—CF3 | C—H | C—H | C—H | F | CF3 | — | H | CH3SO2CH2CO |
| 8-269 | C—CF3 | C—H | C—H | C—H | F | CF3 | — | H | CH3CH2NHCO |
| 8-270 | C—H | C—H | C—CF3 | C—H | F | CF3 | — | H | CH3CO |
| 8-271 | C—H | C—H | C—CF3 | C—H | F | CF3 | — | H | CH3CH2CO |
| 8-272 | C—H | C—H | C—CF3 | C—H | F | CF3 | — | H | n-PrCO |
| 8-273 | C—H | C—H | C—CF3 | C—H | F | CF3 | — | H | cyclo-PrCO |
| 8-274 | C—H | C—H | C—CF3 | C—H | F | CF3 | — | H | cyclo-PrCH2CO |
| 8-275 | C—H | C—H | C—CF3 | C—H | F | CF3 | — | H | CF3CH2CO |
| 8-277 | C—H | C—H | C—CF3 | C—H | F | CF3 | — | H | CH3S(O)CH2CO |
| 8-278 | C—H | C—H | C—CF3 | C—H | F | CF3 | — | H | CH3SO2CH2CO |
| 8-279 | C—H | C—H | C—CF3 | C—H | F | CF3 | — | H | CH3CH2NHCO |
| 8-280 | C—Cl | C—H | C—CF3 | C—H | H | CF3 | — | H | CH3CO |
| 8-281 | C—Cl | C—H | C—CF3 | C—H | H | CF3 | — | H | CH3CH2CO |
| 8-282 | C—Cl | C—H | C—CF3 | C—H | H | CF3 | — | H | n-PrCO |
| 8-283 | C—Cl | C—H | C—CF3 | C—H | H | CF3 | — | H | cyclo-PrCO |
| 8-284 | C—Cl | C—H | C—CF3 | C—H | H | CF3 | — | H | cyclo-PrCH2CO |
| 8-285 | C—Cl | C—H | C—CF3 | C—H | H | CF3 | — | H | CF3CH2CO |
| 8-287 | C—Cl | C—H | C—CF3 | C—H | H | CF3 | — | H | CH3CH2OCH2CH2—CO |
| 8-288 | C—Cl | C—H | C—CF3 | C—H | H | CF3 | — | H | CH3SCH2CO |
| 8-289 | C—Cl | C—H | C—CF3 | C—H | H | CF3 | — | H | CH3S(O)CH2CO |
| 8-290 | C—Cl | C—H | C—CF3 | C—H | H | CF3 | — | H | CH3SO2CH2CO |
| 8-291 | C—Cl | C—H | C—CF3 | C—H | H | CF3 | — | H | CH3CH2NHCO |
| 8-292 | C—CF3 | C—H | C—CF3 | C—H | H | CF3 | — | H | CH3CO |
| 8-293 | C—CF3 | C—H | C—CF3 | C—H | H | CF3 | — | H | CH3CH2CO |
| 8-294 | C—CF3 | C—H | C—CF3 | C—H | H | CF3 | — | H | n-PrCO |
| 8-295 | C—CF3 | C—H | C—CF3 | C—H | H | CF3 | — | H | cyclo-PrCO |
| 8-296 | C—CF3 | C—H | C—CF3 | C—H | H | CF3 | — | H | cyclo-PrCH2CO |
| 8-297 | C—CF3 | C—H | C—CF3 | C—H | H | CF3 | — | H | CF3CH2CO |
| 8-299 | C—CF3 | C—H | C—CF3 | C—H | H | CF3 | — | H | CH3CH2OCH2CH2—CO |
| 8-300 | C—CF3 | C—H | C—CF3 | C—H | H | CF3 | — | H | CH3SCH2CO |
| 8-301 | C—CF3 | C—H | C—CF3 | C—H | H | CF3 | — | H | CH3S(O)CH2CO |
| 8-302 | C—CF3 | C—H | C—CF3 | C—H | H | CF3 | — | H | CH3SO2CH2CO |
| 8-303 | C—CF3 | C—H | C—CF3 | C—H | H | CF3 | — | H | CH3CH2NHCO |
| 8-304 | C—Cl | C—Cl | C—CF3 | C—H | H | CF3 | — | H | CH3CO |
| 8-305 | C—Cl | C—Cl | C—CF3 | C—H | H | CF3 | — | H | CH3CH2CO |
| 8-306 | C—Cl | C—Cl | C—CF3 | C—H | H | CF3 | — | H | n-PrCO |
| 8-307 | C—Cl | C—Cl | C—CF3 | C—H | H | CF3 | — | H | cyclo-PrCO |
| 8-308 | C—Cl | C—Cl | C—CF3 | C—H | H | CF3 | — | H | cyclo-PrCH2CO |
| 8-309 | C—Cl | C—Cl | C—CF3 | C—H | H | CF3 | — | H | CF3CH2CO |
| 8-311 | C—Cl | C—Cl | C—CF3 | C—H | H | CF3 | — | H | CH3S(O)CH2CO |
| 8-312 | C—Cl | C—Cl | C—CF3 | C—H | H | CF3 | — | H | CH3SO2CH2CO |
| 8-313 | C—Cl | C—Cl | C—CF3 | C—H | H | CF3 | — | H | CH3CH2NHCO |
| 8-314 | C—Cl | N | C—H | C—H | H | CF3 | — | H | CH3CO |
| 8-315 | C—Cl | N | C—H | C—H | H | CF3 | — | H | CH3CH2CO |
| 8-316 | C—Cl | N | C—H | C—H | H | CF3 | — | H | n-PrCO |
| 8-317 | C—Cl | N | C—H | C—H | H | CF3 | — | H | cyclo-PrCO |
| 8-318 | C—Cl | N | C—H | C—H | H | CF3 | — | H | cyclo-PrCH2CO |
| 8-319 | C—Cl | N | C—H | C—H | H | CF3 | — | H | CF3CH2CO |
| 8-321 | C—Cl | N | C—H | C—H | H | CF3 | — | H | CH3S(O)CH2CO |
| 8-322 | C—Cl | N | C—H | C—H | H | CF3 | — | H | CH3SO2CH2CO |
| 8-323 | C—Cl | N | C—H | C—H | H | CF3 | — | H | CH3CH2NHCO |
| 8-324 | C—Cl | N | C—Cl | C—H | H | CF3 | — | H | CH3CO |
| 8-325 | C—Cl | N | C—Cl | C—H | H | CF3 | — | H | CH3CH2CO |
| 8-326 | C—Cl | N | C—Cl | C—H | H | CF3 | — | H | n-PrCO |
| 8-327 | C—Cl | N | C—Cl | C—H | H | CF3 | — | H | cyclo-PrCO |
| 8-328 | C—Cl | N | C—Cl | C—H | H | CF3 | — | H | cyclo-PrCH2CO |
| 8-329 | C—Cl | N | C—Cl | C—H | H | CF3 | — | H | CF3CH2CO |
| 8-330 | C—Cl | N | C—Cl | C—H | H | CF3 | — | H | CH3SCH2CO |
| 8-331 | C—Cl | N | C—Cl | C—H | H | CF3 | — | H | CH3S(O)CH2CO |
| 8-332 | C—Cl | N | C—Cl | C—H | H | CF3 | — | H | CH3SO2CH2CO |
| 8-333 | C—Cl | N | C—Cl | C—H | H | CF3 | — | H | CH3CH2NHCO |
| 8-334 | C—CF3 | N | C—H | C—H | H | CF3 | — | H | CH3CO |
| 8-335 | C—CF3 | N | C—H | C—H | H | CF3 | — | H | CH3CH2CO |
| 8-336 | C—CF3 | N | C—H | C—H | H | CF3 | — | H | n-PrCO |
| 8-337 | C—CF3 | N | C—H | C—H | H | CF3 | — | H | cyclo-PrCO |
| 8-338 | C—CF3 | N | C—H | C—H | H | CF3 | — | H | cyclo-PrCH2CO |
| 8-339 | C—CF3 | N | C—H | C—H | H | CF3 | — | H | CF3CH2CO |
| 8-340 | C—CF3 | N | C—H | C—H | H | CF3 | — | H | CH3SCH2CO |
| 8-341 | C—CF3 | N | C—H | C—H | H | CF3 | — | H | CH3S(O)CH2CO |
| 8-342 | C—CF3 | N | C—H | C—H | H | CF3 | — | H | CH3SO2CH2CO |
| 8-343 | C—CF3 | N | C—H | C—H | H | CF3 | — | H | CH3CH2NHCO |
| 8-344 | C—CF3 | N | C—CF3 | C—H | H | CF3 | — | H | CH3CO |
| 8-345 | C—CF3 | N | C—CF3 | C—H | H | CF3 | — | H | CH3CH2CO |
| 8-346 | C—CF3 | N | C—CF3 | C—H | H | CF3 | — | H | n-PrCO |
| 8-347 | C—CF3 | N | C—CF3 | C—H | H | CF3 | — | H | cyclo-PrCO |
| 8-348 | C—CF3 | N | C—CF3 | C—H | H | CF3 | — | H | cyclo-PrCH2CO |
| 8-349 | C—CF3 | N | C—CF3 | C—H | H | CF3 | — | H | CF3CH2CO |
| 8-350 | C—CF3 | N | C—CF3 | C—H | H | CF3 | — | H | CH3SCH2CO |
| 8-351 | C—CF3 | N | C—CF3 | C—H | H | CF3 | — | H | CH3S(O)CH2CO |
| 8-352 | C—CF3 | N | C—CF3 | C—H | H | CF3 | — | H | CH3SO2CH2CO |
| 8-353 | C—CF3 | N | C—CF3 | C—H | H | CF3 | — | H | CH3CH2NHCO |
| 8-354 | C—CF3 | C—H | C—H | N | H | CF3 | — | H | CH3CO |
| 8-355 | C—CF3 | C—H | C—H | N | H | CF3 | — | H | CH3CH2CO |
| 8-356 | C—CF3 | C—H | C—H | N | H | CF3 | — | H | n-PrCO |
| 8-357 | C—CF3 | C—H | C—H | N | H | CF3 | — | H | cyclo-PrCO |
| 8-358 | C—CF3 | C—H | C—H | N | H | CF3 | — | H | cyclo-PrCH2CO |
| 8-359 | C—CF3 | C—H | C—H | N | H | CF3 | — | H | CF3CH2CO |
| 8-360 | C—CF3 | C—H | C—H | N | H | CF3 | — | H | CH3SCH2CO |
| 8-361 | C—CF3 | C—H | C—H | N | H | CF3 | — | H | CH3S(O)CH2CO |
| 8-362 | C—CF3 | C—H | C—H | N | H | CF3 | — | H | CH3SO2CH2CO |
| 8-363 | C—CF3 | C—H | C—H | N | H | CF3 | — | H | CH3CH2NHCO |
| 8-364 | C—H | C—H | C—CF3 | N | H | CF3 | — | H | CH3CO |
| 8-365 | C—H | C—H | C—CF3 | N | H | CF3 | — | H | CH3CH2CO |
| 8-366 | C—H | C—H | C—CF3 | N | H | CF3 | — | H | n-PrCO |
| 8-367 | C—H | C—H | C—CF3 | N | H | CF3 | — | H | cyclo-PrCO |
| 8-368 | C—H | C—H | C—CF3 | N | H | CF3 | — | H | cyclo-PrCH2CO |
| 8-369 | C—H | C—H | C—CF3 | N | H | CF3 | — | H | CF3CH2CO |
| 8-370 | C—H | C—H | C—CF3 | N | H | CF3 | — | H | CH3SCH2CO |
| 8-371 | C—H | C—H | C—CF3 | N | H | CF3 | — | H | CH3S(O)CH2CO |
| 8-372 | C—H | C—H | C—CF3 | N | H | CF3 | — | H | CH3SO2CH2CO |
| 8-373 | C—H | C—H | C—CF3 | N | H | CF3 | — | H | CH3CH2NHCO |
| 8-374 | C—CF3 | C—H | C—Cl | N | H | CF3 | — | H | CH3CO |
| 8-375 | C—CF3 | C—H | C—Cl | N | H | CF3 | — | H | CH3CH2CO |
| 8-376 | C—CF3 | C—H | C—Cl | N | H | CF3 | — | H | n-PrCO |
| 8-377 | C—CF3 | C—H | C—Cl | N | H | CF3 | — | H | cyclo-PrCO |
| 8-378 | C—CF3 | C—H | C—Cl | N | H | CF3 | — | H | cyclo-PrCH2CO |
| 8-379 | C—CF3 | C—H | C—Cl | N | H | CF3 | — | H | CF3CH2CO |
| 8-380 | C—CF3 | C—H | C—Cl | N | H | CF3 | — | H | CH3SCH2CO |
| 8-381 | C—CF3 | C—H | C—Cl | N | H | CF3 | — | H | CH3S(O)CH2CO |
| 8-382 | C—CF3 | C—H | C—Cl | N | H | CF3 | — | H | CH3SO2CH2CO |
| 8-383 | C—CF3 | C—H | C—Cl | N | H | CF3 | — | H | CH3CH2NHCO |
| 8-384 | C—CF3 | C—H | C—Br | N | H | CF3 | — | H | CH3CO |
| 8-385 | C—CF3 | C—H | C—Br | N | H | CF3 | — | H | CH3CH2CO |
| 8-386 | C—CF3 | C—H | C—Br | N | H | CF3 | — | H | n-PrCO |
| 8-387 | C—CF3 | C—H | C—Br | N | H | CF3 | — | H | cyclo-PrCO |
| 8-388 | C—CF3 | C—H | C—Br | N | H | CF3 | — | H | cyclo-PrCH2CO |
| 8-389 | C—CF3 | C—H | C—Br | N | H | CF3 | — | H | CF3CH2CO |
| 8-390 | C—CF3 | C—H | C—Br | N | H | CF3 | — | H | CH3SCH2CO |
| 8-391 | C—CF3 | C—H | C—Br | N | H | CF3 | — | H | CH3S(O)CH2CO |
| 8-392 | C—CF3 | C—H | C—Br | N | H | CF3 | — | H | CH3SO2CH2CO |
| 8-393 | C—CF3 | C—H | C—Br | N | H | CF3 | — | H | CH3CH2NHCO |
| 8-394 | C—CF3 | C—H | C—CF3 | N | H | CF3 | — | H | CH3CO |
| 8-395 | C—CF3 | C—H | C—CF3 | N | H | CF3 | — | H | CH3CH2CO |
| 8-396 | C—CF3 | C—H | C—CF3 | N | H | CF3 | — | H | n-PrCO |
| 8-397 | C—CF3 | C—H | C—CF3 | N | H | CF3 | — | H | cyclo-PrCO |
| 8-398 | C—CF3 | C—H | C—CF3 | N | H | CF3 | — | H | cyclo-PrCH2CO |
| 8-399 | C—CF3 | C—H | C—CF3 | N | H | CF3 | — | H | CF3CH2CO |
| 8-400 | C—CF3 | C—H | C—CF3 | N | H | CF3 | — | H | CH3SCH2CO |
| 8-401 | C—CF3 | C—H | C—CF3 | N | H | CF3 | — | H | CH3S(O)CH2CO |
| 8-402 | C—CF3 | C—H | C—CF3 | N | H | CF3 | — | H | CH3SO2CH2CO |
| 8-403 | C—CF3 | C—H | C—CF3 | N | H | CF3 | — | H | CH3CH2NHCO |
| 8-404 | C—Cl | C—H | C—H | C—H | H | CF3 | CH2 | H | CH3CO |
| 8-405 | C—Cl | C—H | C—H | C—H | H | CF3 | CH2 | H | CH3CH2CO |
| 8-406 | C—Cl | C—H | C—H | C—H | H | CF3 | CH2 | H | n-PrCO |
| 8-407 | C—Cl | C—H | C—H | C—H | H | CF3 | CH2 | H | cyclo-PrCO |
| 8-408 | C—Cl | C—H | C—H | C—H | H | CF3 | CH2 | H | cyclo-PrCH2CO |
| 8-409 | C—Cl | C—H | C—H | C—H | H | CF3 | CH2 | H | CF3CH2CO |
| 8-410 | C—Cl | C—H | C—H | C—H | H | CF3 | CH2 | H | CH3SCH2CO |
| 8-411 | C—Cl | C—H | C—H | C—H | H | CF3 | CH2 | H | CH3S(O)CH2CO |
| 8-412 | C—Cl | C—H | C—H | C—H | H | CF3 | CH2 | H | CH3SO2CH2CO |
| 8-413 | C—Cl | C—H | C—H | C—H | H | CF3 | CH2 | H | CH3CH2NHCO |
| 8-414 | C—Cl | C—H | C—H | C—H | F | CF3 | CH2 | H | CH3CO |
| 8-415 | C—Cl | C—H | C—H | C—H | F | CF3 | CH2 | H | CH3CH2CO |
| 8-416 | C—Cl | C—H | C—H | C—H | F | CF3 | CH2 | H | n-PrCO |
| 8-417 | C—Cl | C—H | C—H | C—H | F | CF3 | CH2 | H | cyclo-PrCO |
| 8-418 | C—Cl | C—H | C—H | C—H | F | CF3 | CH2 | H | cyclo-PrCH2CO |
| 8-419 | C—Cl | C—H | C—H | C—H | F | CF3 | CH2 | H | CF3CH2CO |
| 8-420 | C—Cl | C—H | C—H | C—H | F | CF3 | CH2 | H | CH3SCH2CO |
| 8-421 | C—Cl | C—H | C—H | C—H | F | CF3 | CH2 | H | CH3S(O)CH2CO |
| 8-422 | C—Cl | C—H | C—H | C—H | F | CF3 | CH2 | H | CH3SO2CH2CO |
| 8-423 | C—Cl | C—H | C—H | C—H | F | CF3 | CH2 | H | CH3CH2NHCO |
| 8-424 | C—H | C—H | C—Cl | C—H | F | CF3 | CH2 | H | CH3CO |
| 8-425 | C—H | C—H | C—Cl | C—H | F | CF3 | CH2 | H | CH3CH2CO |
| 8-426 | C—H | C—H | C—Cl | C—H | F | CF3 | CH2 | H | n-PrCO |
| 8-427 | C—H | C—H | C—Cl | C—H | F | CF3 | CH2 | H | cyclo-PrCO |
| 8-428 | C—H | C—H | C—Cl | C—H | F | CF3 | CH2 | H | cyclo-PrCH2CO |
| 8-429 | C—H | C—H | C—Cl | C—H | F | CF3 | CH2 | H | CF3CH2CO |
| 8-430 | C—H | C—H | C—Cl | C—H | F | CF3 | CH2 | H | CH3SCH2CO |
| 8-431 | C—H | C—H | C—Cl | C—H | F | CF3 | CH2 | H | CH3S(O)CH2CO |
| 8-432 | C—H | C—H | C—Cl | C—H | F | CF3 | CH2 | H | CH3SO2CH2CO |
| 8-433 | C—H | C—H | C—Cl | C—H | F | CF3 | CH2 | H | CH3CH2NHCO |
| 8-434 | C—Cl | C—H | C—Cl | C—H | H | CF3 | CH2 | H | H |
| 8-435 | C—Cl | C—H | C—Cl | C—H | H | CF3 | CH2 | H | HCO |
| 8-436 | C—Cl | C—H | C—Cl | C—H | H | CF3 | CH2 | H | CH3CO |
| 8-437 | C—Cl | C—H | C—Cl | C—H | H | CF3 | CH2 | H | CH3CH2CO |
| 8-438 | C—Cl | C—H | C—Cl | C—H | H | CF3 | CH2 | H | n-PrCO |
| 8-439 | C—Cl | C—H | C—Cl | C—H | H | CF3 | CH2 | H | iso-PrCO |
| 8-440 | C—Cl | C—H | C—Cl | C—H | H | CF3 | CH2 | H | cyclo-PrCO |
| 8-441 | C—Cl | C—H | C—Cl | C—H | H | CF3 | CH2 | H | n-BuCO |
| 8-442 | C—Cl | C—H | C—Cl | C—H | H | CF3 | CH2 | H | tert-BuCO |
| 8-443 | C—Cl | C—H | C—Cl | C—H | H | CF3 | CH2 | H | iso-BuCO |
| 8-444 | C—Cl | C—H | C—Cl | C—H | H | CF3 | CH2 | H | cyclo-BuCO |
| 8-445 | C—Cl | C—H | C—Cl | C—H | H | CF3 | CH2 | H | (CH3)3CCH2CO |
| 8-446 | C—Cl | C—H | C—Cl | C—H | H | CF3 | CH2 | H | cyclo-PenCO |
| 8-447 | C—Cl | C—H | C—Cl | C—H | H | CF3 | CH2 | H | cyclo-HexCO |
| 8-448 | C—Cl | C—H | C—Cl | C—H | H | CF3 | CH2 | H | cyclo-PrCH2CO |
| 8-449 | C—Cl | C—H | C—Cl | C—H | H | CF3 | CH2 | H | CF3CO |
| 8-450 | C—Cl | C—H | C—Cl | C—H | H | CF3 | CH2 | H | CCl3CO |
| 8-451 | C—Cl | C—H | C—Cl | C—H | H | CF3 | CH2 | H | ClCH2CO |
| 8-452 | C—Cl | C—H | C—Cl | C—H | H | CF3 | CH2 | H | CF2ClCO |
| 8-453 | C—Cl | C—H | C—Cl | C—H | H | CF3 | CH2 | H | ClCH2CH2CO |
| 8-454 | C—Cl | C—H | C—Cl | C—H | H | CF3 | CH2 | H | HCF2CF2CO |
| 8-455 | C—Cl | C—H | C—Cl | C—H | H | CF3 | CH2 | H | CF3CH2CO |
| 8-456 | C—Cl | C—H | C—Cl | C—H | H | CF3 | CH2 | H | (CF3)2CHCO |
| 8-457 | C—Cl | C—H | C—Cl | C—H | H | CF3 | CH2 | H | HCF2CO |
| 8-458 | C—Cl | C—H | C—Cl | C—H | H | CF3 | CH2 | H | ClCH2CO |
| 8-459 | C—Cl | C—H | C—Cl | C—H | H | CF3 | CH2 | H | 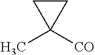 |
| 8-460 | C—Cl | C—H | C—Cl | C—H | H | CF3 | CH2 | H | 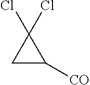 |
| 8-461 | C—Cl | C—H | C—Cl | C—H | H | CF3 | CH2 | H | 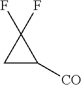 |
| 8-462 | C—Cl | C—H | C—Cl | C—H | H | CF3 | CH2 | H | 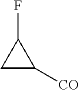 |
| 8-463 | C—Cl | C—H | C—Cl | C—H | H | CF3 | CH2 | H | 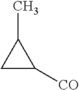 |
| 8-464 | C—Cl | C—H | C—Cl | C—H | H | CF3 | CH2 | H | 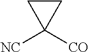 |
| 8-465 | C—Cl | C—H | C—Cl | C—H | H | CF3 | CH2 | H | CH2═CH—CO |
| 8-466 | C—Cl | C—H | C—Cl | C—H | H | CF3 | CH2 | H | CH3CH═CH—CO |
| 8-467 | C—Cl | C—H | C—Cl | C—H | H | CF3 | CH2 | H | CH2═C(CH3)—CO |
| 8-468 | C—Cl | C—H | C—Cl | C—H | H | CF3 | CH2 | H | CH3CH═C(CH3)—CO |
| 8-469 | C—Cl | C—H | C—Cl | C—H | H | CF3 | CH2 | H | (CH3)2C═CH—CO |
| 8-470 | C—Cl | C—H | C—Cl | C—H | H | CF3 | CH2 | H | CH2═C(Cl)—CO |
| 8-471 | C—Cl | C—H | C—Cl | C—H | H | CF3 | CH2 | H | ClCH═C(CH3)—CO |
| 8-472 | C—Cl | C—H | C—Cl | C—H | H | CF3 | CH2 | H | CH2═CHCH2—CO |
| 8-473 | C—Cl | C—H | C—Cl | C—H | H | CF3 | CH2 | H | HCC—CO |
| 8-474 | C—Cl | C—H | C—Cl | C—H | H | CF3 | CH2 | H | CH3CC—CO |
| 8-475 | C—Cl | C—H | C—Cl | C—H | H | CF3 | CH2 | H | NCCH2—CO |
| 8-476 | C—Cl | C—H | C—Cl | C—H | H | CF3 | CH2 | H | CH3OCH2—CO |
| 8-477 | C—Cl | C—H | C—Cl | C—H | H | CF3 | CH2 | H | C2H5OCH2—CO |
| 8-478 | C—Cl | C—H | C—Cl | C—H | H | CF3 | CH2 | H | CH3OCH2CH2—CO |
| 8-479 | C—Cl | C—H | C—Cl | C—H | H | CF3 | CH2 | H | CH3CH2OCH2CH2—CO |
| 8-480 | C—Cl | C—H | C—Cl | C—H | H | CF3 | CH2 | H | CH3SCH2CO |
| 8-481 | C—Cl | C—H | C—Cl | C—H | H | CF3 | CH2 | H | CH3S(O)CH2CO |
| 8-482 | C—Cl | C—H | C—Cl | C—H | H | CF3 | CH2 | H | CH3SO2CH2CO |
| 8-483 | C—Cl | C—H | C—Cl | C—H | H | CF3 | CH2 | H | CH3O(CO)CH2—CO |
| 8-484 | C—Cl | C—H | C—Cl | C—H | H | CF3 | CH2 | H | CH3(CO)OCH2—CO |
| 8-485 | C—Cl | C—H | C—Cl | C—H | H | CF3 | CH2 | H | CH3O(CO)—CO |
| 8-486 | C—Cl | C—H | C—Cl | C—H | H | CF3 | CH2 | H | CF3O(CO)—CO |
| 8-487 | C—Cl | C—H | C—Cl | C—H | H | CF3 | CH2 | H | CH3(CO)CH2—CO |
| 8-488 | C—Cl | C—H | C—Cl | C—H | H | CF3 | CH2 | H | (CH3)2NCH2—CO |
| 8-489 | C—Cl | C—H | C—Cl | C—H | H | CF3 | CH2 | H | PhCO |
| 8-490 | C—Cl | C—H | C—Cl | C—H | H | CF3 | CH2 | H | PhCH2CO |
| 8-491 | C—Cl | C—H | C—Cl | C—H | H | CF3 | CH2 | H | 2-py—CO |
| 8-492 | C—Cl | C—H | C—Cl | C—H | H | CF3 | CH2 | H | 3-py—CO |
| 8-493 | C—Cl | C—H | C—Cl | C—H | H | CF3 | CH2 | H | 4-py—CO |
| 8-494 | C—Cl | C—H | C—Cl | C—H | H | CF3 | CH2 | H | 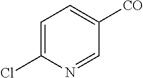 |
| 8-495 | C—Cl | C—H | C—Cl | C—H | H | CF3 | CH2 | H | 2-F—PhCO |
| 8-496 | C—Cl | C—H | C—Cl | C—H | H | CF3 | CH2 | H | 3-F—PhCO |
| 8-497 | C—Cl | C—H | C—Cl | C—H | H | CF3 | CH2 | H | 4-F—PhCO |
| 8-498 | C—Cl | C—H | C—Cl | C—H | H | CF3 | CH2 | H | 2-Cl—PhCO |
| 8-499 | C—Cl | C—H | C—Cl | C—H | H | CF3 | CH2 | H | 3-Cl—PhCO |
| 8-500 | C—Cl | C—H | C—Cl | C—H | H | CF3 | CH2 | H | 4-Cl—PhCO |
| 8-501 | C—Cl | C—H | C—Cl | C—H | H | CF3 | CH2 | H | 2-Br—PhCO |
| 8-502 | C—Cl | C—H | C—Cl | C—H | H | CF3 | CH2 | H | 2-CF3—PhCO |
| 8-503 | C—Cl | C—H | C—Cl | C—H | H | CF3 | CH2 | H | 2-CH3—PhCO |
| 8-504 | C—Cl | C—H | C—Cl | C—H | H | CF3 | CH2 | H | 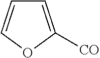 |
| 8-505 | C—Cl | C—H | C—Cl | C—H | H | CF3 | CH2 | H | 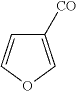 |
| 8-506 | C—Cl | C—H | C—Cl | C—H | H | CF3 | CH2 | H | 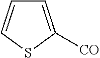 |
| 8-507 | C—Cl | C—H | C—Cl | C—H | H | CF3 | CH2 | H | 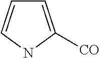 |
| 8-508 | C—Cl | C—H | C—Cl | C—H | H | CF3 | CH2 | H | 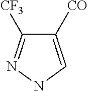 |
| 8-509 | C—Cl | C—H | C—Cl | C—H | H | CF3 | CH2 | H | 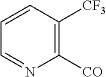 |
| 8-510 | C—Cl | C—H | C—Cl | C—H | H | CF3 | CH2 | H | 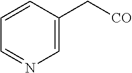 |
| 8-511 | C—Cl | C—H | C—Cl | C—H | H | CF3 | CH2 | H | 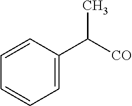 |
| 8-512 | C—Cl | C—H | C—Cl | C—H | H | CF3 | CH2 | H | CH3NHCO |
| 8-513 | C—Cl | C—H | C—Cl | C—H | H | CF3 | CH2 | H | CH3CH2NHCO |
| 8-514 | C—Cl | C—H | C—Cl | C—H | H | CF3 | CH2 | H | CF3CH2NHCO |
| 8-515 | C—Cl | C—H | C—Cl | C—H | H | CF3 | CH2 | H | n-PrNHCO |
| 8-516 | C—Cl | C—H | C—Cl | C—H | H | CF3 | CH2 | H | iso-PrNHCO |
| 8-517 | C—Cl | C—H | C—Cl | C—H | H | CF3 | CH2 | H | cyclo-PrNHCO |
| 8-518 | C—Cl | C—H | C—Cl | C—H | H | CF3 | CH2 | H | n-BuNHCO |
| 8-519 | C—Cl | C—H | C—Cl | C—H | H | CF3 | CH2 | H | tert-BuNHCO |
| 8-520 | C—Cl | C—H | C—Cl | C—H | H | CF3 | CH2 | H | cyclo-BuNHCO |
| 8-521 | C—Cl | C—H | C—Cl | C—H | H | CF3 | CH2 | H | cyclo-PenNHCO |
| 8-522 | C—Cl | C—H | C—Cl | C—H | H | CF3 | CH2 | H | cyclo-HexNHCO |
| 8-523 | C—Cl | C—H | C—Cl | C—H | H | CF3 | CH2 | H | CH2═CHCH2NHCO |
| 8-524 | C—Cl | C—H | C—Cl | C—H | H | CF3 | CH2 | H | HCCCH2NHCO |
| 8-525 | C—Cl | C—H | C—Cl | C—H | H | CF3 | CH2 | H | cyclo-PrCH2NHCO |
| 8-526 | C—Cl | C—H | C—Cl | C—H | H | CF3 | CH2 | H | NCCH2NHCO |
| 8-527 | C—Cl | C—H | C—Cl | C—H | H | CF3 | CH2 | H | CF3(CH3)CHNHCO |
| 8-528 | C—Cl | C—H | C—Cl | C—H | H | CF3 | CH2 | H | CF3(CH3)2CNCO |
| 8-529 | C—Cl | C—H | C—Cl | C—H | H | CF3 | CH2 | H | NCCH2OCO |
| 8-530 | C—Cl | C—H | C—Cl | C—H | H | CF3 | CH2 | H | FCH2CH2NHCO |
| 8-531 | C—Cl | C—H | C—Cl | C—H | H | CF3 | CH2 | H | CF3CH2CH2NHCO |
| 8-532 | C—Cl | C—H | C—Cl | C—H | H | CF3 | CH2 | H | HCF2CF2CH2NHCO |
| 8-533 | C—Cl | C—H | C—Cl | C—H | H | CF3 | CH2 | H | 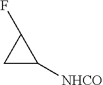 |
| 8-534 | C—Cl | C—H | C—Cl | C—H | H | CF3 | CH2 | H | 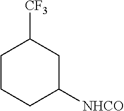 |
| 8-535 | C—Cl | C—H | C—Cl | C—H | H | CF3 | CH2 | H | EtO2CCH2NHCO |
| 8-536 | C—Cl | C—H | C—Cl | C—H | H | CF3 | CH2 | H | 2-CF3—PhNHCO |
| 8-537 | C—Cl | C—H | C—Cl | C—H | H | CF3 | CH2 | H | 3-CF3—PhNHCO |
| 8-538 | C—Cl | C—H | C—Cl | C—H | H | CF3 | CH2 | H | 4-CF3—PhNHCO |
| 8-539 | C—Cl | C—H | C—Cl | C—H | H | CF3 | CH2 | H | 2-py—NHCO |
| 8-540 | C—Cl | C—H | C—Cl | C—H | H | CF3 | CH2 | H | 3-py—NHCO |
| 8-541 | C—Cl | C—H | C—Cl | C—H | H | CF3 | CH2 | H | 4-py—NHCO |
| 8-542 | C—Cl | C—H | C—Cl | C—H | H | CF3 | CH2 | H | 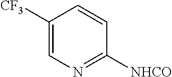 |
| 8-543 | C—Cl | C—H | C—Cl | C—H | H | CF3 | CH2 | H | 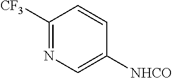 |
| 8-544 | C—Cl | C—H | C—Cl | C—H | H | CF3 | CH2 | H | PhCH2HNCO |
| 8-545 | C—Cl | C—H | C—Cl | C—H | H | CF3 | CH2 | H | 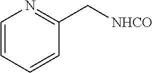 |
| 8-546 | C—Cl | C—H | C—Cl | C—H | H | CF3 | CH2 | H | (CH3)2NCO |
| 8-547 | C—Cl | C—H | C—Cl | C—H | H | CF3 | CH2 | H | pyrrolidineCO |
| 8-548 | C—Cl | C—H | C—Cl | C—H | H | CF3 | CH2 | H | piperidineCO |
| 8-549 | C—Cl | C—H | C—Cl | C—H | H | CF3 | CH2 | H | morpholineCO |
| 8-550 | C—Cl | C—H | C—Cl | C—H | H | CF3 | CH2 | H | CH3OCONH |
| 8-551 | C—Cl | C—H | C—Cl | C—H | H | CF3 | CH2 | H | CH3CH2OCO |
| 8-552 | C—Cl | C—H | C—Cl | C—H | H | CF3 | CH2 | H | n-PrOCO |
| 8-553 | C—Cl | C—H | C—Cl | C—H | H | CF3 | CH2 | H | cyclo-PrOCO |
| 8-554 | C—Cl | C—H | C—Cl | C—H | H | CF3 | CH2 | H | CF3CH2OCO |
| 8-555 | C—Cl | C—H | C—Cl | C—H | H | CF3 | CH2 | H | CH2═CHCH2OCO |
| 8-556 | C—Cl | C—H | C—Cl | C—H | H | CF3 | CH2 | H | HCCCH2OCO |
| 8-557 | C—Cl | C—H | C—Cl | C—H | H | CF3 | CH2 | H | PhOCO |
| 8-558 | C—Cl | C—H | C—Cl | C—H | H | CF3 | CH2 | H | PhCH2OCO |
| 8-559 | C—Cl | C—H | C—Cl | C—H | H | CF3 | CH2 | H | tert-BuOCO |
| 8-560 | C—Cl | C—H | C—Cl | C—H | H | CF3 | CH2 | H | CH3SO2 |
| 8-561 | C—Cl | C—H | C—Cl | C—H | H | CF3 | CH2 | H | CH3CH2SO2 |
| 8-562 | C—Cl | C—H | C—Cl | C—H | H | CF3 | CH2 | H | CF3SO2 |
| 8-563 | C—Cl | C—H | C—Cl | C—H | H | CF3 | CH2 | H | CF3CH2SO2 |
| 8-564 | C—Cl | C—H | C—Cl | C—H | H | CF3 | CH2 | H | PhSO2 |
| 8-565 | C—Cl | C—H | C—Cl | C—H | H | CF3 | CH2 | H | CH3NHCONHSO2 |
| 8-566 | C—Cl | C—H | C—Cl | C—H | H | CF3 | CH2 | H | (CH3)2NCONHSO2 |
| 8-567 | C—Cl | C—H | C—Cl | C—H | H | CF3 | CH2 | H | CH3CS |
| 8-568 | C—Cl | C—H | C—Cl | C—H | H | CF3 | CH2 | H | CH3CH2CS |
| 8-569 | C—Cl | C—H | C—Cl | C—H | H | CF3 | CH2 | H | cyclo-PrCS |
| 8-570 | C—Cl | C—H | C—Cl | C—H | H | CF3 | CH2 | H | CF3CH2CS |
| 8-571 | C—Cl | C—H | C—Br | C—H | H | CF3 | CH2 | H | CH3CO |
| 8-572 | C—Cl | C—H | C—Br | C—H | H | CF3 | CH2 | H | CH3CH2CO |
| 8-573 | C—Cl | C—H | C—Br | C—H | H | CF3 | CH2 | H | n-PrCO |
| 8-574 | C—Cl | C—H | C—Br | C—H | H | CF3 | CH2 | H | cyclo-PrCO |
| 8-575 | C—Cl | C—H | C—Br | C—H | H | CF3 | CH2 | H | cyclo-PrCH2CO |
| 8-576 | C—Cl | C—H | C—Br | C—H | H | CF3 | CH2 | H | CF3CH2CO |
| 8-577 | C—Cl | C—H | C—Br | C—H | H | CF3 | CH2 | H | CH3SCH2CO |
| 8-578 | C—Cl | C—H | C—Br | C—H | H | CF3 | CH2 | H | CH3S(O)CH2CO |
| 8-579 | C—Cl | C—H | C—Br | C—H | H | CF3 | CH2 | H | CH3SO2CH2CO |
| 8-580 | C—Cl | C—H | C—Br | C—H | H | CF3 | CH2 | H | CH3CH2NHCO |
| 8-581 | C—Br | C—H | C—Br | C—H | H | CF3 | CH2 | H | CH3CO |
| 8-582 | C—Br | C—H | C—Br | C—H | H | CF3 | CH2 | H | CH3CH2CO |
| 8-583 | C—Br | C—H | C—Br | C—H | H | CF3 | CH2 | H | n-PrCO |
| 8-584 | C—Br | C—H | C—Br | C—H | H | CF3 | CH2 | H | cyclo-PrCO |
| 8-585 | C—Br | C—H | C—Br | C—H | H | CF3 | CH2 | H | cyclo-PrCH2CO |
| 8-586 | C—Br | C—H | C—Br | C—H | H | CF3 | CH2 | H | CF3CH2CO |
| 8-587 | C—Br | C—H | C—Br | C—H | H | CF3 | CH2 | H | CH3SCH2CO |
| 8-588 | C—Br | C—H | C—Br | C—H | H | CF3 | CH2 | H | CH3S(O)CH2CO |
| 8-589 | C—Br | C—H | C—Br | C—H | H | CF3 | CH2 | H | CH3SO2CH2CO |
| 8-590 | C—Br | C—H | C—Br | C—H | H | CF3 | CH2 | H | CH3CH2NHCO |
| 8-591 | C—Cl | C—F | C—Cl | C—H | H | CF3 | CH2 | H | CH3CO |
| 8-592 | C—Cl | C—F | C—Cl | C—H | H | CF3 | CH2 | H | CH3CH2CO |
| 8-593 | C—Cl | C—F | C—Cl | C—H | H | CF3 | CH2 | H | n-PrCO |
| 8-594 | C—Cl | C—F | C—Cl | C—H | H | CF3 | CH2 | H | cyclo-PrCO |
| 8-595 | C—Cl | C—F | C—Cl | C—H | H | CF3 | CH2 | H | cyclo-PrCH2CO |
| 8-596 | C—Cl | C—F | C—Cl | C—H | H | CF3 | CH2 | H | CF3CH2CO |
| 8-598 | C—Cl | C—F | C—Cl | C—H | H | CF3 | CH2 | H | CH3S(O)CH2CO |
| 8-599 | C—Cl | C—F | C—Cl | C—H | H | CF3 | CH2 | H | CH3SO2CH2CO |
| 8-600 | C—Cl | C—F | C—Cl | C—H | H | CF3 | CH2 | H | CH3CH2NHCO |
| 8-601 | C—Cl | C—Cl | C—Cl | C—H | H | CF3 | CH2 | H | CH3CO |
| 8-602 | C—Cl | C—Cl | C—Cl | C—H | H | CF3 | CH2 | H | CH3CH2CO |
| 8-603 | C—Cl | C—Cl | C—Cl | C—H | H | CF3 | CH2 | H | n-PrCO |
| 8-604 | C—Cl | C—Cl | C—Cl | C—H | H | CF3 | CH2 | H | cyclo-PrCO |
| 8-605 | C—Cl | C—Cl | C—Cl | C—H | H | CF3 | CH2 | H | cyclo-PrCH2CO |
| 8-606 | C—Cl | C—Cl | C—Cl | C—H | H | CF3 | CH2 | H | CF3CH2CO |
| 8-608 | C—Cl | C—Cl | C—Cl | C—H | H | CF3 | CH2 | H | CH3CH2OCH2CH2—CO |
| 8-609 | C—Cl | C—Cl | C—Cl | C—H | H | CF3 | CH2 | H | CH3SCH2CO |
| 8-610 | C—Cl | C—Cl | C—Cl | C—H | H | CF3 | CH2 | H | CH3S(O)CH2CO |
| 8-611 | C—Cl | C—Cl | C—Cl | C—H | H | CF3 | CH2 | H | CH3SO2CH2CO |
| 8-612 | C—Cl | C—Cl | C—Cl | C—H | H | CF3 | CH2 | H | CH3CH2NHCO |
| 8-613 | C—Cl | C—Br | C—Cl | C—H | H | CF3 | CH2 | H | CH3CO |
| 8-614 | C—Cl | C—Br | C—Cl | C—H | H | CF3 | CH2 | H | CH3CH2CO |
| 8-615 | C—Cl | C—Br | C—Cl | C—H | H | CF3 | CH2 | H | n-PrCO |
| 8-616 | C—Cl | C—Br | C—Cl | C—H | H | CF3 | CH2 | H | cyclo-PrCO |
| 8-617 | C—Cl | C—Br | C—Cl | C—H | H | CF3 | CH2 | H | cyclo-PrCH2CO |
| 8-618 | C—Cl | C—Br | C—Cl | C—H | H | CF3 | CH2 | H | CF3CH2CO |
| 8-619 | C—Cl | C—Br | C—Cl | C—H | H | CF3 | CH2 | H | CH3SCH2CO |
| 8-620 | C—Cl | C—Br | C—Cl | C—H | H | CF3 | CH2 | H | CH3S(O)CH2CO |
| 8-621 | C—Cl | C—Br | C—Cl | C—H | H | CF3 | CH2 | H | CH3SO2CH2CO |
| 8-622 | C—Cl | C—Br | C—Cl | C—H | H | CF3 | CH2 | H | CH3CH2NHCO |
| 8-623 | C—Cl | C—Cl | C—Br | C—H | H | CF3 | CH2 | H | CH3CO |
| 8-624 | C—Cl | C—Cl | C—Br | C—H | H | CF3 | CH2 | H | CH3CH2CO |
| 8-625 | C—Cl | C—Cl | C—Br | C—H | H | CF3 | CH2 | H | n-PrCO |
| 8-626 | C—Cl | C—Cl | C—Br | C—H | H | CF3 | CH2 | H | cyclo-PrCO |
| 8-627 | C—Cl | C—Cl | C—Br | C—H | H | CF3 | CH2 | H | cyclo-PrCH2CO |
| 8-628 | C—Cl | C—Cl | C—Br | C—H | H | CF3 | CH2 | H | CF3CH2CO |
| 8-629 | C—Cl | C—Cl | C—Br | C—H | H | CF3 | CH2 | H | CH3SCH2CO |
| 8-630 | C—Cl | C—Cl | C—Br | C—H | H | CF3 | CH2 | H | CH3S(O)CH2CO |
| 8-631 | C—Cl | C—Cl | C—Br | C—H | H | CF3 | CH2 | H | CH3SO2CH2CO |
| 8-632 | C—Cl | C—Cl | C—Br | C—H | H | CF3 | CH2 | H | CH3CH2NHCO |
| 8-633 | C—Cl | C—F | C—Cl | C—H | F | CF3 | CH2 | H | CH3CO |
| 8-634 | C—Cl | C—F | C—Cl | C—H | F | CF3 | CH2 | H | CH3CH2CO |
| 8-635 | C—Cl | C—F | C—Cl | C—H | F | CF3 | CH2 | H | n-PrCO |
| 8-636 | C—Cl | C—F | C—Cl | C—H | F | CF3 | CH2 | H | cyclo-PrCO |
| 8-637 | C—Cl | C—F | C—Cl | C—H | F | CF3 | CH2 | H | cyclo-PrCH2CO |
| 8-638 | C—Cl | C—F | C—Cl | C—H | F | CF3 | CH2 | H | CF3CH2CO |
| 8-639 | C—Cl | C—F | C—Cl | C—H | F | CF3 | CH2 | H | CH3SCH2CO |
| 8-640 | C—Cl | C—F | C—Cl | C—H | F | CF3 | CH2 | H | CH3S(O)CH2CO |
| 8-641 | C—Cl | C—F | C—Cl | C—H | F | CF3 | CH2 | H | CH3SO2CH2CO |
| 8-642 | C—Cl | C—F | C—Cl | C—H | F | CF3 | CH2 | H | CH3CH2NHCO |
| 8-643 | C—CF3 | C—H | C—H | C—H | H | CF3 | CH2 | H | CH3CO |
| 8-644 | C—CF3 | C—H | C—H | C—H | H | CF3 | CH2 | H | CH3CH2CO |
| 8-645 | C—CF3 | C—H | C—H | C—H | H | CF3 | CH2 | H | n-PrCO |
| 8-646 | C—CF3 | C—H | C—H | C—H | H | CF3 | CH2 | H | cyclo-PrCO |
| 8-647 | C—CF3 | C—H | C—H | C—H | H | CF3 | CH2 | H | cyclo-PrCH2CO |
| 8-648 | C—CF3 | C—H | C—H | C—H | H | CF3 | CH2 | H | CF3CH2CO |
| 8-649 | C—CF3 | C—H | C—H | C—H | H | CF3 | CH2 | H | CH3SCH2CO |
| 8-650 | C—CF3 | C—H | C—H | C—H | H | CF3 | CH2 | H | CH3S(O)CH2CO |
| 8-651 | C—CF3 | C—H | C—H | C—H | H | CF3 | CH2 | H | CH3SO2CH2CO |
| 8-652 | C—CF3 | C—H | C—H | C—H | H | CF3 | CH2 | H | CH3CH2NHCO |
| 8-653 | C—CF3 | C—F | C—H | C—H | H | CF3 | CH2 | H | CH3CO |
| 8-654 | C—CF3 | C—F | C—H | C—H | H | CF3 | CH2 | H | CH3CH2CO |
| 8-655 | C—CF3 | C—F | C—H | C—H | H | CF3 | CH2 | H | n-PrCO |
| 8-656 | C—CF3 | C—F | C—H | C—H | H | CF3 | CH2 | H | cyclo-PrCO |
| 8-657 | C—CF3 | C—F | C—H | C—H | H | CF3 | CH2 | H | cyclo-PrCH2CO |
| 8-658 | C—CF3 | C—F | C—H | C—H | H | CF3 | CH2 | H | CF3CH2CO |
| 8-659 | C—CF3 | C—F | C—H | C—H | H | CF3 | CH2 | H | CH3SCH2CO |
| 8-660 | C—CF3 | C—F | C—H | C—H | H | CF3 | CH2 | H | CH3S(O)CH2CO |
| 8-661 | C—CF3 | C—F | C—H | C—H | H | CF3 | CH2 | H | CH3SO2CH2CO |
| 8-662 | C—CF3 | C—F | C—H | C—H | H | CF3 | CH2 | H | CH3CH2NHCO |
| 8-663 | C—CF3 | C—H | C—H | C—H | F | CF3 | CH2 | H | CH3CO |
| 8-664 | C—CF3 | C—H | C—H | C—H | F | CF3 | CH2 | H | CH3CH2CO |
| 8-665 | C—CF3 | C—H | C—H | C—H | F | CF3 | CH2 | H | n-PrCO |
| 8-666 | C—CF3 | C—H | C—H | C—H | F | CF3 | CH2 | H | cyclo-PrCO |
| 8-667 | C—CF3 | C—H | C—H | C—H | F | CF3 | CH2 | H | cyclo-PrCH2CO |
| 8-668 | C—CF3 | C—H | C—H | C—H | F | CF3 | CH2 | H | CF3CH2CO |
| 8-669 | C—CF3 | C—H | C—H | C—H | F | CF3 | CH2 | H | CH3SCH2CO |
| 8-670 | C—CF3 | C—H | C—H | C—H | F | CF3 | CH2 | H | CH3S(O)CH2CO |
| 8-671 | C—CF3 | C—H | C—H | C—H | F | CF3 | CH2 | H | CH3SO2CH2CO |
| 8-672 | C—CF3 | C—H | C—H | C—H | F | CF3 | CH2 | H | CH3CH2NHCO |
| 8-673 | C—H | C—H | C—CF3 | C—H | F | CF3 | CH2 | H | CH3CO |
| 8-674 | C—H | C—H | C—CF3 | C—H | F | CF3 | CH2 | H | CH3CH2CO |
| 8-675 | C—H | C—H | C—CF3 | C—H | F | CF3 | CH2 | H | n-PrCO |
| 8-676 | C—H | C—H | C—CF3 | C—H | F | CF3 | CH2 | H | cyclo-PrCO |
| 8-677 | C—H | C—H | C—CF3 | C—H | F | CF3 | CH2 | H | cyclo-PrCH2CO |
| 8-678 | C—H | C—H | C—CF3 | C—H | F | CF3 | CH2 | H | CF3CH2CO |
| 8-679 | C—H | C—H | C—CF3 | C—H | F | CF3 | CH2 | H | CH3SCH2CO |
| 8-680 | C—H | C—H | C—CF3 | C—H | F | CF3 | CH2 | H | CH3S(O)CH2CO |
| 8-681 | C—H | C—H | C—CF3 | C—H | F | CF3 | CH2 | H | CH3SO2CH2CO |
| 8-682 | C—H | C—H | C—CF3 | C—H | F | CF3 | CH2 | H | CH3CH2NHCO |
| 8-683 | C—Cl | C—H | C—CF3 | C—H | H | CF3 | CH2 | H | CH3CO |
| 8-684 | C—Cl | C—H | C—CF3 | C—H | H | CF3 | CH2 | H | CH3CH2CO |
| 8-685 | C—Cl | C—H | C—CF3 | C—H | H | CF3 | CH2 | H | n-PrCO |
| 8-686 | C—Cl | C—H | C—CF3 | C—H | H | CF3 | CH2 | H | cyclo-PrCO |
| 8-687 | C—Cl | C—H | C—CF3 | C—H | H | CF3 | CH2 | H | cyclo-PrCH2CO |
| 8-688 | C—Cl | C—H | C—CF3 | C—H | H | CF3 | CH2 | H | CF3CH2CO |
| 8-689 | C—Cl | C—H | C—CF3 | C—H | H | CF3 | CH2 | H | CH3OCH2CH2—CO |
| 8-690 | C—Cl | C—H | C—CF3 | C—H | H | CF3 | CH2 | H | CH3CH2OCH2CH2—CO |
| 8-691 | C—Cl | C—H | C—CF3 | C—H | H | CF3 | CH2 | H | CH3SCH2CO |
| 8-692 | C—Cl | C—H | C—CF3 | C—H | H | CF3 | CH2 | H | CH3S(O)CH2CO |
| 8-693 | C—Cl | C—H | C—CF3 | C—H | H | CF3 | CH2 | H | CH3SO2CH2CO |
| 8-694 | C—Cl | C—H | C—CF3 | C—H | H | CF3 | CH2 | H | CH3CH2NHCO |
| 8-695 | C—CF3 | C—H | C—CF3 | C—H | H | CF3 | CH2 | H | CH3CO |
| 8-696 | C—CF3 | C—H | C—CF3 | C—H | H | CF3 | CH2 | H | CH3CH2CO |
| 8-697 | C—CF3 | C—H | C—CF3 | C—H | H | CF3 | CH2 | H | n-PrCO |
| 8-698 | C—CF3 | C—H | C—CF3 | C—H | H | CF3 | CH2 | H | cyclo-PrCO |
| 8-699 | C—CF3 | C—H | C—CF3 | C—H | H | CF3 | CH2 | H | cyclo-PrCH2CO |
| 8-700 | C—CF3 | C—H | C—CF3 | C—H | H | CF3 | CH2 | H | CF3CH2CO |
| 8-701 | C—CF3 | C—H | C—CF3 | C—H | H | CF3 | CH2 | H | CH3OCH2CH2—CO |
| 8-702 | C—CF3 | C—H | C—CF3 | C—H | H | CF3 | CH2 | H | CH3CH2OCH2CH2—CO |
| 8-703 | C—CF3 | C—H | C—CF3 | C—H | H | CF3 | CH2 | H | CH3SCH2CO |
| 8-704 | C—CF3 | C—H | C—CF3 | C—H | H | CF3 | CH2 | H | CH3S(O)CH2CO |
| 8-705 | C—CF3 | C—H | C—CF3 | C—H | H | CF3 | CH2 | H | CH3SO2CH2CO |
| 8-706 | C—CF3 | C—H | C—CF3 | C—H | H | CF3 | CH2 | H | CH3CH2NHCO |
| 8-707 | C—Cl | C—Cl | C—CF3 | C—H | H | CF3 | CH2 | H | CH3CO |
| 8-708 | C—Cl | C—Cl | C—CF3 | C—H | H | CF3 | CH2 | H | CH3CH2CO |
| 8-709 | C—Cl | C—Cl | C—CF3 | C—H | H | CF3 | CH2 | H | n-PrCO |
| 8-710 | C—Cl | C—Cl | C—CF3 | C—H | H | CF3 | CH2 | H | cyclo-PrCO |
| 8-711 | C—Cl | C—Cl | C—CF3 | C—H | H | CF3 | CH2 | H | cyclo-PrCH2CO |
| 8-712 | C—Cl | C—Cl | C—CF3 | C—H | H | CF3 | CH2 | H | CF3CH2CO |
| 8-713 | C—Cl | C—Cl | C—CF3 | C—H | H | CF3 | CH2 | H | CH3SCH2CO |
| 8-714 | C—Cl | C—Cl | C—CF3 | C—H | H | CF3 | CH2 | H | CH3S(O)CH2CO |
| 8-715 | C—Cl | C—Cl | C—CF3 | C—H | H | CF3 | CH2 | H | CH3SO2CH2CO |
| 8-716 | C—Cl | C—Cl | C—CF3 | C—H | H | CF3 | CH2 | H | CH3CH2NHCO |
| 8-717 | C—Cl | N | C—H | C—H | H | CF3 | CH2 | H | CH3CO |
| 8-718 | C—Cl | N | C—H | C—H | H | CF3 | CH2 | H | CH3CH2CO |
| 8-719 | C—Cl | N | C—H | C—H | H | CF3 | CH2 | H | n-PrCO |
| 8-720 | C—Cl | N | C—H | C—H | H | CF3 | CH2 | H | cyclo-PrCO |
| 8-721 | C—Cl | N | C—H | C—H | H | CF3 | CH2 | H | cyclo-PrCH2CO |
| 8-722 | C—Cl | N | C—H | C—H | H | CF3 | CH2 | H | CF3CH2CO |
| 8-723 | C—Cl | N | C—H | C—H | H | CF3 | CH2 | H | CH3SCH2CO |
| 8-724 | C—Cl | N | C—H | C—H | H | CF3 | CH2 | H | CH3S(O)CH2CO |
| 8-725 | C—Cl | N | C—H | C—H | H | CF3 | CH2 | H | CH3SO2CH2CO |
| 8-726 | C—Cl | N | C—H | C—H | H | CF3 | CH2 | H | CH3CH2NHCO |
| 8-727 | C—Cl | N | C—Cl | C—H | H | CF3 | CH2 | H | CH3CO |
| 8-728 | C—Cl | N | C—Cl | C—H | H | CF3 | CH2 | H | CH3CH2CO |
| 8-729 | C—Cl | N | C—Cl | C—H | H | CF3 | CH2 | H | n-PrCO |
| 8-730 | C—Cl | N | C—Cl | C—H | H | CF3 | CH2 | H | cyclo-PrCO |
| 8-731 | C—Cl | N | C—Cl | C—H | H | CF3 | CH2 | H | cyclo-PrCH2CO |
| 8-732 | C—Cl | N | C—Cl | C—H | H | CF3 | CH2 | H | CF3CH2CO |
| 8-733 | C—Cl | N | C—Cl | C—H | H | CF3 | CH2 | H | CH3SCH2CO |
| 8-734 | C—Cl | N | C—Cl | C—H | H | CF3 | CH2 | H | CH3S(O)CH2CO |
| 8-735 | C—Cl | N | C—Cl | C—H | H | CF3 | CH2 | H | CH3SO2CH2CO |
| 8-736 | C—Cl | N | C—Cl | C—H | H | CF3 | CH2 | H | CH3CH2NHCO |
| 8-737 | C—CF3 | N | C—H | C—H | H | CF3 | CH2 | H | CH3CO |
| 8-738 | C—CF3 | N | C—H | C—H | H | CF3 | CH2 | H | CH3CH2CO |
| 8-739 | C—CF3 | N | C—H | C—H | H | CF3 | CH2 | H | n-PrCO |
| 8-740 | C—CF3 | N | C—H | C—H | H | CF3 | CH2 | H | cyclo-PrCO |
| 8-741 | C—CF3 | N | C—H | C—H | H | CF3 | CH2 | H | cyclo-PrCH2CO |
| 8-742 | C—CF3 | N | C—H | C—H | H | CF3 | CH2 | H | CF3CH2CO |
| 8-743 | C—CF3 | N | C—H | C—H | H | CF3 | CH2 | H | CH3SCH2CO |
| 8-744 | C—CF3 | N | C—H | C—H | H | CF3 | CH2 | H | CH3S(O)CH2CO |
| 8-745 | C—CF3 | N | C—H | C—H | H | CF3 | CH2 | H | CH3SO2CH2CO |
| 8-746 | C—CF3 | N | C—H | C—H | H | CF3 | CH2 | H | CH3CH2NHCO |
| 8-747 | C—CF3 | N | C—CF3 | C—H | H | CF3 | CH2 | H | CH3CO |
| 8-748 | C—CF3 | N | C—CF3 | C—H | H | CF3 | CH2 | H | CH3CH2CO |
| 8-749 | C—CF3 | N | C—CF3 | C—H | H | CF3 | CH2 | H | n-PrCO |
| 8-750 | C—CF3 | N | C—CF3 | C—H | H | CF3 | CH2 | H | cyclo-PrCO |
| 8-751 | C—CF3 | N | C—CF3 | C—H | H | CF3 | CH2 | H | cyclo-PrCH2CO |
| 8-752 | C—CF3 | N | C—CF3 | C—H | H | CF3 | CH2 | H | CF3CH2CO |
| 8-753 | C—CF3 | N | C—CF3 | C—H | H | CF3 | CH2 | H | CH3SCH2CO |
| 8-754 | C—CF3 | N | C—CF3 | C—H | H | CF3 | CH2 | H | CH3S(O)CH2CO |
| 8-755 | C—CF3 | N | C—CF3 | C—H | H | CF3 | CH2 | H | CH3SO2CH2CO |
| 8-756 | C—CF3 | N | C—CF3 | C—H | H | CF3 | CH2 | H | CH3CH2NHCO |
| 8-757 | C—CF3 | C—H | C—H | N | H | CF3 | CH2 | H | CH3CO |
| 8-758 | C—CF3 | C—H | C—H | N | H | CF3 | CH2 | H | CH3CH2CO |
| 8-759 | C—CF3 | C—H | C—H | N | H | CF3 | CH2 | H | n-PrCO |
| 8-760 | C—CF3 | C—H | C—H | N | H | CF3 | CH2 | H | cyclo-PrCO |
| 8-761 | C—CF3 | C—H | C—H | N | H | CF3 | CH2 | H | cyclo-PrCH2CO |
| 8-762 | C—CF3 | C—H | C—H | N | H | CF3 | CH2 | H | CF3CH2CO |
| 8-763 | C—CF3 | C—H | C—H | N | H | CF3 | CH2 | H | CH3SCH2CO |
| 8-764 | C—CF3 | C—H | C—H | N | H | CF3 | CH2 | H | CH3S(O)CH2CO |
| 8-765 | C—CF3 | C—H | C—H | N | H | CF3 | CH2 | H | CH3SO2CH2CO |
| 8-766 | C—CF3 | C—H | C—H | N | H | CF3 | CH2 | H | CH3CH2NHCO |
| 8-767 | C—H | C—H | C—CF3 | N | H | CF3 | CH2 | H | CH3CO |
| 8-768 | C—H | C—H | C—CF3 | N | H | CF3 | CH2 | H | CH3CH2CO |
| 8-769 | C—H | C—H | C—CF3 | N | H | CF3 | CH2 | H | n-PrCO |
| 8-770 | C—H | C—H | C—CF3 | N | H | CF3 | CH2 | H | cyclo-PrCO |
| 8-771 | C—H | C—H | C—CF3 | N | H | CF3 | CH2 | H | cyclo-PrCH2CO |
| 8-772 | C—H | C—H | C—CF3 | N | H | CF3 | CH2 | H | CF3CH2CO |
| 8-773 | C—H | C—H | C—CF3 | N | H | CF3 | CH2 | H | CH3SCH2CO |
| 8-774 | C—H | C—H | C—CF3 | N | H | CF3 | CH2 | H | CH3S(O)CH2CO |
| 8-775 | C—H | C—H | C—CF3 | N | H | CF3 | CH2 | H | CH3SO2CH2CO |
| 8-776 | C—H | C—H | C—CF3 | N | H | CF3 | CH2 | H | CH3CH2NHCO |
| 8-777 | C—CF3 | C—H | C—Cl | N | H | CF3 | CH2 | H | CH3CO |
| 8-778 | C—CF3 | C—H | C—Cl | N | H | CF3 | CH2 | H | CH3CH2CO |
| 8-779 | C—CF3 | C—H | C—Cl | N | H | CF3 | CH2 | H | n-PrCO |
| 8-780 | C—CF3 | C—H | C—Cl | N | H | CF3 | CH2 | H | cyclo-PrCO |
| 8-781 | C—CF3 | C—H | C—Cl | N | H | CF3 | CH2 | H | cyclo-PrCH2CO |
| 8-782 | C—CF3 | C—H | C—Cl | N | H | CF3 | CH2 | H | CF3CH2CO |
| 8-783 | C—CF3 | C—H | C—Cl | N | H | CF3 | CH2 | H | CH3SCH2CO |
| 8-784 | C—CF3 | C—H | C—Cl | N | H | CF3 | CH2 | H | CH3S(O)CH2CO |
| 8-785 | C—CF3 | C—H | C—Cl | N | H | CF3 | CH2 | H | CH3SO2CH2CO |
| 8-786 | C—CF3 | C—H | C—Cl | N | H | CF3 | CH2 | H | CH3CH2NHCO |
| 8-787 | C—CF3 | C—H | C—Br | N | H | CF3 | CH2 | H | CH3CO |
| 8-788 | C—CF3 | C—H | C—Br | N | H | CF3 | CH2 | H | CH3CH2CO |
| 8-789 | C—CF3 | C—H | C—Br | N | H | CF3 | CH2 | H | n-PrCO |
| 8-790 | C—CF3 | C—H | C—Br | N | H | CF3 | CH2 | H | cyclo-PrCO |
| 8-791 | C—CF3 | C—H | C—Br | N | H | CF3 | CH2 | H | cyclo-PrCH2CO |
| 8-792 | C—CF3 | C—H | C—Br | N | H | CF3 | CH2 | H | CF3CH2CO |
| 8-794 | C—CF3 | C—H | C—Br | N | H | CF3 | CH2 | H | CH3S(O)CH2CO |
| 8-795 | C—CF3 | C—H | C—Br | N | H | CF3 | CH2 | H | CH3SO2CH2CO |
| 8-796 | C—CF3 | C—H | C—Br | N | H | CF3 | CH2 | H | CH3CH2NHCO |
| 8-797 | C—CF3 | C—H | C—CF3 | N | H | CF3 | CH2 | H | CH3CO |
| 8-798 | C—CF3 | C—H | C—CF3 | N | H | CF3 | CH2 | H | CH3CH2CO |
| 8-799 | C—CF3 | C—H | C—CF3 | N | H | CF3 | CH2 | H | n-PrCO |
| 8-800 | C—CF3 | C—H | C—CF3 | N | H | CF3 | CH2 | H | cyclo-PrCO |
| 8-801 | C—CF3 | C—H | C—CF3 | N | H | CF3 | CH2 | H | cyclo-PrCH2CO |
| 8-802 | C—CF3 | C—H | C—CF3 | N | H | CF3 | CH2 | H | CF3CH2CO |
| 8-804 | C—CF3 | C—H | C—CF3 | N | H | CF3 | CH2 | H | CH3S(O)CH2CO |
| 8-805 | C—CF3 | C—H | C—CF3 | N | H | CF3 | CH2 | H | CH3SO2CH2CO |
| 8-806 | C—CF3 | C—H | C—CF3 | N | H | CF3 | CH2 | H | CH3CH2NHCO |

TABLE 9

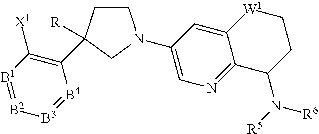

| No. | B¹ | B² | B³ | B⁴ | X¹ | R | W¹ | R⁵ | R⁶ |
|---|---|---|---|---|---|---|---|---|---|
| 9-1 | C—Cl | C—H | C—H | C—H | H | CF3 | — | H | CH3CO |
| 9-2 | C—Cl | C—H | C—H | C—H | H | CF3 | — | H | CH3CH2CO |
| 9-3 | C—Cl | C—H | C—H | C—H | H | CF3 | — | H | n-PrCO |
| 9-4 | C—Cl | C—H | C—H | C—H | H | CF3 | — | H | cyclo-PrCO |
| 9-5 | C—Cl | C—H | C—H | C—H | H | CF3 | — | H | cyclo-PrCH2CO |
| 9-6 | C—Cl | C—H | C—H | C—H | H | CF3 | — | H | CF3CH2CO |
| 9-7 | C—Cl | C—H | C—H | C—H | H | CF3 | — | H | CH3SCH2CO |
| 9-8 | C—Cl | C—H | C—H | C—H | H | CF3 | — | H | CH3S(O)CH2CO |
| 9-9 | C—Cl | C—H | C—H | C—H | H | CF3 | — | H | CH3SO2CH2CO |
| 9-10 | C—Cl | C—H | C—H | C—H | H | CF3 | — | H | CH3CH2NHCO |
| 9-11 | C—Cl | C—H | C—H | C—H | F | CF3 | — | H | CH3CO |
| 9-12 | C—Cl | C—H | C—H | C—H | F | CF3 | — | H | CH3CH2CO |
| 9-13 | C—Cl | C—H | C—H | C—H | F | CF3 | — | H | n-PrCO |
| 9-14 | C—Cl | C—H | C—H | C—H | F | CF3 | — | H | cyclo-PrCO |
| 9-15 | C—Cl | C—H | C—H | C—H | F | CF3 | — | H | cyclo-PrCH2CO |
| 9-16 | C—Cl | C—H | C—H | C—H | F | CF3 | — | H | CF3CH2CO |
| 9-17 | C—Cl | C—H | C—H | C—H | F | CF3 | — | H | CH3SCH2CO |
| 9-18 | C—Cl | C—H | C—H | C—H | F | CF3 | — | H | CH3S(O)CH2CO |
| 9-19 | C—Cl | C—H | C—H | C—H | F | CF3 | — | H | CH3SO2CH2CO |
| 9-20 | C—Cl | C—H | C—H | C—H | F | CF3 | — | H | CH3CH2NHCO |
| 9-21 | C—H | C—H | C—Cl | C—H | F | CF3 | — | H | CH3CO |
| 9-22 | C—H | C—H | C—Cl | C—H | F | CF3 | — | H | CH3CH2CO |
| 9-23 | C—H | C—H | C—Cl | C—H | F | CF3 | — | H | n-PrCO |
| 9-24 | C—H | C—H | C—Cl | C—H | F | CF3 | — | H | cyclo-PrCO |
| 9-25 | C—H | C—H | C—Cl | C—H | F | CF3 | — | H | cyclo-PrCH2CO |
| 9-26 | C—H | C—H | C—Cl | C—H | F | CF3 | — | H | CF3CH2CO |
| 9-27 | C—H | C—H | C—Cl | C—H | F | CF3 | — | H | CH3SCH2CO |
| 9-28 | C—H | C—H | C—Cl | C—H | F | CF3 | — | H | CH3S(O)CH2CO |
| 9-29 | C—H | C—H | C—Cl | C—H | F | CF3 | — | H | CH3SO2CH2CO |
| 9-30 | C—H | C—H | C—Cl | C—H | F | CF3 | — | H | CH3CH2NHCO |
| 9-31 | C—Cl | C—H | C—Cl | C—H | H | CF3 | — | H | H |
| 9-32 | C—Cl | C—H | C—Cl | C—H | H | CF3 | — | H | HCO |
| 9-33 | C—Cl | C—H | C—Cl | C—H | H | CF3 | — | H | CH3CO |
| 9-34 | C—Cl | C—H | C—Cl | C—H | H | CF3 | — | H | CH3CH2CO |
| 9-35 | C—Cl | C—H | C—Cl | C—H | H | CF3 | — | H | n-PrCO |
| 9-36 | C—Cl | C—H | C—Cl | C—H | H | CF3 | — | H | iso-PrCO |
| 9-37 | C—Cl | C—H | C—Cl | C—H | H | CF3 | — | H | cyclo-PrCO |
| 9-38 | C—Cl | C—H | C—Cl | C—H | H | CF3 | — | H | n-BuCO |
| 9-39 | C—Cl | C—H | C—Cl | C—H | H | CF3 | — | H | tert-BuCO |
| 9-40 | C—Cl | C—H | C—Cl | C—H | H | CF3 | — | H | iso-BuCO |
| 9-41 | C—Cl | C—H | C—Cl | C—H | H | CF3 | — | H | cyclo-BuCO |
| 9-42 | C—Cl | C—H | C—Cl | C—H | H | CF3 | — | H | (CH3)3CCH2CO |
| 9-43 | C—Cl | C—H | C—Cl | C—H | H | CF3 | — | H | cyclo-PenCO |
| 9-44 | C—Cl | C—H | C—Cl | C—H | H | CF3 | — | H | cyclo-HexCO |
| 9-45 | C—Cl | C—H | C—Cl | C—H | H | CF3 | — | H | cyclo-PrCH2CO |
| 9-46 | C—Cl | C—H | C—Cl | C—H | H | CF3 | — | H | CF3CO |
| 9-47 | C—Cl | C—H | C—Cl | C—H | H | CF3 | — | H | CCl3CO |
| 9-48 | C—Cl | C—H | C—Cl | C—H | H | CF3 | — | H | ClCH2CO |
| 9-49 | C—Cl | C—H | C—Cl | C—H | H | CF3 | — | H | CF2ClCO |
| 9-50 | C—Cl | C—H | C—Cl | C—H | H | CF3 | — | H | ClCH2CH2CO |
| 9-51 | C—Cl | C—H | C—Cl | C—H | H | CF3 | — | H | HCF2CF2CO |
| 9-52 | C—Cl | C—H | C—Cl | C—H | H | CF3 | — | H | CF3CH2CO |
| 9-54 | C—Cl | C—H | C—Cl | C—H | H | CF3 | — | H | HCF2CO |
| 9-55 | C—Cl | C—H | C—Cl | C—H | H | CF3 | — | H | ClCH2CO |
| 9-56 | C—Cl | C—H | C—Cl | C—H | H | CF3 | — | H | 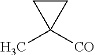 |
| 9-57 | C—Cl | C—H | C—Cl | C—H | H | CF3 | — | H | 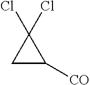 |
| 9-58 | C—Cl | C—H | C—Cl | C—H | H | CF3 | — | H | 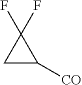 |
| 9-59 | C—Cl | C—H | C—Cl | C—H | H | CF3 | — | H | 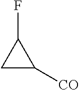 |
| 9-60 | C—Cl | C—H | C—Cl | C—H | H | CF3 | — | H | 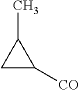 |
| 9-61 | C—Cl | C—H | C—Cl | C—H | H | CF3 | — | H | 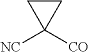 |
| 9-62 | C—Cl | C—H | C—Cl | C—H | H | CF3 | — | H | CH2═CH—CO |
| 9-63 | C—Cl | C—H | C—Cl | C—H | H | CF3 | — | H | CH3CH═CH—CO |
| 9-64 | C—Cl | C—H | C—Cl | C—H | H | CF3 | — | H | CH2═C(CH3)—CO |
| 9-65 | C—Cl | C—H | C—Cl | C—H | H | CF3 | — | H | CH3CH═C(CH3)—CO |
| 9-66 | C—Cl | C—H | C—Cl | C—H | H | CF3 | — | H | (CH3)2C═CH—CO |
| 9-67 | C—Cl | C—H | C—Cl | C—H | H | CF3 | — | H | CH2═C(Cl)—CO |
| 9-68 | C—Cl | C—H | C—Cl | C—H | H | CF3 | — | H | ClCH═C(CH3)—CO |
| 9-69 | C—Cl | C—H | C—Cl | C—H | H | CF3 | — | H | CH2═CHCH2—CO |
| 9-70 | C—Cl | C—H | C—Cl | C—H | H | CF3 | — | H | HCC—CO |
| 9-71 | C—Cl | C—H | C—Cl | C—H | H | CF3 | — | H | CH3CC—CO |
| 9-72 | C—Cl | C—H | C—Cl | C—H | H | CF3 | — | H | NCCH2—CO |
| 9-73 | C—Cl | C—H | C—Cl | C—H | H | CF3 | — | H | CH3OCH2—CO |
| 9-74 | C—Cl | C—H | C—Cl | C—H | H | CF3 | — | H | C2H5OCH2—CO |
| 9-75 | C—Cl | C—H | C—Cl | C—H | H | CF3 | — | H | CH3OCH2CH2—CO |
| 9-76 | C—Cl | C—H | C—Cl | C—H | H | CF3 | — | H | CH3CH2OCH2CH2—CO |
| 9-77 | C—Cl | C—H | C—Cl | C—H | H | CF3 | — | H | CH3SCH2CO |
| 9-78 | C—Cl | C—H | C—Cl | C—H | H | CF3 | — | H | CH3S(O)CH2CO |
| 9-79 | C—Cl | C—H | C—Cl | C—H | H | CF3 | — | H | CH3SO2CH2CO |
| 9-80 | C—Cl | C—H | C—Cl | C—H | H | CF3 | — | H | CH3O(CO)CH2—CO |
| 9-81 | C—Cl | C—H | C—Cl | C—H | H | CF3 | — | H | CH3(CO)OCH2—CO |
| 9-82 | C—Cl | C—H | C—Cl | C—H | H | CF3 | — | H | CH3O(CO)—CO |
| 9-83 | C—Cl | C—H | C—Cl | C—H | H | CF3 | — | H | CF3O(CO)—CO |
| 9-84 | C—Cl | C—H | C—Cl | C—H | H | CF3 | — | H | CH3(CO)CH2—CO |
| 9-85 | C—Cl | C—H | C—Cl | C—H | H | CF3 | — | H | (CH3)2NCH2—CO |
| 9-86 | C—Cl | C—H | C—Cl | C—H | H | CF3 | — | H | PhCO |
| 9-87 | C—Cl | C—H | C—Cl | C—H | H | CF3 | — | H | PhCH2CO |
| 9-88 | C—Cl | C—H | C—Cl | C—H | H | CF3 | — | H | 2-py-CO |
| 9-89 | C—Cl | C—H | C—Cl | C—H | H | CF3 | — | H | 3-py-CO |
| 9-90 | C—Cl | C—H | C—Cl | C—H | H | CF3 | — | H | 4-py-CO |
| 9-91 | C—Cl | C—H | C—Cl | C—H | H | CF3 | — | H | 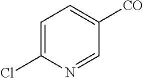 |
| 9-92 | C—Cl | C—H | C—Cl | C—H | H | CF3 | — | H | 2-F—PhCO |
| 9-93 | C—Cl | C—H | C—Cl | C—H | H | CF3 | — | H | 3-F—PhCO |
| 9-94 | C—Cl | C—H | C—Cl | C—H | H | CF3 | — | H | 4-F—PhCO |
| 9-95 | C—Cl | C—H | C—Cl | C—H | H | CF3 | — | H | 2-Cl—PhCO |
| 9-96 | C—Cl | C—H | C—Cl | C—H | H | CF3 | — | H | 3-Cl—PhCO |
| 9-97 | C—Cl | C—H | C—Cl | C—H | H | CF3 | — | H | 4-Cl—PhCO |
| 9-98 | C—Cl | C—H | C—Cl | C—H | H | CF3 | — | H | 2-Br—PhCO |
| 9-99 | C—Cl | C—H | C—Cl | C—H | H | CF3 | — | H | 2-CF3—PhCO |
| 9-100 | C—Cl | C—H | C—Cl | C—H | H | CF3 | — | H | 2-CH3—PhCO |
| 9-101 | C—Cl | C—H | C—Cl | C—H | H | CF3 | — | H | 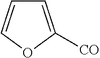 |
| 9-102 | C—Cl | C—H | C—Cl | C—H | H | CF3 | — | H | 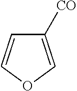 |
| 9-103 | C—Cl | C—H | C—Cl | C—H | H | CF3 | — | H | 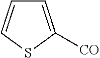 |
| 9-104 | C—Cl | C—H | C—Cl | C—H | H | CF3 | — | H | 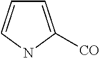 |
| 9-105 | C—Cl | C—H | C—Cl | C—H | H | CF3 | — | H | 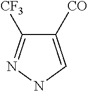 |
| 9-106 | C—Cl | C—H | C—Cl | C—H | H | CF3 | — | H | 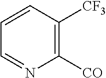 |
| 9-107 | C—Cl | C—H | C—Cl | C—H | H | CF3 | — | H | 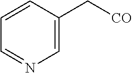 |
| 9-108 | C—Cl | C—H | C—Cl | C—H | H | CF3 | — | H | 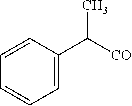 |
| 9-109 | C—Cl | C—H | C—Cl | C—H | H | CF3 | — | H | CH3NHCO |
| 9-110 | C—Cl | C—H | C—Cl | C—H | H | CF3 | — | H | CH3CH2NHCO |
| 9-111 | C—Cl | C—H | C—Cl | C—H | H | CF3 | — | H | CF3CH2NHCO |
| 9-112 | C—Cl | C—H | C—Cl | C—H | H | CF3 | — | H | n-PrNHCO |
| 9-113 | C—Cl | C—H | C—Cl | C—H | H | CF3 | — | H | iso-PrNHCO |
| 9-114 | C—Cl | C—H | C—Cl | C—H | H | CF3 | — | H | cyclo-PrNHCO |
| 9-115 | C—Cl | C—H | C—Cl | C—H | H | CF3 | — | H | n-BuNHCO |
| 9-116 | C—Cl | C—H | C—Cl | C—H | H | CF3 | — | H | tert-BuNHCO |
| 9-117 | C—Cl | C—H | C—Cl | C—H | H | CF3 | — | H | cyclo-BuNHCO |
| 9-118 | C—Cl | C—H | C—Cl | C—H | H | CF3 | — | H | cyclo-PenNHCO |
| 9-119 | C—Cl | C—H | C—Cl | C—H | H | CF3 | — | H | cyclo-HexNHCO |
| 9-120 | C—Cl | C—H | C—Cl | C—H | H | CF3 | — | H | CH2═CHCH2NHCO |
| 9-121 | C—Cl | C—H | C—Cl | C—H | H | CF3 | — | H | HCCCH2NHCO |
| 9-122 | C—Cl | C—H | C—Cl | C—H | H | CF3 | — | H | cyclo-PrCH2NHCO |
| 9-123 | C—Cl | C—H | C—Cl | C—H | H | CF3 | — | H | NCCH2NHCO |
| 9-124 | C—Cl | C—H | C—Cl | C—H | H | CF3 | — | H | CF3(CH3)CHNHCO |
| 9-125 | C—Cl | C—H | C—Cl | C—H | H | CF3 | — | H | CF3(CH3)2CNCO |
| 9-126 | C—Cl | C—H | C—Cl | C—H | H | CF3 | — | H | NCCH2OCO |
| 9-127 | C—Cl | C—H | C—Cl | C—H | H | CF3 | — | H | FCH2CH2NHCO |
| 9-128 | C—Cl | C—H | C—Cl | C—H | H | CF3 | — | H | CF3CH2CH2NHCO |
| 9-129 | C—Cl | C—H | C—Cl | C—H | H | CF3 | — | H | HCF2CF2CH2NHCO |
| 9-130 | C—Cl | C—H | C—Cl | C—H | H | CF3 | — | H | 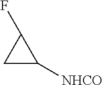 |
| 9-131 | C—Cl | C—H | C—Cl | C—H | H | CF3 | — | H | 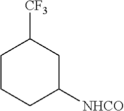 |
| 9-132 | C—Cl | C—H | C—Cl | C—H | H | CF3 | — | H | EtO2CCH2NHCO |
| 9-133 | C—Cl | C—H | C—Cl | C—H | H | CF3 | — | H | 2-CF3—PhNHCO |
| 9-134 | C—Cl | C—H | C—Cl | C—H | H | CF3 | — | H | 3-CF3—PhNHCO |
| 9-135 | C—Cl | C—H | C—Cl | C—H | H | CF3 | — | H | 4-CF3—PhNHCO |
| 9-136 | C—Cl | C—H | C—Cl | C—H | H | CF3 | — | H | 2-py-NHCO |
| 9-137 | C—Cl | C—H | C—Cl | C—H | H | CF3 | — | H | 3-py-NHCO |
| 9-138 | C—Cl | C—H | C—Cl | C—H | H | CF3 | — | H | 4-py-NHCO |
| 9-139 | C—Cl | C—H | C—Cl | C—H | H | CF3 | — | H | 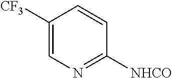 |
| 9-140 | C—Cl | C—H | C—Cl | C—H | H | CF3 | — | H | 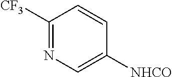 |
| 9-141 | C—Cl | C—H | C—Cl | C—H | H | CF3 | — | H | PhCH2HNCO |
| 9-142 | C—Cl | C—H | C—Cl | C—H | H | CF3 | — | H | 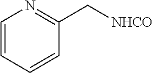 |
| 9-143 | C—Cl | C—H | C—Cl | C—H | H | CF3 | — | H | (CH3)2NCO |
| 9-144 | C—Cl | C—H | C—Cl | C—H | H | CF3 | — | H | pyrrolidineCO |
| 9-145 | C—Cl | C—H | C—Cl | C—H | H | CF3 | — | H | piperidineCO |
| 9-146 | C—Cl | C—H | C—Cl | C—H | H | CF3 | — | H | morpholineCO |
| 9-147 | C—Cl | C—H | C—Cl | C—H | H | CF3 | — | H | CH3OCONH |
| 9-148 | C—Cl | C—H | C—Cl | C—H | H | CF3 | — | H | CH3CH2OCO |
| 9-149 | C—Cl | C—H | C—Cl | C—H | H | CF3 | — | H | n-PrOCO |
| 9-150 | C—Cl | C—H | C—Cl | C—H | H | CF3 | — | H | cyclo-PrOCO |
| 9-151 | C—Cl | C—H | C—Cl | C—H | H | CF3 | — | H | CF3CH2OCO |
| 9-152 | C—Cl | C—H | C—Cl | C—H | H | CF3 | — | H | CH2═CHCH2OCO |
| 9-153 | C—Cl | C—H | C—Cl | C—H | H | CF3 | — | H | HCCCH2OCO |
| 9-154 | C—Cl | C—H | C—Cl | C—H | H | CF3 | — | H | PhOCO |
| 9-155 | C—Cl | C—H | C—Cl | C—H | H | CF3 | — | H | PhCH2OCO |
| 9-156 | C—Cl | C—H | C—Cl | C—H | H | CF3 | — | H | tert-BuOCO |
| 9-157 | C—Cl | C—H | C—Cl | C—H | H | CF3 | — | H | CH3SO2 |
| 9-158 | C—Cl | C—H | C—Cl | C—H | H | CF3 | — | H | CH3CH2SO2 |
| 9-159 | C—Cl | C—H | C—Cl | C—H | H | CF3 | — | H | CF3SO2 |
| 9-160 | C—Cl | C—H | C—Cl | C—H | H | CF3 | — | H | CF3CH2SO2 |
| 9-161 | C—Cl | C—H | C—Cl | C—H | H | CF3 | — | H | PhSO2 |
| 9-162 | C—Cl | C—H | C—Cl | C—H | H | CF3 | — | H | CH3NHCONHSO2 |
| 9-163 | C—Cl | C—H | C—Cl | C—H | H | CF3 | — | H | (CH3)2NCONHSO2 |
| 9-164 | C—Cl | C—H | C—Cl | C—H | H | CF3 | — | H | CH3CS |
| 9-165 | C—Cl | C—H | C—Cl | C—H | H | CF3 | — | H | CH3CH2CS |
| 9-166 | C—Cl | C—H | C—Cl | C—H | H | CF3 | — | H | cyclo-PrCS |
| 9-167 | C—Cl | C—H | C—Cl | C—H | H | CF3 | — | H | CF3CH2CS |
| 9-168 | C—Cl | C—H | C—Br | C—H | H | CF3 | — | H | CH3CO |
| 9-169 | C—Cl | C—H | C—Br | C—H | H | CF3 | — | H | CH3CH2CO |
| 9-170 | C—Cl | C—H | C—Br | C—H | H | CF3 | — | H | n-PrCO |
| 9-171 | C—Cl | C—H | C—Br | C—H | H | CF3 | — | H | cyclo-PrCO |
| 9-172 | C—Cl | C—H | C—Br | C—H | H | CF3 | — | H | cyclo-PrCH2CO |
| 9-173 | C—Cl | C—H | C—Br | C—H | H | CF3 | — | H | CF3CH2CO |
| 9-175 | C—Cl | C—H | C—Br | C—H | H | CF3 | — | H | CH3S(O)CH2CO |
| 9-176 | C—Cl | C—H | C—Br | C—H | H | CF3 | — | H | CH3SO2CH2CO |
| 9-177 | C—Cl | C—H | C—Br | C—H | H | CF3 | — | H | CH3CH2NHCO |
| 9-178 | C—Br | C—H | C—Br | C—H | H | CF3 | — | H | CH3CO |
| 9-179 | C—Br | C—H | C—Br | C—H | H | CF3 | — | H | CH3CH2CO |
| 9-180 | C—Br | C—H | C—Br | C—H | H | CF3 | — | H | n-PrCO |
| 9-181 | C—Br | C—H | C—Br | C—H | H | CF3 | — | H | cyclo-PrCO |
| 9-182 | C—Br | C—H | C—Br | C—H | H | CF3 | — | H | cyclo-PrCH2CO |
| 9-183 | C—Br | C—H | C—Br | C—H | H | CF3 | — | H | CF3CH2CO |
| 9-185 | C—Br | C—H | C—Br | C—H | H | CF3 | — | H | CH3S(O)CH2CO |
| 9-186 | C—Br | C—H | C—Br | C—H | H | CF3 | — | H | CH3SO2CH2CO |
| 9-187 | C—Br | C—H | C—Br | C—H | H | CF3 | — | H | CH3CH2NHCO |
| 9-188 | C—Cl | C—F | C—Cl | C—H | H | CF3 | — | H | CH3CO |
| 9-189 | C—Cl | C—F | C—Cl | C—H | H | CF3 | — | H | CH3CH2CO |
| 9-190 | C—Cl | C—F | C—Cl | C—H | H | CF3 | — | H | n-PrCO |
| 9-191 | C—Cl | C—F | C—Cl | C—H | H | CF3 | — | H | cyclo-PrCO |
| 9-192 | C—Cl | C—F | C—Cl | C—H | H | CF3 | — | H | cyclo-PrCH2CO |
| 9-193 | C—Cl | C—F | C—Cl | C—H | H | CF3 | — | H | CF3CH2CO |
| 9-195 | C—Cl | C—F | C—Cl | C—H | H | CF3 | — | H | CH3S(O)CH2CO |
| 9-196 | C—Cl | C—F | C—Cl | C—H | H | CF3 | — | H | CH3SO2CH2CO |
| 9-197 | C—Cl | C—F | C—Cl | C—H | H | CF3 | — | H | CH3CH2NHCO |
| 9-198 | C—Cl | C—Cl | C—Cl | C—H | H | CF3 | — | H | CH3CO |
| 9-199 | C—Cl | C—Cl | C—Cl | C—H | H | CF3 | — | H | CH3CH2CO |
| 9-200 | C—Cl | C—Cl | C—Cl | C—H | H | CF3 | — | H | n-PrCO |
| 9-201 | C—Cl | C—Cl | C—Cl | C—H | H | CF3 | — | H | cyclo-PrCO |
| 9-202 | C—Cl | C—Cl | C—Cl | C—H | H | CF3 | — | H | cyclo-PrCH2CO |
| 9-203 | C—Cl | C—Cl | C—Cl | C—H | H | CF3 | — | H | CF3CH2CO |
| 9-205 | C—Cl | C—Cl | C—Cl | C—H | H | CF3 | — | H | CH3CH2OCH2CH2—CO |
| 9-206 | C—Cl | C—Cl | C—Cl | C—H | H | CF3 | — | H | CH3SCH2CO |
| 9-207 | C—Cl | C—Cl | C—Cl | C—H | H | CF3 | — | H | CH3S(O)CH2CO |
| 9-208 | C—Cl | C—Cl | C—Cl | C—H | H | CF3 | — | H | CH3SO2CH2CO |
| 9-209 | C—Cl | C—Cl | C—Cl | C—H | H | CF3 | — | H | CH3CH2NHCO |
| 9-210 | C—Cl | C—Br | C—Cl | C—H | H | CF3 | — | H | CH3CO |
| 9-211 | C—Cl | C—Br | C—Cl | C—H | H | CF3 | — | H | CH3CH2CO |
| 9-212 | C—Cl | C—Br | C—Cl | C—H | H | CF3 | — | H | n-PrCO |
| 9-213 | C—Cl | C—Br | C—Cl | C—H | H | CF3 | — | H | cyclo-PrCO |
| 9-214 | C—Cl | C—Br | C—Cl | C—H | H | CF3 | — | H | cyclo-PrCH2CO |
| 9-215 | C—Cl | C—Br | C—Cl | C—H | H | CF3 | — | H | CF3CH2CO |
| 9-217 | C—Cl | C—Br | C—Cl | C—H | H | CF3 | — | H | CH3S(O)CH2CO |
| 9-218 | C—Cl | C—Br | C—Cl | C—H | H | CF3 | — | H | CH3SO2CH2CO |
| 9-219 | C—Cl | C—Br | C—Cl | C—H | H | CF3 | — | H | CH3CH2NHCO |
| 9-220 | C—Cl | C—Cl | C—Br | C—H | H | CF3 | — | H | CH3CO |
| 9-221 | C—Cl | C—Cl | C—Br | C—H | H | CF3 | — | H | CH3CH2CO |
| 9-222 | C—Cl | C—Cl | C—Br | C—H | H | CF3 | — | H | n-PrCO |
| 9-223 | C—Cl | C—Cl | C—Br | C—H | H | CF3 | — | H | cyclo-PrCO |
| 9-224 | C—Cl | C—Cl | C—Br | C—H | H | CF3 | — | H | cyclo-PrCH2CO |
| 9-225 | C—Cl | C—Cl | C—Br | C—H | H | CF3 | — | H | CF3CH2CO |
| 9-227 | C—Cl | C—Cl | C—Br | C—H | H | CF3 | — | H | CH3S(O)CH2CO |
| 9-228 | C—Cl | C—Cl | C—Br | C—H | H | CF3 | — | H | CH3SO2CH2CO |
| 9-229 | C—Cl | C—Cl | C—Br | C—H | H | CF3 | — | H | CH3CH2NHCO |
| 9-230 | C—Cl | C—F | C—Cl | C—H | F | CF3 | — | H | CH3CO |
| 9-231 | C—Cl | C—F | C—Cl | C—H | F | CF3 | — | H | CH3CH2CO |
| 9-232 | C—Cl | C—F | C—Cl | C—H | F | CF3 | — | H | n-PrCO |
| 9-233 | C—Cl | C—F | C—Cl | C—H | F | CF3 | — | H | cyclo-PrCO |
| 9-234 | C—Cl | C—F | C—Cl | C—H | F | CF3 | — | H | cyclo-PrCH2CO |
| 9-235 | C—Cl | C—F | C—Cl | C—H | F | CF3 | — | H | CF3CH2CO |
| 9-237 | C—Cl | C—F | C—Cl | C—H | F | CF3 | — | H | CH3S(O)CH2CO |
| 9-238 | C—Cl | C—F | C—Cl | C—H | F | CF3 | — | H | CH3SO2CH2CO |
| 9-239 | C—Cl | C—F | C—Cl | C—H | F | CF3 | — | H | CH3CH2NHCO |
| 9-240 | C—CF3 | C—H | C—H | C—H | H | CF3 | — | H | CH3CO |
| 9-241 | C—CF3 | C—H | C—H | C—H | H | CF3 | — | H | CH3CH2CO |
| 9-242 | C—CF3 | C—H | C—H | C—H | H | CF3 | — | H | n-PrCO |
| 9-243 | C—CF3 | C—H | C—H | C—H | H | CF3 | — | H | cyclo-PrCO |
| 9-244 | C—CF3 | C—H | C—H | C—H | H | CF3 | — | H | cyclo-PrCH2CO |
| 9-245 | C—CF3 | C—H | C—H | C—H | H | CF3 | — | H | CF3CH2CO |
| 9-247 | C—CF3 | C—H | C—H | C—H | H | CF3 | — | H | CH3S(O)CH2CO |
| 9-248 | C—CF3 | C—H | C—H | C—H | H | CF3 | — | H | CH3SO2CH2CO |
| 9-249 | C—CF3 | C—H | C—H | C—H | H | CF3 | — | H | CH3CH2NHCO |
| 9-250 | C—CF3 | C—F | C—H | C—H | H | CF3 | — | H | CH3CO |
| 9-251 | C—CF3 | C—F | C—H | C—H | H | CF3 | — | H | CH3CH2CO |
| 9-252 | C—CF3 | C—F | C—H | C—H | H | CF3 | — | H | n-PrCO |
| 9-253 | C—CF3 | C—F | C—H | C—H | H | CF3 | — | H | cyclo-PrCO |
| 9-254 | C—CF3 | C—F | C—H | C—H | H | CF3 | — | H | cyclo-PrCH2CO |
| 9-255 | C—CF3 | C—F | C—H | C—H | H | CF3 | — | H | CF3CH2CO |
| 9-257 | C—CF3 | C—F | C—H | C—H | H | CF3 | — | H | CH3S(O)CH2CO |
| 9-258 | C—CF3 | C—F | C—H | C—H | H | CF3 | — | H | CH3SO2CH2CO |
| 9-259 | C—CF3 | C—F | C—H | C—H | H | CF3 | — | H | CH3CH2NHCO |
| 9-260 | C—CF3 | C—H | C—H | C—H | F | CF3 | — | H | CH3CO |
| 9-261 | C—CF3 | C—H | C—H | C—H | F | CF3 | — | H | CH3CH2CO |
| 9-262 | C—CF3 | C—H | C—H | C—H | F | CF3 | — | H | n-PrCO |
| 9-263 | C—CF3 | C—H | C—H | C—H | F | CF3 | — | H | cyclo-PrCO |
| 9-264 | C—CF3 | C—H | C—H | C—H | F | CF3 | — | H | cyclo-PrCH2CO |
| 9-265 | C—CF3 | C—H | C—H | C—H | F | CF3 | — | H | CF3CH2CO |
| 9-267 | C—CF3 | C—H | C—H | C—H | F | CF3 | — | H | CH3S(O)CH2CO |
| 9-268 | C—CF3 | C—H | C—H | C—H | F | CF3 | — | H | CH3SO2CH2CO |
| 9-269 | C—CF3 | C—H | C—H | C—H | F | CF3 | — | H | CH3CH2NHCO |
| 9-270 | C—H | C—H | C—CF3 | C—H | F | CF3 | — | H | CH3CO |
| 9-271 | C—H | C—H | C—CF3 | C—H | F | CF3 | — | H | CH3CH2CO |
| 9-272 | C—H | C—H | C—CF3 | C—H | F | CF3 | — | H | n-PrCO |
| 9-273 | C—H | C—H | C—CF3 | C—H | F | CF3 | — | H | cyclo-PrCO |
| 9-274 | C—H | C—H | C—CF3 | C—H | F | CF3 | — | H | cyclo-PrCH2CO |
| 9-275 | C—H | C—H | C—CF3 | C—H | F | CF3 | — | H | CF3CH2CO |
| 9-277 | C—H | C—H | C—CF3 | C—H | F | CF3 | — | H | CH3S(O)CH2CO |
| 9-278 | C—H | C—H | C—CF3 | C—H | F | CF3 | — | H | CH3SO2CH2CO |
| 9-279 | C—H | C—H | C—CF3 | C—H | F | CF3 | — | H | CH3CH2NHCO |
| 9-280 | C—Cl | C—H | C—CF3 | C—H | H | CF3 | — | H | CH3CO |
| 9-281 | C—Cl | C—H | C—CF3 | C—H | H | CF3 | — | H | CH3CH2CO |
| 9-282 | C—Cl | C—H | C—CF3 | C—H | H | CF3 | — | H | n-PrCO |
| 9-283 | C—Cl | C—H | C—CF3 | C—H | H | CF3 | — | H | cyclo-PrCO |
| 9-284 | C—Cl | C—H | C—CF3 | C—H | H | CF3 | — | H | cyclo-PrCH2CO |
| 9-285 | C—Cl | C—H | C—CF3 | C—H | H | CF3 | — | H | CF3CH2CO |
| 9-287 | C—Cl | C—H | C—CF3 | C—H | H | CF3 | — | H | CH3CH2OCH2CH2—CO |
| 9-288 | C—Cl | C—H | C—CF3 | C—H | H | CF3 | — | H | CH3SCH2CO |
| 9-289 | C—Cl | C—H | C—CF3 | C—H | H | CF3 | — | H | CH3S(O)CH2CO |
| 9-290 | C—Cl | C—H | C—CF3 | C—H | H | CF3 | — | H | CH3SO2CH2CO |
| 9-291 | C—Cl | C—H | C—CF3 | C—H | H | CF3 | — | H | CH3CH2NHCO |
| 9-292 | C—CF3 | C—H | C—CF3 | C—H | H | CF3 | — | H | CH3CO |
| 9-293 | C—CF3 | C—H | C—CF3 | C—H | H | CF3 | — | H | CH3CH2CO |
| 9-294 | C—CF3 | C—H | C—CF3 | C—H | H | CF3 | — | H | n-PrCO |
| 9-295 | C—CF3 | C—H | C—CF3 | C—H | H | CF3 | — | H | cyclo-PrCO |
| 9-296 | C—CF3 | C—H | C—CF3 | C—H | H | CF3 | — | H | cy clo-PrCH2CO |
| 9-297 | C—CF3 | C—H | C—CF3 | C—H | H | CF3 | — | H | CF3CH2CO |
| 9-299 | C—CF3 | C—H | C—CF3 | C—H | H | CF3 | — | H | CH3CH2OCH2CH2—CO |
| 9-300 | C—CF3 | C—H | C—CF3 | C—H | H | CF3 | — | H | CH3SCH2CO |
| 9-301 | C—CF3 | C—H | C—CF3 | C—H | H | CF3 | — | H | CH3S(O)CH2CO |
| 9-302 | C—CF3 | C—H | C—CF3 | C—H | H | CF3 | — | H | CH3SO2CH2CO |
| 9-303 | C—CF3 | C—H | C—CF3 | C—H | H | CF3 | — | H | CH3CH2NHCO |
| 9-304 | C—Cl | C—Cl | C—CF3 | C—H | H | CF3 | — | H | CH3CO |
| 9-305 | C—Cl | C—Cl | C—CF3 | C—H | H | CF3 | — | H | CH3CH2CO |
| 9-306 | C—Cl | C—Cl | C—CF3 | C—H | H | CF3 | — | H | n-PrCO |
| 9-307 | C—Cl | C—Cl | C—CF3 | C—H | H | CF3 | — | H | cyclo-PrCO |
| 9-308 | C—Cl | C—Cl | C—CF3 | C—H | H | CF3 | — | H | cyclo-PrCH2CO |
| 9-309 | C—Cl | C—Cl | C—CF3 | C—H | H | CF3 | — | H | CF3CH2CO |
| 9-311 | C—Cl | C—Cl | C—CF3 | C—H | H | CF3 | — | H | CH3S(O)CH2CO |
| 9-312 | C—Cl | C—Cl | C—CF3 | C—H | H | CF3 | — | H | CH3SO2CH2CO |
| 9-313 | C—Cl | C—Cl | C—CF3 | C—H | H | CF3 | — | H | CH3CH2NHCO |
| 9-314 | C—Cl | N | C—H | C—H | H | CF3 | — | H | CH3CO |
| 9-315 | C—Cl | N | C—H | C—H | H | CF3 | — | H | CH3CH2CO |
| 9-316 | C—Cl | N | C—H | C—H | H | CF3 | — | H | n-PrCO |
| 9-317 | C—Cl | N | C—H | C—H | H | CF3 | — | H | cyclo-PrCO |
| 9-318 | C—Cl | N | C—H | C—H | H | CF3 | — | H | cyclo-PrCH2CO |
| 9-319 | C—Cl | N | C—H | C—H | H | CF3 | — | H | CF3CH2CO |
| 9-321 | C—Cl | N | C—H | C—H | H | CF3 | — | H | CH3S(O)CH2CO |
| 9-322 | C—Cl | N | C—H | C—H | H | CF3 | — | H | CH3SO2CH2CO |
| 9-323 | C—Cl | N | C—H | C—H | H | CF3 | — | H | CH3CH2NHCO |
| 9-324 | C—Cl | N | C—Cl | C—H | H | CF3 | — | H | CH3CO |
| 9-325 | C—Cl | N | C—Cl | C—H | H | CF3 | — | H | CH3CH2CO |
| 9-326 | C—Cl | N | C—Cl | C—H | H | CF3 | — | H | n-PrCO |
| 9-327 | C—Cl | N | C—Cl | C—H | H | CF3 | — | H | cyclo-PrCO |
| 9-328 | C—Cl | N | C—Cl | C—H | H | CF3 | — | H | cyclo-PrCH2CO |
| 9-329 | C—Cl | N | C—Cl | C—H | H | CF3 | — | H | CF3CH2CO |
| 9-330 | C—Cl | N | C—Cl | C—H | H | CF3 | — | H | CH3SCH2CO |
| 9-331 | C—Cl | N | C—Cl | C—H | H | CF3 | — | H | CH3S(O)CH2CO |
| 9-332 | C—Cl | N | C—Cl | C—H | H | CF3 | — | H | CH3SO2CH2CO |
| 9-333 | C—Cl | N | C—Cl | C—H | H | CF3 | — | H | CH3CH2NHCO |
| 9-334 | C—CF3 | N | C—H | C—H | H | CF3 | — | H | CH3CO |
| 9-335 | C—CF3 | N | C—H | C—H | H | CF3 | — | H | CH3CH2CO |
| 9-336 | C—CF3 | N | C—H | C—H | H | CF3 | — | H | n-PrCO |
| 9-337 | C—CF3 | N | C—H | C—H | H | CF3 | — | H | cyclo-PrCO |
| 9-338 | C—CF3 | N | C—H | C—H | H | CF3 | — | H | cyclo-PrCH2CO |
| 9-339 | C—CF3 | N | C—H | C—H | H | CF3 | — | H | CF3CH2CO |
| 9-340 | C—CF3 | N | C—H | C—H | H | CF3 | — | H | CH3SCH2CO |
| 9-341 | C—CF3 | N | C—H | C—H | H | CF3 | — | H | CH3S(O)CH2CO |
| 9-342 | C—CF3 | N | C—H | C—H | H | CF3 | — | H | CH3SO2CH2CO |
| 9-343 | C—CF3 | N | C—H | C—H | H | CF3 | — | H | CH3CH2NHCO |
| 9-344 | C—CF3 | N | C—CF3 | C—H | H | CF3 | — | H | CH3CO |
| 9-345 | C—CF3 | N | C—CF3 | C—H | H | CF3 | — | H | CH3CH2CO |
| 9-346 | C—CF3 | N | C—CF3 | C—H | H | CF3 | — | H | n-PrCO |
| 9-347 | C—CF3 | N | C—CF3 | C—H | H | CF3 | — | H | cyclo-PrCO |
| 9-348 | C—CF3 | N | C—CF3 | C—H | H | CF3 | — | H | cyclo-PrCH2CO |
| 9-349 | C—CF3 | N | C—CF3 | C—H | H | CF3 | — | H | CF3CH2CO |
| 9-350 | C—CF3 | N | C—CF3 | C—H | H | CF3 | — | H | CH3SCH2CO |
| 9-351 | C—CF3 | N | C—CF3 | C—H | H | CF3 | — | H | CH3S(O)CH2CO |
| 9-352 | C—CF3 | N | C—CF3 | C—H | H | CF3 | — | H | CH3SO2CH2CO |
| 9-353 | C—CF3 | N | C—CF3 | C—H | H | CF3 | — | H | CH3CH2NHCO |
| 9-354 | C—CF3 | C—H | C—H | N | H | CF3 | — | H | CH3CO |
| 9-355 | C—CF3 | C—H | C—H | N | H | CF3 | — | H | CH3CH2CO |
| 9-356 | C—CF3 | C—H | C—H | N | H | CF3 | — | H | n-PrCO |
| 9-357 | C—CF3 | C—H | C—H | N | H | CF3 | — | H | cyclo-PrCO |
| 9-358 | C—CF3 | C—H | C—H | N | H | CF3 | — | H | cyclo-PrCH2CO |
| 9-359 | C—CF3 | C—H | C—H | N | H | CF3 | — | H | CF3CH2CO |
| 9-360 | C—CF3 | C—H | C—H | N | H | CF3 | — | H | CH3SCH2CO |
| 9-361 | C—CF3 | C—H | C—H | N | H | CF3 | — | H | CH3S(O)CH2CO |
| 9-362 | C—CF3 | C—H | C—H | N | H | CF3 | — | H | CH3SO2CH2CO |
| 9-363 | C—CF3 | C—H | C—H | N | H | CF3 | — | H | CH3CH2NHCO |
| 9-364 | C—H | C—H | C—CF3 | N | H | CF3 | — | H | CH3CO |
| 9-365 | C—H | C—H | C—CF3 | N | H | CF3 | — | H | CH3CH2CO |
| 9-366 | C—H | C—H | C—CF3 | N | H | CF3 | — | H | n-PrCO |
| 9-367 | C—H | C—H | C—CF3 | N | H | CF3 | — | H | cyclo-PrCO |
| 9-368 | C—H | C—H | C—CF3 | N | H | CF3 | — | H | cyclo-PrCH2CO |
| 9-369 | C—H | C—H | C—CF3 | N | H | CF3 | — | H | CF3CH2CO |
| 9-370 | C—H | C—H | C—CF3 | N | H | CF3 | — | H | CH3SCH2CO |
| 9-371 | C—H | C—H | C—CF3 | N | H | CF3 | — | H | CH3S(O)CH2CO |
| 9-372 | C—H | C—H | C—CF3 | N | H | CF3 | — | H | CH3SO2CH2CO |
| 9-373 | C—H | C—H | C—CF3 | N | H | CF3 | — | H | CH3CH2NHCO |
| 9-374 | C—CF3 | C—H | C—Cl | N | H | CF3 | — | H | CH3CO |
| 9-375 | C—CF3 | C—H | C—Cl | N | H | CF3 | — | H | CH3CH2CO |
| 9-376 | C—CF3 | C—H | C—Cl | N | H | CF3 | — | H | n-PrCO |
| 9-377 | C—CF3 | C—H | C—Cl | N | H | CF3 | — | H | cyclo-PrCO |
| 9-378 | C—CF3 | C—H | C—Cl | N | H | CF3 | — | H | cyclo-PrCH2CO |
| 9-379 | C—CF3 | C—H | C—Cl | N | H | CF3 | — | H | CF3CH2CO |
| 9-380 | C—CF3 | C—H | C—Cl | N | H | CF3 | — | H | CH3SCH2CO |
| 9-381 | C—CF3 | C—H | C—Cl | N | H | CF3 | — | H | CH3S(O)CH2CO |
| 9-382 | C—CF3 | C—H | C—Cl | N | H | CF3 | — | H | CH3SO2CH2CO |
| 9-383 | C—CF3 | C—H | C—Cl | N | H | CF3 | — | H | CH3CH2NHCO |
| 9-384 | C—CF3 | C—H | C—Br | N | H | CF3 | — | H | CH3CO |
| 9-385 | C—CF3 | C—H | C—Br | N | H | CF3 | — | H | CH3CH2CO |
| 9-386 | C—CF3 | C—H | C—Br | N | H | CF3 | — | H | n-PrCO |
| 9-387 | C—CF3 | C—H | C—Br | N | H | CF3 | — | H | cyclo-PrCO |
| 9-388 | C—CF3 | C—H | C—Br | N | H | CF3 | — | H | cyclo-PrCH2CO |
| 9-389 | C—CF3 | C—H | C—Br | N | H | CF3 | — | H | CF3CH2CO |
| 9-390 | C—CF3 | C—H | C—Br | N | H | CF3 | — | H | CH3SCH2CO |
| 9-391 | C—CF3 | C—H | C—Br | N | H | CF3 | — | H | CH3S(O)CH2CO |
| 9-392 | C—CF3 | C—H | C—Br | N | H | CF3 | — | H | CH3SO2CH2CO |
| 9-393 | C—CF3 | C—H | C—Br | N | H | CF3 | — | H | CH3CH2NHCO |
| 9-394 | C—CF3 | C—H | C—CF3 | N | H | CF3 | — | H | CH3CO |
| 9-395 | C—CF3 | C—H | C—CF3 | N | H | CF3 | — | H | CH3CH2CO |
| 9-396 | C—CF3 | C—H | C—CF3 | N | H | CF3 | — | H | n-PrCO |
| 9-397 | C—CF3 | C—H | C—CF3 | N | H | CF3 | — | H | cyclo-PrCO |
| 9-398 | C—CF3 | C—H | C—CF3 | N | H | CF3 | — | H | cyclo-PrCH2CO |
| 9-399 | C—CF3 | C—H | C—CF3 | N | H | CF3 | — | H | CF3CH2CO |
| 9-400 | C—CF3 | C—H | C—CF3 | N | H | CF3 | — | H | CH3SCH2CO |
| 9-401 | C—CF3 | C—H | C—CF3 | N | H | CF3 | — | H | CH3S(O)CH2CO |
| 9-402 | C—CF3 | C—H | C—CF3 | N | H | CF3 | — | H | CH3SO2CH2CO |
| 9-403 | C—CF3 | C—H | C—CF3 | N | H | CF3 | — | H | CH3CH2NHCO |
| 9-404 | C—Cl | C—H | C—H | C—H | H | CF3 | CH2 | H | CH3CO |
| 9-405 | C—Cl | C—H | C—H | C—H | H | CF3 | CH2 | H | CH3CH2CO |
| 9-406 | C—Cl | C—H | C—H | C—H | H | CF3 | CH2 | H | n-PrCO |
| 9-407 | C—Cl | C—H | C—H | C—H | H | CF3 | CH2 | H | cyclo-PrCO |
| 9-408 | C—Cl | C—H | C—H | C—H | H | CF3 | CH2 | H | cyclo-PrCH2CO |
| 9-409 | C—Cl | C—H | C—H | C—H | H | CF3 | CH2 | H | CF3CH2CO |
| 9-410 | C—Cl | C—H | C—H | C—H | H | CF3 | CH2 | H | CH3SCH2CO |
| 9-411 | C—Cl | C—H | C—H | C—H | H | CF3 | CH2 | H | CH3S(O)CH2CO |
| 9-412 | C—Cl | C—H | C—H | C—H | H | CF3 | CH2 | H | CH3SO2CH2CO |
| 9-413 | C—Cl | C—H | C—H | C—H | H | CF3 | CH2 | H | CH3CH2NHCO |
| 9-414 | C—Cl | C—H | C—H | C—H | F | CF3 | CH2 | H | CH3CO |
| 9-415 | C—Cl | C—H | C—H | C—H | F | CF3 | CH2 | H | CH3CH2CO |
| 9-416 | C—Cl | C—H | C—H | C—H | F | CF3 | CH2 | H | n-PrCO |
| 9-417 | C—Cl | C—H | C—H | C—H | F | CF3 | CH2 | H | cyclo-PrCO |
| 9-418 | C—Cl | C—H | C—H | C—H | F | CF3 | CH2 | H | cyclo-PrCH2CO |
| 9-419 | C—Cl | C—H | C—H | C—H | F | CF3 | CH2 | H | CF3CH2CO |
| 9-420 | C—Cl | C—H | C—H | C—H | F | CF3 | CH2 | H | CH3SCH2CO |
| 9-421 | C—Cl | C—H | C—H | C—H | F | CF3 | CH2 | H | CH3S(O)CH2CO |
| 9-422 | C—Cl | C—H | C—H | C—H | F | CF3 | CH2 | H | CH3SO2CH2CO |
| 9-423 | C—Cl | C—H | C—H | C—H | F | CF3 | CH2 | H | CH3CH2NHCO |
| 9-424 | C—H | C—H | C—Cl | C—H | F | CF3 | CH2 | H | CH3CO |
| 9-425 | C—H | C—H | C—Cl | C—H | F | CF3 | CH2 | H | CH3CH2CO |
| 9-426 | C—H | C—H | C—Cl | C—H | F | CF3 | CH2 | H | n-PrCO |
| 9-427 | C—H | C—H | C—Cl | C—H | F | CF3 | CH2 | H | cyclo-PrCO |
| 9-428 | C—H | C—H | C—Cl | C—H | F | CF3 | CH2 | H | cyclo-PrCH2CO |
| 9-429 | C—H | C—H | C—Cl | C—H | F | CF3 | CH2 | H | CF3CH2CO |
| 9-430 | C—H | C—H | C—Cl | C—H | F | CF3 | CH2 | H | CH3SCH2CO |
| 9-431 | C—H | C—H | C—Cl | C—H | F | CF3 | CH2 | H | CH3S(O)CH2CO |
| 9-432 | C—H | C—H | C—Cl | C—H | F | CF3 | CH2 | H | CH3SO2CH2CO |
| 9-433 | C—H | C—H | C—Cl | C—H | F | CF3 | CH2 | H | CH3CH2NHCO |
| 9-434 | C—Cl | C—H | C—Cl | C—H | H | CF3 | CH2 | H | H |
| 9-435 | C—Cl | C—H | C—Cl | C—H | H | CF3 | CH2 | H | HCO |
| 9-436 | C—Cl | C—H | C—Cl | C—H | H | CF3 | CH2 | H | CH3CO |
| 9-437 | C—Cl | C—H | C—Cl | C—H | H | CF3 | CH2 | H | CH3CH2CO |
| 9-438 | C—Cl | C—H | C—Cl | C—H | H | CF3 | CH2 | H | n-PrCO |
| 9-439 | C—Cl | C—H | C—Cl | C—H | H | CF3 | CH2 | H | iso-PrCO |
| 9-440 | C—Cl | C—H | C—Cl | C—H | H | CF3 | CH2 | H | cyclo-PrCO |
| 9-441 | C—Cl | C—H | C—Cl | C—H | H | CF3 | CH2 | H | n-BuCO |
| 9-442 | C—Cl | C—H | C—Cl | C—H | H | CF3 | CH2 | H | tert-BuCO |
| 9-443 | C—Cl | C—H | C—Cl | C—H | H | CF3 | CH2 | H | iso-BuCO |
| 9-444 | C—Cl | C—H | C—Cl | C—H | H | CF3 | CH2 | H | cyclo-BuCO |
| 9-445 | C—Cl | C—H | C—Cl | C—H | H | CF3 | CH2 | H | (CH3)3CCH2CO |
| 9-446 | C—Cl | C—H | C—Cl | C—H | H | CF3 | CH2 | H | cyclo-PenCO |
| 9-447 | C—Cl | C—H | C—Cl | C—H | H | CF3 | CH2 | H | cyclo-HexCO |
| 9-448 | C—Cl | C—H | C—Cl | C—H | H | CF3 | CH2 | H | cyclo-PrCH2CO |
| 9-449 | C—Cl | C—H | C—Cl | C—H | H | CF3 | CH2 | H | CF3CO |
| 9-450 | C—Cl | C—H | C—Cl | C—H | H | CF3 | CH2 | H | CCl3CO |
| 9-451 | C—Cl | C—H | C—Cl | C—H | H | CF3 | CH2 | H | ClCH2CO |
| 9-452 | C—Cl | C—H | C—Cl | C—H | H | CF3 | CH2 | H | CF2ClCO |
| 9-453 | C—Cl | C—H | C—Cl | C—H | H | CF3 | CH2 | H | ClCH2CH2CO |
| 9-454 | C—Cl | C—H | C—Cl | C—H | H | CF3 | CH2 | H | HCF2CF2CO |
| 9-455 | C—Cl | C—H | C—Cl | C—H | H | CF3 | CH2 | H | CF3CH2CO |
| 9-456 | C—Cl | C—H | C—Cl | C—H | H | CF3 | CH2 | H | (CF3)2CHCO |
| 9-457 | C—Cl | C—H | C—Cl | C—H | H | CF3 | CH2 | H | HCF2CO |
| 9-458 | C—Cl | C—H | C—Cl | C—H | H | CF3 | CH2 | H | ClCH2CO |
| 9-459 | C—Cl | C—H | C—Cl | C—H | H | CF3 | CH2 | H | 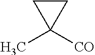 |
| 9-460 | C—Cl | C—H | C—Cl | C—H | H | CF3 | CH2 | H | 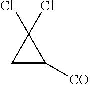 |
| 9-461 | C—Cl | C—H | C—Cl | C—H | H | CF3 | CH2 | H | 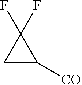 |
| 9-462 | C—Cl | C—H | C—Cl | C—H | H | CF3 | CH2 | H | 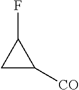 |
| 9-463 | C—Cl | C—H | C—Cl | C—H | H | CF3 | CH2 | H | 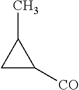 |
| 9-464 | C—Cl | C—H | C—Cl | C—H | H | CF3 | CH2 | H | 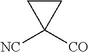 |
| 9-465 | C—Cl | C—H | C—Cl | C—H | H | CF3 | CH2 | H | CH2═CH—CO |
| 9-466 | C—Cl | C—H | C—Cl | C—H | H | CF3 | CH2 | H | CH3CH═CH—CO |
| 9-467 | C—Cl | C—H | C—Cl | C—H | H | CF3 | CH2 | H | CH2═C(CH3)—CO |
| 9-468 | C—Cl | C—H | C—Cl | C—H | H | CF3 | CH2 | H | CH3CH═C(CH3)—CO |
| 9-469 | C—Cl | C—H | C—Cl | C—H | H | CF3 | CH2 | H | (CH3)2C═CH—CO |
| 9-470 | C—Cl | C—H | C—Cl | C—H | H | CF3 | CH2 | H | CH2═C(Cl)—CO |
| 9-471 | C—Cl | C—H | C—Cl | C—H | H | CF3 | CH2 | H | ClCH═C(CH3)—CO |
| 9-472 | C—Cl | C—H | C—Cl | C—H | H | CF3 | CH2 | H | CH2═CHCH2—CO |
| 9-473 | C—Cl | C—H | C—Cl | C—H | H | CF3 | CH2 | H | HCC—CO |
| 9-474 | C—Cl | C—H | C—Cl | C—H | H | CF3 | CH2 | H | CH3CC—CO |
| 9-475 | C—Cl | C—H | C—Cl | C—H | H | CF3 | CH2 | H | NCCH2—CO |
| 9-476 | C—Cl | C—H | C—Cl | C—H | H | CF3 | CH2 | H | CH3OCH2—CO |
| 9-477 | C—Cl | C—H | C—Cl | C—H | H | CF3 | CH2 | H | C2H5OCH2—CO |
| 9-478 | C—Cl | C—H | C—Cl | C—H | H | CF3 | CH2 | H | CH3OCH2CH2—CO |
| 9-479 | C—Cl | C—H | C—Cl | C—H | H | CF3 | CH2 | H | CH3CH2OCH2CH2—CO |
| 9-480 | C—Cl | C—H | C—Cl | C—H | H | CF3 | CH2 | H | CH3SCH2CO |
| 9-481 | C—Cl | C—H | C—Cl | C—H | H | CF3 | CH2 | H | CH3S(O)CH2CO |
| 9-482 | C—Cl | C—H | C—Cl | C—H | H | CF3 | CH2 | H | CH3SO2CH2CO |
| 9-483 | C—Cl | C—H | C—Cl | C—H | H | CF3 | CH2 | H | CH3O(CO)CH2—CO |
| 9-484 | C—Cl | C—H | C—Cl | C—H | H | CF3 | CH2 | H | CH3(CO)OCH2—CO |
| 9-485 | C—Cl | C—H | C—Cl | C—H | H | CF3 | CH2 | H | CH3O(CO)—CO |
| 9-486 | C—Cl | C—H | C—Cl | C—H | H | CF3 | CH2 | H | CF3O(CO)—CO |
| 9-487 | C—Cl | C—H | C—Cl | C—H | H | CF3 | CH2 | H | CH3(CO)CH2—CO |
| 9-488 | C—Cl | C—H | C—Cl | C—H | H | CF3 | CH2 | H | (CH3)2NCH2—CO |
| 9-489 | C—Cl | C—H | C—Cl | C—H | H | CF3 | CH2 | H | PhCO |
| 9-490 | C—Cl | C—H | C—Cl | C—H | H | CF3 | CH2 | H | PhCH2CO |
| 9-491 | C—Cl | C—H | C—Cl | C—H | H | CF3 | CH2 | H | 2-py-CO |
| 9-492 | C—Cl | C—H | C—Cl | C—H | H | CF3 | CH2 | H | 3-py-CO |
| 9-493 | C—Cl | C—H | C—Cl | C—H | H | CF3 | CH2 | H | 4-py-CO |
| 9-494 | C—Cl | C—H | C—Cl | C—H | H | CF3 | CH2 | H | 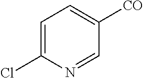 |
| 9-495 | C—Cl | C—H | C—Cl | C—H | H | CF3 | CH2 | H | 2-F—PhCO |
| 9-496 | C—Cl | C—H | C—Cl | C—H | H | CF3 | CH2 | H | 3-F—PhCO |
| 9-497 | C—Cl | C—H | C—Cl | C—H | H | CF3 | CH2 | H | 4-F—PhCO |
| 9-498 | C—Cl | C—H | C—Cl | C—H | H | CF3 | CH2 | H | 2-Cl—PhCO |
| 9-499 | C—Cl | C—H | C—Cl | C—H | H | CF3 | CH2 | H | 3-Cl—PhCO |
| 9-500 | C—Cl | C—H | C—Cl | C—H | H | CF3 | CH2 | H | 4-Cl—PhCO |
| 9-501 | C—Cl | C—H | C—Cl | C—H | H | CF3 | CH2 | H | 2-Br—PhCO |
| 9-502 | C—Cl | C—H | C—Cl | C—H | H | CF3 | CH2 | H | 2-CF3—PhCO |
| 9-503 | C—Cl | C—H | C—Cl | C—H | H | CF3 | CH2 | H | 2-CH3—PhCO |
| 9-504 | C—Cl | C—H | C—Cl | C—H | H | CF3 | CH2 | H | 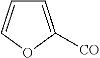 |
| 9-505 | C—Cl | C—H | C—Cl | C—H | H | CF3 | CH2 | H | 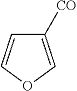 |
| 9-506 | C—Cl | C—H | C—Cl | C—H | H | CF3 | CH2 | H | 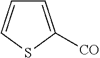 |
| 9-507 | C—Cl | C—H | C—Cl | C—H | H | CF3 | CH2 | H | 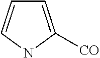 |
| 9-508 | C—Cl | C—H | C—Cl | C—H | H | CF3 | CH2 | H | 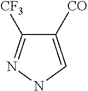 |
| 9-509 | C—Cl | C—H | C—Cl | C—H | H | CF3 | CH2 | H | 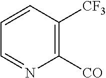 |
| 9-510 | C—Cl | C—H | C—Cl | C—H | H | CF3 | CH2 | H | 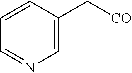 |
| 9-511 | C—Cl | C—H | C—Cl | C—H | H | CF3 | CH2 | H | 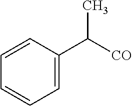 |
| 9-512 | C—Cl | C—H | C—Cl | C—H | H | CF3 | CH2 | H | CH3NHCO |
| 9-513 | C—Cl | C—H | C—Cl | C—H | H | CF3 | CH2 | H | CH3CH2NHCO |
| 9-514 | C—Cl | C—H | C—Cl | C—H | H | CF3 | CH2 | H | CF3CH2NHCO |
| 9-515 | C—Cl | C—H | C—Cl | C—H | H | CF3 | CH2 | H | n-PrNHCO |
| 9-516 | C—Cl | C—H | C—Cl | C—H | H | CF3 | CH2 | H | iso-PrNHCO |
| 9-517 | C—Cl | C—H | C—Cl | C—H | H | CF3 | CH2 | H | cyclo-PrNHCO |
| 9-518 | C—Cl | C—H | C—Cl | C—H | H | CF3 | CH2 | H | n-BuNHCO |
| 9-519 | C—Cl | C—H | C—Cl | C—H | H | CF3 | CH2 | H | tert-BuNHCO |
| 9-520 | C—Cl | C—H | C—Cl | C—H | H | CF3 | CH2 | H | cyclo-BuNHCO |
| 9-521 | C—Cl | C—H | C—Cl | C—H | H | CF3 | CH2 | H | cyclo-PenNHCO |
| 9-522 | C—Cl | C—H | C—Cl | C—H | H | CF3 | CH2 | H | cyclo-HexNHCO |
| 9-523 | C—Cl | C—H | C—Cl | C—H | H | CF3 | CH2 | H | CH2═CHCH2NHCO |
| 9-524 | C—Cl | C—H | C—Cl | C—H | H | CF3 | CH2 | H | HCCCH2NHCO |
| 9-525 | C—Cl | C—H | C—Cl | C—H | H | CF3 | CH2 | H | cyclo-PrCH2NHCO |
| 9-526 | C—Cl | C—H | C—Cl | C—H | H | CF3 | CH2 | H | NCCH2NHCO |
| 9-527 | C—Cl | C—H | C—Cl | C—H | H | CF3 | CH2 | H | CF3(CH3)CHNHCO |
| 9-528 | C—Cl | C—H | C—Cl | C—H | H | CF3 | CH2 | H | CF3(CH3)2CNCO |
| 9-529 | C—Cl | C—H | C—Cl | C—H | H | CF3 | CH2 | H | NCCH2OCO |
| 9-530 | C—Cl | C—H | C—Cl | C—H | H | CF3 | CH2 | H | FCH2CH2NHCO |
| 9-531 | C—Cl | C—H | C—Cl | C—H | H | CF3 | CH2 | H | CF3CH2CH2NHCO |
| 9-532 | C—Cl | C—H | C—Cl | C—H | H | CF3 | CH2 | H | HCF2CF2CH2NHCO |
| 9-533 | C—Cl | C—H | C—Cl | C—H | H | CF3 | CH2 | H | 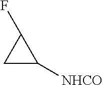 |
| 9-534 | C—Cl | C—H | C—Cl | C—H | H | CF3 | CH2 | H | 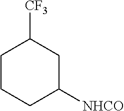 |
| 9-535 | C—Cl | C—H | C—Cl | C—H | H | CF3 | CH2 | H | EtO2CCH2NHCO |
| 9-536 | C—Cl | C—H | C—Cl | C—H | H | CF3 | CH2 | H | 2-CF3—PhNHCO |
| 9-537 | C—Cl | C—H | C—Cl | C—H | H | CF3 | CH2 | H | 3-CF3—PhNHCO |
| 9-538 | C—Cl | C—H | C—Cl | C—H | H | CF3 | CH2 | H | 4-CF3—PhNHCO |
| 9-539 | C—Cl | C—H | C—Cl | C—H | H | CF3 | CH2 | H | 2-py-NHCO |
| 9-540 | C—Cl | C—H | C—Cl | C—H | H | CF3 | CH2 | H | 3-py-NHCO |
| 9-541 | C—Cl | C—H | C—Cl | C—H | H | CF3 | CH2 | H | 4-py-NHCO |
| 9-542 | C—Cl | C—H | C—Cl | C—H | H | CF3 | CH2 | H | 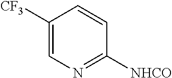 |
| 9-543 | C—Cl | C—H | C—Cl | C—H | H | CF3 | CH2 | H | 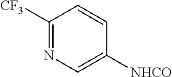 |
| 9-544 | C—Cl | C—H | C—Cl | C—H | H | CF3 | CH2 | H | PhCH2HNCO |
| 9-545 | C—Cl | C—H | C—Cl | C—H | H | CF3 | CH2 | H | 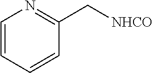 |
| 9-546 | C—Cl | C—H | C—Cl | C—H | H | CF3 | CH2 | H | (CH3)2NCO |
| 9-547 | C—Cl | C—H | C—Cl | C—H | H | CF3 | CH2 | H | pvrrolidineCO |
| 9-548 | C—Cl | C—H | C—Cl | C—H | H | CF3 | CH2 | H | piperidineCO |
| 9-549 | C—Cl | C—H | C—Cl | C—H | H | CF3 | CH2 | H | morpholineCO |
| 9-550 | C—Cl | C—H | C—Cl | C—H | H | CF3 | CH2 | H | CH3OCONH |
| 9-551 | C—Cl | C—H | C—Cl | C—H | H | CF3 | CH2 | H | CH3CH2OCO |
| 9-552 | C—Cl | C—H | C—Cl | C—H | H | CF3 | CH2 | H | n-PrOCO |
| 9-553 | C—Cl | C—H | C—Cl | C—H | H | CF3 | CH2 | H | cyclo-PrOCO |
| 9-554 | C—Cl | C—H | C—Cl | C—H | H | CF3 | CH2 | H | CF3CH2OCO |
| 9-555 | C—Cl | C—H | C—Cl | C—H | H | CF3 | CH2 | H | CH2═CHCH2OCO |
| 9-556 | C—Cl | C—H | C—Cl | C—H | H | CF3 | CH2 | H | HCCCH2OCO |
| 9-557 | C—Cl | C—H | C—Cl | C—H | H | CF3 | CH2 | H | PhOCO |
| 9-558 | C—Cl | C—H | C—Cl | C—H | H | CF3 | CH2 | H | PhCH2OCO |
| 9-559 | C—Cl | C—H | C—Cl | C—H | H | CF3 | CH2 | H | tert-BuOCO |
| 9-560 | C—Cl | C—H | C—Cl | C—H | H | CF3 | CH2 | H | CH3SO2 |
| 9-561 | C—Cl | C—H | C—Cl | C—H | H | CF3 | CH2 | H | CH3CH2SO2 |
| 9-562 | C—Cl | C—H | C—Cl | C—H | H | CF3 | CH2 | H | CF3SO2 |
| 9-563 | C—Cl | C—H | C—Cl | C—H | H | CF3 | CH2 | H | CF3CH2SO2 |
| 9-564 | C—Cl | C—H | C—Cl | C—H | H | CF3 | CH2 | H | PhSO2 |
| 9-565 | C—Cl | C—H | C—Cl | C—H | H | CF3 | CH2 | H | CH3NHCONHSO2 |
| 9-566 | C—Cl | C—H | C—Cl | C—H | H | CF3 | CH2 | H | (CH3)2NCONHSO2 |
| 9-567 | C—Cl | C—H | C—Cl | C—H | H | CF3 | CH2 | H | CH3CS |
| 9-568 | C—Cl | C—H | C—Cl | C—H | H | CF3 | CH2 | H | CH3CH2CS |
| 9-569 | C—Cl | C—H | C—Cl | C—H | H | CF3 | CH2 | H | cyclo-PrCS |
| 9-570 | C—Cl | C—H | C—Cl | C—H | H | CF3 | CH2 | H | CF3CH2CS |
| 9-571 | C—Cl | C—H | C—Br | C—H | H | CF3 | CH2 | H | CH3CO |
| 9-572 | C—Cl | C—H | C—Br | C—H | H | CF3 | CH2 | H | CH3CH2CO |
| 9-573 | C—Cl | C—H | C—Br | C—H | H | CF3 | CH2 | H | n-PrCO |
| 9-574 | C—Cl | C—H | C—Br | C—H | H | CF3 | CH2 | H | cyclo-PrCO |
| 9-575 | C—Cl | C—H | C—Br | C—H | H | CF3 | CH2 | H | cyclo-PrCH2CO |
| 9-576 | C—Cl | C—H | C—Br | C—H | H | CF3 | CH2 | H | CF3CH2CO |
| 9-577 | C—Cl | C—H | C—Br | C—H | H | CF3 | CH2 | H | CH3SCH2CO |
| 9-578 | C—Cl | C—H | C—Br | C—H | H | CF3 | CH2 | H | CH3S(O)CH2CO |
| 9-579 | C—Cl | C—H | C—Br | C—H | H | CF3 | CH2 | H | CH3SO2CH2CO |
| 9-580 | C—Cl | C—H | C—Br | C—H | H | CF3 | CH2 | H | CH3CH2NHCO |
| 9-581 | C—Br | C—H | C—Br | C—H | H | CF3 | CH2 | H | CH3CO |
| 9-582 | C—Br | C—H | C—Br | C—H | H | CF3 | CH2 | H | CH3CH2CO |
| 9-583 | C—Br | C—H | C—Br | C—H | H | CF3 | CH2 | H | n-PrCO |
| 9-584 | C—Br | C—H | C—Br | C—H | H | CF3 | CH2 | H | cyclo-PrCO |
| 9-585 | C—Br | C—H | C—Br | C—H | H | CF3 | CH2 | H | cyclo-PrCH2CO |
| 9-586 | C—Br | C—H | C—Br | C—H | H | CF3 | CH2 | H | CF3CH2CO |
| 9-587 | C—Br | C—H | C—Br | C—H | H | CF3 | CH2 | H | CH3SCH2CO |
| 9-588 | C—Br | C—H | C—Br | C—H | H | CF3 | CH2 | H | CH3S(O)CH2CO |
| 9-589 | C—Br | C—H | C—Br | C—H | H | CF3 | CH2 | H | CH3SO2CH2CO |
| 9-590 | C—Br | C—H | C—Br | C—H | H | CF3 | CH2 | H | CH3CH2NHCO |
| 9-591 | C—Cl | C—F | C—Cl | C—H | H | CF3 | CH2 | H | CH3CO |
| 9-592 | C—Cl | C—F | C—Cl | C—H | H | CF3 | CH2 | H | CH3CH2CO |
| 9-593 | C—Cl | C—F | C—Cl | C—H | H | CF3 | CH2 | H | n-PrCO |
| 9-594 | C—Cl | C—F | C—Cl | C—H | H | CF3 | CH2 | H | cyclo-PrCO |
| 9-595 | C—Cl | C—F | C—Cl | C—H | H | CF3 | CH2 | H | cyclo-PrCH2CO |
| 9-596 | C—Cl | C—F | C—Cl | C—H | H | CF3 | CH2 | H | CF3CH2CO |
| 9-598 | C—Cl | C—F | C—Cl | C—H | H | CF3 | CH2 | H | CH3S(O)CH2CO |
| 9-599 | C—Cl | C—F | C—Cl | C—H | H | CF3 | CH2 | H | CH3SO2CH2CO |
| 9-600 | C—Cl | C—F | C—Cl | C—H | H | CF3 | CH2 | H | CH3CH2NHCO |
| 9-601 | C—Cl | C—Cl | C—Cl | C—H | H | CF3 | CH2 | H | CH3CO |
| 9-602 | C—Cl | C—Cl | C—Cl | C—H | H | CF3 | CH2 | H | CH3CH2CO |
| 9-603 | C—Cl | C—Cl | C—Cl | C—H | H | CF3 | CH2 | H | n-PrCO |
| 9-604 | C—Cl | C—Cl | C—Cl | C—H | H | CF3 | CH2 | H | cyclo-PrCO |
| 9-605 | C—Cl | C—Cl | C—Cl | C—H | H | CF3 | CH2 | H | cyclo-PrCH2CO |
| 9-606 | C—Cl | C—Cl | C—Cl | C—H | H | CF3 | CH2 | H | CF3CH2CO |
| 9-608 | C—Cl | C—Cl | C—Cl | C—H | H | CF3 | CH2 | H | CH3CH2OCH2CH2—CO |
| 9-609 | C—Cl | C—Cl | C—Cl | C—H | H | CF3 | CH2 | H | CH3SCH2CO |
| 9-610 | C—Cl | C—Cl | C—Cl | C—H | H | CF3 | CH2 | H | CH3S(O)CH2CO |
| 9-611 | C—Cl | C—Cl | C—Cl | C—H | H | CF3 | CH2 | H | CH3SO2CH2CO |
| 9-612 | C—Cl | C—Cl | C—Cl | C—H | H | CF3 | CH2 | H | CH3CH2NHCO |
| 9-613 | C—Cl | C—Br | C—Cl | C—H | H | CF3 | CH2 | H | CH3CO |
| 9-614 | C—Cl | C—Br | C—Cl | C—H | H | CF3 | CH2 | H | CH3CH2CO |
| 9-615 | C—Cl | C—Br | C—Cl | C—H | H | CF3 | CH2 | H | n-PrCO |
| 9-616 | C—Cl | C—Br | C—Cl | C—H | H | CF3 | CH2 | H | cyclo-PrCO |
| 9-617 | C—Cl | C—Br | C—Cl | C—H | H | CF3 | CH2 | H | cyclo-PrCH2CO |
| 9-618 | C—Cl | C—Br | C—Cl | C—H | H | CF3 | CH2 | H | CF3CH2CO |
| 9-619 | C—Cl | C—Br | C—Cl | C—H | H | CF3 | CH2 | H | CH3SCH2CO |
| 9-620 | C—Cl | C—Br | C—Cl | C—H | H | CF3 | CH2 | H | CH3S(O)CH2CO |
| 9-621 | C—Cl | C—Br | C—Cl | C—H | H | CF3 | CH2 | H | CH3SO2CH2CO |
| 9-622 | C—Cl | C—Br | C—Cl | C—H | H | CF3 | CH2 | H | CH3CH2NHCO |
| 9-623 | C—Cl | C—Cl | C—Br | C—H | H | CF3 | CH2 | H | CH3CO |
| 9-624 | C—Cl | C—Cl | C—Br | C—H | H | CF3 | CH2 | H | CH3CH2CO |
| 9-625 | C—Cl | C—Cl | C—Br | C—H | H | CF3 | CH2 | H | n-PrCO |
| 9-626 | C—Cl | C—Cl | C—Br | C—H | H | CF3 | CH2 | H | cyclo-PrCO |
| 9-627 | C—Cl | C—Cl | C—Br | C—H | H | CF3 | CH2 | H | cyclo-PrCH2CO |
| 9-628 | C—Cl | C—Cl | C—Br | C—H | H | CF3 | CH2 | H | CF3CH2CO |
| 9-629 | C—Cl | C—Cl | C—Br | C—H | H | CF3 | CH2 | H | CH3SCH2CO |
| 9-630 | C—Cl | C—Cl | C—Br | C—H | H | CF3 | CH2 | H | CH3S(O)CH2CO |
| 9-631 | C—Cl | C—Cl | C—Br | C—H | H | CF3 | CH2 | H | CH3SO2CH2CO |
| 9-632 | C—Cl | C—Cl | C—Br | C—H | H | CF3 | CH2 | H | CH3CH2NHCO |
| 9-633 | C—Cl | C—F | C—Cl | C—H | F | CF3 | CH2 | H | CH3CO |
| 9-634 | C—Cl | C—F | C—Cl | C—H | F | CF3 | CH2 | H | CH3CH2CO |
| 9-635 | C—Cl | C—F | C—Cl | C—H | F | CF3 | CH2 | H | n-PrCO |
| 9-636 | C—Cl | C—F | C—Cl | C—H | F | CF3 | CH2 | H | cyclo-PrCO |
| 9-637 | C—Cl | C—F | C—Cl | C—H | F | CF3 | CH2 | H | cyclo-PrCH2CO |
| 9-638 | C—Cl | C—F | C—Cl | C—H | F | CF3 | CH2 | H | CF3CH2CO |
| 9-639 | C—Cl | C—F | C—Cl | C—H | F | CF3 | CH2 | H | CH3SCH2CO |
| 9-640 | C—Cl | C—F | C—Cl | C—H | F | CF3 | CH2 | H | CH3S(O)CH2CO |
| 9-641 | C—Cl | C—F | C—Cl | C—H | F | CF3 | CH2 | H | CH3SO2CH2CO |
| 9-642 | C—Cl | C—F | C—Cl | C—H | F | CF3 | CH2 | H | CH3CH2NHCO |
| 9-643 | C—CF3 | C—H | C—H | C—H | H | CF3 | CH2 | H | CH3CO |
| 9-644 | C—CF3 | C—H | C—H | C—H | H | CF3 | CH2 | H | CH3CH2CO |
| 9-645 | C—CF3 | C—H | C—H | C—H | H | CF3 | CH2 | H | n-PrCO |
| 9-646 | C—CF3 | C—H | C—H | C—H | H | CF3 | CH2 | H | cyclo-PrCO |
| 9-647 | C—CF3 | C—H | C—H | C—H | H | CF3 | CH2 | H | cyclo-PrCH2CO |
| 9-648 | C—CF3 | C—H | C—H | C—H | H | CF3 | CH2 | H | CF3CH2CO |
| 9-649 | C—CF3 | C—H | C—H | C—H | H | CF3 | CH2 | H | CH3SCH2CO |
| 9-650 | C—CF3 | C—H | C—H | C—H | H | CF3 | CH2 | H | CH3S(O)CH2CO |
| 9-651 | C—CF3 | C—H | C—H | C—H | H | CF3 | CH2 | H | CH3SO2CH2CO |
| 9-652 | C—CF3 | C—H | C—H | C—H | H | CF3 | CH2 | H | CH3CH2NHCO |
| 9-653 | C—CF3 | C—F | C—H | C—H | H | CF3 | CH2 | H | CH3CO |
| 9-654 | C—CF3 | C—F | C—H | C—H | H | CF3 | CH2 | H | CH3CH2CO |
| 9-655 | C—CF3 | C—F | C—H | C—H | H | CF3 | CH2 | H | n-PrCO |
| 9-656 | C—CF3 | C—F | C—H | C—H | H | CF3 | CH2 | H | cyclo-PrCO |
| 9-657 | C—CF3 | C—F | C—H | C—H | H | CF3 | CH2 | H | cyclo-PrCH2CO |
| 9-658 | C—CF3 | C—F | C—H | C—H | H | CF3 | CH2 | H | CF3CH2CO |
| 9-659 | C—CF3 | C—F | C—H | C—H | H | CF3 | CH2 | H | CH3SCH2CO |
| 9-660 | C—CF3 | C—F | C—H | C—H | H | CF3 | CH2 | H | CH3S(O)CH2CO |
| 9-661 | C—CF3 | C—F | C—H | C—H | H | CF3 | CH2 | H | CH3SO2CH2CO |
| 9-662 | C—CF3 | C—F | C—H | C—H | H | CF3 | CH2 | H | CH3CH2NHCO |
| 9-663 | C—CF3 | C—H | C—H | C—H | F | CF3 | CH2 | H | CH3CO |
| 9-664 | C—CF3 | C—H | C—H | C—H | F | CF3 | CH2 | H | CH3CH2CO |
| 9-665 | C—CF3 | C—H | C—H | C—H | F | CF3 | CH2 | H | n-PrCO |
| 9-666 | C—CF3 | C—H | C—H | C—H | F | CF3 | CH2 | H | cyclo-PrCO |
| 9-667 | C—CF3 | C—H | C—H | C—H | F | CF3 | CH2 | H | cyclo-PrCH2CO |
| 9-668 | C—CF3 | C—H | C—H | C—H | F | CF3 | CH2 | H | CF3CH2CO |
| 9-669 | C—CF3 | C—H | C—H | C—H | F | CF3 | CH2 | H | CH3SCH2CO |
| 9-670 | C—CF3 | C—H | C—H | C—H | F | CF3 | CH2 | H | CH3S(O)CH2CO |
| 9-671 | C—CF3 | C—H | C—H | C—H | F | CF3 | CH2 | H | CH3SO2CH2CO |
| 9-672 | C—CF3 | C—H | C—H | C—H | F | CF3 | CH2 | H | CH3CH2NHCO |
| 9-673 | C—H | C—H | C—CF3 | C—H | F | CF3 | CH2 | H | CH3CO |
| 9-674 | C—H | C—H | C—CF3 | C—H | F | CF3 | CH2 | H | CH3CH2CO |
| 9-675 | C—H | C—H | C—CF3 | C—H | F | CF3 | CH2 | H | n-PrCO |
| 9-676 | C—H | C—H | C—CF3 | C—H | F | CF3 | CH2 | H | cyclo-PrCO |
| 9-677 | C—H | C—H | C—CF3 | C—H | F | CF3 | CH2 | H | cyclo-PrCH2CO |
| 9-678 | C—H | C—H | C—CF3 | C—H | F | CF3 | CH2 | H | CF3CH2CO |
| 9-679 | C—H | C—H | C—CF3 | C—H | F | CF3 | CH2 | H | CH3SCH2CO |
| 9-680 | C—H | C—H | C—CF3 | C—H | F | CF3 | CH2 | H | CH3S(O)CH2CO |
| 9-681 | C—H | C—H | C—CF3 | C—H | F | CF3 | CH2 | H | CH3SO2CH2CO |
| 9-682 | C—H | C—H | C—CF3 | C—H | F | CF3 | CH2 | H | CH3CH2NHCO |
| 9-683 | C—Cl | C—H | C—CF3 | C—H | H | CF3 | CH2 | H | CH3CO |
| 9-684 | C—Cl | C—H | C—CF3 | C—H | H | CF3 | CH2 | H | CH3CH2CO |
| 9-685 | C—Cl | C—H | C—CF3 | C—H | H | CF3 | CH2 | H | n-PrCO |
| 9-686 | C—Cl | C—H | C—CF3 | C—H | H | CF3 | CH2 | H | cyclo-PrCO |
| 9-687 | C—Cl | C—H | C—CF3 | C—H | H | CF3 | CH2 | H | cyclo-PrCH2CO |
| 9-688 | C—Cl | C—H | C—CF3 | C—H | H | CF3 | CH2 | H | CF3CH2CO |
| 9-689 | C—Cl | C—H | C—CF3 | C—H | H | CF3 | CH2 | H | CH3OCH2CH2—CO |
| 9-690 | C—Cl | C—H | C—CF3 | C—H | H | CF3 | CH2 | H | CH3CH2OCH2CH2—CO |
| 9-691 | C—Cl | C—H | C—CF3 | C—H | H | CF3 | CH2 | H | CH3SCH2CO |
| 9-692 | C—Cl | C—H | C—CF3 | C—H | H | CF3 | CH2 | H | CH3S(O)CH2CO |
| 9-693 | C—Cl | C—H | C—CF3 | C—H | H | CF3 | CH2 | H | CH3SO2CH2CO |
| 9-694 | C—Cl | C—H | C—CF3 | C—H | H | CF3 | CH2 | H | CH3CH2NHCO |
| 9-695 | C—CF3 | C—H | C—CF3 | C—H | H | CF3 | CH2 | H | CH3CO |
| 9-696 | C—CF3 | C—H | C—CF3 | C—H | H | CF3 | CH2 | H | CH3CH2CO |
| 9-697 | C—CF3 | C—H | C—CF3 | C—H | H | CF3 | CH2 | H | n-PrCO |
| 9-698 | C—CF3 | C—H | C—CF3 | C—H | H | CF3 | CH2 | H | cyclo-PrCO |
| 9-699 | C—CF3 | C—H | C—CF3 | C—H | H | CF3 | CH2 | H | cyclo-PrCH2CO |
| 9-700 | C—CF3 | C—H | C—CF3 | C—H | H | CF3 | CH2 | H | CF3CH2CO |
| 9-701 | C—CF3 | C—H | C—CF3 | C—H | H | CF3 | CH2 | H | CH3OCH2CH2—CO |
| 9-702 | C—CF3 | C—H | C—CF3 | C—H | H | CF3 | CH2 | H | CH3CH2OCH2CH2—CO |
| 9-703 | C—CF3 | C—H | C—CF3 | C—H | H | CF3 | CH2 | H | CH3SCH2CO |
| 9-704 | C—CF3 | C—H | C—CF3 | C—H | H | CF3 | CH2 | H | CH3S(O)CH2CO |
| 9-705 | C—CF3 | C—H | C—CF3 | C—H | H | CF3 | CH2 | H | CH3SO2CH2CO |
| 9-706 | C—CF3 | C—H | C—CF3 | C—H | H | CF3 | CH2 | H | CH3CH2NHCO |
| 9-707 | C—Cl | C—Cl | C—CF3 | C—H | H | CF3 | CH2 | H | CH3CO |
| 9-708 | C—Cl | C—Cl | C—CF3 | C—H | H | CF3 | CH2 | H | CH3CH2CO |
| 9-709 | C—Cl | C—Cl | C—CF3 | C—H | H | CF3 | CH2 | H | n-PrCO |
| 9-710 | C—Cl | C—Cl | C—CF3 | C—H | H | CF3 | CH2 | H | cyclo-PrCO |
| 9-711 | C—Cl | C—Cl | C—CF3 | C—H | H | CF3 | CH2 | H | cyclo-PrCH2CO |
| 9-712 | C—Cl | C—Cl | C—CF3 | C—H | H | CF3 | CH2 | H | CF3CH2CO |
| 9-713 | C—Cl | C—Cl | C—CF3 | C—H | H | CF3 | CH2 | H | CH3SCH2CO |
| 9-714 | C—Cl | C—Cl | C—CF3 | C—H | H | CF3 | CH2 | H | CH3S(O)CH2CO |
| 9-715 | C—Cl | C—Cl | C—CF3 | C—H | H | CF3 | CH2 | H | CH3SO2CH2CO |
| 9-716 | C—Cl | C—Cl | C—CF3 | C—H | H | CF3 | CH2 | H | CH3CH2NHCO |
| 9-717 | C—Cl | N | C—H | C—H | H | CF3 | CH2 | H | CH3CO |
| 9-718 | C—Cl | N | C—H | C—H | H | CF3 | CH2 | H | CH3CH2CO |
| 9-719 | C—Cl | N | C—H | C—H | H | CF3 | CH2 | H | n-PrCO |
| 9-720 | C—Cl | N | C—H | C—H | H | CF3 | CH2 | H | cyclo-PrCO |
| 9-721 | C—Cl | N | C—H | C—H | H | CF3 | CH2 | H | cyclo-PrCH2CO |
| 9-722 | C—Cl | N | C—H | C—H | H | CF3 | CH2 | H | CF3CH2CO |
| 9-723 | C—Cl | N | C—H | C—H | H | CF3 | CH2 | H | CH3SCH2CO |
| 9-724 | C—Cl | N | C—H | C—H | H | CF3 | CH2 | H | CH3S(O)CH2CO |
| 9-725 | C—Cl | N | C—H | C—H | H | CF3 | CH2 | H | CH3SO2CH2CO |
| 9-726 | C—Cl | N | C—H | C—H | H | CF3 | CH2 | H | CH3CH2NHCO |
| 9-727 | C—Cl | N | C—Cl | C—H | H | CF3 | CH2 | H | CH3CO |
| 9-728 | C—Cl | N | C—Cl | C—H | H | CF3 | CH2 | H | CH3CH2CO |
| 9-729 | C—Cl | N | C—Cl | C—H | H | CF3 | CH2 | H | n-PrCO |
| 9-730 | C—Cl | N | C—Cl | C—H | H | CF3 | CH2 | H | cyclo-PrCO |
| 9-731 | C—Cl | N | C—Cl | C—H | H | CF3 | CH2 | H | cyclo-PrCH2CO |
| 9-732 | C—Cl | N | C—Cl | C—H | H | CF3 | CH2 | H | CF3CH2CO |
| 9-733 | C—Cl | N | C—Cl | C—H | H | CF3 | CH2 | H | CH3SCH2CO |
| 9-734 | C—Cl | N | C—Cl | C—H | H | CF3 | CH2 | H | CH3S(O)CH2CO |
| 9-735 | C—Cl | N | C—Cl | C—H | H | CF3 | CH2 | H | CH3SO2CH2CO |
| 9-736 | C—Cl | N | C—Cl | C—H | H | CF3 | CH2 | H | CH3CH2NHCO |
| 9-737 | C—CF3 | N | C—H | C—H | H | CF3 | CH2 | H | CH3CO |
| 9-738 | C—CF3 | N | C—H | C—H | H | CF3 | CH2 | H | CH3CH2CO |
| 9-739 | C—CF3 | N | C—H | C—H | H | CF3 | CH2 | H | n-PrCO |
| 9-740 | C—CF3 | N | C—H | C—H | H | CF3 | CH2 | H | cyclo-PrCO |
| 9-741 | C—CF3 | N | C—H | C—H | H | CF3 | CH2 | H | cyclo-PrCH2CO |
| 9-742 | C—CF3 | N | C—H | C—H | H | CF3 | CH2 | H | CF3CH2CO |
| 9-743 | C—CF3 | N | C—H | C—H | H | CF3 | CH2 | H | CH3SCH2CO |
| 9-744 | C—CF3 | N | C—H | C—H | H | CF3 | CH2 | H | CH3S(O)CH2CO |
| 9-745 | C—CF3 | N | C—H | C—H | H | CF3 | CH2 | H | CH3SO2CH2CO |
| 9-746 | C—CF3 | N | C—H | C—H | H | CF3 | CH2 | H | CH3CH2NHCO |
| 9-747 | C—CF3 | N | C—CF3 | C—H | H | CF3 | CH2 | H | CH3CO |
| 9-748 | C—CF3 | N | C—CF3 | C—H | H | CF3 | CH2 | H | CH3CH2CO |
| 9-749 | C—CF3 | N | C—CF3 | C—H | H | CF3 | CH2 | H | n-PrCO |
| 9-750 | C—CF3 | N | C—CF3 | C—H | H | CF3 | CH2 | H | cyclo-PrCO |
| 9-751 | C—CF3 | N | C—CF3 | C—H | H | CF3 | CH2 | H | cyclo-PrCH2CO |
| 9-752 | C—CF3 | N | C—CF3 | C—H | H | CF3 | CH2 | H | CF3CH2CO |
| 9-753 | C—CF3 | N | C—CF3 | C—H | H | CF3 | CH2 | H | CH3SCH2CO |
| 9-754 | C—CF3 | N | C—CF3 | C—H | H | CF3 | CH2 | H | CH3S(O)CH2CO |
| 9-755 | C—CF3 | N | C—CF3 | C—H | H | CF3 | CH2 | H | CH3SO2CH2CO |
| 9-756 | C—CF3 | N | C—CF3 | C—H | H | CF3 | CH2 | H | CH3CH2NHCO |
| 9-757 | C—CF3 | C—H | C—H | N | H | CF3 | CH2 | H | CH3CO |
| 9-758 | C—CF3 | C—H | C—H | N | H | CF3 | CH2 | H | CH3CH2CO |
| 9-759 | C—CF3 | C—H | C—H | N | H | CF3 | CH2 | H | n-PrCO |
| 9-760 | C—CF3 | C—H | C—H | N | H | CF3 | CH2 | H | cyclo-PrCO |
| 9-761 | C—CF3 | C—H | C—H | N | H | CF3 | CH2 | H | cyclo-PrCH2CO |
| 9-762 | C—CF3 | C—H | C—H | N | H | CF3 | CH2 | H | CF3CH2CO |
| 9-763 | C—CF3 | C—H | C—H | N | H | CF3 | CH2 | H | CH3SCH2CO |
| 9-764 | C—CF3 | C—H | C—H | N | H | CF3 | CH2 | H | CH3S(O)CH2CO |
| 9-765 | C—CF3 | C—H | C—H | N | H | CF3 | CH2 | H | CH3SO2CH2CO |
| 9-766 | C—CF3 | C—H | C—H | N | H | CF3 | CH2 | H | CH3CH2NHCO |
| 9-767 | C—H | C—H | C—CF3 | N | H | CF3 | CH2 | H | CH3CO |
| 9-768 | C—H | C—H | C—CF3 | N | H | CF3 | CH2 | H | CH3CH2CO |
| 9-769 | C—H | C—H | C—CF3 | N | H | CF3 | CH2 | H | n-PrCO |
| 9-770 | C—H | C—H | C—CF3 | N | H | CF3 | CH2 | H | cyclo-PrCO |
| 9-771 | C—H | C—H | C—CF3 | N | H | CF3 | CH2 | H | cyclo-PrCH2CO |
| 9-772 | C—H | C—H | C—CF3 | N | H | CF3 | CH2 | H | CF3CH2CO |
| 9-773 | C—H | C—H | C—CF3 | N | H | CF3 | CH2 | H | CH3SCH2CO |
| 9-774 | C—H | C—H | C—CF3 | N | H | CF3 | CH2 | H | CH3S(O)CH2CO |
| 9-775 | C—H | C—H | C—CF3 | N | H | CF3 | CH2 | H | CH3SO2CH2CO |
| 9-776 | C—H | C—H | C—CF3 | N | H | CF3 | CH2 | H | CH3CH2NHCO |
| 9-777 | C—CF3 | C—H | C—Cl | N | H | CF3 | CH2 | H | CH3CO |
| 9-778 | C—CF3 | C—H | C—Cl | N | H | CF3 | CH2 | H | CH3CH2CO |
| 9-779 | C—CF3 | C—H | C—Cl | N | H | CF3 | CH2 | H | n-PrCO |
| 9-780 | C—CF3 | C—H | C—Cl | N | H | CF3 | CH2 | H | cyclo-PrCO |
| 9-781 | C—CF3 | C—H | C—Cl | N | H | CF3 | CH2 | H | cyclo-PrCH2CO |
| 9-782 | C—CF3 | C—H | C—Cl | N | H | CF3 | CH2 | H | CF3CH2CO |
| 9-783 | C—CF3 | C—H | C—Cl | N | H | CF3 | CH2 | H | CH3SCH2CO |
| 9-784 | C—CF3 | C—H | C—Cl | N | H | CF3 | CH2 | H | CH3S(O)CH2CO |
| 9-785 | C—CF3 | C—H | C—Cl | N | H | CF3 | CH2 | H | CH3SO2CH2CO |
| 9-786 | C—CF3 | C—H | C—Cl | N | H | CF3 | CH2 | H | CH3CH2NHCO |
| 9-787 | C—CF3 | C—H | C—Br | N | H | CF3 | CH2 | H | CH3CO |
| 9-788 | C—CF3 | C—H | C—Br | N | H | CF3 | CH2 | H | CH3CH2CO |
| 9-789 | C—CF3 | C—H | C—Br | N | H | CF3 | CH2 | H | n-PrCO |
| 9-790 | C—CF3 | C—H | C—Br | N | H | CF3 | CH2 | H | cyclo-PrCO |
| 9-791 | C—CF3 | C—H | C—Br | N | H | CF3 | CH2 | H | cyclo-PrCH2CO |
| 9-792 | C—CF3 | C—H | C—Br | N | H | CF3 | CH2 | H | CF3CH2CO |
| 9-794 | C—CF3 | C—H | C—Br | N | H | CF3 | CH2 | H | CH3S(O)CH2CO |
| 9-795 | C—CF3 | C—H | C—Br | N | H | CF3 | CH2 | H | CH3SO2CH2CO |
| 9-796 | C—CF3 | C—H | C—Br | N | H | CF3 | CH2 | H | CH3CH2NHCO |
| 9-797 | C—CF3 | C—H | C—CF3 | N | H | CF3 | CH2 | H | CH3CO |
| 9-798 | C—CF3 | C—H | C—CF3 | N | H | CF3 | CH2 | H | CH3CH2CO |
| 9-799 | C—CF3 | C—H | C—CF3 | N | H | CF3 | CH2 | H | n-PrCO |
| 9-800 | C—CF3 | C—H | C—CF3 | N | H | CF3 | CH2 | H | cyclo-PrCO |
| 9-801 | C—CF3 | C—H | C—CF3 | N | H | CF3 | CH2 | H | cyclo-PrCH2CO |
| 9-802 | C—CF3 | C—H | C—CF3 | N | H | CF3 | CH2 | H | CF3CH2CO |
| 9-804 | C—CF3 | C—H | C—CF3 | N | H | CF3 | CH2 | H | CH3S(O)CH2CO |
| 9-805 | C—CF3 | C—H | C—CF3 | N | H | CF3 | CH2 | H | CH3SO2CH2CO |
| 9-806 | C—CF3 | C—H | C—CF3 | N | H | CF3 | CH2 | H | CH3CH2NHCO |

TABLE 10

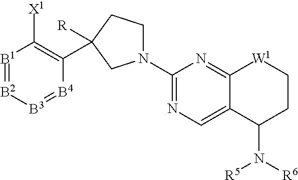

| No. | B¹ | B² | B³ | B⁴ | X¹ | R | W¹ | R⁵ | R⁶ |
|---|---|---|---|---|---|---|---|---|---|
| 10-1 | C—Cl | C—H | C—H | C—H | H | CF3 | — | H | CH3CO |
| 10-2 | C—Cl | C—H | C—H | C—H | H | CF3 | — | H | CH3CH2CO |
| 10-3 | C—Cl | C—H | C—H | C—H | H | CF3 | — | H | n-PrCO |
| 10-4 | C—Cl | C—H | C—H | C—H | H | CF3 | — | H | cyclo-PrCO |
| 10-5 | C—Cl | C—H | C—H | C—H | H | CF3 | — | H | cyclo-PrCH2CO |
| 10-6 | C—Cl | C—H | C—H | C—H | H | CF3 | — | H | CF3CH2CO |
| 10-7 | C—Cl | C—H | C—H | C—H | H | CF3 | — | H | CH3SCH2CO |
| 10-8 | C—Cl | C—H | C—H | C—H | H | CF3 | — | H | CH3S(O)CH2CO |
| 10-9 | C—Cl | C—H | C—H | C—H | H | CF3 | — | H | CH3SO2CH2CO |
| 10-10 | C—Cl | C—H | C—H | C—H | H | CF3 | — | H | CH3CH2NHCO |
| 10-11 | C—Cl | C—H | C—H | C—H | F | CF3 | — | H | CH3CO |
| 10-12 | C—Cl | C—H | C—H | C—H | F | CF3 | — | H | CH3CH2CO |
| 10-13 | C—Cl | C—H | C—H | C—H | F | CF3 | — | H | n-PrCO |
| 10-14 | C—Cl | C—H | C—H | C—H | F | CF3 | — | H | cyclo-PrCO |
| 10-15 | C—Cl | C—H | C—H | C—H | F | CF3 | — | H | cyclo-PrCH2CO |
| 10-16 | C—Cl | C—H | C—H | C—H | F | CF3 | — | H | CF3CH2CO |
| 10-17 | C—Cl | C—H | C—H | C—H | F | CF3 | — | H | CH3SCH2CO |
| 10-18 | C—Cl | C—H | C—H | C—H | F | CF3 | — | H | CH3S(O)CH2CO |
| 10-19 | C—Cl | C—H | C—H | C—H | F | CF3 | — | H | CH3SO2CH2CO |
| 10-20 | C—Cl | C—H | C—H | C—H | F | CF3 | — | H | CH3CH2NHCO |
| 10-21 | C—H | C—H | C—Cl | C—H | F | CF3 | — | H | CH3CO |
| 10-22 | C—H | C—H | C—Cl | C—H | F | CF3 | — | H | CH3CH2CO |
| 10-23 | C—H | C—H | C—Cl | C—H | F | CF3 | — | H | n-PrCO |
| 10-24 | C—H | C—H | C—Cl | C—H | F | CF3 | — | H | cyclo-PrCO |
| 10-25 | C—H | C—H | C—Cl | C—H | F | CF3 | — | H | cyclo-PrCH2CO |
| 10-26 | C—H | C—H | C—Cl | C—H | F | CF3 | — | H | CF3CH2CO |
| 10-27 | C—H | C—H | C—Cl | C—H | F | CF3 | — | H | CH3SCH2CO |
| 10-28 | C—H | C—H | C—Cl | C—H | F | CF3 | — | H | CH3S(O)CH2CO |
| 10-29 | C—H | C—H | C—Cl | C—H | F | CF3 | — | H | CH3SO2CH2CO |
| 10-30 | C—H | C—H | C—Cl | C—H | F | CF3 | — | H | CH3CH2NHCO |
| 10-31 | C—Cl | C—H | C—Cl | C—H | H | CF3 | — | H | H |
| 10-32 | C—Cl | C—H | C—Cl | C—H | H | CF3 | — | H | HCO |
| 10-33 | C—Cl | C—H | C—Cl | C—H | H | CF3 | — | H | CH3CO |
| 10-34 | C—Cl | C—H | C—Cl | C—H | H | CF3 | — | H | CH3CH2CO |
| 10-35 | C—Cl | C—H | C—Cl | C—H | H | CF3 | — | H | n-PrCO |
| 10-36 | C—Cl | C—H | C—Cl | C—H | H | CF3 | — | H | iso-PrCO |
| 10-37 | C—Cl | C—H | C—Cl | C—H | H | CF3 | — | H | cyclo-PrCO |
| 10-38 | C—Cl | C—H | C—Cl | C—H | H | CF3 | — | H | n-BuCO |
| 10-39 | C—Cl | C—H | C—Cl | C—H | H | CF3 | — | H | tert-BuCO |
| 10-40 | C—Cl | C—H | C—Cl | C—H | H | CF3 | — | H | iso-BuCO |
| 10-41 | C—Cl | C—H | C—Cl | C—H | H | CF3 | — | H | cyclo-BuCO |
| 10-42 | C—Cl | C—H | C—Cl | C—H | H | CF3 | — | H | (CH3)3CCH2CO |
| 10-43 | C—Cl | C—H | C—Cl | C—H | H | CF3 | — | H | cyclo-PenCO |
| 10-44 | C—Cl | C—H | C—Cl | C—H | H | CF3 | — | H | cyclo-HexCO |
| 10-45 | C—Cl | C—H | C—Cl | C—H | H | CF3 | — | H | cyclo-PrCH2CO |
| 10-46 | C—Cl | C—H | C—Cl | C—H | H | CF3 | — | H | CF3CO |
| 10-47 | C—Cl | C—H | C—Cl | C—H | H | CF3 | — | H | CCl3CO |
| 10-48 | C—Cl | C—H | C—Cl | C—H | H | CF3 | — | H | ClCH2CO |
| 10-49 | C—Cl | C—H | C—Cl | C—H | H | CF3 | — | H | CF2ClCO |
| 10-50 | C—Cl | C—H | C—Cl | C—H | H | CF3 | — | H | ClCH2CH2CO |
| 10-51 | C—Cl | C—H | C—Cl | C—H | H | CF3 | — | H | HCF2CF2CO |
| 10-52 | C—Cl | C—H | C—Cl | C—H | H | CF3 | — | H | CF3CH2CO |
| 10-54 | C—Cl | C—H | C—Cl | C—H | H | CF3 | — | H | HCF2CO |
| 10-55 | C—Cl | C—H | C—Cl | C—H | H | CF3 | — | H | ClCH2CO |
| 10-56 | C—Cl | C—H | C—Cl | C—H | H | CF3 | — | H | 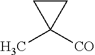 |
| 10-57 | C—Cl | C—H | C—Cl | C—H | H | CF3 | — | H | 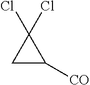 |
| 10-58 | C—Cl | C—H | C—Cl | C—H | H | CF3 | — | H | 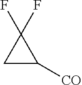 |
| 10-59 | C—Cl | C—H | C—Cl | C—H | H | CF3 | — | H | 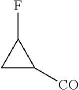 |
| 10-60 | C—Cl | C—H | C—Cl | C—H | H | CF3 | — | H | 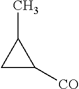 |
| 10-61 | C—Cl | C—H | C—Cl | C—H | H | CF3 | — | H | 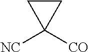 |
| 10-62 | C—Cl | C—H | C—Cl | C—H | H | CF3 | — | H | CH2═CH—CO |
| 10-63 | C—Cl | C—H | C—Cl | C—H | H | CF3 | — | H | CH3CH═CH—CO |
| 10-64 | C—Cl | C—H | C—Cl | C—H | H | CF3 | — | H | CH2═C(CH3)—CO |
| 10-65 | C—Cl | C—H | C—Cl | C—H | H | CF3 | — | H | CH3CH═C(CH3)—CO |
| 10-66 | C—Cl | C—H | C—Cl | C—H | H | CF3 | — | H | (CH3)2C═CH—CO |
| 10-67 | C—Cl | C—H | C—Cl | C—H | H | CF3 | — | H | CH2═C(Cl)—CO |
| 10-68 | C—Cl | C—H | C—Cl | C—H | H | CF3 | — | H | ClCH═C(CH3)—CO |
| 10-69 | C—Cl | C—H | C—Cl | C—H | H | CF3 | — | H | CH2═CHCH2—CO |
| 10-70 | C—Cl | C—H | C—Cl | C—H | H | CF3 | — | H | HCC—CO |
| 10-71 | C—Cl | C—H | C—Cl | C—H | H | CF3 | — | H | CH3CC—CO |
| 10-72 | C—Cl | C—H | C—Cl | C—H | H | CF3 | — | H | NCCH2—CO |
| 10-73 | C—Cl | C—H | C—Cl | C—H | H | CF3 | — | H | CH3OCH2—CO |
| 10-74 | C—Cl | C—H | C—Cl | C—H | H | CF3 | — | H | C2H5OCH2—CO |
| 10-75 | C—Cl | C—H | C—Cl | C—H | H | CF3 | — | H | CH3OCH2CH2—CO |
| 10-76 | C—Cl | C—H | C—Cl | C—H | H | CF3 | — | H | CH3CH2OCH2CH2—CO |
| 10-77 | C—Cl | C—H | C—Cl | C—H | H | CF3 | — | H | CH3SCH2CO |
| 10-78 | C—Cl | C—H | C—Cl | C—H | H | CF3 | — | H | CH3S(O)CH2CO |
| 10-79 | C—Cl | C—H | C—Cl | C—H | H | CF3 | — | H | CH3SO2CH2CO |
| 10-80 | C—Cl | C—H | C—Cl | C—H | H | CF3 | — | H | CH3O(CO)CH2—CO |
| 10-81 | C—Cl | C—H | C—Cl | C—H | H | CF3 | — | H | CH3(CO)OCH2—CO |
| 10-82 | C—Cl | C—H | C—Cl | C—H | H | CF3 | — | H | CH3O(CO)—CO |
| 10-83 | C—Cl | C—H | C—Cl | C—H | H | CF3 | — | H | CF3O(CO)—CO |
| 10-84 | C—Cl | C—H | C—Cl | C—H | H | CF3 | — | H | CH3(CO)CH2—CO |
| 10-85 | C—Cl | C—H | C—Cl | C—H | H | CF3 | — | H | (CH3)2NCH2—CO |
| 10-86 | C—Cl | C—H | C—Cl | C—H | H | CF3 | — | H | PhCO |
| 10-87 | C—Cl | C—H | C—Cl | C—H | H | CF3 | — | H | PhCH2CO |
| 10-88 | C—Cl | C—H | C—Cl | C—H | H | CF3 | — | H | 2-py-CO |
| 10-89 | C—Cl | C—H | C—Cl | C—H | H | CF3 | — | H | 3-py-CO |
| 10-90 | C—Cl | C—H | C—Cl | C—H | H | CF3 | — | H | 4-py-CO |
| 10-91 | C—Cl | C—H | C—Cl | C—H | H | CF3 | — | H | 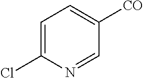 |
| 10-92 | C—Cl | C—H | C—Cl | C—H | H | CF3 | — | H | 2-F—PhCO |
| 10-93 | C—Cl | C—H | C—Cl | C—H | H | CF3 | — | H | 3-F—PhCO |
| 10-94 | C—Cl | C—H | C—Cl | C—H | H | CF3 | — | H | 4-F—PhCO |
| 10-95 | C—Cl | C—H | C—Cl | C—H | H | CF3 | — | H | 2-Cl—PhCO |
| 10-96 | C—Cl | C—H | C—Cl | C—H | H | CF3 | — | H | 3-Cl—PhCO |
| 10-97 | C—Cl | C—H | C—Cl | C—H | H | CF3 | — | H | 4-Cl—PhCO |
| 10-98 | C—Cl | C—H | C—Cl | C—H | H | CF3 | — | H | 2-Br—PhCO |
| 10-99 | C—Cl | C—H | C—Cl | C—H | H | CF3 | — | H | 2-CF3—PhCO |
| 10-100 | C—Cl | C—H | C—Cl | C—H | H | CF3 | — | H | 2-CH3—PhCO |
| 10-101 | C—Cl | C—H | C—Cl | C—H | H | CF3 | — | H | 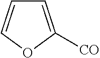 |
| 10-102 | C—Cl | C—H | C—Cl | C—H | H | CF3 | — | H | 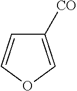 |
| 10-103 | C—Cl | C—H | C—Cl | C—H | H | CF3 | — | H | 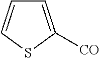 |
| 10-104 | C—Cl | C—H | C—Cl | C—H | H | CF3 | — | H | 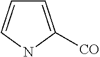 |
| 10-105 | C—Cl | C—H | C—Cl | C—H | H | CF3 | — | H | 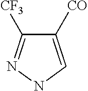 |
| 10-106 | C—Cl | C—H | C—Cl | C—H | H | CF3 | — | H | 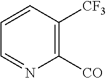 |
| 10-107 | C—Cl | C—H | C—Cl | C—H | H | CF3 | — | H | 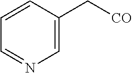 |
| 10-108 | C—Cl | C—H | C—Cl | C—H | H | CF3 | — | H | 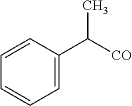 |
| 10-109 | C—Cl | C—H | C—Cl | C—H | H | CF3 | — | H | CH3NHCO |
| 10-110 | C—Cl | C—H | C—Cl | C—H | H | CF3 | — | H | CH3CH2NHCO |
| 10-111 | C—Cl | C—H | C—Cl | C—H | H | CF3 | — | H | CF3CH2NHCO |
| 10-112 | C—Cl | C—H | C—Cl | C—H | H | CF3 | — | H | n-PrNHCO |
| 10-113 | C—Cl | C—H | C—Cl | C—H | H | CF3 | — | H | iso-PrNHCO |
| 10-114 | C—Cl | C—H | C—Cl | C—H | H | CF3 | — | H | cyclo-PrNHCO |
| 10-115 | C—Cl | C—H | C—Cl | C—H | H | CF3 | — | H | n-BuNHCO |
| 10-116 | C—Cl | C—H | C—Cl | C—H | H | CF3 | — | H | tert-BuNHCO |
| 10-117 | C—Cl | C—H | C—Cl | C—H | H | CF3 | — | H | cyclo-BuNHCO |
| 10-118 | C—Cl | C—H | C—Cl | C—H | H | CF3 | — | H | cyclo-PenNHCO |
| 10-119 | C—Cl | C—H | C—Cl | C—H | H | CF3 | — | H | cyclo-HexNHCO |
| 10-120 | C—Cl | C—H | C—Cl | C—H | H | CF3 | — | H | CH2═CHCH2NHCO |
| 10-121 | C—Cl | C—H | C—Cl | C—H | H | CF3 | — | H | HCCCH2NHCO |
| 10-122 | C—Cl | C—H | C—Cl | C—H | H | CF3 | — | H | cyclo-PrCH2NHCO |
| 10-123 | C—Cl | C—H | C—Cl | C—H | H | CF3 | — | H | NCCH2NHCO |
| 10-124 | C—Cl | C—H | C—Cl | C—H | H | CF3 | — | H | CF3(CH3)CHNHCO |
| 10-125 | C—Cl | C—H | C—Cl | C—H | H | CF3 | — | H | CF3(CH3)2CNCO |
| 10-126 | C—Cl | C—H | C—Cl | C—H | H | CF3 | — | H | NCCH2OCO |
| 10-127 | C—Cl | C—H | C—Cl | C—H | H | CF3 | — | H | FCH2CH2NHCO |
| 10-128 | C—Cl | C—H | C—Cl | C—H | H | CF3 | — | H | CF3CH2CH2NHCO |
| 10-129 | C—Cl | C—H | C—Cl | C—H | H | CF3 | — | H | HCF2CF2CH2NHCO |
| 10-130 | C—Cl | C—H | C—Cl | C—H | H | CF3 | — | H | 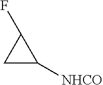 |
| 10-131 | C—Cl | C—H | C—Cl | C—H | H | CF3 | — | H | 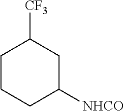 |
| 10-132 | C—Cl | C—H | C—Cl | C—H | H | CF3 | — | H | EtO2CCH2NHCO |
| 10-133 | C—Cl | C—H | C—Cl | C—H | H | CF3 | — | H | 2-CF3—PhNHCO |
| 10-134 | C—Cl | C—H | C—Cl | C—H | H | CF3 | — | H | 3-CF3—PhNHCO |
| 10-135 | C—Cl | C—H | C—Cl | C—H | H | CF3 | — | H | 4-CF3—PhNHCO |
| 10-136 | C—Cl | C—H | C—Cl | C—H | H | CF3 | — | H | 2-py-NHCO |
| 10-137 | C—Cl | C—H | C—Cl | C—H | H | CF3 | — | H | 3-py-NHCO |
| 10-138 | C—Cl | C—H | C—Cl | C—H | H | CF3 | — | H | 4-py-NHCO |
| 10-139 | C—Cl | C—H | C—Cl | C—H | H | CF3 | — | H | 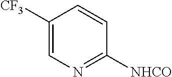 |
| 10-140 | C—Cl | C—H | C—Cl | C—H | H | CF3 | — | H | 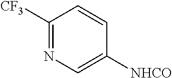 |
| 10-141 | C—Cl | C—H | C—Cl | C—H | H | CF3 | — | H | PhCH2HNCO |
| 10-142 | C—Cl | C—H | C—Cl | C—H | H | CF3 | — | H | 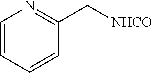 |
| 10-143 | C—Cl | C—H | C—Cl | C—H | H | CF3 | — | H | (CH3)2NCO |
| 10-144 | C—Cl | C—H | C—Cl | C—H | H | CF3 | — | H | pyrrolidineCO |
| 10-145 | C—Cl | C—H | C—Cl | C—H | H | CF3 | — | H | piperidineCO |
| 10-146 | C—Cl | C—H | C—Cl | C—H | H | CF3 | — | H | morpholineCO |
| 10-147 | C—Cl | C—H | C—Cl | C—H | H | CF3 | — | H | CH3OCONH |
| 10-148 | C—Cl | C—H | C—Cl | C—H | H | CF3 | — | H | CH3CH2OCO |
| 10-149 | C—Cl | C—H | C—Cl | C—H | H | CF3 | — | H | n-PrOCO |
| 10-150 | C—Cl | C—H | C—Cl | C—H | H | CF3 | — | H | cyclo-PrOCO |
| 10-151 | C—Cl | C—H | C—Cl | C—H | H | CF3 | — | H | CF3CH2OCO |
| 10-152 | C—Cl | C—H | C—Cl | C—H | H | CF3 | — | H | CH2═CHCH2OCO |
| 10-153 | C—Cl | C—H | C—Cl | C—H | H | CF3 | — | H | HCCCH2OCO |
| 10-154 | C—Cl | C—H | C—Cl | C—H | H | CF3 | — | H | PhOCO |
| 10-155 | C—Cl | C—H | C—Cl | C—H | H | CF3 | — | H | PhCH2OCO |
| 10-156 | C—Cl | C—H | C—Cl | C—H | H | CF3 | — | H | tert-BuOCO |
| 10-157 | C—Cl | C—H | C—Cl | C—H | H | CF3 | — | H | CH3SO2 |
| 10-158 | C—Cl | C—H | C—Cl | C—H | H | CF3 | — | H | CH3CH2SO2 |
| 10-159 | C—Cl | C—H | C—Cl | C—H | H | CF3 | — | H | CF3SO2 |
| 10-160 | C—Cl | C—H | C—Cl | C—H | H | CF3 | — | H | CF3CH2SO2 |
| 10-161 | C—Cl | C—H | C—Cl | C—H | H | CF3 | — | H | PhSO2 |
| 10-162 | C—Cl | C—H | C—Cl | C—H | H | CF3 | — | H | CH3NHCONHSO2 |
| 10-163 | C—Cl | C—H | C—Cl | C—H | H | CF3 | — | H | (CH3)2NCONHSO2 |
| 10-164 | C—Cl | C—H | C—Cl | C—H | H | CF3 | — | H | CH3CS |
| 10-165 | C—Cl | C—H | C—Cl | C—H | H | CF3 | — | H | CH3CH2CS |
| 10-166 | C—Cl | C—H | C—Cl | C—H | H | CF3 | — | H | cyclo-PrCS |
| 10-167 | C—Cl | C—H | C—Cl | C—H | H | CF3 | — | H | CF3CH2CS |
| 10-168 | C—Cl | C—H | C—Br | C—H | H | CF3 | — | H | CH3CO |
| 10-169 | C—Cl | C—H | C—Br | C—H | H | CF3 | — | H | CH3CH2CO |
| 10-170 | C—Cl | C—H | C—Br | C—H | H | CF3 | — | H | n-PrCO |
| 10-171 | C—Cl | C—H | C—Br | C—H | H | CF3 | — | H | cyclo-PrCO |
| 10-172 | C—Cl | C—H | C—Br | C—H | H | CF3 | — | H | cyclo-PrCH2CO |
| 10-173 | C—Cl | C—H | C—Br | C—H | H | CF3 | — | H | CF3CH2CO |
| 10-175 | C—Cl | C—H | C—Br | C—H | H | CF3 | — | H | CH3S(O)CH2CO |
| 10-176 | C—Cl | C—H | C—Br | C—H | H | CF3 | — | H | CH3SO2CH2CO |
| 10-177 | C—Cl | C—H | C—Br | C—H | H | CF3 | — | H | CH3CH2NHCO |
| 10-178 | C—Br | C—H | C—Br | C—H | H | CF3 | — | H | CH3CO |
| 10-179 | C—Br | C—H | C—Br | C—H | H | CF3 | — | H | CH3CH2CO |
| 10-180 | C—Br | C—H | C—Br | C—H | H | CF3 | — | H | n-PrCO |
| 10-181 | C—Br | C—H | C—Br | C—H | H | CF3 | — | H | cyclo-PrCO |
| 10-182 | C—Br | C—H | C—Br | C—H | H | CF3 | — | H | cyclo-PrCH2CO |
| 10-183 | C—Br | C—H | C—Br | C—H | H | CF3 | — | H | CF3CH2CO |
| 10-185 | C—Br | C—H | C—Br | C—H | H | CF3 | — | H | CH3S(O)CH2CO |
| 10-186 | C—Br | C—H | C—Br | C—H | H | CF3 | — | H | CH3SO2CH2CO |
| 10-187 | C—Br | C—H | C—Br | C—H | H | CF3 | — | H | CH3CH2NHCO |
| 10-188 | C—Cl | C—F | C—Cl | C—H | H | CF3 | — | H | CH3CO |
| 10-189 | C—Cl | C—F | C—Cl | C—H | H | CF3 | — | H | CH3CH2CO |
| 10-190 | C—Cl | C—F | C—Cl | C—H | H | CF3 | — | H | n-PrCO |
| 10-191 | C—Cl | C—F | C—Cl | C—H | H | CF3 | — | H | cyclo-PrCO |
| 10-192 | C—Cl | C—F | C—Cl | C—H | H | CF3 | — | H | cyclo-PrCH2CO |
| 10-193 | C—Cl | C—F | C—Cl | C—H | H | CF3 | — | H | CF3CH2CO |
| 10-195 | C—Cl | C—F | C—Cl | C—H | H | CF3 | — | H | CH3S(O)CH2CO |
| 10-196 | C—Cl | C—F | C—Cl | C—H | H | CF3 | — | H | CH3SO2CH2CO |
| 10-197 | C—Cl | C—F | C—Cl | C—H | H | CF3 | — | H | CH3CH2NHCO |
| 10-198 | C—Cl | C—Cl | C—Cl | C—H | H | CF3 | — | H | CH3CO |
| 10-199 | C—Cl | C—Cl | C—Cl | C—H | H | CF3 | — | H | CH3CH2CO |
| 10-200 | C—Cl | C—Cl | C—Cl | C—H | H | CF3 | — | H | n-PrCO |
| 10-201 | C—Cl | C—Cl | C—Cl | C—H | H | CF3 | — | H | cyclo-PrCO |
| 10-202 | C—Cl | C—Cl | C—Cl | C—H | H | CF3 | — | H | cyclo-PrCH2CO |
| 10-203 | C—Cl | C—Cl | C—Cl | C—H | H | CF3 | — | H | CF3CH2CO |
| 10-205 | C—Cl | C—Cl | C—Cl | C—H | H | CF3 | — | H | CH3CH2OCH2CH2—CO |
| 10-206 | C—Cl | C—Cl | C—Cl | C—H | H | CF3 | — | H | CH3SCH2CO |
| 10-207 | C—Cl | C—Cl | C—Cl | C—H | H | CF3 | — | H | CH3S(O)CH2CO |
| 10-208 | C—Cl | C—Cl | C—Cl | C—H | H | CF3 | — | H | CH3SO2CH2CO |
| 10-209 | C—Cl | C—Cl | C—Cl | C—H | H | CF3 | — | H | CH3CH2NHCO |
| 10-210 | C—Cl | C—Br | C—Cl | C—H | H | CF3 | — | H | CH3CO |
| 10-211 | C—Cl | C—Br | C—Cl | C—H | H | CF3 | — | H | CH3CH2CO |
| 10-212 | C—Cl | C—Br | C—Cl | C—H | H | CF3 | — | H | n-PrCO |
| 10-213 | C—Cl | C—Br | C—Cl | C—H | H | CF3 | — | H | cyclo-PrCO |
| 10-214 | C—Cl | C—Br | C—Cl | C—H | H | CF3 | — | H | cyclo-PrCH2CO |
| 10-215 | C—Cl | C—Br | C—Cl | C—H | H | CF3 | — | H | CF3CH2CO |
| 10-217 | C—Cl | C—Br | C—Cl | C—H | H | CF3 | — | H | CH3S(O)CH2CO |
| 10-218 | C—Cl | C—Br | C—Cl | C—H | H | CF3 | — | H | CH3SO2CH2CO |
| 10-219 | C—Cl | C—Br | C—Cl | C—H | H | CF3 | — | H | CH3CH2NHCO |
| 10-220 | C—Cl | C—Cl | C—Br | C—H | H | CF3 | — | H | CH3CO |
| 10-221 | C—Cl | C—Cl | C—Br | C—H | H | CF3 | — | H | CH3CH2CO |
| 10-222 | C—Cl | C—Cl | C—Br | C—H | H | CF3 | — | H | n-PrCO |
| 10-223 | C—Cl | C—Cl | C—Br | C—H | H | CF3 | — | H | cyclo-PrCO |
| 10-224 | C—Cl | C—Cl | C—Br | C—H | H | CF3 | — | H | cyclo-PrCH2CO |
| 10-225 | C—Cl | C—Cl | C—Br | C—H | H | CF3 | — | H | CF3CH2CO |
| 10-227 | C—Cl | C—Cl | C—Br | C—H | H | CF3 | — | H | CH3S(O)CH2CO |
| 10-228 | C—Cl | C—Cl | C—Br | C—H | H | CF3 | — | H | CH3SO2CH2CO |
| 10-229 | C—Cl | C—Cl | C—Br | C—H | H | CF3 | — | H | CH3CH2NHCO |
| 10-230 | C—Cl | C—F | C—Cl | C—H | F | CF3 | — | H | CH3CO |
| 10-231 | C—Cl | C—F | C—Cl | C—H | F | CF3 | — | H | CH3CH2CO |
| 10-232 | C—Cl | C—F | C—Cl | C—H | F | CF3 | — | H | n-PrCO |
| 10-233 | C—Cl | C—F | C—Cl | C—H | F | CF3 | — | H | cyclo-PrCO |
| 10-234 | C—Cl | C—F | C—Cl | C—H | F | CF3 | — | H | cyclo-PrCH2CO |
| 10-235 | C—Cl | C—F | C—Cl | C—H | F | CF3 | — | H | CF3CH2CO |
| 10-237 | C—Cl | C—F | C—Cl | C—H | F | CF3 | — | H | CH3S(O)CH2CO |
| 10-238 | C—Cl | C—F | C—Cl | C—H | F | CF3 | — | H | CH3SO2CH2CO |
| 10-239 | C—Cl | C—F | C—Cl | C—H | F | CF3 | — | H | CH3CH2NHCO |
| 10-240 | C—CF3 | C—H | C—H | C—H | H | CF3 | — | H | CH3CO |
| 10-241 | C—CF3 | C—H | C—H | C—H | H | CF3 | — | H | CH3CH2CO |
| 10-242 | C—CF3 | C—H | C—H | C—H | H | CF3 | — | H | n-PrCO |
| 10-243 | C—CF3 | C—H | C—H | C—H | H | CF3 | — | H | cyclo-PrCO |
| 10-244 | C—CF3 | C—H | C—H | C—H | H | CF3 | — | H | cyclo-PrCH2CO |
| 10-245 | C—CF3 | C—H | C—H | C—H | H | CF3 | — | H | CF3CH2CO |
| 10-247 | C—CF3 | C—H | C—H | C—H | H | CF3 | — | H | CH3S(O)CH2CO |
| 10-248 | C—CF3 | C—H | C—H | C—H | H | CF3 | — | H | CH3SO2CH2CO |
| 10-249 | C—CF3 | C—H | C—H | C—H | H | CF3 | — | H | CH3CH2NHCO |
| 10-250 | C—CF3 | C—F | C—H | C—H | H | CF3 | — | H | CH3CO |
| 10-251 | C—CF3 | C—F | C—H | C—H | H | CF3 | — | H | CH3CH2CO |
| 10-252 | C—CF3 | C—F | C—H | C—H | H | CF3 | — | H | n-PrCO |
| 10-253 | C—CF3 | C—F | C—H | C—H | H | CF3 | — | H | cyclo-PrCO |
| 10-254 | C—CF3 | C—F | C—H | C—H | H | CF3 | — | H | cyclo-PrCH2CO |
| 10-255 | C—CF3 | C—F | C—H | C—H | H | CF3 | — | H | CF3CH2CO |
| 10-257 | C—CF3 | C—F | C—H | C—H | H | CF3 | — | H | CH3S(O)CH2CO |
| 10-258 | C—CF3 | C—F | C—H | C—H | H | CF3 | — | H | CH3SO2CH2CO |
| 10-259 | C—CF3 | C—F | C—H | C—H | H | CF3 | — | H | CH3CH2NHCO |
| 10-260 | C—CF3 | C—H | C—H | C—H | F | CF3 | — | H | CH3CO |
| 10-261 | C—CF3 | C—H | C—H | C—H | F | CF3 | — | H | CH3CH2CO |
| 10-262 | C—CF3 | C—H | C—H | C—H | F | CF3 | — | H | n-PrCO |
| 10-263 | C—CF3 | C—H | C—H | C—H | F | CF3 | — | H | cyclo-PrCO |
| 10-264 | C—CF3 | C—H | C—H | C—H | F | CF3 | — | H | cyclo-PrCH2CO |
| 10-265 | C—CF3 | C—H | C—H | C—H | F | CF3 | — | H | CF3CH2CO |
| 10-267 | C—CF3 | C—H | C—H | C—H | F | CF3 | — | H | CH3S(O)CH2CO |
| 10-268 | C—CF3 | C—H | C—H | C—H | F | CF3 | — | H | CH3SO2CH2CO |
| 10-269 | C—CF3 | C—H | C—H | C—H | F | CF3 | — | H | CH3CH2NHCO |
| 10-270 | C—H | C—H | C—CF3 | C—H | F | CF3 | — | H | CH3CO |
| 10-271 | C—H | C—H | C—CF3 | C—H | F | CF3 | — | H | CH3CH2CO |
| 10-272 | C—H | C—H | C—CF3 | C—H | F | CF3 | — | H | n-PrCO |
| 10-273 | C—H | C—H | C—CF3 | C—H | F | CF3 | — | H | cyclo-PrCO |
| 10-274 | C—H | C—H | C—CF3 | C—H | F | CF3 | — | H | cyclo-PrCH2CO |
| 10-275 | C—H | C—H | C—CF3 | C—H | F | CF3 | — | H | CF3CH2CO |
| 10-277 | C—H | C—H | C—CF3 | C—H | F | CF3 | — | H | CH3S(O)CH2CO |
| 10-278 | C—H | C—H | C—CF3 | C—H | F | CF3 | — | H | CH3SO2CH2CO |
| 10-279 | C—H | C—H | C—CF3 | C—H | F | CF3 | — | H | CH3CH2NHCO |
| 10-280 | C—Cl | C—H | C—CF3 | C—H | H | CF3 | — | H | CH3CO |
| 10-281 | C—Cl | C—H | C—CF3 | C—H | H | CF3 | — | H | CH3CH2CO |
| 10-282 | C—Cl | C—H | C—CF3 | C—H | H | CF3 | — | H | n-PrCO |
| 10-283 | C—Cl | C—H | C—CF3 | C—H | H | CF3 | — | H | cyclo-PrCO |
| 10-284 | C—Cl | C—H | C—CF3 | C—H | H | CF3 | — | H | cyclo-PrCH2CO |
| 10-285 | C—Cl | C—H | C—CF3 | C—H | H | CF3 | — | H | CF3CH2CO |
| 10-287 | C—Cl | C—H | C—CF3 | C—H | H | CF3 | — | H | CH3CH2OCH2CH2—CO |
| 10-288 | C—Cl | C—H | C—CF3 | C—H | H | CF3 | — | H | CH3SCH2CO |
| 10-289 | C—Cl | C—H | C—CF3 | C—H | H | CF3 | — | H | CH3S(O)CH2CO |
| 10-290 | C—Cl | C—H | C—CF3 | C—H | H | CF3 | — | H | CH3SO2CH2CO |
| 10-291 | C—Cl | C—H | C—CF3 | C—H | H | CF3 | — | H | CH3CH2NHCO |
| 10-292 | C—CF3 | C—H | C—CF3 | C—H | H | CF3 | — | H | CH3CO |
| 10-293 | C—CF3 | C—H | C—CF3 | C—H | H | CF3 | — | H | CH3CH2CO |
| 10-294 | C—CF3 | C—H | C—CF3 | C—H | H | CF3 | — | H | n-PrCO |
| 10-295 | C—CF3 | C—H | C—CF3 | C—H | H | CF3 | — | H | cyclo-PrCO |
| 10-296 | C—CF3 | C—H | C—CF3 | C—H | H | CF3 | — | H | cyclo-PrCH2CO |
| 10-297 | C—CF3 | C—H | C—CF3 | C—H | H | CF3 | — | H | CF3CH2CO |
| 10-299 | C—CF3 | C—H | C—CF3 | C—H | H | CF3 | — | H | CH3CH2OCH2CH2—CO |
| 10-300 | C—CF3 | C—H | C—CF3 | C—H | H | CF3 | — | H | CH3SCH2CO |
| 10-301 | C—CF3 | C—H | C—CF3 | C—H | H | CF3 | — | H | CH3S(O)CH2CO |
| 10-302 | C—CF3 | C—H | C—CF3 | C—H | H | CF3 | — | H | CH3SO2CH2CO |
| 10-303 | C—CF3 | C—H | C—CF3 | C—H | H | CF3 | — | H | CH3CH2NHCO |
| 10-304 | C—Cl | C—Cl | C—CF3 | C—H | H | CF3 | — | H | CH3CO |
| 10-305 | C—Cl | C—Cl | C—CF3 | C—H | H | CF3 | — | H | CH3CH2CO |
| 10-306 | C—Cl | C—Cl | C—CF3 | C—H | H | CF3 | — | H | n-PrCO |
| 10-307 | C—Cl | C—Cl | C—CF3 | C—H | H | CF3 | — | H | cyclo-PrCO |
| 10-308 | C—Cl | C—Cl | C—CF3 | C—H | H | CF3 | — | H | cyclo-PrCH2CO |
| 10-309 | C—Cl | C—Cl | C—CF3 | C—H | H | CF3 | — | H | CF3CH2CO |
| 10-311 | C—Cl | C—Cl | C—CF3 | C—H | H | CF3 | — | H | CH3S(O)CH2CO |
| 10-312 | C—Cl | C—Cl | C—CF3 | C—H | H | CF3 | — | H | CH3SO2CH2CO |
| 10-313 | C—Cl | C—Cl | C—CF3 | C—H | H | CF3 | — | H | CH3CH2NHCO |
| 10-314 | C—Cl | N | C—H | C—H | H | CF3 | — | H | CH3CO |
| 10-315 | C—Cl | N | C—H | C—H | H | CF3 | — | H | CH3CH2CO |
| 10-316 | C—Cl | N | C—H | C—H | H | CF3 | — | H | n-PrCO |
| 10-317 | C—Cl | N | C—H | C—H | H | CF3 | — | H | cyclo-PrCO |
| 10-318 | C—Cl | N | C—H | C—H | H | CF3 | — | H | cyclo-PrCH2CO |
| 10-319 | C—Cl | N | C—H | C—H | H | CF3 | — | H | CF3CH2CO |
| 10-321 | C—Cl | N | C—H | C—H | H | CF3 | — | H | CH3S(O)CH2CO |
| 10-322 | C—Cl | N | C—H | C—H | H | CF3 | — | H | CH3SO2CH2CO |
| 10-323 | C—Cl | N | C—H | C—H | H | CF3 | — | H | CH3CH2NHCO |
| 10-324 | C—Cl | N | C—Cl | C—H | H | CF3 | — | H | CH3CO |
| 10-325 | C—Cl | N | C—Cl | C—H | H | CF3 | — | H | CH3CH2CO |
| 10-326 | C—Cl | N | C—Cl | C—H | H | CF3 | — | H | n-PrCO |
| 10-327 | C—Cl | N | C—Cl | C—H | H | CF3 | — | H | cyclo-PrCO |
| 10-328 | C—Cl | N | C—Cl | C—H | H | CF3 | — | H | cyclo-PrCH2CO |
| 10-329 | C—Cl | N | C—Cl | C—H | H | CF3 | — | H | CF3CH2CO |
| 10-330 | C—Cl | N | C—Cl | C—H | H | CF3 | — | H | CH3SCH2CO |
| 10-331 | C—Cl | N | C—Cl | C—H | H | CF3 | — | H | CH3S(O)CH2CO |
| 10-332 | C—Cl | N | C—Cl | C—H | H | CF3 | — | H | CH3SO2CH2CO |
| 10-333 | C—Cl | N | C—Cl | C—H | H | CF3 | — | H | CH3CH2NHCO |
| 10-334 | C—CF3 | N | C—H | C—H | H | CF3 | — | H | CH3CO |
| 10-335 | C—CF3 | N | C—H | C—H | H | CF3 | — | H | CH3CH2CO |
| 10-336 | C—CF3 | N | C—H | C—H | H | CF3 | — | H | n-PrCO |
| 10-337 | C—CF3 | N | C—H | C—H | H | CF3 | — | H | cyclo-PrCO |
| 10-338 | C—CF3 | N | C—H | C—H | H | CF3 | — | H | cyclo-PrCH2CO |
| 10-339 | C—CF3 | N | C—H | C—H | H | CF3 | — | H | CF3CH2CO |
| 10-340 | C—CF3 | N | C—H | C—H | H | CF3 | — | H | CH3SCH2CO |
| 10-341 | C—CF3 | N | C—H | C—H | H | CF3 | — | H | CH3S(O)CH2CO |
| 10-342 | C—CF3 | N | C—H | C—H | H | CF3 | — | H | CH3SO2CH2CO |
| 10-343 | C—CF3 | N | C—H | C—H | H | CF3 | — | H | CH3CH2NHCO |
| 10-344 | C—CF3 | N | C—CF3 | C—H | H | CF3 | — | H | CH3CO |
| 10-345 | C—CF3 | N | C—CF3 | C—H | H | CF3 | — | H | CH3CH2CO |
| 10-346 | C—CF3 | N | C—CF3 | C—H | H | CF3 | — | H | n-PrCO |
| 10-347 | C—CF3 | N | C—CF3 | C—H | H | CF3 | — | H | cyclo-PrCO |
| 10-348 | C—CF3 | N | C—CF3 | C—H | H | CF3 | — | H | cyclo-PrCH2CO |
| 10-349 | C—CF3 | N | C—CF3 | C—H | H | CF3 | — | H | CF3CH2CO |
| 10-350 | C—CF3 | N | C—CF3 | C—H | H | CF3 | — | H | CH3SCH2CO |
| 10-351 | C—CF3 | N | C—CF3 | C—H | H | CF3 | — | H | CH3S(O)CH2CO |
| 10-352 | C—CF3 | N | C—CF3 | C—H | H | CF3 | — | H | CH3SO2CH2CO |
| 10-353 | C—CF3 | N | C—CF3 | C—H | H | CF3 | — | H | CH3CH2NHCO |
| 10-354 | C—CF3 | C—H | C—H | N | H | CF3 | — | H | CH3CO |
| 10-355 | C—CF3 | C—H | C—H | N | H | CF3 | — | H | CH3CH2CO |
| 10-356 | C—CF3 | C—H | C—H | N | H | CF3 | — | H | n-PrCO |
| 10-357 | C—CF3 | C—H | C—H | N | H | CF3 | — | H | cyclo-PrCO |
| 10-358 | C—CF3 | C—H | C—H | N | H | CF3 | — | H | cyclo-PrCH2CO |
| 10-359 | C—CF3 | C—H | C—H | N | H | CF3 | — | H | CF3CH2CO |
| 10-360 | C—CF3 | C—H | C—H | N | H | CF3 | — | H | CH3SCH2CO |
| 10-361 | C—CF3 | C—H | C—H | N | H | CF3 | — | H | CH3S(O)CH2CO |
| 10-362 | C—CF3 | C—H | C—H | N | H | CF3 | — | H | CH3SO2CH2CO |
| 10-363 | C—CF3 | C—H | C—H | N | H | CF3 | — | H | CH3CH2NHCO |
| 10-364 | C—H | C—H | C—CF3 | N | H | CF3 | — | H | CH3CO |
| 10-365 | C—H | C—H | C—CF3 | N | H | CF3 | — | H | CH3CH2CO |
| 10-366 | C—H | C—H | C—CF3 | N | H | CF3 | — | H | n-PrCO |
| 10-367 | C—H | C—H | C—CF3 | N | H | CF3 | — | H | cyclo-PrCO |
| 10-368 | C—H | C—H | C—CF3 | N | H | CF3 | — | H | cyclo-PrCH2CO |
| 10-369 | C—H | C—H | C—CF3 | N | H | CF3 | — | H | CF3CH2CO |
| 10-370 | C—H | C—H | C—CF3 | N | H | CF3 | — | H | CH3SCH2CO |
| 10-371 | C—H | C—H | C—CF3 | N | H | CF3 | — | H | CH3S(O)CH2CO |
| 10-372 | C—H | C—H | C—CF3 | N | H | CF3 | — | H | CH3SO2CH2CO |
| 10-373 | C—H | C—H | C—CF3 | N | H | CF3 | — | H | CH3CH2NHCO |
| 10-374 | C—CF3 | C—H | C—Cl | N | H | CF3 | — | H | CH3CO |
| 10-375 | C—CF3 | C—H | C—Cl | N | H | CF3 | — | H | CH3CH2CO |
| 10-376 | C—CF3 | C—H | C—Cl | N | H | CF3 | — | H | n-PrCO |
| 10-377 | C—CF3 | C—H | C—Cl | N | H | CF3 | — | H | cyclo-PrCO |
| 10-378 | C—CF3 | C—H | C—Cl | N | H | CF3 | — | H | cyclo-PrCH2CO |
| 10-379 | C—CF3 | C—H | C—Cl | N | H | CF3 | — | H | CF3CH2CO |
| 10-380 | C—CF3 | C—H | C—Cl | N | H | CF3 | — | H | CH3SCH2CO |
| 10-381 | C—CF3 | C—H | C—Cl | N | H | CF3 | — | H | CH3S(O)CH2CO |
| 10-382 | C—CF3 | C—H | C—Cl | N | H | CF3 | — | H | CH3SO2CH2CO |
| 10-383 | C—CF3 | C—H | C—Cl | N | H | CF3 | — | H | CH3CH2NHCO |
| 10-384 | C—CF3 | C—H | C—Br | N | H | CF3 | — | H | CH3CO |
| 10-385 | C—CF3 | C—H | C—Br | N | H | CF3 | — | H | CH3CH2CO |
| 10-386 | C—CF3 | C—H | C—Br | N | H | CF3 | — | H | n-PrCO |
| 10-387 | C—CF3 | C—H | C—Br | N | H | CF3 | — | H | cyclo-PrCO |
| 10-388 | C—CF3 | C—H | C—Br | N | H | CF3 | — | H | cyclo-PrCH2CO |
| 10-389 | C—CF3 | C—H | C—Br | N | H | CF3 | — | H | CF3CH2CO |
| 10-390 | C—CF3 | C—H | C—Br | N | H | CF3 | — | H | CH3SCH2CO |
| 10-391 | C—CF3 | C—H | C—Br | N | H | CF3 | — | H | CH3S(O)CH2CO |
| 10-392 | C—CF3 | C—H | C—Br | N | H | CF3 | — | H | CH3SO2CH2CO |
| 10-393 | C—CF3 | C—H | C—Br | N | H | CF3 | — | H | CH3CH2NHCO |
| 10-394 | C—CF3 | C—H | C—CF3 | N | H | CF3 | — | H | CH3CO |
| 10-395 | C—CF3 | C—H | C—CF3 | N | H | CF3 | — | H | CH3CH2CO |
| 10-396 | C—CF3 | C—H | C—CF3 | N | H | CF3 | — | H | n-PrCO |
| 10-397 | C—CF3 | C—H | C—CF3 | N | H | CF3 | — | H | cyclo-PrCO |
| 10-398 | C—CF3 | C—H | C—CF3 | N | H | CF3 | — | H | cyclo-PrCH2CO |
| 10-399 | C—CF3 | C—H | C—CF3 | N | H | CF3 | — | H | CF3CH2CO |
| 10-400 | C—CF3 | C—H | C—CF3 | N | H | CF3 | — | H | CH3SCH2CO |
| 10-401 | C—CF3 | C—H | C—CF3 | N | H | CF3 | — | H | CH3S(O)CH2CO |
| 10-402 | C—CF3 | C—H | C—CF3 | N | H | CF3 | — | H | CH3SO2CH2CO |
| 10-403 | C—CF3 | C—H | C—CF3 | N | H | CF3 | — | H | CH3CH2NI ICO |
| 10-404 | C—Cl | C—H | C—H | C—H | H | CF3 | CH2 | H | CH3CO |
| 10-405 | C—Cl | C—H | C—H | C—H | H | CF3 | CH2 | H | CH3CH2CO |
| 10-406 | C—Cl | C—H | C—H | C—H | H | CF3 | CH2 | H | n-PrCO |
| 10-407 | C—Cl | C—H | C—H | C—H | H | CF3 | CH2 | H | cyclo-PrCO |
| 10-408 | C—Cl | C—H | C—H | C—H | H | CF3 | CH2 | H | cyclo-PrCH2CO |
| 10-409 | C—Cl | C—H | C—H | C—H | H | CF3 | CH2 | H | CF3CH2CO |
| 10-410 | C—Cl | C—H | C—H | C—H | H | CF3 | CH2 | H | CH3SCH2CO |
| 10-411 | C—Cl | C—H | C—H | C—H | H | CF3 | CH2 | H | CH3S(O)CH2CO |
| 10-412 | C—Cl | C—H | C—H | C—H | H | CF3 | CH2 | H | CH3SO2CH2CO |
| 10-413 | C—Cl | C—H | C—H | C—H | H | CF3 | CH2 | H | CH3CH2NHCO |
| 10-414 | C—Cl | C—H | C—H | C—H | F | CF3 | CH2 | H | CH3CO |
| 10-415 | C—Cl | C—H | C—H | C—H | F | CF3 | CH2 | H | CH3CH2CO |
| 10-416 | C—Cl | C—H | C—H | C—H | F | CF3 | CH2 | H | n-PrCO |
| 10-417 | C—Cl | C—H | C—H | C—H | F | CF3 | CH2 | H | cyclo-PrCO |
| 10-418 | C—Cl | C—H | C—H | C—H | F | CF3 | CH2 | H | cyclo-PrCH2CO |
| 10-419 | C—Cl | C—H | C—H | C—H | F | CF3 | CH2 | H | CF3CH2CO |
| 10-420 | C—Cl | C—H | C—H | C—H | F | CF3 | CH2 | H | CH3SCH2CO |
| 10-421 | C—Cl | C—H | C—H | C—H | F | CF3 | CH2 | H | CH3S(O)CH2CO |
| 10-422 | C—Cl | C—H | C—H | C—H | F | CF3 | CH2 | H | CH3SO2CH2CO |
| 10-423 | C—Cl | C—H | C—H | C—H | F | CF3 | CH2 | H | CH3CH2NHCO |
| 10-424 | C—H | C—H | C—Cl | C—H | F | CF3 | CH2 | H | CH3CO |
| 10-425 | C—H | C—H | C—Cl | C—H | F | CF3 | CH2 | H | CH3CH2CO |
| 10-426 | C—H | C—H | C—Cl | C—H | F | CF3 | CH2 | H | n-PrCO |
| 10-427 | C—H | C—H | C—Cl | C—H | F | CF3 | CH2 | H | cyclo-PrCO |
| 10-428 | C—H | C—H | C—Cl | C—H | F | CF3 | CH2 | H | cyclo-PrCH2CO |
| 10-429 | C—H | C—H | C—Cl | C—H | F | CF3 | CH2 | H | CF3CH2CO |
| 10-430 | C—H | C—H | C—Cl | C—H | F | CF3 | CH2 | H | CH3SCH2CO |
| 10-431 | C—H | C—H | C—Cl | C—H | F | CF3 | CH2 | H | CH3S(O)CH2CO |
| 10-432 | C—H | C—H | C—Cl | C—H | F | CF3 | CH2 | H | CH3SO2CH2CO |
| 10-433 | C—H | C—H | C—Cl | C—H | F | CF3 | CH2 | H | CH3CH2NHCO |
| 10-434 | C—Cl | C—H | C—Cl | C—H | H | CF3 | CH2 | H | H |
| 10-435 | C—Cl | C—H | C—Cl | C—H | H | CF3 | CH2 | H | HCO |
| 10-436 | C—Cl | C—H | C—Cl | C—H | H | CF3 | CH2 | H | CH3CO |
| 10-437 | C—Cl | C—H | C—Cl | C—H | H | CF3 | CH2 | H | CH3CH2CO |
| 10-438 | C—Cl | C—H | C—Cl | C—H | H | CF3 | CH2 | H | n-PrCO |
| 10-439 | C—Cl | C—H | C—Cl | C—H | H | CF3 | CH2 | H | iso-PrCO |
| 10-440 | C—Cl | C—H | C—Cl | C—H | H | CF3 | CH2 | H | cyclo-PrCO |
| 10-441 | C—Cl | C—H | C—Cl | C—H | H | CF3 | CH2 | H | n-BuCO |
| 10-442 | C—Cl | C—H | C—Cl | C—H | H | CF3 | CH2 | H | tert-BuCO |
| 10-443 | C—Cl | C—H | C—Cl | C—H | H | CF3 | CH2 | H | iso-BuCO |
| 10-444 | C—Cl | C—H | C—Cl | C—H | H | CF3 | CH2 | H | cyclo-BuCO |
| 10-445 | C—Cl | C—H | C—Cl | C—H | H | CF3 | CH2 | H | (CH3)3CCH2CO |
| 10-446 | C—Cl | C—H | C—Cl | C—H | H | CF3 | CH2 | H | cyclo-PenCO |
| 10-447 | C—Cl | C—H | C—Cl | C—H | H | CF3 | CH2 | H | cyclo-HexCO |
| 10-448 | C—Cl | C—H | C—Cl | C—H | H | CF3 | CH2 | H | cyclo-PrCH2CO |
| 10-449 | C—Cl | C—H | C—Cl | C—H | H | CF3 | CH2 | H | CF3CO |
| 10-450 | C—Cl | C—H | C—Cl | C—H | H | CF3 | CH2 | H | CCl3CO |
| 10-451 | C—Cl | C—H | C—Cl | C—H | H | CF3 | CH2 | H | ClCH2CO |
| 10-452 | C—Cl | C—H | C—Cl | C—H | H | CF3 | CH2 | H | CF2ClCO |
| 10-453 | C—Cl | C—H | C—Cl | C—H | H | CF3 | CH2 | H | ClCH2CH2CO |
| 10-454 | C—Cl | C—H | C—Cl | C—H | H | CF3 | CH2 | H | HCF2CF2CO |
| 10-455 | C—Cl | C—H | C—Cl | C—H | H | CF3 | CH2 | H | CF3CH2CO |
| 10-456 | C—Cl | C—H | C—Cl | C—H | H | CF3 | CH2 | H | (CF3)2CHCO |
| 10-457 | C—Cl | C—H | C—Cl | C—H | H | CF3 | CH2 | H | HCF2CO |
| 10-458 | C—Cl | C—H | C—Cl | C—H | H | CF3 | CH2 | H | ClCH2CO |
| 10-459 | C—Cl | C—H | C—Cl | C—H | H | CF3 | CH2 | H | 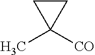 |
| 10-460 | C—Cl | C—H | C—Cl | C—H | H | CF3 | CH2 | H | 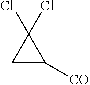 |
| 10-461 | C—Cl | C—H | C—Cl | C—H | H | CF3 | CH2 | H | 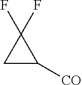 |
| 10-462 | C—Cl | C—H | C—Cl | C—H | H | CF3 | CH2 | H | 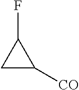 |
| 10-463 | C—Cl | C—H | C—Cl | C—H | H | CF3 | CH2 | H | 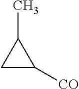 |
| 10-464 | C—Cl | C—H | C—Cl | C—H | H | CF3 | CH2 | H | 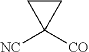 |
| 10-465 | C—Cl | C—H | C—Cl | C—H | H | CF3 | CH2 | H | CH2═CH—CO |
| 10-466 | C—Cl | C—H | C—Cl | C—H | H | CF3 | CH2 | H | CH3CH═CH—CO |
| 10-467 | C—Cl | C—H | C—Cl | C—H | H | CF3 | CH2 | H | CH2═C(CH3)—CO |
| 10-468 | C—Cl | C—H | C—Cl | C—H | H | CF3 | CH2 | H | CH3CH═C(CH3)—CO |
| 10-469 | C—Cl | C—H | C—Cl | C—H | H | CF3 | CH2 | H | (CH3)2C═CH—CO |
| 10-470 | C—Cl | C—H | C—Cl | C—H | H | CF3 | CH2 | H | CH2═C(Cl)—CO |
| 10-471 | C—Cl | C—H | C—Cl | C—H | H | CF3 | CH2 | H | ClCH═C(CH3)—CO |
| 10-472 | C—Cl | C—H | C—Cl | C—H | H | CF3 | CH2 | H | CH2═CHCH2—CO |
| 10-473 | C—Cl | C—H | C—Cl | C—H | H | CF3 | CH2 | H | HCC—CO |
| 10-474 | C—Cl | C—H | C—Cl | C—H | H | CF3 | CH2 | H | CH3CC—CO |
| 10-475 | C—Cl | C—H | C—Cl | C—H | H | CF3 | CH2 | H | NCCH2—CO |
| 10-476 | C—Cl | C—H | C—Cl | C—H | H | CF3 | CH2 | H | CH3OCH2—CO |
| 10-477 | C—Cl | C—H | C—Cl | C—H | H | CF3 | CH2 | H | C2H5OCH2—CO |
| 10-478 | C—Cl | C—H | C—Cl | C—H | H | CF3 | CH2 | H | CH3OCH2CH2—CO |
| 10-479 | C—Cl | C—H | C—Cl | C—H | H | CF3 | CH2 | H | CH3CH2OCH2CH2—CO |
| 10-480 | C—Cl | C—H | C—Cl | C—H | H | CF3 | CH2 | H | CH3SCH2CO |
| 10-481 | C—Cl | C—H | C—Cl | C—H | H | CF3 | CH2 | H | CH3S(O)CH2CO |
| 10-482 | C—Cl | C—H | C—Cl | C—H | H | CF3 | CH2 | H | CH3SO2CH2CO |
| 10-483 | C—Cl | C—H | C—Cl | C—H | H | CF3 | CH2 | H | CH3O(CO)CH2—CO |
| 10-484 | C—Cl | C—H | C—Cl | C—H | H | CF3 | CH2 | H | CH3(CO)OCH2—CO |
| 10-485 | C—Cl | C—H | C—Cl | C—H | H | CF3 | CH2 | H | CH3O(CO)—CO |
| 10-486 | C—Cl | C—H | C—Cl | C—H | H | CF3 | CH2 | H | CF3O(CO)—CO |
| 10-487 | C—Cl | C—H | C—Cl | C—H | H | CF3 | CH2 | H | CH3(CO)CH2—CO |
| 10-488 | C—Cl | C—H | C—Cl | C—H | H | CF3 | CH2 | H | (CH3)2NCH2—CO |
| 10-489 | C—Cl | C—H | C—Cl | C—H | H | CF3 | CH2 | H | PhCO |
| 10-490 | C—Cl | C—H | C—Cl | C—H | H | CF3 | CH2 | H | PhCH2CO |
| 10-491 | C—Cl | C—H | C—Cl | C—H | H | CF3 | CH2 | H | 2-py-CO |
| 10-492 | C—Cl | C—H | C—Cl | C—H | H | CF3 | CH2 | H | 3-py-CO |
| 10-493 | C—Cl | C—H | C—Cl | C—H | H | CF3 | CH2 | H | 4-py-CO |
| 10-494 | C—Cl | C—H | C—Cl | C—H | H | CF3 | CH2 | H | 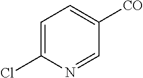 |
| 10-495 | C—Cl | C—H | C—Cl | C—H | H | CF3 | CH2 | H | 2-F—PhCO |
| 10-496 | C—Cl | C—H | C—Cl | C—H | H | CF3 | CH2 | H | 3-F—PhCO |
| 10-497 | C—Cl | C—H | C—Cl | C—H | H | CF3 | CH2 | H | 4-F—PhCO |
| 10-498 | C—Cl | C—H | C—Cl | C—H | H | CF3 | CH2 | H | 2-Cl—PhCO |
| 10-499 | C—Cl | C—H | C—Cl | C—H | H | CF3 | CH2 | H | 3-Cl—PhCO |
| 10-500 | C—Cl | C—H | C—Cl | C—H | H | CF3 | CH2 | H | 4-Cl—PhCO |
| 10-501 | C—Cl | C—H | C—Cl | C—H | H | CF3 | CH2 | H | 2-Br—PhCO |
| 10-502 | C—Cl | C—H | C—Cl | C—H | H | CF3 | CH2 | H | 2-CF3—PhCO |
| 10-503 | C—Cl | C—H | C—Cl | C—H | H | CF3 | CH2 | H | 2-CH3—PhCO |
| 10-504 | C—Cl | C—H | C—Cl | C—H | H | CF3 | CH2 | H | 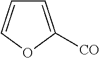 |
| 10-505 | C—Cl | C—H | C—Cl | C—H | H | CF3 | CH2 | H | 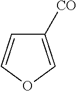 |
| 10-506 | C—Cl | C—H | C—Cl | C—H | H | CF3 | CH2 | H | 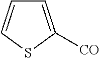 |
| 10-507 | C—Cl | C—H | C—Cl | C—H | H | CF3 | CH2 | H | 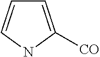 |
| 10-508 | C—Cl | C—H | C—Cl | C—H | H | CF3 | CH2 | H | 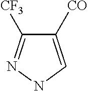 |
| 10-509 | C—Cl | C—H | C—Cl | C—H | H | CF3 | CH2 | H | 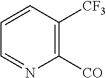 |
| 10-510 | C—Cl | C—H | C—Cl | C—H | H | CF3 | CH2 | H | 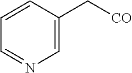 |
| 10-511 | C—Cl | C—H | C—Cl | C—H | H | CF3 | CH2 | H | 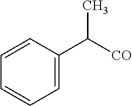 |
| 10-512 | C—Cl | C—H | C—Cl | C—H | H | CF3 | CH2 | H | CH3NHCO |
| 10-513 | C—Cl | C—H | C—Cl | C—H | H | CF3 | CH2 | H | CH3CH2NHCO |
| 10-514 | C—Cl | C—H | C—Cl | C—H | H | CF3 | CH2 | H | CF3CH2NHCO |
| 10-515 | C—Cl | C—H | C—Cl | C—H | H | CF3 | CH2 | H | n-PrNHCO |
| 10-516 | C—Cl | C—H | C—Cl | C—H | H | CF3 | CH2 | H | iso-PrNHCO |
| 10-517 | C—Cl | C—H | C—Cl | C—H | H | CF3 | CH2 | H | cyclo-PrNHCO |
| 10-518 | C—Cl | C—H | C—Cl | C—H | H | CF3 | CH2 | H | n-BuNHCO |
| 10-519 | C—Cl | C—H | C—Cl | C—H | H | CF3 | CH2 | H | tert-BuNHCO |
| 10-520 | C—Cl | C—H | C—Cl | C—H | H | CF3 | CH2 | H | cyclo-BuNHCO |
| 10-521 | C—Cl | C—H | C—Cl | C—H | H | CF3 | CH2 | H | cyclo-PenNHCO |
| 10-522 | C—Cl | C—H | C—Cl | C—H | H | CF3 | CH2 | H | cyclo-HexNHCO |
| 10-523 | C—Cl | C—H | C—Cl | C—H | H | CF3 | CH2 | H | CH2═CHCH2NHCO |
| 10-524 | C—Cl | C—H | C—Cl | C—H | H | CF3 | CH2 | H | HCCCH2NHCO |
| 10-525 | C—Cl | C—H | C—Cl | C—H | H | CF3 | CH2 | H | cyclo-PrCH2NHCO |
| 10-526 | C—Cl | C—H | C—Cl | C—H | H | CF3 | CH2 | H | NCCH2NHCO |
| 10-527 | C—Cl | C—H | C—Cl | C—H | H | CF3 | CH2 | H | CF3(CH3)CHNHCO |
| 10-528 | C—Cl | C—H | C—Cl | C—H | H | CF3 | CH2 | H | CF3(CH3)2CNCO |
| 10-529 | C—Cl | C—H | C—Cl | C—H | H | CF3 | CH2 | H | NCCH2OCO |
| 10-530 | C—Cl | C—H | C—Cl | C—H | H | CF3 | CH2 | H | FCH2CH2NHCO |
| 10-531 | C—Cl | C—H | C—Cl | C—H | H | CF3 | CH2 | H | CF3CH2CH2NHCO |
| 10-532 | C—Cl | C—H | C—Cl | C—H | H | CF3 | CH2 | H | HCF2CF2CH2NHCO |
| 10-533 | C—Cl | C—H | C—Cl | C—H | H | CF3 | CH2 | H | 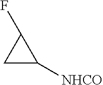 |
| 10-534 | C—Cl | C—H | C—Cl | C—H | H | CF3 | CH2 | H | 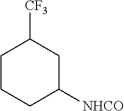 |
| 10-535 | C—Cl | C—H | C—Cl | C—H | H | CF3 | CH2 | H | EtO2CCH2NHCO |
| 10-536 | C—Cl | C—H | C—Cl | C—H | H | CF3 | CH2 | H | 2-CF3—PhNHCO |
| 10-537 | C—Cl | C—H | C—Cl | C—H | H | CF3 | CH2 | H | 3-CF3—PhNHCO |
| 10-538 | C—Cl | C—H | C—Cl | C—H | H | CF3 | CH2 | H | 4-CF3—PhNHCO |
| 10-539 | C—Cl | C—H | C—Cl | C—H | H | CF3 | CH2 | H | 2-py-NHCO |
| 10-540 | C—Cl | C—H | C—Cl | C—H | H | CF3 | CH2 | H | 3-py-NHCO |
| 10-541 | C—Cl | C—H | C—Cl | C—H | H | CF3 | CH2 | H | 4-py-NHCO |
| 10-542 | C—Cl | C—H | C—Cl | C—H | H | CF3 | CH2 | H | 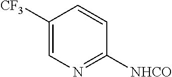 |
| 10-543 | C—Cl | C—H | C—Cl | C—H | H | CF3 | CH2 | H | 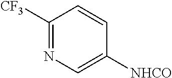 |
| 10-544 | C—Cl | C—H | C—Cl | C—H | H | CF3 | CH2 | H | PhCH2HNCO |
| 10-545 | C—Cl | C—H | C—Cl | C—H | H | CF3 | CH2 | H | 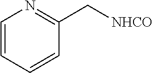 |
| 10-546 | C—Cl | C—H | C—Cl | C—H | H | CF3 | CH2 | H | (CH3)2NCO |
| 10-547 | C—Cl | C—H | C—Cl | C—H | H | CF3 | CH2 | H | pyrrolidineCO |
| 10-548 | C—Cl | C—H | C—Cl | C—H | H | CF3 | CH2 | H | piperidineCO |
| 10-549 | C—Cl | C—H | C—Cl | C—H | H | CF3 | CH2 | H | morpholineCO |
| 10-550 | C—Cl | C—H | C—Cl | C—H | H | CF3 | CH2 | H | CH3OCONH |
| 10-551 | C—Cl | C—H | C—Cl | C—H | H | CF3 | CH2 | H | CH3CH2OCO |
| 10-552 | C—Cl | C—H | C—Cl | C—H | H | CF3 | CH2 | H | n-PrOCO |
| 10-553 | C—Cl | C—H | C—Cl | C—H | H | CF3 | CH2 | H | cyclo-PrOCO |
| 10-554 | C—Cl | C—H | C—Cl | C—H | H | CF3 | CH2 | H | CF3CH2OCO |
| 10-555 | C—Cl | C—H | C—Cl | C—H | H | CF3 | CH2 | H | CH2═CHCH2OCO |
| 10-556 | C—Cl | C—H | C—Cl | C—H | H | CF3 | CH2 | H | HCCCH2OCO |
| 10-557 | C—Cl | C—H | C—Cl | C—H | H | CF3 | CH2 | H | PhOCO |
| 10-558 | C—Cl | C—H | C—Cl | C—H | H | CF3 | CH2 | H | PhCH2OCO |
| 10-559 | C—Cl | C—H | C—Cl | C—H | H | CF3 | CH2 | H | tert-BuOCO |
| 10-560 | C—Cl | C—H | C—Cl | C—H | H | CF3 | CH2 | H | CH3SO2 |
| 10-561 | C—Cl | C—H | C—Cl | C—H | H | CF3 | CH2 | H | CH3CH2SO2 |
| 10-562 | C—Cl | C—H | C—Cl | C—H | H | CF3 | CH2 | H | CF3SO2 |
| 10-563 | C—Cl | C—H | C—Cl | C—H | H | CF3 | CH2 | H | CF3CH2SO2 |
| 10-564 | C—Cl | C—H | C—Cl | C—H | H | CF3 | CH2 | H | PhSO2 |
| 10-565 | C—Cl | C—H | C—Cl | C—H | H | CF3 | CH2 | H | CH3NHCONHSO2 |
| 10-566 | C—Cl | C—H | C—Cl | C—H | H | CF3 | CH2 | H | (CH3)2NCONHSO2 |
| 10-567 | C—Cl | C—H | C—Cl | C—H | H | CF3 | CH2 | H | CH3CS |
| 10-568 | C—Cl | C—H | C—Cl | C—H | H | CF3 | CH2 | H | CH3CH2CS |
| 10-569 | C—Cl | C—H | C—Cl | C—H | H | CF3 | CH2 | H | cyclo-PrCS |
| 10-570 | C—Cl | C—H | C—Cl | C—H | H | CF3 | CH2 | H | CF3CH2CS |
| 10-571 | C—Cl | C—H | C—Br | C—H | H | CF3 | CH2 | H | CH3CO |
| 10-572 | C—Cl | C—H | C—Br | C—H | H | CF3 | CH2 | H | CH3CH2CO |
| 10-573 | C—Cl | C—H | C—Br | C—H | H | CF3 | CH2 | H | n-PrCO |
| 10-574 | C—Cl | C—H | C—Br | C—H | H | CF3 | CH2 | H | cyclo-PrCO |
| 10-575 | C—Cl | C—H | C—Br | C—H | H | CF3 | CH2 | H | cyclo-PrCH2CO |
| 10-576 | C—Cl | C—H | C—Br | C—H | H | CF3 | CH2 | H | CF3CH2CO |
| 10-577 | C—Cl | C—H | C—Br | C—H | H | CF3 | CH2 | H | CH3SCH2CO |
| 10-578 | C—Cl | C—H | C—Br | C—H | H | CF3 | CH2 | H | CH3S(O)CH2CO |
| 10-579 | C—Cl | C—H | C—Br | C—H | H | CF3 | CH2 | H | CH3SO2CH2CO |
| 10-580 | C—Cl | C—H | C—Br | C—H | H | CF3 | CH2 | H | CH3CH2NHCO |
| 10-581 | C—Br | C—H | C—Br | C—H | H | CF3 | CH2 | H | CH3CO |
| 10-582 | C—Br | C—H | C—Br | C—H | H | CF3 | CH2 | H | CH3CH2CO |
| 10-583 | C—Br | C—H | C—Br | C—H | H | CF3 | CH2 | H | n-PrCO |
| 10-584 | C—Br | C—H | C—Br | C—H | H | CF3 | CH2 | H | cyclo-PrCO |
| 10-585 | C—Br | C—H | C—Br | C—H | H | CF3 | CH2 | H | cyclo-PrCH2CO |
| 10-586 | C—Br | C—H | C—Br | C—H | H | CF3 | CH2 | H | CF3CH2CO |
| 10-587 | C—Br | C—H | C—Br | C—H | H | CF3 | CH2 | H | CH3SCH2CO |
| 10-588 | C—Br | C—H | C—Br | C—H | H | CF3 | CH2 | H | CH3S(O)CH2CO |
| 10-589 | C—Br | C—H | C—Br | C—H | H | CF3 | CH2 | H | CH3SO2CH2CO |
| 10-590 | C—Br | C—H | C—Br | C—H | H | CF3 | CH2 | H | CH3CH2NHCO |
| 10-591 | C—Cl | C—F | C—Cl | C—H | H | CF3 | CH2 | H | CH3CO |
| 10-592 | C—Cl | C—F | C—Cl | C—H | H | CF3 | CH2 | H | CH3CH2CO |
| 10-593 | C—Cl | C—F | C—Cl | C—H | H | CF3 | CH2 | H | n-PrCO |
| 10-594 | C—Cl | C—F | C—Cl | C—H | H | CF3 | CH2 | H | cyclo-PrCO |
| 10-595 | C—Cl | C—F | C—Cl | C—H | H | CF3 | CH2 | H | cyclo-PrCH2CO |
| 10-596 | C—Cl | C—F | C—Cl | C—H | H | CF3 | CH2 | H | CF3CH2CO |
| 10-598 | C—Cl | C—F | C—Cl | C—H | H | CF3 | CH2 | H | CH3S(O)CH2CO |
| 10-599 | C—Cl | C—F | C—Cl | C—H | H | CF3 | CH2 | H | CH3SO2CH2CO |
| 10-600 | C—Cl | C—F | C—Cl | C—H | H | CF3 | CH2 | H | CH3CH2NHCO |
| 10-601 | C—Cl | C—Cl | C—Cl | C—H | H | CF3 | CH2 | H | CH3CO |
| 10-602 | C—Cl | C—Cl | C—Cl | C—H | H | CF3 | CH2 | H | CH3CH2CO |
| 10-603 | C—Cl | C—Cl | C—Cl | C—H | H | CF3 | CH2 | H | n-PrCO |
| 10-604 | C—Cl | C—Cl | C—Cl | C—H | H | CF3 | CH2 | H | cyclo-PrCO |
| 10-605 | C—Cl | C—Cl | C—Cl | C—H | H | CF3 | CH2 | H | cyclo-PrCH2CO |
| 10-606 | C—Cl | C—Cl | C—Cl | C—H | H | CF3 | CH2 | H | CF3CH2CO |
| 10-608 | C—Cl | C—Cl | C—Cl | C—H | H | CF3 | CH2 | H | CH3CH2OCH2CH2—CO |
| 10-609 | C—Cl | C—Cl | C—Cl | C—H | H | CF3 | CH2 | H | CH3SCH2CO |
| 10-610 | C—Cl | C—Cl | C—Cl | C—H | H | CF3 | CH2 | H | CH3S(O)CH2CO |
| 10-611 | C—Cl | C—Cl | C—Cl | C—H | H | CF3 | CH2 | H | CH3SO2CH2CO |
| 10-612 | C—Cl | C—Cl | C—Cl | C—H | H | CF3 | CH2 | H | CH3CH2NHCO |
| 10-613 | C—Cl | C—Br | C—Cl | C—H | H | CF3 | CH2 | H | CH3CO |
| 10-614 | C—Cl | C—Br | C—Cl | C—H | H | CF3 | CH2 | H | CH3CH2CO |
| 10-615 | C—Cl | C—Br | C—Cl | C—H | H | CF3 | CH2 | H | n-PrCO |
| 10-616 | C—Cl | C—Br | C—Cl | C—H | H | CF3 | CH2 | H | cyclo-PrCO |
| 10-617 | C—Cl | C—Br | C—Cl | C—H | H | CF3 | CH2 | H | cyclo-PrCH2CO |
| 10-618 | C—Cl | C—Br | C—Cl | C—H | H | CF3 | CH2 | H | CF3CH2CO |
| 10-619 | C—Cl | C—Br | C—Cl | C—H | H | CF3 | CH2 | H | CH3SCH2CO |
| 10-620 | C—Cl | C—Br | C—Cl | C—H | H | CF3 | CH2 | H | CH3S(O)CH2CO |
| 10-621 | C—Cl | C—Br | C—Cl | C—H | H | CF3 | CH2 | H | CH3SO2CH2CO |
| 10-622 | C—Cl | C—Br | C—Cl | C—H | H | CF3 | CH2 | H | CH3CH2NHCO |
| 10-623 | C—Cl | C—Cl | C—Br | C—H | H | CF3 | CH2 | H | CH3CO |
| 10-624 | C—Cl | C—Cl | C—Br | C—H | H | CF3 | CH2 | H | CH3CH2CO |
| 10-625 | C—Cl | C—Cl | C—Br | C—H | H | CF3 | CH2 | H | n-PrCO |
| 10-626 | C—Cl | C—Cl | C—Br | C—H | H | CF3 | CH2 | H | cyclo-PrCO |
| 10-627 | C—Cl | C—Cl | C—Br | C—H | H | CF3 | CH2 | H | cyclo-PrCH2CO |
| 10-628 | C—Cl | C—Cl | C—Br | C—H | H | CF3 | CH2 | H | CF3CH2CO |
| 10-629 | C—Cl | C—Cl | C—Br | C—H | H | CF3 | CH2 | H | CH3SCH2CO |
| 10-630 | C—Cl | C—Cl | C—Br | C—H | H | CF3 | CH2 | H | CH3S(O)CH2CO |
| 10-631 | C—Cl | C—Cl | C—Br | C—H | H | CF3 | CH2 | H | CH3SO2CH2CO |
| 10-632 | C—Cl | C—Cl | C—Br | C—H | H | CF3 | CH2 | H | CH3CH2NHCO |
| 10-633 | C—Cl | C—F | C—Cl | C—H | F | CF3 | CH2 | H | CH3CO |
| 10-634 | C—Cl | C—F | C—Cl | C—H | F | CF3 | CH2 | H | CH3CH2CO |
| 10-635 | C—Cl | C—F | C—Cl | C—H | F | CF3 | CH2 | H | n-PrCO |
| 10-636 | C—Cl | C—F | C—Cl | C—H | F | CF3 | CH2 | H | cyclo-PrCO |
| 10-637 | C—Cl | C—F | C—Cl | C—H | F | CF3 | CH2 | H | cyclo-PrCH2CO |
| 10-638 | C—Cl | C—F | C—Cl | C—H | F | CF3 | CH2 | H | CF3CH2CO |
| 10-639 | C—Cl | C—F | C—Cl | C—H | F | CF3 | CH2 | H | CH3SCH2CO |
| 10-640 | C—Cl | C—F | C—Cl | C—H | F | CF3 | CH2 | H | CH3S(O)CH2CO |
| 10-641 | C—Cl | C—F | C—Cl | C—H | F | CF3 | CH2 | H | CH3SO2CH2CO |
| 10-642 | C—Cl | C—F | C—Cl | C—H | F | CF3 | CH2 | H | CH3CH2NHCO |
| 10-643 | C—CF3 | C—H | C—H | C—H | H | CF3 | CH2 | H | CH3CO |
| 10-644 | C—CF3 | C—H | C—H | C—H | H | CF3 | CH2 | H | CH3CH2CO |
| 10-645 | C—CF3 | C—H | C—H | C—H | H | CF3 | CH2 | H | n-PrCO |
| 10-646 | C—CF3 | C—H | C—H | C—H | H | CF3 | CH2 | H | cyclo-PrCO |
| 10-647 | C—CF3 | C—H | C—H | C—H | H | CF3 | CH2 | H | cyclo-PrCH2CO |
| 10-648 | C—CF3 | C—H | C—H | C—H | H | CF3 | CH2 | H | CF3CH2CO |
| 10-649 | C—CF3 | C—H | C—H | C—H | H | CF3 | CH2 | H | CH3SCH2CO |
| 10-650 | C—CF3 | C—H | C—H | C—H | H | CF3 | CH2 | H | CH3S(O)CH2CO |
| 10-651 | C—CF3 | C—H | C—H | C—H | H | CF3 | CH2 | H | CH3SO2CH2CO |
| 10-652 | C—CF3 | C—H | C—H | C—H | H | CF3 | CH2 | H | CH3CH2NHCO |
| 10-653 | C—CF3 | C—F | C—H | C—H | H | CF3 | CH2 | H | CH3CO |
| 10-654 | C—CF3 | C—F | C—H | C—H | H | CF3 | CH2 | H | CH3CH2CO |
| 10-655 | C—CF3 | C—F | C—H | C—H | H | CF3 | CH2 | H | n-PrCO |
| 10-656 | C—CF3 | C—F | C—H | C—H | H | CF3 | CH2 | H | cyclo-PrCO |
| 10-657 | C—CF3 | C—F | C—H | C—H | H | CF3 | CH2 | H | cyclo-PrCH2CO |
| 10-658 | C—CF3 | C—F | C—H | C—H | H | CF3 | CH2 | H | CF3CH2CO |
| 10-659 | C—CF3 | C—F | C—H | C—H | H | CF3 | CH2 | H | CH3SCH2CO |
| 10-660 | C—CF3 | C—F | C—H | C—H | H | CF3 | CH2 | H | CH3S(O)CH2CO |
| 10-661 | C—CF3 | C—F | C—H | C—H | H | CF3 | CH2 | H | CH3SO2CH2CO |
| 10-662 | C—CF3 | C—F | C—H | C—H | H | CF3 | CH2 | H | CH3CH2NHCO |
| 10-663 | C—CF3 | C—H | C—H | C—H | F | CF3 | CH2 | H | CH3CO |
| 10-664 | C—CF3 | C—H | C—H | C—H | F | CF3 | CH2 | H | CH3CH2CO |
| 10-665 | C—CF3 | C—H | C—H | C—H | F | CF3 | CH2 | H | n-PrCO |
| 10-666 | C—CF3 | C—H | C—H | C—H | F | CF3 | CH2 | H | cyclo-PrCO |
| 10-667 | C—CF3 | C—H | C—H | C—H | F | CF3 | CH2 | H | cyclo-PrCH2CO |
| 10-668 | C—CF3 | C—H | C—H | C—H | F | CF3 | CH2 | H | CF3CH2CO |
| 10-669 | C—CF3 | C—H | C—H | C—H | F | CF3 | CH2 | H | CH3SCH2CO |
| 10-670 | C—CF3 | C—H | C—H | C—H | F | CF3 | CH2 | H | CH3S(O)CH2CO |
| 10-671 | C—CF3 | C—H | C—H | C—H | F | CF3 | CH2 | H | CH3SO2CH2CO |
| 10-672 | C—CF3 | C—H | C—H | C—H | F | CF3 | CH2 | H | CH3CH2NHCO |
| 10-673 | C—H | C—H | C—CF3 | C—H | F | CF3 | CH2 | H | CH3CO |
| 10-674 | C—H | C—H | C—CF3 | C—H | F | CF3 | CH2 | H | CH3CH2CO |
| 10-675 | C—H | C—H | C—CF3 | C—H | F | CF3 | CH2 | H | n-PrCO |
| 10-676 | C—H | C—H | C—CF3 | C—H | F | CF3 | CH2 | H | cyclo-PrCO |
| 10-677 | C—H | C—H | C—CF3 | C—H | F | CF3 | CH2 | H | cyclo-PrCH2CO |
| 10-678 | C—H | C—H | C—CF3 | C—H | F | CF3 | CH2 | H | CF3CH2CO |
| 10-679 | C—H | C—H | C—CF3 | C—H | F | CF3 | CH2 | H | CH3SCH2CO |
| 10-680 | C—H | C—H | C—CF3 | C—H | F | CF3 | CH2 | H | CH3S(O)CH2CO |
| 10-681 | C—H | C—H | C—CF3 | C—H | F | CF3 | CH2 | H | CH3SO2CH2CO |
| 10-682 | C—H | C—H | C—CF3 | C—H | F | CF3 | CH2 | H | CH3CH2NHCO |
| 10-683 | C—Cl | C—H | C—CF3 | C—H | H | CF3 | CH2 | H | CH3CO |
| 10-684 | C—Cl | C—H | C—CF3 | C—H | H | CF3 | CH2 | H | CH3CH2CO |
| 10-685 | C—Cl | C—H | C—CF3 | C—H | H | CF3 | CH2 | H | n-PrCO |
| 10-686 | C—Cl | C—H | C—CF3 | C—H | H | CF3 | CH2 | H | cyclo-PrCO |
| 10-687 | C—Cl | C—H | C—CF3 | C—H | H | CF3 | CH2 | H | cyclo-PrCH2CO |
| 10-688 | C—Cl | C—H | C—CF3 | C—H | H | CF3 | CH2 | H | CF3CH2CO |
| 10-689 | C—Cl | C—H | C—CF3 | C—H | H | CF3 | CH2 | H | CH3OCH2CH2—CO |
| 10-690 | C—Cl | C—H | C—CF3 | C—H | H | CF3 | CH2 | H | CH3CH2OCH2CH2—CO |
| 10-691 | C—Cl | C—H | C—CF3 | C—H | H | CF3 | CH2 | H | CH3SCH2CO |
| 10-692 | C—Cl | C—H | C—CF3 | C—H | H | CF3 | CH2 | H | CH3S(O)CH2CO |
| 10-693 | C—Cl | C—H | C—CF3 | C—H | H | CF3 | CH2 | H | CH3SO2CH2CO |
| 10-694 | C—Cl | C—H | C—CF3 | C—H | H | CF3 | CH2 | H | CH3CH2NHCO |
| 10-695 | C—CF3 | C—H | C—CF3 | C—H | H | CF3 | CH2 | H | CH3CO |
| 10-696 | C—CF3 | C—H | C—CF3 | C—H | H | CF3 | CH2 | H | CH3CH2CO |
| 10-697 | C—CF3 | C—H | C—CF3 | C—H | H | CF3 | CH2 | H | n-PrCO |
| 10-698 | C—CF3 | C—H | C—CF3 | C—H | H | CF3 | CH2 | H | cyclo-PrCO |
| 10-699 | C—CF3 | C—H | C—CF3 | C—H | H | CF3 | CH2 | H | cyclo-PrCH2CO |
| 10-700 | C—CF3 | C—H | C—CF3 | C—H | H | CF3 | CH2 | H | CF3CH2CO |
| 10-701 | C—CF3 | C—H | C—CF3 | C—H | H | CF3 | CH2 | H | CH3OCH2CH2—CO |
| 10-702 | C—CF3 | C—H | C—CF3 | C—H | H | CF3 | CH2 | H | CH3CH2OCH2CH2—CO |
| 10-703 | C—CF3 | C—H | C—CF3 | C—H | H | CF3 | CH2 | H | CH3SCH2CO |
| 10-704 | C—CF3 | C—H | C—CF3 | C—H | H | CF3 | CH2 | H | CH3S(O)CH2CO |
| 10-705 | C—CF3 | C—H | C—CF3 | C—H | H | CF3 | CH2 | H | CH3SO2CH2CO |
| 10-706 | C—CF3 | C—H | C—CF3 | C—H | H | CF3 | CH2 | H | CH3CH2NHCO |
| 10-707 | C—Cl | C—Cl | C—CF3 | C—H | H | CF3 | CH2 | H | CH3CO |
| 10-708 | C—Cl | C—Cl | C—CF3 | C—H | H | CF3 | CH2 | H | CH3CH2CO |
| 10-709 | C—Cl | C—Cl | C—CF3 | C—H | H | CF3 | CH2 | H | n-PrCO |
| 10-710 | C—Cl | C—Cl | C—CF3 | C—H | H | CF3 | CH2 | H | cyclo-PrCO |
| 10-711 | C—Cl | C—Cl | C—CF3 | C—H | H | CF3 | CH2 | H | cyclo-PrCH2CO |
| 10-712 | C—Cl | C—Cl | C—CF3 | C—H | H | CF3 | CH2 | H | CF3CH2CO |
| 10-713 | C—Cl | C—Cl | C—CF3 | C—H | H | CF3 | CH2 | H | CH3SCH2CO |
| 10-714 | C—Cl | C—Cl | C—CF3 | C—H | H | CF3 | CH2 | H | CH3S(O)CH2CO |
| 10-715 | C—Cl | C—Cl | C—CF3 | C—H | H | CF3 | CH2 | H | CH3SO2CH2CO |
| 10-716 | C—Cl | C—Cl | C—CF3 | C—H | H | CF3 | CH2 | H | CH3CH2NHCO |
| 10-717 | C—Cl | N | C—H | C—H | H | CF3 | CH2 | H | CH3CO |
| 10-718 | C—Cl | N | C—H | C—H | H | CF3 | CH2 | H | CH3CH2CO |
| 10-719 | C—Cl | N | C—H | C—H | H | CF3 | CH2 | H | n-PrCO |
| 10-720 | C—Cl | N | C—H | C—H | H | CF3 | CH2 | H | cyclo-PrCO |
| 10-721 | C—Cl | N | C—H | C—H | H | CF3 | CH2 | H | cyclo-PrCH2CO |
| 10-722 | C—Cl | N | C—H | C—H | H | CF3 | CH2 | H | CF3CH2CO |
| 10-723 | C—Cl | N | C—H | C—H | H | CF3 | CH2 | H | CH3SCH2CO |
| 10-724 | C—Cl | N | C—H | C—H | H | CF3 | CH2 | H | CH3S(O)CH2CO |
| 10-725 | C—Cl | N | C—H | C—H | H | CF3 | CH2 | H | CH3SO2CH2CO |
| 10-726 | C—Cl | N | C—H | C—H | H | CF3 | CH2 | H | CH3CH2NHCO |
| 10-727 | C—Cl | N | C—Cl | C—H | H | CF3 | CH2 | H | CH3CO |
| 10-728 | C—Cl | N | C—Cl | C—H | H | CF3 | CH2 | H | CH3CH2CO |
| 10-729 | C—Cl | N | C—Cl | C—H | H | CF3 | CH2 | H | n-PrCO |
| 10-730 | C—Cl | N | C—Cl | C—H | H | CF3 | CH2 | H | cyclo-PrCO |
| 10-731 | C—Cl | N | C—Cl | C—H | H | CF3 | CH2 | H | cyclo-PrCH2CO |
| 10-732 | C—Cl | N | C—Cl | C—H | H | CF3 | CH2 | H | CF3CH2CO |
| 10-733 | C—Cl | N | C—Cl | C—H | H | CF3 | CH2 | H | CH3SCH2CO |
| 10-734 | C—Cl | N | C—Cl | C—H | H | CF3 | CH2 | H | CH3S(O)CH2CO |
| 10-735 | C—Cl | N | C—Cl | C—H | H | CF3 | CH2 | H | CH3SO2CH2CO |
| 10-736 | C—Cl | N | C—Cl | C—H | H | CF3 | CH2 | H | CH3CH2NHCO |
| 10-737 | C—CF3 | N | C—H | C—H | H | CF3 | CH2 | H | CH3CO |
| 10-738 | C—CF3 | N | C—H | C—H | H | CF3 | CH2 | H | CH3CH2CO |
| 10-739 | C—CF3 | N | C—H | C—H | H | CF3 | CH2 | H | n-PrCO |
| 10-740 | C—CF3 | N | C—H | C—H | H | CF3 | CH2 | H | cyclo-PrCO |
| 10-741 | C—CF3 | N | C—H | C—H | H | CF3 | CH2 | H | cyclo-PrCH2CO |
| 10-742 | C—CF3 | N | C—H | C—H | H | CF3 | CH2 | H | CF3CH2CO |
| 10-743 | C—CF3 | N | C—H | C—H | H | CF3 | CH2 | H | CH3SCH2CO |
| 10-744 | C—CF3 | N | C—H | C—H | H | CF3 | CH2 | H | CH3S(O)CH2CO |
| 10-745 | C—CF3 | N | C—H | C—H | H | CF3 | CH2 | H | CH3SO2CH2CO |
| 10-746 | C—CF3 | N | C—H | C—H | H | CF3 | CH2 | H | CH3CH2NHCO |
| 10-747 | C—CF3 | N | C—CF3 | C—H | H | CF3 | CH2 | H | CH3CO |
| 10-748 | C—CF3 | N | C—CF3 | C—H | H | CF3 | CH2 | H | CH3CH2CO |
| 10-749 | C—CF3 | N | C—CF3 | C—H | H | CF3 | CH2 | H | n-PrCO |
| 10-750 | C—CF3 | N | C—CF3 | C—H | H | CF3 | CH2 | H | cyclo-PrCO |
| 10-751 | C—CF3 | N | C—CF3 | C—H | H | CF3 | CH2 | H | cyclo-PrCH2CO |
| 10-752 | C—CF3 | N | C—CF3 | C—H | H | CF3 | CH2 | H | CF3CH2CO |
| 10-753 | C—CF3 | N | C—CF3 | C—H | H | CF3 | CH2 | H | CH3SCH2CO |
| 10-754 | C—CF3 | N | C—CF3 | C—H | H | CF3 | CH2 | H | CH3S(O)CH2CO |
| 10-755 | C—CF3 | N | C—CF3 | C—H | H | CF3 | CH2 | H | CH3SO2CH2CO |
| 10-756 | C—CF3 | N | C—CF3 | C—H | H | CF3 | CH2 | H | CH3CH2NHCO |
| 10-757 | C—CF3 | C—H | C—H | N | H | CF3 | CH2 | H | CH3CO |
| 10-758 | C—CF3 | C—H | C—H | N | H | CF3 | CH2 | H | CH3CH2CO |
| 10-759 | C—CF3 | C—H | C—H | N | H | CF3 | CH2 | H | n-PrCO |
| 10-760 | C—CF3 | C—H | C—H | N | H | CF3 | CH2 | H | cyclo-PrCO |
| 10-761 | C—CF3 | C—H | C—H | N | H | CF3 | CH2 | H | cyclo-PrCH2CO |
| 10-762 | C—CF3 | C—H | C—H | N | H | CF3 | CH2 | H | CF3CH2CO |
| 10-763 | C—CF3 | C—H | C—H | N | H | CF3 | CH2 | H | CH3SCH2CO |
| 10-764 | C—CF3 | C—H | C—H | N | H | CF3 | CH2 | H | CH3S(O)CH2CO |
| 10-765 | C—CF3 | C—H | C—H | N | H | CF3 | CH2 | H | CH3SO2CH2CO |
| 10-766 | C—CF3 | C—H | C—H | N | H | CF3 | CH2 | H | CH3CH2NHCO |
| 10-767 | C—H | C—H | C—CF3 | N | H | CF3 | CH2 | H | CH3CO |
| 10-768 | C—H | C—H | C—CF3 | N | H | CF3 | CH2 | H | CH3CH2CO |
| 10-769 | C—H | C—H | C—CF3 | N | H | CF3 | CH2 | H | n-PrCO |
| 10-770 | C—H | C—H | C—CF3 | N | H | CF3 | CH2 | H | cyclo-PrCO |
| 10-771 | C—H | C—H | C—CF3 | N | H | CF3 | CH2 | H | cyclo-PrCH2CO |
| 10-772 | C—H | C—H | C—CF3 | N | H | CF3 | CH2 | H | CF3CH2CO |
| 10-773 | C—H | C—H | C—CF3 | N | H | CF3 | CH2 | H | CH3SCH2CO |
| 10-774 | C—H | C—H | C—CF3 | N | H | CF3 | CH2 | H | CH3S(O)CH2CO |
| 10-775 | C—H | C—H | C—CF3 | N | H | CF3 | CH2 | H | CH3SO2CH2CO |
| 10-776 | C—H | C—H | C—CF3 | N | H | CF3 | CH2 | H | CH3CH2NHCO |
| 10-777 | C—CF3 | C—H | C—Cl | N | H | CF3 | CH2 | H | CH3CO |
| 10-778 | C—CF3 | C—H | C—Cl | N | H | CF3 | CH2 | H | CH3CH2CO |
| 10-779 | C—CF3 | C—H | C—Cl | N | H | CF3 | CH2 | H | n-PrCO |
| 10-780 | C—CF3 | C—H | C—Cl | N | H | CF3 | CH2 | H | cyclo-PrCO |
| 10-781 | C—CF3 | C—H | C—Cl | N | H | CF3 | CH2 | H | cyclo-PrCH2CO |
| 10-782 | C—CF3 | C—H | C—Cl | N | H | CF3 | CH2 | H | CF3CH2CO |
| 10-783 | C—CF3 | C—H | C—Cl | N | H | CF3 | CH2 | H | CH3SCH2CO |
| 10-784 | C—CF3 | C—H | C—Cl | N | H | CF3 | CH2 | H | CH3S(O)CH2CO |
| 10-785 | C—CF3 | C—H | C—Cl | N | H | CF3 | CH2 | H | CH3SO2CH2CO |
| 10-786 | C—CF3 | C—H | C—Cl | N | H | CF3 | CH2 | H | CH3CH2NHCO |
| 10-787 | C—CF3 | C—H | C—Br | N | H | CF3 | CH2 | H | CH3CO |
| 10-788 | C—CF3 | C—H | C—Br | N | H | CF3 | CH2 | H | CH3CH2CO |
| 10-789 | C—CF3 | C—H | C—Br | N | H | CF3 | CH2 | H | n-PrCO |
| 10-790 | C—CF3 | C—H | C—Br | N | H | CF3 | CH2 | H | cyclo-PrCO |
| 10-791 | C—CF3 | C—H | C—Br | N | H | CF3 | CH2 | H | cyclo-PrCH2CO |
| 10-792 | C—CF3 | C—H | C—Br | N | H | CF3 | CH2 | H | CF3CH2CO |
| 10-794 | C—CF3 | C—H | C—Br | N | H | CF3 | CH2 | H | CH3S(O)CH2CO |
| 10-795 | C—CF3 | C—H | C—Br | N | H | CF3 | CH2 | H | CH3SO2CH2CO |
| 10-796 | C—CF3 | C—H | C—Br | N | H | CF3 | CH2 | H | CH3CH2NHCO |
| 10-797 | C—CF3 | C—H | C—CF3 | N | H | CF3 | CH2 | H | CH3CO |
| 10-798 | C—CF3 | C—H | C—CF3 | N | H | CF3 | CH2 | H | CH3CH2CO |
| 10-799 | C—CF3 | C—H | C—CF3 | N | H | CF3 | CH2 | H | n-PrCO |
| 10-800 | C—CF3 | C—H | C—CF3 | N | H | CF3 | CH2 | H | cyclo-PrCO |
| 10-801 | C—CF3 | C—H | C—CF3 | N | H | CF3 | CH2 | H | cyclo-PrCH2CO |
| 10-802 | C—CF3 | C—H | C—CF3 | N | H | CF3 | CH2 | H | CF3CH2CO |
| 10-804 | C—CF3 | C—H | C—CF3 | N | H | CF3 | CH2 | H | CH3S(O)CH2CO |
| 10-805 | C—CF3 | C—H | C—CF3 | N | H | CF3 | CH2 | H | CH3SO2CH2CO |
| 10-806 | C—CF3 | C—H | C—CF3 | N | H | CF3 | CH2 | H | CH3CH2NHCO |

TABLE 11

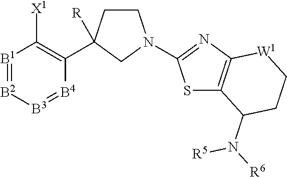

| No. | B¹ | B² | B³ | B⁴ | X¹ | R | W¹ | R⁵ | R⁶ |
|---|---|---|---|---|---|---|---|---|---|
| 11-1 | C—Cl | C—H | C—H | C—H | H | CF3 | — | H | CH3CO |
| 11-2 | C—Cl | C—H | C—H | C—H | H | CF3 | — | H | CH3CH2CO |
| 11-3 | C—Cl | C—H | C—H | C—H | H | CF3 | — | H | n-PrCO |
| 11-4 | C—Cl | C—H | C—H | C—H | H | CF3 | — | H | cyclo-PrCO |
| 11-5 | C—Cl | C—H | C—H | C—H | H | CF3 | — | H | cyclo-PrCH2CO |
| 11-6 | C—Cl | C—H | C—H | C—H | H | CF3 | — | H | CF3CH2CO |
| 11-7 | C—Cl | C—H | C—H | C—H | H | CF3 | — | H | CH3SCH2CO |
| 11-8 | C—Cl | C—H | C—H | C—H | H | CF3 | — | H | CH3S(O)CH2CO |
| 11-9 | C—Cl | C—H | C—H | C—H | H | CF3 | — | H | CH3SO2CH2CO |
| 11-10 | C—Cl | C—H | C—H | C—H | H | CF3 | — | H | CH3CH2NHCO |
| 11-11 | C—Cl | C—H | C—H | C—H | F | CF3 | — | H | CH3CO |
| 11-12 | C—Cl | C—H | C—H | C—H | F | CF3 | — | H | CH3CH2CO |
| 11-13 | C—Cl | C—H | C—H | C—H | F | CF3 | — | H | n-PrCO |
| 11-14 | C—Cl | C—H | C—H | C—H | F | CF3 | — | H | cyclo-PrCO |
| 11-15 | C—Cl | C—H | C—H | C—H | F | CF3 | — | H | cyclo-PrCH2CO |
| 11-16 | C—Cl | C—H | C—H | C—H | F | CF3 | — | H | CF3CH2CO |
| 11-17 | C—Cl | C—H | C—H | C—H | F | CF3 | — | H | CH3SCH2CO |
| 11-18 | C—Cl | C—H | C—H | C—H | F | CF3 | — | H | CH3S(O)CH2CO |
| 11-19 | C—Cl | C—H | C—H | C—H | F | CF3 | — | H | CH3SO2CH2CO |
| 11-20 | C—Cl | C—H | C—H | C—H | F | CF3 | — | H | CH3CH2NHCO |
| 11-21 | C—H | C—H | C—Cl | C—H | F | CF3 | — | H | CH3CO |
| 11-22 | C—H | C—H | C—Cl | C—H | F | CF3 | — | H | CH3CH2CO |
| 11-23 | C—H | C—H | C—Cl | C—H | F | CF3 | — | H | n-PrCO |
| 11-24 | C—H | C—H | C—Cl | C—H | F | CF3 | — | H | cyclo-PrCO |
| 11-25 | C—H | C—H | C—Cl | C—H | F | CF3 | — | H | cyclo-PrCH2CO |
| 11-26 | C—H | C—H | C—Cl | C—H | F | CF3 | — | H | CF3CH2CO |
| 11-27 | C—H | C—H | C—Cl | C—H | F | CF3 | — | H | CH3SCH2CO |
| 11-28 | C—H | C—H | C—Cl | C—H | F | CF3 | — | H | CH3S(O)CH2CO |
| 11-29 | C—H | C—H | C—Cl | C—H | F | CF3 | — | H | CH3SO2CH2CO |
| 11-30 | C—H | C—H | C—Cl | C—H | F | CF3 | — | H | CH3CH2NHCO |
| 11-31 | C—Cl | C—H | C—Cl | C—H | H | CF3 | — | H | H |
| 11-32 | C—Cl | C—H | C—Cl | C—H | H | CF3 | — | H | HCO |
| 11-33 | C—Cl | C—H | C—Cl | C—H | H | CF3 | — | H | CH3CO |
| 11-34 | C—Cl | C—H | C—Cl | C—H | H | CF3 | — | H | CH3CH2O |
| 11-35 | C—Cl | C—H | C—Cl | C—H | H | CF3 | — | H | n-PrCO |
| 11-36 | C—Cl | C—H | C—Cl | C—H | H | CF3 | — | H | iso-PrCO |
| 11-37 | C—Cl | C—H | C—Cl | C—H | H | CF3 | — | H | cyclo-PrCO |
| 11-38 | C—Cl | C—H | C—Cl | C—H | H | CF3 | — | H | n-BuCO |
| 11-39 | C—Cl | C—H | C—Cl | C—H | H | CF3 | — | H | tert-BuCO |
| 11-40 | C—Cl | C—H | C—Cl | C—H | H | CF3 | — | H | iso-BuCO |
| 11-41 | C—Cl | C—H | C—Cl | C—H | H | CF3 | — | H | cyclo-BuCO |
| 11-42 | C—Cl | C—H | C—Cl | C—H | H | CF3 | — | H | (CH3)3CCH2CO |
| 11-43 | C—Cl | C—H | C—Cl | C—H | H | CF3 | — | H | cyclo-PenCO |
| 11-44 | C—Cl | C—H | C—Cl | C—H | H | CF3 | — | H | cyclo-HexCO |
| 11-45 | C—Cl | C—H | C—Cl | C—H | H | CF3 | — | H | cyclo-PrCH2CO |
| 11-46 | C—Cl | C—H | C—Cl | C—H | H | CF3 | — | H | CF3CO |
| 11-47 | C—Cl | C—H | C—Cl | C—H | H | CF3 | — | H | CCl3CO |
| 11-48 | C—Cl | C—H | C—Cl | C—H | H | CF3 | — | H | ClCH2CO |
| 11-49 | C—Cl | C—H | C—Cl | C—H | H | CF3 | — | H | CF2ClCO |
| 11-50 | C—Cl | C—H | C—Cl | C—H | H | CF3 | — | H | ClCH2CH2CO |
| 11-51 | C—Cl | C—H | C—Cl | C—H | H | CF3 | — | H | HCF2CF2CO |
| 11-52 | C—Cl | C—H | C—Cl | C—H | H | CF3 | — | H | CF3CH2CO |
| 11-54 | C—Cl | C—H | C—Cl | C—H | H | CF3 | — | H | HCF2CO |
| 11-55 | C—Cl | C—H | C—Cl | C—H | H | CF3 | — | H | ClCH2CO |
| 11-56 | C—Cl | C—H | C—Cl | C—H | H | CF3 | — | H | 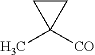 |
| 11-57 | C—Cl | C—H | C—Cl | C—H | H | CF3 | — | H | 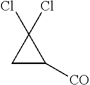 |
| 11-58 | C—Cl | C—H | C—Cl | C—H | H | CF3 | — | H | 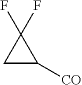 |
| 11-59 | C—Cl | C—H | C—Cl | C—H | H | CF3 | — | H | 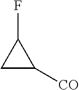 |
| 11-60 | C—Cl | C—H | C—Cl | C—H | H | CF3 | — | H | 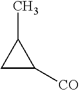 |
| 11-61 | C—Cl | C—H | C—Cl | C—H | H | CF3 | — | H | 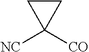 |
| 11-62 | C—Cl | C—H | C—Cl | C—H | H | CF3 | — | H | CH2═CH—CO |
| 11-63 | C—Cl | C—H | C—Cl | C—H | H | CF3 | — | H | CH3CH═CH—CO |
| 11-64 | C—Cl | C—H | C—Cl | C—H | H | CF3 | — | H | CH2═C(CH3)—CO |
| 11-65 | C—Cl | C—H | C—Cl | C—H | H | CF3 | — | H | CH3CH═C(CH3)—CO |
| 11-66 | C—Cl | C—H | C—Cl | C—H | H | CF3 | — | H | (CH3)2C═CH—CO |
| 11-67 | C—Cl | C—H | C—Cl | C—H | H | CF3 | — | H | CH2═C(Cl)—CO |
| 11-68 | C—Cl | C—H | C—Cl | C—H | H | CF3 | — | H | ClCH═C(CH3)—CO |
| 11-69 | C—Cl | C—H | C—Cl | C—H | H | CF3 | — | H | CH2═CHCH2—CO |
| 11-70 | C—Cl | C—H | C—Cl | C—H | H | CF3 | — | H | HCC—CO |
| 11-71 | C—Cl | C—H | C—Cl | C—H | H | CF3 | — | H | CH3CC—CO |
| 11-72 | C—Cl | C—H | C—Cl | C—H | H | CF3 | — | H | NCCH2—CO |
| 11-73 | C—Cl | C—H | C—Cl | C—H | H | CF3 | — | H | CH3OCH2—CO |
| 11-74 | C—Cl | C—H | C—Cl | C—H | H | CF3 | — | H | C2H5OCH2—CO |
| 11-75 | C—Cl | C—H | C—Cl | C—H | H | CF3 | — | H | CH3OCH2CH2—CO |
| 11-76 | C—Cl | C—H | C—Cl | C—H | H | CF3 | — | H | CH3CH2OCH2CH2—CO |
| 11-77 | C—Cl | C—H | C—Cl | C—H | H | CF3 | — | H | CH3SCH2CO |
| 11-78 | C—Cl | C—H | C—Cl | C—H | H | CF3 | — | H | CH3S(O)CH2CO |
| 11-79 | C—Cl | C—H | C—Cl | C—H | H | CF3 | — | H | CH3SO2CH2CO |
| 11-80 | C—Cl | C—H | C—Cl | C—H | H | CF3 | — | H | CH3O(CO)CH2—CO |
| 11-81 | C—Cl | C—H | C—Cl | C—H | H | CF3 | — | H | CH3(CO)OCH2—CO |
| 11-82 | C—Cl | C—H | C—Cl | C—H | H | CF3 | — | H | CH3O(CO)—CO |
| 11-83 | C—Cl | C—H | C—Cl | C—H | H | CF3 | — | H | CF3O(CO)—CO |
| 11-84 | C—Cl | C—H | C—Cl | C—H | H | CF3 | — | H | CH3(CO)CH2—CO |
| 11-85 | C—Cl | C—H | C—Cl | C—H | H | CF3 | — | H | (CH3)2NCH2—CO |
| 11-86 | C—Cl | C—H | C—Cl | C—H | H | CF3 | — | H | PhCO |
| 11-87 | C—Cl | C—H | C—Cl | C—H | H | CF3 | — | H | PhCH2CO |
| 11-88 | C—Cl | C—H | C—Cl | C—H | H | CF3 | — | H | 2-py—CO |
| 11-89 | C—Cl | C—H | C—Cl | C—H | H | CF3 | — | H | 3-py—CO |
| 11-90 | C—Cl | C—H | C—Cl | C—H | H | CF3 | — | H | 4-py—CO |
| 11-91 | C—Cl | C—H | C—Cl | C—H | H | CF3 | — | H | 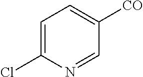 |
| 11-92 | C—Cl | C—H | C—Cl | C—H | H | CF3 | — | H | 2-F—PhCO |
| 11-93 | C—Cl | C—H | C—Cl | C—H | H | CF3 | — | H | 3-F—PhCO |
| 11-94 | C—Cl | C—H | C—Cl | C—H | H | CF3 | — | H | 4-F—PhCO |
| 11-95 | C—Cl | C—H | C—Cl | C—H | H | CF3 | — | H | 2-Cl—PhCO |
| 11-96 | C—Cl | C—H | C—Cl | C—H | H | CF3 | — | H | 3-Cl—PhCO |
| 11-97 | C—Cl | C—H | C—Cl | C—H | H | CF3 | — | H | 4-Cl—PhCO |
| 11-98 | C—Cl | C—H | C—Cl | C—H | H | CF3 | — | H | 2-Br—PhCO |
| 11-99 | C—Cl | C—H | C—Cl | C—H | H | CF3 | — | H | 2-CF3—PhCO |
| 11-100 | C—Cl | C—H | C—Cl | C—H | H | CF3 | — | H | 2-CH3—PhCO |
| 11-101 | C—Cl | C—H | C—Cl | C—H | H | CF3 | — | H | 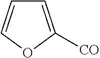 |
| 11-102 | C—Cl | C—H | C—Cl | C—H | H | CF3 | — | H | 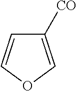 |
| 11-103 | C—Cl | C—H | C—Cl | C—H | H | CF3 | — | H | 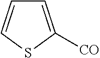 |
| 11-104 | C—Cl | C—H | C—Cl | C—H | H | CF3 | — | H | 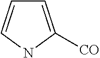 |
| 11-105 | C—Cl | C—H | C—Cl | C—H | H | CF3 | — | H | 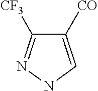 |
| 11-106 | C—Cl | C—H | C—Cl | C—H | H | CF3 | — | H | 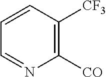 |
| 11-107 | C—Cl | C—H | C—Cl | C—H | H | CF3 | — | H | 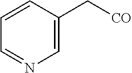 |
| 11-108 | C—Cl | C—H | C—Cl | C—H | H | CF3 | — | H | 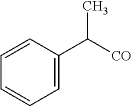 |
| 11-109 | C—Cl | C—H | C—Cl | C—H | H | CF3 | — | H | CH3NHCO |
| 11-110 | C—Cl | C—H | C—Cl | C—H | H | CF3 | — | H | CH3CH2NHCO |
| 11-111 | C—Cl | C—H | C—Cl | C—H | H | CF3 | — | H | CF3CH2NHCO |
| 11-112 | C—Cl | C—H | C—Cl | C—H | H | CF3 | — | H | n-PrNHCO |
| 11-113 | C—Cl | C—H | C—Cl | C—H | H | CF3 | — | H | iso-PrNHCO |
| 11-114 | C—Cl | C—H | C—Cl | C—H | H | CF3 | — | H | cyclo-PrNHCO |
| 11-115 | C—Cl | C—H | C—Cl | C—H | H | CF3 | — | H | n-BuNHCO |
| 11-116 | C—Cl | C—H | C—Cl | C—H | H | CF3 | — | H | tert-BuNHCO |
| 11-117 | C—Cl | C—H | C—Cl | C—H | H | CF3 | — | H | cyclo-BuNHCO |
| 11-118 | C—Cl | C—H | C—Cl | C—H | H | CF3 | — | H | cyclo-PenNHCO |
| 11-119 | C—Cl | C—H | C—Cl | C—H | H | CF3 | — | H | cyclo-HexNHCO |
| 11-120 | C—Cl | C—H | C—Cl | C—H | H | CF3 | — | H | CH2═CHCH2NHCO |
| 11-121 | C—Cl | C—H | C—Cl | C—H | H | CF3 | — | H | HCCCH2NHCO |
| 11-122 | C—Cl | C—H | C—Cl | C—H | H | CF3 | — | H | cyclo-PrCH2NHCO |
| 11-123 | C—Cl | C—H | C—Cl | C—H | H | CF3 | — | H | NCCH2NHCO |
| 11-124 | C—Cl | C—H | C—Cl | C—H | H | CF3 | — | H | CF3(CH3)CHNHCO |
| 11-125 | C—Cl | C—H | C—Cl | C—H | H | CF3 | — | H | CF3(CH3)2CNCO |
| 11-126 | C—Cl | C—H | C—Cl | C—H | H | CF3 | — | H | NCCH2OCO |
| 11-127 | C—Cl | C—H | C—Cl | C—H | H | CF3 | — | H | FCH2CH2NHCO |
| 11-128 | C—Cl | C—H | C—Cl | C—H | H | CF3 | — | H | CF3CH2CH2NHCO |
| 11-129 | C—Cl | C—H | C—Cl | C—H | H | CF3 | — | H | HCF2CF2CH2NHCO |
| 11-130 | C—Cl | C—H | C—Cl | C—H | H | CF3 | — | H | 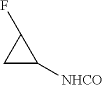 |
| 11-131 | C—Cl | C—H | C—Cl | C—H | H | CF3 | — | H | 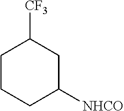 |
| 11-132 | C—Cl | C—H | C—Cl | C—H | H | CF3 | — | H | EtO2CCH2NHCO |
| 11-133 | C—Cl | C—H | C—Cl | C—H | H | CF3 | — | H | 2-CF3—PhNHCO |
| 11-134 | C—Cl | C—H | C—Cl | C—H | H | CF3 | — | H | 3-CF3—PhNHCO |
| 11-135 | C—Cl | C—H | C—Cl | C—H | H | CF3 | — | H | 4-CF3—PhNHCO |
| 11-136 | C—Cl | C—H | C—Cl | C—H | H | CF3 | — | H | 2-py—NHCO |
| 11-137 | C—Cl | C—H | C—Cl | C—H | H | CF3 | — | H | 3-py—NHCO |
| 11-138 | C—Cl | C—H | C—Cl | C—H | H | CF3 | — | H | 4-py—NHCO |
| 11-139 | C—Cl | C—H | C—Cl | C—H | H | CF3 | — | H | 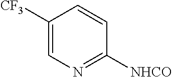 |
| 11-140 | C—Cl | C—H | C—Cl | C—H | H | CF3 | — | H | 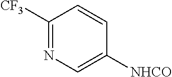 |
| 11-141 | C—Cl | C—H | C—Cl | C—H | H | CF3 | — | H | PhCH2HNCO |
| 11-142 | C—Cl | C—H | C—Cl | C—H | H | CF3 | — | H | 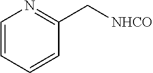 |
| 11-143 | C—Cl | C—H | C—Cl | C—H | H | CF3 | — | H | (CH3)2NCO |
| 11-144 | C—Cl | C—H | C—Cl | C—H | H | CF3 | — | H | pyrrolidineCO |
| 11-145 | C—Cl | C—H | C—Cl | C—H | H | CF3 | — | H | piperidineCO |
| 11-146 | C—Cl | C—H | C—Cl | C—H | H | CF3 | — | H | morpholineCO |
| 11-147 | C—Cl | C—H | C—Cl | C—H | H | CF3 | — | H | CH3OCONH |
| 11-148 | C—Cl | C—H | C—Cl | C—H | H | CF3 | — | H | CH3CH2OCO |
| 11-149 | C—Cl | C—H | C—Cl | C—H | H | CF3 | — | H | n-PrOCO |
| 11-150 | C—Cl | C—H | C—Cl | C—H | H | CF3 | — | H | cyclo-PrOCO |
| 11-151 | C—Cl | C—H | C—Cl | C—H | H | CF3 | — | H | CF3CH2OCO |
| 11-152 | C—Cl | C—H | C—Cl | C—H | H | CF3 | — | H | CH2═CHCH2OCO |
| 11-153 | C—Cl | C—H | C—Cl | C—H | H | CF3 | — | H | HCCCH2OCO |
| 11-154 | C—Cl | C—H | C—Cl | C—H | H | CF3 | — | H | PhOCO |
| 11-155 | C—Cl | C—H | C—Cl | C—H | H | CF3 | — | H | PhCH2OCO |
| 11-156 | C—Cl | C—H | C—Cl | C—H | H | CF3 | — | H | tert-BuOCO |
| 11-157 | C—Cl | C—H | C—Cl | C—H | H | CF3 | — | H | CH3SO2 |
| 11-158 | C—Cl | C—H | C—Cl | C—H | H | CF3 | — | H | CH3CH2SO2 |
| 11-159 | C—Cl | C—H | C—Cl | C—H | H | CF3 | — | H | CF3SO2 |
| 11-160 | C—Cl | C—H | C—Cl | C—H | H | CF3 | — | H | CF3CH2SO2 |
| 11-161 | C—Cl | C—H | C—Cl | C—H | H | CF3 | — | H | PhSO2 |
| 11-162 | C—Cl | C—H | C—Cl | C—H | H | CF3 | — | H | CH3NHCONHSO2 |
| 11-163 | C—Cl | C—H | C—Cl | C—H | H | CF3 | — | H | (CH3)2NCONHSO2 |
| 11-164 | C—Cl | C—H | C—Cl | C—H | H | CF3 | — | H | CH3CS |
| 11-165 | C—Cl | C—H | C—Cl | C—H | H | CF3 | — | H | CH3CH2CS |
| 11-166 | C—Cl | C—H | C—Cl | C—H | H | CF3 | — | H | cyclo-PrCS |
| 11-167 | C—Cl | C—H | C—Cl | C—H | H | CF3 | — | H | CF3CH2CS |
| 11-168 | C—Cl | C—H | C—Br | C—H | H | CF3 | — | H | CH3CO |
| 11-169 | C—Cl | C—H | C—Br | C—H | H | CF3 | — | H | CH3CH2CO |
| 11-170 | C—Cl | C—H | C—Br | C—H | H | CF3 | — | H | n-PrCO |
| 11-171 | C—Cl | C—H | C—Br | C—H | H | CF3 | — | H | cyclo-PrCO |
| 11-172 | C—Cl | C—H | C—Br | C—H | H | CF3 | — | H | cyclo-PrCH2CO |
| 11-173 | C—Cl | C—H | C—Br | C—H | H | CF3 | — | H | CF3CH2CO |
| 11-175 | C—Cl | C—H | C—Br | C—H | H | CF3 | — | H | CH3S(O)CH2CO |
| 11-176 | C—Cl | C—H | C—Br | C—H | H | CF3 | — | H | CH3SO2CH2CO |
| 11-177 | C—Cl | C—H | C—Br | C—H | H | CF3 | — | H | CH3CH2NHCO |
| 11-178 | C—Br | C—H | C—Br | C—H | H | CF3 | — | H | CH3CO |
| 11-179 | C—Br | C—H | C—Br | C—H | H | CF3 | — | H | CH3CH2CO |
| 11-180 | C—Br | C—H | C—Br | C—H | H | CF3 | — | H | n-PrCO |
| 11-181 | C—Br | C—H | C—Br | C—H | H | CF3 | — | H | cyclo-PrCO |
| 11-182 | C—Br | C—H | C—Br | C—H | H | CF3 | — | H | cyclo-PrCH2CO |
| 11-183 | C—Br | C—H | C—Br | C—H | H | CF3 | — | H | CF3CH2CO |
| 11-185 | C—Br | C—H | C—Br | C—H | H | CF3 | — | H | CH3S(O)CH2CO |
| 11-186 | C—Br | C—H | C—Br | C—H | H | CF3 | — | H | CH3SO2CH2CO |
| 11-187 | C—Br | C—H | C—Br | C—H | H | CF3 | — | H | CH3CH2NHCO |
| 11-188 | C—Cl | C—F | C—Cl | C—H | H | CF3 | — | H | CH3CO |
| 11-189 | C—Cl | C—F | C—Cl | C—H | H | CF3 | — | H | CH3CH2CO |
| 11-190 | C—Cl | C—F | C—Cl | C—H | H | CF3 | — | H | n-PrCO |
| 11-191 | C—Cl | C—F | C—Cl | C—H | H | CF3 | — | H | cyclo-PrCO |
| 11-192 | C—Cl | C—F | C—Cl | C—H | H | CF3 | — | H | cyclo-PrCH2CO |
| 11-193 | C—Cl | C—F | C—Cl | C—H | H | CF3 | — | H | CF3CH2CO |
| 11-195 | C—Cl | C—F | C—Cl | C—H | H | CF3 | — | H | CH3S(O)CH2CO |
| 11-196 | C—Cl | C—F | C—Cl | C—H | H | CF3 | — | H | CH3SO2CH2CO |
| 11-197 | C—Cl | C—F | C—Cl | C—H | H | CF3 | — | H | CH3CH2NHCO |
| 11-198 | C—Cl | C—Cl | C—Cl | C—H | H | CF3 | — | H | CH3CO |
| 11-199 | C—Cl | C—Cl | C—Cl | C—H | H | CF3 | — | H | CH3CH2CO |
| 11-200 | C—Cl | C—Cl | C—Cl | C—H | H | CF3 | — | H | n-PrCO |
| 11-201 | C—Cl | C—Cl | C—Cl | C—H | H | CF3 | — | H | cyclo-PrCO |
| 11-202 | C—Cl | C—Cl | C—Cl | C—H | H | CF3 | — | H | cyclo-PrCH2CO |
| 11-203 | C—Cl | C—Cl | C—Cl | C—H | H | CF3 | — | H | CF3CH2CO |
| 11-205 | C—Cl | C—Cl | C—Cl | C—H | H | CF3 | — | H | CH3CH2OCH2CH2—CO |
| 11-206 | C—Cl | C—Cl | C—Cl | C—H | H | CF3 | — | H | CH3SCH2CO |
| 11-207 | C—Cl | C—Cl | C—Cl | C—H | H | CF3 | — | H | CH3S(O)CH2CO |
| 11-208 | C—Cl | C—Cl | C—Cl | C—H | H | CF3 | — | H | CH3SO2CH2CO |
| 11-209 | C—Cl | C—Cl | C—Cl | C—H | H | CF3 | — | H | CH3CH2NHCO |
| 11-210 | C—Cl | C—Br | C—Cl | C—H | H | CF3 | — | H | CH3CO |
| 11-211 | C—Cl | C—Br | C—Cl | C—H | H | CF3 | — | H | CH3CH2CO |
| 11-212 | C—Cl | C—Br | C—Cl | C—H | H | CF3 | — | H | n-PrCO |
| 11-213 | C—Cl | C—Br | C—Cl | C—H | H | CF3 | — | H | cyclo-PrCO |
| 11-214 | C—Cl | C—Br | C—Cl | C—H | H | CF3 | — | H | cyclo-PrCH2CO |
| 11-215 | C—Cl | C—Br | C—Cl | C—H | H | CF3 | — | H | CF3CH2CO |
| 11-217 | C—Cl | C—Br | C—Cl | C—H | H | CF3 | — | H | CH3S(O)CH2CO |
| 11-218 | C—Cl | C—Br | C—Cl | C—H | H | CF3 | — | H | CH3SO2CH2CO |
| 11-219 | C—Cl | C—Br | C—Cl | C—H | H | CF3 | — | H | CH3CH2NHCO |
| 11-220 | C—Cl | C—Cl | C—Br | C—H | H | CF3 | — | H | CH3CO |
| 11-221 | C—Cl | C—Cl | C—Br | C—H | H | CF3 | — | H | CH3CH2CO |
| 11-222 | C—Cl | C—Cl | C—Br | C—H | H | CF3 | — | H | n-PrCO |
| 11-223 | C—Cl | C—Cl | C—Br | C—H | H | CF3 | — | H | cyclo-PrCO |
| 11-224 | C—Cl | C—Cl | C—Br | C—H | H | CF3 | — | H | cyclo-PrCH2CO |
| 11-225 | C—Cl | C—Cl | C—Br | C—H | H | CF3 | — | H | CF3CH2CO |
| 11-227 | C—Cl | C—Cl | C—Br | C—H | H | CF3 | — | H | CH3S(O)CH2CO |
| 11-228 | C—Cl | C—Cl | C—Br | C—H | H | CF3 | — | H | CH3SO2CH2CO |
| 11-229 | C—Cl | C—Cl | C—Br | C—H | H | CF3 | — | H | CH3CH2NHCO |
| 11-230 | C—Cl | C—F | C—Cl | C—H | F | CF3 | — | H | CH3CO |
| 11-231 | C—Cl | C—F | C—Cl | C—H | F | CF3 | — | H | CH3CH2CO |
| 11-232 | C—Cl | C—F | C—Cl | C—H | F | CF3 | — | H | n-PrCO |
| 11-233 | C—Cl | C—F | C—Cl | C—H | F | CF3 | — | H | cyclo-PrCO |
| 11-234 | C—Cl | C—F | C—Cl | C—H | F | CF3 | — | H | cyclo-PrCH2CO |
| 11-235 | C—Cl | C—F | C—Cl | C—H | F | CF3 | — | H | CF3CH2CO |
| 11-237 | C—Cl | C—F | C—Cl | C—H | F | CF3 | — | H | CH3S(O)CH2CO |
| 11-238 | C—Cl | C—F | C—Cl | C—H | F | CF3 | — | H | CH3SO2CH2CO |
| 11-239 | C—Cl | C—F | C—Cl | C—H | F | CF3 | — | H | CH3CH2NHCO |
| 11-240 | C—CF3 | C—H | C—H | C—H | H | CF3 | — | H | CH3CO |
| 11-241 | C—CF3 | C—H | C—H | C—H | H | CF3 | — | H | CH3CH2CO |
| 11-242 | C—CF3 | C—H | C—H | C—H | H | CF3 | — | H | n-PrCO |
| 11-243 | C—CF3 | C—H | C—H | C—H | H | CF3 | — | H | cyclo-PrCO |
| 11-244 | C—CF3 | C—H | C—H | C—H | H | CF3 | — | H | cyclo-PrCH2CO |
| 11-245 | C—CF3 | C—H | C—H | C—H | H | CF3 | — | H | CF3CH2CO |
| 11-247 | C—CF3 | C—H | C—H | C—H | H | CF3 | — | H | CH3S(O)CH2CO |
| 11-248 | C—CF3 | C—H | C—H | C—H | H | CF3 | — | H | CH3SO2CH2CO |
| 11-249 | C—CF3 | C—H | C—H | C—H | H | CF3 | — | H | CH3CH2NHCO |
| 11-250 | C—CF3 | C—F | C—H | C—H | H | CF3 | — | H | CH3CO |
| 11-251 | C—CF3 | C—F | C—H | C—H | H | CF3 | — | H | CH3CH2CO |
| 11-252 | C—CF3 | C—F | C—H | C—H | H | CF3 | — | H | n-PrCO |
| 11-253 | C—CF3 | C—F | C—H | C—H | H | CF3 | — | H | cyclo-PrCO |
| 11-254 | C—CF3 | C—F | C—H | C—H | H | CF3 | — | H | cyclo-PrCH2CO |
| 11-255 | C—CF3 | C—F | C—H | C—H | H | CF3 | — | H | CF3CH2CO |
| 11-257 | C—CF3 | C—F | C—H | C—H | H | CF3 | — | H | CH3S(O)CH2CO |
| 11-258 | C—CF3 | C—F | C—H | C—H | H | CF3 | — | H | CH3SO2CH2CO |
| 11-259 | C—CF3 | C—F | C—H | C—H | H | CF3 | — | H | CH3CH2NHCO |
| 11-260 | C—CF3 | C—H | C—H | C—H | F | CF3 | — | H | CH3CO |
| 11-261 | C—CF3 | C—H | C—H | C—H | F | CF3 | — | H | CH3CH2CO |
| 11-262 | C—CF3 | C—H | C—H | C—H | F | CF3 | — | H | n-PrCO |
| 11-263 | C—CF3 | C—H | C—H | C—H | F | CF3 | — | H | cyclo-PrCO |
| 11-264 | C—CF3 | C—H | C—H | C—H | F | CF3 | — | H | cyclo-PrCH2CO |
| 11-265 | C—CF3 | C—H | C—H | C—H | F | CF3 | — | H | CF3CH2CO |
| 11-267 | C—CF3 | C—H | C—H | C—H | F | CF3 | — | H | CH3S(O)CH2CO |
| 11-268 | C—CF3 | C—H | C—H | C—H | F | CF3 | — | H | CH3SO2CH2CO |
| 11-269 | C—CF3 | C—H | C—H | C—H | F | CF3 | — | H | CH3CH2NHCO |
| 11-270 | C—H | C—H | C—CF3 | C—H | F | CF3 | — | H | CH3CO |
| 11-271 | C—H | C—H | C—CF3 | C—H | F | CF3 | — | H | CH3CH2CO |
| 11-272 | C—H | C—H | C—CF3 | C—H | F | CF3 | — | H | n-PrCO |
| 11-273 | C—H | C—H | C—CF3 | C—H | F | CF3 | — | H | cyclo-PrCO |
| 11-274 | C—H | C—H | C—CF3 | C—H | F | CF3 | — | H | cyclo-PrCH2CO |
| 11-275 | C—H | C—H | C—CF3 | C—H | F | CF3 | — | H | CF3CH2CO |
| 11-277 | C—H | C—H | C—CF3 | C—H | F | CF3 | — | H | CH3S(O)CH2CO |
| 11-278 | C—H | C—H | C—CF3 | C—H | F | CF3 | — | H | CH3SO2CH2CO |
| 11-279 | C—H | C—H | C—CF3 | C—H | F | CF3 | — | H | CH3CH2NHCO |
| 11-280 | C—Cl | C—H | C—CF3 | C—H | H | CF3 | — | H | CH3CO |
| 11-281 | C—Cl | C—H | C—CF3 | C—H | H | CF3 | — | H | CH3CH2CO |
| 11-282 | C—Cl | C—H | C—CF3 | C—H | H | CF3 | — | H | n-PrCO |
| 11-283 | C—Cl | C—H | C—CF3 | C—H | H | CF3 | — | H | cyclo-PrCO |
| 11-284 | C—Cl | C—H | C—CF3 | C—H | H | CF3 | — | H | cyclo-PrCH2CO |
| 11-285 | C—Cl | C—H | C—CF3 | C—H | H | CF3 | — | H | CF3CH2CO |
| 11-287 | C—Cl | C—H | C—CF3 | C—H | H | CF3 | — | H | CH3CH2OCH2CH2—CO |
| 11-288 | C—Cl | C—H | C—CF3 | C—H | H | CF3 | — | H | CH3SCH2CO |
| 11-289 | C—Cl | C—H | C—CF3 | C—H | H | CF3 | — | H | CH3S(O)CH2CO |
| 11-290 | C—Cl | C—H | C—CF3 | C—H | H | CF3 | — | H | CH3SO2CH2CO |
| 11-291 | C—Cl | C—H | C—CF3 | C—H | H | CF3 | — | H | CH3CH2NHCO |
| 11-292 | C—CF3 | C—H | C—CF3 | C—H | H | CF3 | — | H | CH3CO |
| 11-293 | C—CF3 | C—H | C—CF3 | C—H | H | CF3 | — | H | CH3CH2CO |
| 11-294 | C—CF3 | C—H | C—CF3 | C—H | H | CF3 | — | H | n-PrCO |
| 11-295 | C—CF3 | C—H | C—CF3 | C—H | H | CF3 | — | H | cyclo-PrCO |
| 11-296 | C—CF3 | C—H | C—CF3 | C—H | H | CF3 | — | H | cyclo-PrCH2CO |
| 11-297 | C—CF3 | C—H | C—CF3 | C—H | H | CF3 | — | H | CF3CH2CO |
| 11-299 | C—CF3 | C—H | C—CF3 | C—H | H | CF3 | — | H | CH3CH2OCH2CH2—CO |
| 11-300 | C—CF3 | C—H | C—CF3 | C—H | H | CF3 | — | H | CH3SCH2CO |
| 11-301 | C—CF3 | C—H | C—CF3 | C—H | H | CF3 | — | H | CH3S(O)CH2CO |
| 11-302 | C—CF3 | C—H | C—CF3 | C—H | H | CF3 | — | H | CH3SO2CH2CO |
| 11-303 | C—CF3 | C—H | C—CF3 | C—H | H | CF3 | — | H | CH3CH2NHCO |
| 11-304 | C—Cl | C—Cl | C—CF3 | C—H | H | CF3 | — | H | CH3CO |
| 11-305 | C—Cl | C—Cl | C—CF3 | C—H | H | CF3 | — | H | CH3CH2CO |
| 11-306 | C—Cl | C—Cl | C—CF3 | C—H | H | CF3 | — | H | n-PrCO |
| 11-307 | C—Cl | C—Cl | C—CF3 | C—H | H | CF3 | — | H | cyclo-PrCO |
| 11-308 | C—Cl | C—Cl | C—CF3 | C—H | H | CF3 | — | H | cyclo-PrCH2CO |
| 11-309 | C—Cl | C—Cl | C—CF3 | C—H | H | CF3 | — | H | CF3CH2CO |
| 11-311 | C—Cl | C—Cl | C—CF3 | C—H | H | CF3 | — | H | CH3S(O)CH2CO |
| 11-312 | C—Cl | C—Cl | C—CF3 | C—H | H | CF3 | — | H | CH3SO2CH2CO |
| 11-313 | C—Cl | C—Cl | C—CF3 | C—H | H | CF3 | — | H | CH3CH2NHCO |
| 11-314 | C—Cl | N | C—H | C—H | H | CF3 | — | H | CH3CO |
| 11-315 | C—Cl | N | C—H | C—H | H | CF3 | — | H | CH3CH2CO |
| 11-316 | C—Cl | N | C—H | C—H | H | CF3 | — | H | n-PrCO |
| 11-317 | C—Cl | N | C—H | C—H | H | CF3 | — | H | cyclo-PrCO |
| 11-318 | C—Cl | N | C—H | C—H | H | CF3 | — | H | cyclo-PrCH2CO |
| 11-319 | C—Cl | N | C—H | C—H | H | CF3 | — | H | CF3CH2CO |
| 11-321 | C—Cl | N | C—H | C—H | H | CF3 | — | H | CH3S(O)CH2CO |
| 11-322 | C—Cl | N | C—H | C—H | H | CF3 | — | H | CH3SO2CH2CO |
| 11-323 | C—Cl | N | C—H | C—H | H | CF3 | — | H | CH3CH2NHCO |
| 11-324 | C—Cl | N | C—Cl | C—H | H | CF3 | — | H | CH3CO |
| 11-325 | C—Cl | N | C—Cl | C—H | H | CF3 | — | H | CH3CH2CO |
| 11-326 | C—Cl | N | C—Cl | C—H | H | CF3 | — | H | n-PrCO |
| 11-327 | C—Cl | N | C—Cl | C—H | H | CF3 | — | H | cyclo-PrCO |
| 11-328 | C—Cl | N | C—Cl | C—H | H | CF3 | — | H | cyclo-PrCH2CO |
| 11-339 | C—Cl | N | C—Cl | C—H | H | CF3 | — | H | CF3CH2CO |
| 11-330 | C—Cl | N | C—Cl | C—H | H | CF3 | — | H | CH3SCH2CO |
| 11-331 | C—Cl | N | C—Cl | C—H | H | CF3 | — | H | CH3S(O)CH2CO |
| 11-332 | C—Cl | N | C—Cl | C—H | H | CF3 | — | H | CH3SO2CH2CO |
| 11-333 | C—Cl | N | C—Cl | C—H | H | CF3 | — | H | CH3CH2NHCO |
| 11-334 | C—CF3 | N | C—H | C—H | H | CF3 | — | H | CH3CO |
| 11-335 | C—CF3 | N | C—H | C—H | H | CF3 | — | H | CH3CH2CO |
| 11-336 | C—CF3 | N | C—H | C—H | H | CF3 | — | H | n-PrCO |
| 11-337 | C—CF3 | N | C—H | C—H | H | CF3 | — | H | cyclo-PrCO |
| 11-338 | C—CF3 | N | C—H | C—H | H | CF3 | — | H | cyclo-PrCH2CO |
| 11-339 | C—CF3 | N | C—H | C—H | H | CF3 | — | H | CF3CH2CO |
| 11-340 | C—CF3 | N | C—H | C—H | H | CF3 | — | H | CH3SCH2CO |
| 11-341 | C—CF3 | N | C—H | C—H | H | CF3 | — | H | CH3S(O)CH2CO |
| 11-342 | C—CF3 | N | C—H | C—H | H | CF3 | — | H | CH3SO2CH2CO |
| 11-343 | C—CF3 | N | C—H | C—H | H | CF3 | — | H | CH3CH2NHCO |
| 11-344 | C—CF3 | N | C—CF3 | C—H | H | CF3 | — | H | CH3CO |
| 11-345 | C—CF3 | N | C—CF3 | C—H | H | CF3 | — | H | CH3CH2CO |
| 11-346 | C—CF3 | N | C—CF3 | C—H | H | CF3 | — | H | n-PrCO |
| 11-347 | C—CF3 | N | C—CF3 | C—H | H | CF3 | — | H | cyclo-PrCO |
| 11-348 | C—CF3 | N | C—CF3 | C—H | H | CF3 | — | H | cyclo-PrCH2CO |
| 11-349 | C—CF3 | N | C—CF3 | C—H | H | CF3 | — | H | CF3CH2CO |
| 11-350 | C—CF3 | N | C—CF3 | C—H | H | CF3 | — | H | CH3SCH2CO |
| 11-351 | C—CF3 | N | C—CF3 | C—H | H | CF3 | — | H | CH3S(O)CH2CO |
| 11-352 | C—CF3 | N | C—CF3 | C—H | H | CF3 | — | H | CH3SO2CH2CO |
| 11-353 | C—CF3 | N | C—CF3 | C—H | H | CF3 | — | H | CH3CH2NHCO |
| 11-354 | C—CF3 | C—H | C—H | N | H | CF3 | — | H | CH3CO |
| 11-355 | C—CF3 | C—H | C—H | N | H | CF3 | — | H | CH3CH2CO |
| 11-356 | C—CF3 | C—H | C—H | N | H | CF3 | — | H | n-PrCO |
| 11-357 | C—CF3 | C—H | C—H | N | H | CF3 | — | H | cyclo-PrCO |
| 11-358 | C—CF3 | C—H | C—H | N | H | CF3 | — | H | cyclo-PrCH2CO |
| 11-359 | C—CF3 | C—H | C—H | N | H | CF3 | — | H | CF3CH2CO |
| 11-360 | C—CF3 | C—H | C—H | N | H | CF3 | — | H | CH3SCH2CO |
| 11-361 | C—CF3 | C—H | C—H | N | H | CF3 | — | H | CH3S(O)CH2CO |
| 11-362 | C—CF3 | C—H | C—H | N | H | CF3 | — | H | CH3SO2CH2CO |
| 11-363 | C—CF3 | C—H | C—H | N | H | CF3 | — | H | CH3CH2NHCO |
| 11-364 | C—H | C—H | C—CF3 | N | H | CF3 | — | H | CH3CO |
| 11-365 | C—H | C—H | C—CF3 | N | H | CF3 | — | H | CH3CH2CO |
| 11-366 | C—H | C—H | C—CF3 | N | H | CF3 | — | H | n-PrCO |
| 11-367 | C—H | C—H | C—CF3 | N | H | CF3 | — | H | cyclo-PrCO |
| 11-368 | C—H | C—H | C—CF3 | N | H | CF3 | — | H | cyclo-PrCH2CO |
| 11-369 | C—H | C—H | C—CF3 | N | H | CF3 | — | H | CF3CH2CO |
| 11-370 | C—H | C—H | C—CF3 | N | H | CF3 | — | H | CH3SCH2CO |
| 11-371 | C—H | C—H | C—CF3 | N | H | CF3 | — | H | CH3S(O)CH2CO |
| 11-372 | C—H | C—H | C—CF3 | N | H | CF3 | — | H | CH3SO2CH2CO |
| 11-373 | C—H | C—H | C—CF3 | N | H | CF3 | — | H | CH3CH2NHCO |
| 11-374 | C—CF3 | C—H | C—Cl | N | H | CF3 | — | H | CH3CO |
| 11-375 | C—CF3 | C—H | C—Cl | N | H | CF3 | — | H | CH3CH2CO |
| 11-376 | C—CF3 | C—H | C—Cl | N | H | CF3 | — | H | n-PrCO |
| 11-377 | C—CF3 | C—H | C—Cl | N | H | CF3 | — | H | cyclo-PrCO |
| 11-378 | C—CF3 | C—H | C—Cl | N | H | CF3 | — | H | cyclo-PrCH2CO |
| 11-379 | C—CF3 | C—H | C—Cl | N | H | CF3 | — | H | CF3CH2CO |
| 11-380 | C—CF3 | C—H | C—Cl | N | H | CF3 | — | H | CH3SCH2CO |
| 11-381 | C—CF3 | C—H | C—Cl | N | H | CF3 | — | H | CH3S(O)CH2CO |
| 11-382 | C—CF3 | C—H | C—Cl | N | H | CF3 | — | H | CH3SO2CH2CO |
| 11-383 | C—CF3 | C—H | C—Cl | N | H | CF3 | — | H | CH3CH2NHCO |
| 11-384 | C—CF3 | C—H | C—Br | N | H | CF3 | — | H | CH3CO |
| 11-385 | C—CF3 | C—H | C—Br | N | H | CF3 | — | H | CH3CH2CO |
| 11-386 | C—CF3 | C—H | C—Br | N | H | CF3 | — | H | n-PrCO |
| 11-387 | C—CF3 | C—H | C—Br | N | H | CF3 | — | H | cyclo-PrCO |
| 11-388 | C—CF3 | C—H | C—Br | N | H | CF3 | — | H | cyclo-PrCH2CO |
| 11-389 | C—CF3 | C—H | C—Br | N | H | CF3 | — | H | CF3CH2CO |
| 11-390 | C—CF3 | C—H | C—Br | N | H | CF3 | — | H | CH3SCH2CO |
| 11-391 | C—CF3 | C—H | C—Br | N | H | CF3 | — | H | CH3S(O)CH2CO |
| 11-392 | C—CF3 | C—H | C—Br | N | H | CF3 | — | H | CH3SO2CH2CO |
| 11-393 | C—CF3 | C—H | C—Br | N | H | CF3 | — | H | CH3CH2NHCO |
| 11-394 | C—CF3 | C—H | C—CF3 | N | H | CF3 | — | H | CH3CO |
| 11-395 | C—CF3 | C—H | C—CF3 | N | H | CF3 | — | H | CH3CH2CO |
| 11-396 | C—CF3 | C—H | C—CF3 | N | H | CF3 | — | H | n-PrCO |
| 11-397 | C—CF3 | C—H | C—CF3 | N | H | CF3 | — | H | cyclo-PrCO |
| 11-398 | C—CF3 | C—H | C—CF3 | N | H | CF3 | — | H | cyclo-PrCH2CO |
| 11-399 | C—CF3 | C—H | C—CF3 | N | H | CF3 | — | H | CF3CH2CO |
| 11-400 | C—CF3 | C—H | C—CF3 | N | H | CF3 | — | H | CH3SCH2CO |
| 11-401 | C—CF3 | C—H | C—CF3 | N | H | CF3 | — | H | CH3S(O)CH2CO |
| 11-402 | C—CF3 | C—H | C—CF3 | N | H | CF3 | — | H | CH3SO2CH2CO |
| 11-403 | C—CF3 | C—H | C—CF3 | N | H | CF3 | — | H | CH3CH2NHCO |
| 11-404 | C—Cl | C—H | C—H | C—H | H | CF3 | CH2 | H | CH3CO |
| 11-405 | C—Cl | C—H | C—H | C—H | H | CF3 | CH2 | H | CH3CH2CO |
| 11-406 | C—Cl | C—H | C—H | C—H | H | CF3 | CH2 | H | n-PrCO |
| 11-407 | C—Cl | C—H | C—H | C—H | H | CF3 | CH2 | H | cyclo-PrCO |
| 11-408 | C—Cl | C—H | C—H | C—H | H | CF3 | CH2 | H | cyclo-PrCH2CO |
| 11-409 | C—Cl | C—H | C—H | C—H | H | CF3 | CH2 | H | CF3CH2CO |
| 11-410 | C—Cl | C—H | C—H | C—H | H | CF3 | CH2 | H | CH3SCH2CO |
| 11-411 | C—Cl | C—H | C—H | C—H | H | CF3 | CH2 | H | CH3S(O)CH2CO |
| 11-412 | C—Cl | C—H | C—H | C—H | H | CF3 | CH2 | H | CH3SO2CH2CO |
| 11-413 | C—Cl | C—H | C—H | C—H | H | CF3 | CH2 | H | CH3CH2NHCO |
| 11-414 | C—Cl | C—H | C—H | C—H | F | CF3 | CH2 | H | CH3CO |
| 11-415 | C—Cl | C—H | C—H | C—H | F | CF3 | CH2 | H | CH3CH2CO |
| 11-416 | C—Cl | C—H | C—H | C—H | F | CF3 | CH2 | H | n-PrCO |
| 11-417 | C—Cl | C—H | C—H | C—H | F | CF3 | CH2 | H | cyclo-PrCO |
| 11-418 | C—Cl | C—H | C—H | C—H | F | CF3 | CH2 | H | cyclo-PrCH2CO |
| 11-419 | C—Cl | C—H | C—H | C—H | F | CF3 | CH2 | H | CF3CH2CO |
| 11-420 | C—Cl | C—H | C—H | C—H | F | CF3 | CH2 | H | CH3SCH2CO |
| 11-421 | C—Cl | C—H | C—H | C—H | F | CF3 | CH2 | H | CH3S(O)CH2CO |
| 11-422 | C—Cl | C—H | C—H | C—H | F | CF3 | CH2 | H | CH3SO2CH2CO |
| 11-423 | C—Cl | C—H | C—H | C—H | F | CF3 | CH2 | H | CH3CH2NHCO |
| 11-424 | C—H | C—H | C—Cl | C—H | F | CF3 | CH2 | H | CH3CO |
| 11-425 | C—H | C—H | C—Cl | C—H | F | CF3 | CH2 | H | CH3CH2CO |
| 11-426 | C—H | C—H | C—Cl | C—H | F | CF3 | CH2 | H | n-PrCO |
| 11-427 | C—H | C—H | C—Cl | C—H | F | CF3 | CH2 | H | cyclo-PrCO |
| 11-428 | C—H | C—H | C—Cl | C—H | F | CF3 | CH2 | H | cyclo-PrCH2CO |
| 11-429 | C—H | C—H | C—Cl | C—H | F | CF3 | CH2 | H | CF3CH2CO |
| 11-430 | C—H | C—H | C—Cl | C—H | F | CF3 | CH2 | H | CH3SCH2CO |
| 11-431 | C—H | C—H | C—Cl | C—H | F | CF3 | CH2 | H | CH3S(O)CH2CO |
| 11-432 | C—H | C—H | C—Cl | C—H | F | CF3 | CH2 | H | CH3SO2CH2CO |
| 11-433 | C—H | C—H | C—Cl | C—H | F | CF3 | CH2 | H | CH3CH2NHCO |
| 11-434 | C—Cl | C—H | C—Cl | C—H | H | CF3 | CH2 | H | H |
| 11-435 | C—Cl | C—H | C—Cl | C—H | H | CF3 | CH2 | H | HCO |
| 11-436 | C—Cl | C—H | C—Cl | C—H | H | CF3 | CH2 | H | CH3CO |
| 11-437 | C—Cl | C—H | C—Cl | C—H | H | CF3 | CH2 | H | CH3CH2CO |
| 11-438 | C—Cl | C—H | C—Cl | C—H | H | CF3 | CH2 | H | n-PrCO |
| 11-439 | C—Cl | C—H | C—Cl | C—H | H | CF3 | CH2 | H | iso-PrCO |
| 11-440 | C—Cl | C—H | C—Cl | C—H | H | CF3 | CH2 | H | cyclo-PrCO |
| 11-441 | C—Cl | C—H | C—Cl | C—H | H | CF3 | CH2 | H | n-BuCO |
| 11-442 | C—Cl | C—H | C—Cl | C—H | H | CF3 | CH2 | H | tert-BuCO |
| 11-443 | C—Cl | C—H | C—Cl | C—H | H | CF3 | CH2 | H | iso-BuCO |
| 11-444 | C—Cl | C—H | C—Cl | C—H | H | CF3 | CH2 | H | cyclo-BuCO |
| 11-445 | C—Cl | C—H | C—Cl | C—H | H | CF3 | CH2 | H | (CH3)3CCH2CO |
| 11-446 | C—Cl | C—H | C—Cl | C—H | H | CF3 | CH2 | H | cyclo-PenCO |
| 11-447 | C—Cl | C—H | C—Cl | C—H | H | CF3 | CH2 | H | cyclo-HexCO |
| 11-448 | C—Cl | C—H | C—Cl | C—H | H | CF3 | CH2 | H | cyclo-PrCH2CO |
| 11-449 | C—Cl | C—H | C—Cl | C—H | H | CF3 | CH2 | H | CF3CO |
| 11-450 | C—Cl | C—H | C—Cl | C—H | H | CF3 | CH2 | H | CCl3CO |
| 11-451 | C—Cl | C—H | C—Cl | C—H | H | CF3 | CH2 | H | ClCH2CO |
| 11-452 | C—Cl | C—H | C—Cl | C—H | H | CF3 | CH2 | H | CF2ClCO |
| 11-453 | C—Cl | C—H | C—Cl | C—H | H | CF3 | CH2 | H | ClCH2CH2CO |
| 11-454 | C—Cl | C—H | C—Cl | C—H | H | CF3 | CH2 | H | HCF2CF2CO |
| 11-455 | C—Cl | C—H | C—Cl | C—H | H | CF3 | CH2 | H | CF3CH2CO |
| 11-456 | C—Cl | C—H | C—Cl | C—H | H | CF3 | CH2 | H | (CF3)2CHCO |
| 11-457 | C—Cl | C—H | C—Cl | C—H | H | CF3 | CH2 | H | HCF2CO |
| 11-458 | C—Cl | C—H | C—Cl | C—H | H | CF3 | CH2 | H | ClCH2CO |
| 11-459 | C—Cl | C—H | C—Cl | C—H | H | CF3 | CH2 | H | 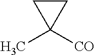 |
| 11-460 | C—Cl | C—H | C—Cl | C—H | H | CF3 | CH2 | H | 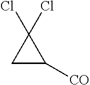 |
| 11-461 | C—Cl | C—H | C—Cl | C—H | H | CF3 | CH2 | H | 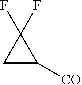 |
| 11-462 | C—Cl | C—H | C—Cl | C—H | H | CF3 | CH2 | H | 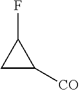 |
| 11-463 | C—Cl | C—H | C—Cl | C—H | H | CF3 | CH2 | H | 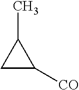 |
| 11-464 | C—Cl | C—H | C—Cl | C—H | H | CF3 | CH2 | H | 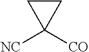 |
| 11-465 | C—Cl | C—H | C—Cl | C—H | H | CF3 | CH2 | H | CH2═CH—CO |
| 11-466 | C—Cl | C—H | C—Cl | C—H | H | CF3 | CH2 | H | CH3CH═CH—CO |
| 11-467 | C—Cl | C—H | C—Cl | C—H | H | CF3 | CH2 | H | CH2═C(CH3)—CO |
| 11-468 | C—Cl | C—H | C—Cl | C—H | H | CF3 | CH2 | H | CH3CH═C(CH3)—CO |
| 11-469 | C—Cl | C—H | C—Cl | C—H | H | CF3 | CH2 | H | (CH3)2C═CH—CO |
| 11-470 | C—Cl | C—H | C—Cl | C—H | H | CF3 | CH2 | H | CH2═C(Cl)—CO |
| 11-471 | C—Cl | C—H | C—Cl | C—H | H | CF3 | CH2 | H | ClCH═C(CH3)—CO |
| 11-472 | C—Cl | C—H | C—Cl | C—H | H | CF3 | CH2 | H | CH2═CHCH2—CO |
| 11-473 | C—Cl | C—H | C—Cl | C—H | H | CF3 | CH2 | H | HCC—CO |
| 11-474 | C—Cl | C—H | C—Cl | C—H | H | CF3 | CH2 | H | CH3CC—CO |
| 11-475 | C—Cl | C—H | C—Cl | C—H | H | CF3 | CH2 | H | NCCH2—CO |
| 11-476 | C—Cl | C—H | C—Cl | C—H | H | CF3 | CH2 | H | CH3OCH2—CO |
| 11-477 | C—Cl | C—H | C—Cl | C—H | H | CF3 | CH2 | H | C2H5OCH2—CO |
| 11-478 | C—Cl | C—H | C—Cl | C—H | H | CF3 | CH2 | H | CH3OCH2CH2—CO |
| 11-479 | C—Cl | C—H | C—Cl | C—H | H | CF3 | CH2 | H | CH3CH2OCH2CH2—CO |
| 11-480 | C—Cl | C—H | C—Cl | C—H | H | CF3 | CH2 | H | CH3SCH2CO |
| 11-481 | C—Cl | C—H | C—Cl | C—H | H | CF3 | CH2 | H | CH3S(O)CH2CO |
| 11-482 | C—Cl | C—H | C—Cl | C—H | H | CF3 | CH2 | H | CH3SO2CH2CO |
| 11-483 | C—Cl | C—H | C—Cl | C—H | H | CF3 | CH2 | H | CH3O(CO)CH2—CO |
| 11-484 | C—Cl | C—H | C—Cl | C—H | H | CF3 | CH2 | H | CH3(CO)OCH2—CO |
| 11-485 | C—Cl | C—H | C—Cl | C—H | H | CF3 | CH2 | H | CH3O(CO)—CO |
| 11-486 | C—Cl | C—H | C—Cl | C—H | H | CF3 | CH2 | H | CF3O(CO)—CO |
| 11-487 | C—Cl | C—H | C—Cl | C—H | H | CF3 | CH2 | H | CH3(CO)CH2—CO |
| 11-488 | C—Cl | C—H | C—Cl | C—H | H | CF3 | CH2 | H | (CH3)2NCH2—CO |
| 11-489 | C—Cl | C—H | C—Cl | C—H | H | CF3 | CH2 | H | PhCO |
| 11-490 | C—Cl | C—H | C—Cl | C—H | H | CF3 | CH2 | H | PhCH2CO |
| 11-491 | C—Cl | C—H | C—Cl | C—H | H | CF3 | CH2 | H | 2-py—CO |
| 11-492 | C—Cl | C—H | C—Cl | C—H | H | CF3 | CH2 | H | 3-py—CO |
| 11-493 | C—Cl | C—H | C—Cl | C—H | H | CF3 | CH2 | H | 4-py—CO |
| 11-494 | C—Cl | C—H | C—Cl | C—H | H | CF3 | CH2 | H | 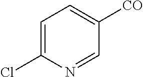 |
| 11-495 | C—Cl | C—H | C—Cl | C—H | H | CF3 | CH2 | H | 2-F—PhCO |
| 11-496 | C—Cl | C—H | C—Cl | C—H | H | CF3 | CH2 | H | 3-F—PhCO |
| 11-497 | C—Cl | C—H | C—Cl | C—H | H | CF3 | CH2 | H | 4-F—PhCO |
| 11-498 | C—Cl | C—H | C—Cl | C—H | H | CF3 | CH2 | H | 2-Cl—PhCO |
| 11-499 | C—Cl | C—H | C—Cl | C—H | H | CF3 | CH2 | H | 3-Cl—PhCO |
| 11-500 | C—Cl | C—H | C—Cl | C—H | H | CF3 | CH2 | H | 4-Cl—PhCO |
| 11-501 | C—Cl | C—H | C—Cl | C—H | H | CF3 | CH2 | H | 2-Br—PhCO |
| 11-502 | C—Cl | C—H | C—Cl | C—H | H | CF3 | CH2 | H | 2-CF3—PhCO |
| 11-503 | C—Cl | C—H | C—Cl | C—H | H | CF3 | CH2 | H | 2-CH3—PhCO |
| 11-504 | C—Cl | C—H | C—Cl | C—H | H | CF3 | CH2 | H | 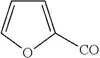 |
| 11-505 | C—Cl | C—H | C—Cl | C—H | H | CF3 | CH2 | H | 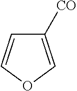 |
| 11-506 | C—Cl | C—H | C—Cl | C—H | H | CF3 | CH2 | H | 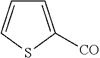 |
| 11-507 | C—Cl | C—H | C—Cl | C—H | H | CF3 | CH2 | H | 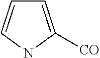 |
| 11-508 | C—Cl | C—H | C—Cl | C—H | H | CF3 | CH2 | H | 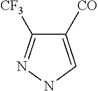 |
| 11-509 | C—Cl | C—H | C—Cl | C—H | H | CF3 | CH2 | H | 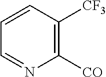 |
| 11-510 | C—Cl | C—H | C—Cl | C—H | H | CF3 | CH2 | H | 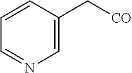 |
| 11-511 | C—Cl | C—H | C—Cl | C—H | H | CF3 | CH2 | H | 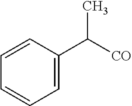 |
| 11-512 | C—Cl | C—H | C—Cl | C—H | H | CF3 | CH2 | H | CH3NHCO |
| 11-513 | C—Cl | C—H | C—Cl | C—H | H | CF3 | CH2 | H | CH3CH2NHCO |
| 11-514 | C—Cl | C—H | C—Cl | C—H | H | CF3 | CH2 | H | CF3CH2NHCO |
| 11-515 | C—Cl | C—H | C—Cl | C—H | H | CF3 | CH2 | H | n-PrNHCO |
| 11-516 | C—Cl | C—H | C—Cl | C—H | H | CF3 | CH2 | H | iso-PrNHCO |
| 11-517 | C—Cl | C—H | C—Cl | C—H | H | CF3 | CH2 | H | cyclo-PrNHCO |
| 11-518 | C—Cl | C—H | C—Cl | C—H | H | CF3 | CH2 | H | n-BuNHCO |
| 11-519 | C—Cl | C—H | C—Cl | C—H | H | CF3 | CH2 | H | tert-BuNHCO |
| 11-520 | C—Cl | C—H | C—Cl | C—H | H | CF3 | CH2 | H | cyclo-BuNHCO |
| 11-521 | C—Cl | C—H | C—Cl | C—H | H | CF3 | CH2 | H | cyclo-PenNHCO |
| 11-522 | C—Cl | C—H | C—Cl | C—H | H | CF3 | CH2 | H | cyclo-HexNHCO |
| 11-523 | C—Cl | C—H | C—Cl | C—H | H | CF3 | CH2 | H | CH2═CHCH2NHCO |
| 11-524 | C—Cl | C—H | C—Cl | C—H | H | CF3 | CH2 | H | HCCCH2NHCO |
| 11-525 | C—Cl | C—H | C—Cl | C—H | H | CF3 | CH2 | H | cyclo-PrCH2NHCO |
| 11-526 | C—Cl | C—H | C—Cl | C—H | H | CF3 | CH2 | H | NCCH2NHCO |
| 11-527 | C—Cl | C—H | C—Cl | C—H | H | CF3 | CH2 | H | CF3(CH3)CHNHCO |
| 11-528 | C—Cl | C—H | C—Cl | C—H | H | CF3 | CH2 | H | CF3(CH3)2CNCO |
| 11-529 | C—Cl | C—H | C—Cl | C—H | H | CF3 | CH2 | H | NCCH2OCO |
| 11-530 | C—Cl | C—H | C—Cl | C—H | H | CF3 | CH2 | H | FCH2CH2NHCO |
| 11-531 | C—Cl | C—H | C—Cl | C—H | H | CF3 | CH2 | H | CF3CH2CH2NHCO |
| 11-532 | C—Cl | C—H | C—Cl | C—H | H | CF3 | CH2 | H | HCF2CF2CH2NHCO |
| 11-533 | C—Cl | C—H | C—Cl | C—H | H | CF3 | CH2 | H | 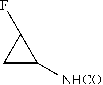 |
| 11-534 | C—Cl | C—H | C—Cl | C—H | H | CF3 | CH2 | H | 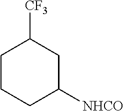 |
| 11-535 | C—Cl | C—H | C—Cl | C—H | H | CF3 | CH2 | H | EtO2CCH2NHCO |
| 11-536 | C—Cl | C—H | C—Cl | C—H | H | CF3 | CH2 | H | 2-CF3—PhNHCO |
| 11-537 | C—Cl | C—H | C—Cl | C—H | H | CF3 | CH2 | H | 3-CF3—PhNHCO |
| 11-538 | C—Cl | C—H | C—Cl | C—H | H | CF3 | CH2 | H | 4-CF3—PhNHCO |
| 11-539 | C—Cl | C—H | C—Cl | C—H | H | CF3 | CH2 | H | 2-py—NHCO |
| 11-540 | C—Cl | C—H | C—Cl | C—H | H | CF3 | CH2 | H | 3-py—NHCO |
| 11-541 | C—Cl | C—H | C—Cl | C—H | H | CF3 | CH2 | H | 4-py—NHCO |
| 11-542 | C—Cl | C—H | C—Cl | C—H | H | CF3 | CH2 | H | 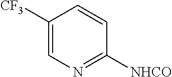 |
| 11-543 | C—Cl | C—H | C—Cl | C—H | H | CF3 | CH2 | H | 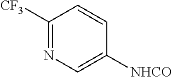 |
| 11-544 | C—Cl | C—H | C—Cl | C—H | H | CF3 | CH2 | H | PhCH2HNCO |
| 11-545 | C—Cl | C—H | C—Cl | C—H | H | CF3 | CH2 | H | 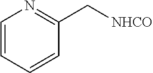 |
| 11-546 | C—Cl | C—H | C—Cl | C—H | H | CF3 | CH2 | H | (CH3)2NCO |
| 11-547 | C—Cl | C—H | C—Cl | C—H | H | CF3 | CH2 | H | pyrrolidineCO |
| 11-548 | C—Cl | C—H | C—Cl | C—H | H | CF3 | CH2 | H | piperidineCO |
| 11-549 | C—Cl | C—H | C—Cl | C—H | H | CF3 | CH2 | H | morpholineCO |
| 11-550 | C—Cl | C—H | C—Cl | C—H | H | CF3 | CH2 | H | CH3OCONH |
| 11-551 | C—Cl | C—H | C—Cl | C—H | H | CF3 | CH2 | H | CH3CH2OCO |
| 11-552 | C—Cl | C—H | C—Cl | C—H | H | CF3 | CH2 | H | n-PrOCO |
| 11-553 | C—Cl | C—H | C—Cl | C—H | H | CF3 | CH2 | H | cyclo-PrOCO |
| 11-554 | C—Cl | C—H | C—Cl | C—H | H | CF3 | CH2 | H | CF3CH2OCO |
| 11-555 | C—Cl | C—H | C—Cl | C—H | H | CF3 | CH2 | H | CH2═CHCH2OCO |
| 11-556 | C—Cl | C—H | C—Cl | C—H | H | CF3 | CH2 | H | HCCCH2OCO |
| 11-557 | C—Cl | C—H | C—Cl | C—H | H | CF3 | CH2 | H | PhOCO |
| 11-558 | C—Cl | C—H | C—Cl | C—H | H | CF3 | CH2 | H | PhCH2OCO |
| 11-559 | C—Cl | C—H | C—Cl | C—H | H | CF3 | CH2 | H | tert-BuOCO |
| 11-560 | C—Cl | C—H | C—Cl | C—H | H | CF3 | CH2 | H | CH3SO2 |
| 11-561 | C—Cl | C—H | C—Cl | C—H | H | CF3 | CH2 | H | CH3CH2SO2 |
| 11-562 | C—Cl | C—H | C—Cl | C—H | H | CF3 | CH2 | H | CF3SO2 |
| 11-563 | C—Cl | C—H | C—Cl | C—H | H | CF3 | CH2 | H | CF3CH2SO2 |
| 11-564 | C—Cl | C—H | C—Cl | C—H | H | CF3 | CH2 | H | PhSO2 |
| 11-565 | C—Cl | C—H | C—Cl | C—H | H | CF3 | CH2 | H | CH3NHCONHSO2 |
| 11-566 | C—Cl | C—H | C—Cl | C—H | H | CF3 | CH2 | H | (CH3)2NCONHSO2 |
| 11-567 | C—Cl | C—H | C—Cl | C—H | H | CF3 | CH2 | H | CH3CS |
| 11-568 | C—Cl | C—H | C—Cl | C—H | H | CF3 | CH2 | H | CH3CH2CS |
| 11-569 | C—Cl | C—H | C—Cl | C—H | H | CF3 | CH2 | H | cyclo-PrCS |
| 11-570 | C—Cl | C—H | C—Cl | C—H | H | CF3 | CH2 | H | CF3CH2CS |
| 11-571 | C—Cl | C—H | C—Br | C—H | H | CF3 | CH2 | H | CH3CO |
| 11-572 | C—Cl | C—H | C—Br | C—H | H | CF3 | CH2 | H | CH3CH2CO |
| 11-573 | C—Cl | C—H | C—Br | C—H | H | CF3 | CH2 | H | n-PrCO |
| 11-574 | C—Cl | C—H | C—Br | C—H | H | CF3 | CH2 | H | cyclo-PrCO |
| 11-575 | C—Cl | C—H | C—Br | C—H | H | CF3 | CH2 | H | cyclo-PrCH2CO |
| 11-576 | C—Cl | C—H | C—Br | C—H | H | CF3 | CH2 | H | CF3CH2CO |
| 11-577 | C—Cl | C—H | C—Br | C—H | H | CF3 | CH2 | H | CH3SCH2CO |
| 11-578 | C—Cl | C—H | C—Br | C—H | H | CF3 | CH2 | H | CH3S(O)CH2CO |
| 11-579 | C—Cl | C—H | C—Br | C—H | H | CF3 | CH2 | H | CH3SO2CH2CO |
| 11-580 | C—Cl | C—H | C—Br | C—H | H | CF3 | CH2 | H | CH3CH2NHCO |
| 11-581 | C—Br | C—H | C—Br | C—H | H | CF3 | CH2 | H | CH3CO |
| 11-582 | C—Br | C—H | C—Br | C—H | H | CF3 | CH2 | H | CH3CH2CO |
| 11-583 | C—Br | C—H | C—Br | C—H | H | CF3 | CH2 | H | n-PrCO |
| 11-584 | C—Br | C—H | C—Br | C—H | H | CF3 | CH2 | H | cyclo-PrCO |
| 11-585 | C—Br | C—H | C—Br | C—H | H | CF3 | CH2 | H | cyclo-PrCH2CO |
| 11-586 | C—Br | C—H | C—Br | C—H | H | CF3 | CH2 | H | CF3CH2CO |
| 11-587 | C—Br | C—H | C—Br | C—H | H | CF3 | CH2 | H | CH3SCH2CO |
| 11-588 | C—Br | C—H | C—Br | C—H | H | CF3 | CH2 | H | CH3S(O)CH2CO |
| 11-589 | C—Br | C—H | C—Br | C—H | H | CF3 | CH2 | H | CH3SO2CH2CO |
| 11-590 | C—Br | C—H | C—Br | C—H | H | CF3 | CH2 | H | CH3CH2NHCO |
| 11-591 | C—Cl | C—F | C—Cl | C—H | H | CF3 | CH2 | H | CH3CO |
| 11-592 | C—Cl | C—F | C—Cl | C—H | H | CF3 | CH2 | H | CH3CH2CO |
| 11-593 | C—Cl | C—F | C—Cl | C—H | H | CF3 | CH2 | H | n-PrCO |
| 11-594 | C—Cl | C—F | C—Cl | C—H | H | CF3 | CH2 | H | cyclo-PrCO |
| 11-595 | C—Cl | C—F | C—Cl | C—H | H | CF3 | CH2 | H | cyclo-PrCH2CO |
| 11-596 | C—Cl | C—F | C—Cl | C—H | H | CF3 | CH2 | H | CF3CH2CO |
| 11-598 | C—Cl | C—F | C—Cl | C—H | H | CF3 | CH2 | H | CH3S(O)CH2CO |
| 11-599 | C—Cl | C—F | C—Cl | C—H | H | CF3 | CH2 | H | CH3SO2CH2CO |
| 11-600 | C—Cl | C—F | C—Cl | C—H | H | CF3 | CH2 | H | CH3CH2NHCO |
| 11-601 | C—Cl | C—Cl | C—Cl | C—H | H | CF3 | CH2 | H | CH3CO |
| 11-602 | C—Cl | C—Cl | C—Cl | C—H | H | CF3 | CH2 | H | CH3CH2CO |
| 11-603 | C—Cl | C—Cl | C—Cl | C—H | H | CF3 | CH2 | H | n-PrCO |
| 11-604 | C—Cl | C—Cl | C—Cl | C—H | H | CF3 | CH2 | H | cyclo-PrCO |
| 11-605 | C—Cl | C—Cl | C—Cl | C—H | H | CF3 | CH2 | H | cyclo-PrCH2CO |
| 11-606 | C—Cl | C—Cl | C—Cl | C—H | H | CF3 | CH2 | H | CF3CH2CO |
| 11-608 | C—Cl | C—Cl | C—Cl | C—H | H | CF3 | CH2 | H | CH3CH2OCH2CH2—CO |
| 11-609 | C—Cl | C—Cl | C—Cl | C—H | H | CF3 | CH2 | H | CH3SCH2CO |
| 11-610 | C—Cl | C—Cl | C—Cl | C—H | H | CF3 | CH2 | H | CH3S(O)CH2CO |
| 11-611 | C—Cl | C—Cl | C—Cl | C—H | H | CF3 | CH2 | H | CH3SO2CH2CO |
| 11-612 | C—Cl | C—Cl | C—Cl | C—H | H | CF3 | CH2 | H | CH3CH2NHCO |
| 11-613 | C—Cl | C—Br | C—Cl | C—H | H | CF3 | CH2 | H | CH3CO |
| 11-614 | C—Cl | C—Br | C—Cl | C—H | H | CF3 | CH2 | H | CH3CH2CO |
| 11-615 | C—Cl | C—Br | C—Cl | C—H | H | CF3 | CH2 | H | n-PrCO |
| 11-616 | C—Cl | C—Br | C—Cl | C—H | H | CF3 | CH2 | H | cyclo-PrCO |
| 11-617 | C—Cl | C—Br | C—Cl | C—H | H | CF3 | CH2 | H | cyclo-PrCH2CO |
| 11-618 | C—Cl | C—Br | C—Cl | C—H | H | CF3 | CH2 | H | CF3CH2CO |
| 11-619 | C—Cl | C—Br | C—Cl | C—H | H | CF3 | CH2 | H | CH3SCH2CO |
| 11-620 | C—Cl | C—Br | C—Cl | C—H | H | CF3 | CH2 | H | CH3S(O)CH2CO |
| 11-621 | C—Cl | C—Br | C—Cl | C—H | H | CF3 | CH2 | H | CH3SO2CH2CO |
| 11-622 | C—Cl | C—Br | C—Cl | C—H | H | CF3 | CH2 | H | CH3CH2NHCO |
| 11-623 | C—Cl | C—Cl | C—Br | C—H | H | CF3 | CH2 | H | CH3CO |
| 11-624 | C—Cl | C—Cl | C—Br | C—H | H | CF3 | CH2 | H | CH3CH2CO |
| 11-625 | C—Cl | C—Cl | C—Br | C—H | H | CF3 | CH2 | H | n-PrCO |
| 11-626 | C—Cl | C—Cl | C—Br | C—H | H | CF3 | CH2 | H | cyclo-PrCO |
| 11-627 | C—Cl | C—Cl | C—Br | C—H | H | CF3 | CH2 | H | cyclo-PrCH2CO |
| 11-628 | C—Cl | C—Cl | C—Br | C—H | H | CF3 | CH2 | H | CF3CH2CO |
| 11-629 | C—Cl | C—Cl | C—Br | C—H | H | CF3 | CH2 | H | CH3SCH2CO |
| 11-630 | C—Cl | C—Cl | C—Br | C—H | H | CF3 | CH2 | H | CH3S(O)CH2CO |
| 11-631 | C—Cl | C—Cl | C—Br | C—H | H | CF3 | CH2 | H | CH3SO2CH2CO |
| 11-632 | C—Cl | C—Cl | C—Br | C—H | H | CF3 | CH2 | H | CH3CH2NHCO |
| 11-633 | C—Cl | C—F | C—Cl | C—H | F | CF3 | CH2 | H | CH3CO |
| 11-634 | C—Cl | C—F | C—Cl | C—H | F | CF3 | CH2 | H | CH3CH2CO |
| 11-635 | C—Cl | C—F | C—Cl | C—H | F | CF3 | CH2 | H | n-PrCO |
| 11-636 | C—Cl | C—F | C—Cl | C—H | F | CF3 | CH2 | H | cyclo-PrCO |
| 11-637 | C—Cl | C—F | C—Cl | C—H | F | CF3 | CH2 | H | cyclo-PrCH2CO |
| 11-638 | C—Cl | C—F | C—Cl | C—H | F | CF3 | CH2 | H | CF3CH2CO |
| 11-639 | C—Cl | C—F | C—Cl | C—H | F | CF3 | CH2 | H | CH3SCH2CO |
| 11-640 | C—Cl | C—F | C—Cl | C—H | F | CF3 | CH2 | H | CH3S(O)CH2CO |
| 11-641 | C—Cl | C—F | C—Cl | C—H | F | CF3 | CH2 | H | CH3SO2CH2CO |
| 11-642 | C—Cl | C—F | C—Cl | C—H | F | CF3 | CH2 | H | CH3CH2NHCO |
| 11-643 | C—CF3 | C—H | C—H | C—H | H | CF3 | CH2 | H | CH3CO |
| 11-644 | C—CF3 | C—H | C—H | C—H | H | CF3 | CH2 | H | CH3CH2CO |
| 11-645 | C—CF3 | C—H | C—H | C—H | H | CF3 | CH2 | H | n-PrCO |
| 11-646 | C—CF3 | C—H | C—H | C—H | H | CF3 | CH2 | H | cyclo-PrCO |
| 11-647 | C—CF3 | C—H | C—H | C—H | H | CF3 | CH2 | H | cyclo-PrCH2CO |
| 11-648 | C—CF3 | C—H | C—H | C—H | H | CF3 | CH2 | H | CF3CH2CO |
| 11-649 | C—CF3 | C—H | C—H | C—H | H | CF3 | CH2 | H | CH3SCH2CO |
| 11-650 | C—CF3 | C—H | C—H | C—H | H | CF3 | CH2 | H | CH3S(O)CH2CO |
| 11-651 | C—CF3 | C—H | C—H | C—H | H | CF3 | CH2 | H | CH3SO2CH2CO |
| 11-652 | C—CF3 | C—H | C—H | C—H | H | CF3 | CH2 | H | CH3CH2NHCO |
| 11-653 | C—CF3 | C—F | C—H | C—H | H | CF3 | CH2 | H | CH3CO |
| 11-654 | C—CF3 | C—F | C—H | C—H | H | CF3 | CH2 | H | CH3CH2CO |
| 11-655 | C—CF3 | C—F | C—H | C—H | H | CF3 | CH2 | H | n-PrCO |
| 11-656 | C—CF3 | C—F | C—H | C—H | H | CF3 | CH2 | H | cyclo-PrCO |
| 11-657 | C—CF3 | C—F | C—H | C—H | H | CF3 | CH2 | H | cyclo-PrCH2CO |
| 11-658 | C—CF3 | C—F | C—H | C—H | H | CF3 | CH2 | H | CF3CH2CO |
| 11-659 | C—CF3 | C—F | C—H | C—H | H | CF3 | CH2 | H | CH3SCH2CO |
| 11-660 | C—CF3 | C—F | C—H | C—H | H | CF3 | CH2 | H | CH3S(O)CH2CO |
| 11-661 | C—CF3 | C—F | C—H | C—H | H | CF3 | CH2 | H | CH3SO2CH2CO |
| 11-662 | C—CF3 | C—F | C—H | C—H | H | CF3 | CH2 | H | CH3CH2NHCO |
| 11-663 | C—CF3 | C—H | C—H | C—H | F | CF3 | CH2 | H | CH3CO |
| 11-664 | C—CF3 | C—H | C—H | C—H | F | CF3 | CH2 | H | CH3CH2CO |
| 11-665 | C—CF3 | C—H | C—H | C—H | F | CF3 | CH2 | H | n-PrCO |
| 11-666 | C—CF3 | C—H | C—H | C—H | F | CF3 | CH2 | H | cyclo-PrCO |
| 11-667 | C—CF3 | C—H | C—H | C—H | F | CF3 | CH2 | H | cyclo-PrCH2CO |
| 11-668 | C—CF3 | C—H | C—H | C—H | F | CF3 | CH2 | H | CF3CH2CO |
| 11-669 | C—CF3 | C—H | C—H | C—H | F | CF3 | CH2 | H | CH3SCH2CO |
| 11-670 | C—CF3 | C—H | C—H | C—H | F | CF3 | CH2 | H | CH3S(O)CH2CO |
| 11-671 | C—CF3 | C—H | C—H | C—H | F | CF3 | CH2 | H | CH3SO2CH2CO |
| 11-672 | C—CF3 | C—H | C—H | C—H | F | CF3 | CH2 | H | CH3CH2NHCO |
| 11-673 | C—H | C—H | C—CF3 | C—H | F | CF3 | CH2 | H | CH3CO |
| 11-674 | C—H | C—H | C—CF3 | C—H | F | CF3 | CH2 | H | CH3CH2CO |
| 11-675 | C—H | C—H | C—CF3 | C—H | F | CF3 | CH2 | H | n-PrCO |
| 11-676 | C—H | C—H | C—CF3 | C—H | F | CF3 | CH2 | H | cyclo-PrCO |
| 11-677 | C—H | C—H | C—CF3 | C—H | F | CF3 | CH2 | H | cyclo-PrCH2CO |
| 11-678 | C—H | C—H | C—CF3 | C—H | F | CF3 | CH2 | H | CF3CH2CO |
| 11-679 | C—H | C—H | C—CF3 | C—H | F | CF3 | CH2 | H | CH3SCH2CO |
| 11-680 | C—H | C—H | C—CF3 | C—H | F | CF3 | CH2 | H | CH3S(O)CH2CO |
| 11-681 | C—H | C—H | C—CF3 | C—H | F | CF3 | CH2 | H | CH3SO2CH2CO |
| 11-682 | C—H | C—H | C—CF3 | C—H | F | CF3 | CH2 | H | CH3CH2NHCO |
| 11-683 | C—Cl | C—H | C—CF3 | C—H | H | CF3 | CH2 | H | CH3CO |
| 11-684 | C—Cl | C—H | C—CF3 | C—H | H | CF3 | CH2 | H | CH3CH2CO |
| 11-685 | C—Cl | C—H | C—CF3 | C—H | H | CF3 | CH2 | H | n-PrCO |
| 11-686 | C—Cl | C—H | C—CF3 | C—H | H | CF3 | CH2 | H | cyclo-PrCO |
| 11-687 | C—Cl | C—H | C—CF3 | C—H | H | CF3 | CH2 | H | cyclo-PrCH2CO |
| 11-688 | C—Cl | C—H | C—CF3 | C—H | H | CF3 | CH2 | H | CF3CH2CO |
| 11-689 | C—Cl | C—H | C—CF3 | C—H | H | CF3 | CH2 | H | CH3OCH2CH2—CO |
| 11-690 | C—Cl | C—H | C—CF3 | C—H | H | CF3 | CH2 | H | CH3CH2OCH2CH2—CO |
| 11-691 | C—Cl | C—H | C—CF3 | C—H | H | CF3 | CH2 | H | CH3SCH2CO |
| 11-692 | C—Cl | C—H | C—CF3 | C—H | H | CF3 | CH2 | H | CH3S(O)CH2CO |
| 11-693 | C—Cl | C—H | C—CF3 | C—H | H | CF3 | CH2 | H | CH3SO2CH2CO |
| 11-694 | C—Cl | C—H | C—CF3 | C—H | H | CF3 | CH2 | H | CH3CH2NHCO |
| 11-695 | C—CF3 | C—H | C—CF3 | C—H | H | CF3 | CH2 | H | CH3CO |
| 11-696 | C—CF3 | C—H | C—CF3 | C—H | H | CF3 | CH2 | H | CH3CH2CO |
| 11-697 | C—CF3 | C—H | C—CF3 | C—H | H | CF3 | CH2 | H | n-PrCO |
| 11-698 | C—CF3 | C—H | C—CF3 | C—H | H | CF3 | CH2 | H | cyclo-PrCO |
| 11-699 | C—CF3 | C—H | C—CF3 | C—H | H | CF3 | CH2 | H | cyclo-PrCH2CO |
| 11-700 | C—CF3 | C—H | C—CF3 | C—H | H | CF3 | CH2 | H | CF3CH2CO |
| 11-701 | C—CF3 | C—H | C—CF3 | C—H | H | CF3 | CH2 | H | CH3OCH2CH2—CO |
| 11-702 | C—CF3 | C—H | C—CF3 | C—H | H | CF3 | CH2 | H | CH3CH2OCH2CH2—CO |
| 11-703 | C—CF3 | C—H | C—CF3 | C—H | H | CF3 | CH2 | H | CH3SCH2CO |
| 11-704 | C—CF3 | C—H | C—CF3 | C—H | H | CF3 | CH2 | H | CH3S(O)CH2CO |
| 11-705 | C—CF3 | C—H | C—CF3 | C—H | H | CF3 | CH2 | H | CH3SO2CH2CO |
| 11-706 | C—CF3 | C—H | C—CF3 | C—H | H | CF3 | CH2 | H | CH3CH2NHCO |
| 11-707 | C—Cl | C—Cl | C—CF3 | C—H | H | CF3 | CH2 | H | CH3CO |
| 11-708 | C—Cl | C—Cl | C—CF3 | C—H | H | CF3 | CH2 | H | CH3CH2CO |
| 11-709 | C—Cl | C—Cl | C—CF3 | C—H | H | CF3 | CH2 | H | n-PrCO |
| 11-710 | C—Cl | C—Cl | C—CF3 | C—H | H | CF3 | CH2 | H | cyclo-PrCO |
| 11-711 | C—Cl | C—Cl | C—CF3 | C—H | H | CF3 | CH2 | H | cyclo-PrCH2CO |
| 11-712 | C—Cl | C—Cl | C—CF3 | C—H | H | CF3 | CH2 | H | CF3CH2CO |
| 11-713 | C—Cl | C—Cl | C—CF3 | C—H | H | CF3 | CH2 | H | CH3SCH2CO |
| 11-714 | C—Cl | C—Cl | C—CF3 | C—H | H | CF3 | CH2 | H | CH3S(O)CH2CO |
| 11-715 | C—Cl | C—Cl | C—CF3 | C—H | H | CF3 | CH2 | H | CH3SO2CH2CO |
| 11-716 | C—Cl | C—Cl | C—CF3 | C—H | H | CF3 | CH2 | H | CH3CH2NHCO |
| 11-717 | C—Cl | N | C—H | C—H | H | CF3 | CH2 | H | CH3CO |
| 11-718 | C—Cl | N | C—H | C—H | H | CF3 | CH2 | H | CH3CH2CO |
| 11-719 | C—Cl | N | C—H | C—H | H | CF3 | CH2 | H | n-PrCO |
| 11-720 | C—Cl | N | C—H | C—H | H | CF3 | CH2 | H | cyclo-PrCO |
| 11-721 | C—Cl | N | C—H | C—H | H | CF3 | CH2 | H | cyclo-PrCH2CO |
| 11-722 | C—Cl | N | C—H | C—H | H | CF3 | CH2 | H | CF3CH2CO |
| 11-723 | C—Cl | N | C—H | C—H | H | CF3 | CH2 | H | CH3SCH2CO |
| 11-724 | C—Cl | N | C—H | C—H | H | CF3 | CH2 | H | CH3S(O)CH2CO |
| 11-725 | C—Cl | N | C—H | C—H | H | CF3 | CH2 | H | CH3SO2CH2CO |
| 11-726 | C—Cl | N | C—H | C—H | H | CF3 | CH2 | H | CH3CH2NHCO |
| 11-727 | C—Cl | N | C—Cl | C—H | H | CF3 | CH2 | H | CH3CO |
| 11-728 | C—Cl | N | C—Cl | C—H | H | CF3 | CH2 | H | CH3CH2CO |
| 11-729 | C—Cl | N | C—Cl | C—H | H | CF3 | CH2 | H | n-PrCO |
| 11-730 | C—Cl | N | C—Cl | C—H | H | CF3 | CH2 | H | cyclo-PrCO |
| 11-731 | C—Cl | N | C—Cl | C—H | H | CF3 | CH2 | H | cyclo-PrCH2CO |
| 11-732 | C—Cl | N | C—Cl | C—H | H | CF3 | CH2 | H | CF3CH2CO |
| 11-733 | C—Cl | N | C—Cl | C—H | H | CF3 | CH2 | H | CH3SCH2CO |
| 11-734 | C—Cl | N | C—Cl | C—H | H | CF3 | CH2 | H | CH3S(O)CH2CO |
| 11-735 | C—Cl | N | C—Cl | C—H | H | CF3 | CH2 | H | CH3SO2CH2CO |
| 11-736 | C—Cl | N | C—Cl | C—H | H | CF3 | CH2 | H | CH3CH2NHCO |
| 11-737 | C—CF3 | N | C—H | C—H | H | CF3 | CH2 | H | CH3CO |
| 11-738 | C—CF3 | N | C—H | C—H | H | CF3 | CH2 | H | CH3CH2CO |
| 11-739 | C—CF3 | N | C—H | C—H | H | CF3 | CH2 | H | n-PrCO |
| 11-740 | C—CF3 | N | C—H | C—H | H | CF3 | CH2 | H | cyclo-PrCO |
| 11-741 | C—CF3 | N | C—H | C—H | H | CF3 | CH2 | H | cyclo-PrCH2CO |
| 11-742 | C—CF3 | N | C—H | C—H | H | CF3 | CH2 | H | CF3CH2CO |
| 11-743 | C—CF3 | N | C—H | C—H | H | CF3 | CH2 | H | CH3SCH2CO |
| 11-744 | C—CF3 | N | C—H | C—H | H | CF3 | CH2 | H | CH3S(O)CH2CO |
| 11-745 | C—CF3 | N | C—H | C—H | H | CF3 | CH2 | H | CH3SO2CH2CO |
| 11-746 | C—CF3 | N | C—H | C—H | H | CF3 | CH2 | H | CH3CH2NHCO |
| 11-747 | C—CF3 | N | C—CF3 | C—H | H | CF3 | CH2 | H | CH3CO |
| 11-748 | C—CF3 | N | C—CF3 | C—H | H | CF3 | CH2 | H | CH3CH2CO |
| 11-749 | C—CF3 | N | C—CF3 | C—H | H | CF3 | CH2 | H | n-PrCO |
| 11-750 | C—CF3 | N | C—CF3 | C—H | H | CF3 | CH2 | H | cyclo-PrCO |
| 11-751 | C—CF3 | N | C—CF3 | C—H | H | CF3 | CH2 | H | cyclo-PrCH2CO |
| 11-752 | C—CF3 | N | C—CF3 | C—H | H | CF3 | CH2 | H | CF3CH2CO |
| 11-753 | C—CF3 | N | C—CF3 | C—H | H | CF3 | CH2 | H | CH3SCH2CO |
| 11-754 | C—CF3 | N | C—CF3 | C—H | H | CF3 | CH2 | H | CH3S(O)CH2CO |
| 11-755 | C—CF3 | N | C—CF3 | C—H | H | CF3 | CH2 | H | CH3SO2CH2CO |
| 11-756 | C—CF3 | N | C—CF3 | C—H | H | CF3 | CH2 | H | CH3CH2NHCO |
| 11-757 | C—CF3 | C—H | C—H | N | H | CF3 | CH2 | H | CH3CO |
| 11-758 | C—CF3 | C—H | C—H | N | H | CF3 | CH2 | H | CH3CH2CO |
| 11-759 | C—CF3 | C—H | C—H | N | H | CF3 | CH2 | H | n-PrCO |
| 11-760 | C—CF3 | C—H | C—H | N | H | CF3 | CH2 | H | cyclo-PrCO |
| 11-761 | C—CF3 | C—H | C—H | N | H | CF3 | CH2 | H | cyclo-PrCH2CO |
| 11-762 | C—CF3 | C—H | C—H | N | H | CF3 | CH2 | H | CF3CH2CO |
| 11-763 | C—CF3 | C—H | C—H | N | H | CF3 | CH2 | H | CH3SCH2CO |
| 11-764 | C—CF3 | C—H | C—H | N | H | CF3 | CH2 | H | CH3S(O)CH2CO |
| 11-765 | C—CF3 | C—H | C—H | N | H | CF3 | CH2 | H | CH3SO2CH2CO |
| 11-766 | C—CF3 | C—H | C—H | N | H | CF3 | CH2 | H | CH3CH2NHCO |
| 11-767 | C—H | C—H | C—CF3 | N | H | CF3 | CH2 | H | CH3CO |
| 11-768 | C—H | C—H | C—CF3 | N | H | CF3 | CH2 | H | CH3CH2CO |
| 11-769 | C—H | C—H | C—CF3 | N | H | CF3 | CH2 | H | n-PrCO |
| 11-770 | C—H | C—H | C—CF3 | N | H | CF3 | CH2 | H | cyclo-PrCO |
| 11-771 | C—H | C—H | C—CF3 | N | H | CF3 | CH2 | H | cyclo-PrCH2CO |
| 11-772 | C—H | C—H | C—CF3 | N | H | CF3 | CH2 | H | CF3CH2CO |
| 11-773 | C—H | C—H | C—CF3 | N | H | CF3 | CH2 | H | CH3SCH2CO |
| 11-774 | C—H | C—H | C—CF3 | N | H | CF3 | CH2 | H | CH3S(O)CH2CO |
| 11-775 | C—H | C—H | C—CF3 | N | H | CF3 | CH2 | H | CH3SO2CH2CO |
| 11-776 | C—H | C—H | C—CF3 | N | H | CF3 | CH2 | H | CH3CH2NHCO |
| 11-777 | C—CF3 | C—H | C—Cl | N | H | CF3 | CH2 | H | CH3CO |
| 11-778 | C—CF3 | C—H | C—Cl | N | H | CF3 | CH2 | H | CH3CH2CO |
| 11-779 | C—CF3 | C—H | C—Cl | N | H | CF3 | CH2 | H | n-PrCO |
| 11-780 | C—CF3 | C—H | C—Cl | N | H | CF3 | CH2 | H | cyclo-PrCO |
| 11-781 | C—CF3 | C—H | C—Cl | N | H | CF3 | CH2 | H | cyclo-PrCH2CO |
| 11-782 | C—CF3 | C—H | C—Cl | N | H | CF3 | CH2 | H | CF3CH2CO |
| 11-783 | C—CF3 | C—H | C—Cl | N | H | CF3 | CH2 | H | CH3SCH2CO |
| 11-784 | C—CF3 | C—H | C—Cl | N | H | CF3 | CH2 | H | CH3S(O)CH2CO |
| 11-785 | C—CF3 | C—H | C—Cl | N | H | CF3 | CH2 | H | CH3SO2CH2CO |
| 11-786 | C—CF3 | C—H | C—Cl | N | H | CF3 | CH2 | H | CH3CH2NHCO |
| 11-787 | C—CF3 | C—H | C—Br | N | H | CF3 | CH2 | H | CH3CO |
| 11-788 | C—CF3 | C—H | C—Br | N | H | CF3 | CH2 | H | CH3CH2CO |
| 11-789 | C—CF3 | C—H | C—Br | N | H | CF3 | CH2 | H | n-PrCO |
| 11-790 | C—CF3 | C—H | C—Br | N | H | CF3 | CH2 | H | cyclo-PrCO |
| 11-791 | C—CF3 | C—H | C—Br | N | H | CF3 | CH2 | H | cyclo-PrCH2CO |
| 11-792 | C—CF3 | C—H | C—Br | N | H | CF3 | CH2 | H | CF3CH2CO |
| 11-794 | C—CF3 | C—H | C—Br | N | H | CF3 | CH2 | H | CH3S(O)CH2CO |
| 11-795 | C—CF3 | C—H | C—Br | N | H | CF3 | CH2 | H | CH3SO2CH2CO |
| 11-796 | C—CF3 | C—H | C—Br | N | H | CF3 | CH2 | H | CH3CH2NHCO |
| 11-797 | C—CF3 | C—H | C—CF3 | N | H | CF3 | CH2 | H | CH3CO |
| 11-798 | C—CF3 | C—H | C—CF3 | N | H | CF3 | CH2 | H | CH3CH2CO |
| 11-799 | C—CF3 | C—H | C—CF3 | N | H | CF3 | CH2 | H | n-PrCO |
| 11-800 | C—CF3 | C—H | C—CF3 | N | H | CF3 | CH2 | H | cyclo-PrCO |
| 11-801 | C—CF3 | C—H | C—CF3 | N | H | CF3 | CH2 | H | cyclo-PrCH2CO |
| 11-802 | C—CF3 | C—H | C—CF3 | N | H | CF3 | CH2 | H | CF3CH2CO |
| 11-804 | C—CF3 | C—H | C—CF3 | N | H | CF3 | CH2 | H | CH3S(O)CH2CO |
| 11-805 | C—CF3 | C—H | C—CF3 | N | H | CF3 | CH2 | H | CH3SO2CH2CO |
| 11-806 | C—CF3 | C—H | C—CF3 | N | H | CF3 | CH2 | H | CH3CH2NHCO |

TABLE 12

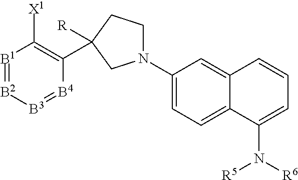

| No. | B¹ | B² | B³ | B⁴ | X¹ | R | R⁵ | R⁶ |
|---|---|---|---|---|---|---|---|---|
| 12-1 | C—Cl | C—H | C—H | C—H | H | CF3 | H | CH3CO |
| 12-2 | C—Cl | C—H | C—H | C—H | H | CF3 | H | CH3CH2CO |
| 12-3 | C—Cl | C—H | C—H | C—H | H | CF3 | H | n-PrCO |
| 12-4 | C—Cl | C—H | C—H | C—H | H | CF3 | H | cyclo-PrCO |
| 12-5 | C—Cl | C—H | C—H | C—H | H | CF3 | H | cyclo-PrCH2CO |
| 12-6 | C—Cl | C—H | C—H | C—H | H | CF3 | H | CF3CH2CO |
| 12-7 | C—Cl | C—H | C—H | C—H | H | CF3 | H | CH3SCH2CO |
| 12-8 | C—Cl | C—H | C—H | C—H | H | CF3 | H | CH3S(O)CH2CO |
| 12-9 | C—Cl | C—H | C—H | C—H | H | CF3 | H | CH3SO2CH2CO |
| 12-10 | C—Cl | C—H | C—H | C—H | H | CF3 | H | CH3CH2NHCO |
| 12-11 | C—Cl | C—H | C—H | C—H | F | CF3 | H | CH3CO |
| 12-12 | C—Cl | C—H | C—H | C—H | F | CF3 | H | CH3CH2CO |
| 12-13 | C—Cl | C—H | C—H | C—H | F | CF3 | H | n-PrCO |
| 12-14 | C—Cl | C—H | C—H | C—H | F | CF3 | H | cyclo-PrCO |
| 12-15 | C—Cl | C—H | C—H | C—H | F | CF3 | H | cyclo-PrCH2CO |
| 12-16 | C—Cl | C—H | C—H | C—H | F | CF3 | H | CF3CH2CO |
| 12-17 | C—Cl | C—H | C—H | C—H | F | CF3 | H | CH3SCH2CO |
| 12-18 | C—Cl | C—H | C—H | C—H | F | CF3 | H | CH3S(O)CH2CO |
| 12-19 | C—Cl | C—H | C—H | C—H | F | CF3 | H | CH3SO2CH2CO |
| 12-20 | C—Cl | C—H | C—H | C—H | F | CF3 | H | CH3CH2NHCO |
| 12-21 | C—H | C—H | C—Cl | C—H | F | CF3 | H | CH3CO |
| 12-22 | C—H | C—H | C—Cl | C—H | F | CF3 | H | CH3CH2CO |
| 12-23 | C—H | C—H | C—Cl | C—H | F | CF3 | H | n-PrCO |
| 12-24 | C—H | C—H | C—Cl | C—H | F | CF3 | H | cyclo-PrCO |
| 12-25 | C—H | C—H | C—Cl | C—H | F | CF3 | H | cyclo-PrCH2CO |
| 12-26 | C—H | C—H | C—Cl | C—H | F | CF3 | H | CF3CH2CO |
| 12-27 | C—H | C—H | C—Cl | C—H | F | CF3 | H | CH3SCH2CO |
| 12-28 | C—H | C—H | C—Cl | C—H | F | CF3 | H | CH3S(O)CH2CO |
| 12-29 | C—H | C—H | C—Cl | C—H | F | CF3 | H | CH3SO2CH2CO |
| 12-30 | C—H | C—H | C—Cl | C—H | F | CF3 | H | CH3CH2NHCO |
| 12-31 | C—Cl | C—H | C—Cl | C—H | H | CF3 | H | H |
| 12-32 | C—Cl | C—H | C—Cl | C—H | H | CF3 | H | HCO |
| 12-33 | C—Cl | C—H | C—Cl | C—H | H | CF3 | H | CH3CO |
| 12-34 | C—Cl | C—H | C—Cl | C—H | H | CF3 | H | CH3CH2CO |
| 12-35 | C—Cl | C—H | C—Cl | C—H | H | CF3 | H | n-PrCO |
| 12-36 | C—Cl | C—H | C—Cl | C—H | H | CF3 | H | iso-PrCO |
| 12-37 | C—Cl | C—H | C—Cl | C—H | H | CF3 | H | cyclo-PrCO |
| 12-38 | C—Cl | C—H | C—Cl | C—H | H | CF3 | H | n-BuCO |
| 12-39 | C—Cl | C—H | C—Cl | C—H | H | CF3 | H | tert-BuCO |
| 12-40 | C—Cl | C—H | C—Cl | C—H | H | CF3 | H | iso-BuCO |
| 12-41 | C—Cl | C—H | C—Cl | C—H | H | CF3 | H | cyclo-BuCO |
| 12-42 | C—Cl | C—H | C—Cl | C—H | H | CF3 | H | (CH3)3CCH2CO |
| 12-43 | C—Cl | C—H | C—Cl | C—H | H | CF3 | H | cyclo-PenCO |
| 12-44 | C—Cl | C—H | C—Cl | C—H | H | CF3 | H | cyclo-HexCO |
| 12-45 | C—Cl | C—H | C—Cl | C—H | H | CF3 | H | cyclo-PrCH2CO |
| 12-46 | C—Cl | C—H | C—Cl | C—H | H | CF3 | H | CF3CO |
| 12-47 | C—Cl | C—H | C—Cl | C—H | H | CF3 | H | CCl3CO |
| 12-48 | C—Cl | C—H | C—Cl | C—H | H | CF3 | H | ClCH2CO |
| 12-49 | C—Cl | C—H | C—Cl | C—H | H | CF3 | H | CF2ClCO |
| 12-50 | C—Cl | C—H | C—Cl | C—H | H | CF3 | H | ClCH2CH2CO |
| 12-51 | C—Cl | C—H | C—Cl | C—H | H | CF3 | H | HCF2CF2CO |
| 12-52 | C—Cl | C—H | C—Cl | C—H | H | CF3 | H | CF3CH2CO |
| 12-54 | C—Cl | C—H | C—Cl | C—H | H | CF3 | H | HCF2CO |
| 12-55 | C—Cl | C—H | C—Cl | C—H | H | CF3 | H | ClCH2CO |
| 12-56 | C—Cl | C—H | C—Cl | C—H | H | CF3 | H | 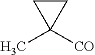 |
| 12-57 | C—Cl | C—H | C—Cl | C—H | H | CF3 | H | 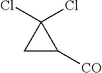 |
| 12-58 | C—Cl | C—H | C—Cl | C—H | H | CF3 | H | 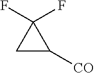 |
| 12-59 | C—Cl | C—H | C—Cl | C—H | H | CF3 | H | 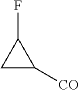 |
| 12-60 | C—Cl | C—H | C—Cl | C—H | H | CF3 | H | 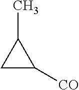 |
| 12-61 | C—Cl | C—H | C—Cl | C—H | H | CF3 | H | 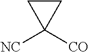 |
| 12-62 | C—Cl | C—H | C—Cl | C—H | H | CF3 | H | CH2═CH—CO |
| 12-63 | C—Cl | C—H | C—Cl | C—H | H | CF3 | H | CH3CH═CH—CO |
| 12-64 | C—Cl | C—H | C—Cl | C—H | H | CF3 | H | CH2═C(CH3)—CO |
| 12-65 | C—Cl | C—H | C—Cl | C—H | H | CF3 | H | CH3CH═C(CH3)—CO |
| 12-66 | C—Cl | C—H | C—Cl | C—H | H | CF3 | H | (CH3)2C═CH—CO |
| 12-67 | C—Cl | C—H | C—Cl | C—H | H | CF3 | H | CH2═C(Cl)—CO |
| 12-68 | C—Cl | C—H | C—Cl | C—H | H | CF3 | H | ClCH═C(CH3)—CO |
| 12-69 | C—Cl | C—H | C—Cl | C—H | H | CF3 | H | CH2═CHCH2—CO |
| 12-70 | C—Cl | C—H | C—Cl | C—H | H | CF3 | H | HCC—CO |
| 12-71 | C—Cl | C—H | C—Cl | C—H | H | CF3 | H | CH3CC—CO |
| 12-72 | C—Cl | C—H | C—Cl | C—H | H | CF3 | H | NCCH2—CO |
| 12-73 | C—Cl | C—H | C—Cl | C—H | H | CF3 | H | CH3OCH2—CO |
| 12-74 | C—Cl | C—H | C—Cl | C—H | H | CF3 | H | C2H5OCH2—CO |
| 12-75 | C—Cl | C—H | C—Cl | C—H | H | CF3 | H | CH3OCH2CH2—CO |
| 12-76 | C—Cl | C—H | C—Cl | C—H | H | CF3 | H | CH3CH2OCH2CH2—CO |
| 12-77 | C—Cl | C—H | C—Cl | C—H | H | CF3 | H | CH3SCH2CO |
| 12-78 | C—Cl | C—H | C—Cl | C—H | H | CF3 | H | CH3S(O)CH2CO |
| 12-79 | C—Cl | C—H | C—Cl | C—H | H | CF3 | H | CH3SO2CH2CO |
| 12-80 | C—Cl | C—H | C—Cl | C—H | H | CF3 | H | CH3O(CO)CH2—CO |
| 12-81 | C—Cl | C—H | C—Cl | C—H | H | CF3 | H | CH3(CO)OCH2—CO |
| 12-82 | C—Cl | C—H | C—Cl | C—H | H | CF3 | H | CH3O(CO)—CO |
| 12-83 | C—Cl | C—H | C—Cl | C—H | H | CF3 | H | CF3O(CO)—CO |
| 12-84 | C—Cl | C—H | C—Cl | C—H | H | CF3 | H | CH3(CO)CH2—CO |
| 12-85 | C—Cl | C—H | C—Cl | C—H | H | CF3 | H | (CH3)2NCH2—CO |
| 12-86 | C—Cl | C—H | C—Cl | C—H | H | CF3 | H | PhCO |
| 12-87 | C—Cl | C—H | C—Cl | C—H | H | CF3 | H | PhCH2CO |
| 12-88 | C—Cl | C—H | C—Cl | C—H | H | CF3 | H | 2-py-CO |
| 12-89 | C—Cl | C—H | C—Cl | C—H | H | CF3 | H | 3-py-CO |
| 12-90 | C—Cl | C—H | C—Cl | C—H | H | CF3 | H | 4-py-CO |
| 12-91 | C—Cl | C—H | C—Cl | C—H | H | CF3 | H | 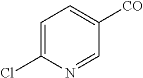 |
| 12-92 | C—Cl | C—H | C—Cl | C—H | H | CF3 | H | 2-F—PhCO |
| 12-93 | C—Cl | C—H | C—Cl | C—H | H | CF3 | H | 3-F—PhCO |
| 12-94 | C—Cl | C—H | C—Cl | C—H | H | CF3 | H | 4-F—PhCO |
| 12-95 | C—Cl | C—H | C—Cl | C—H | H | CF3 | H | 2-Cl—PhCO |
| 12-96 | C—Cl | C—H | C—Cl | C—H | H | CF3 | H | 3-Cl—PhCO |
| 12-97 | C—Cl | C—H | C—Cl | C—H | H | CF3 | H | 4-Cl—PhCO |
| 12-98 | C—Cl | C—H | C—Cl | C—H | H | CF3 | H | 2-Br—PhCO |
| 12-99 | C—Cl | C—H | C—Cl | C—H | H | CF3 | H | 2-CF3—PhCO |
| 12-100 | C—Cl | C—H | C—Cl | C—H | H | CF3 | H | 2-CH3—PhCO |
| 12-101 | C—Cl | C—H | C—Cl | C—H | H | CF3 | H | 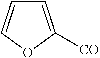 |
| 12-102 | C—Cl | C—H | C—Cl | C—H | H | CF3 | H | 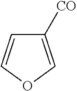 |
| 12-103 | C—Cl | C—H | C—Cl | C—H | H | CF3 | H | 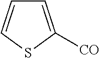 |
| 12-104 | C—Cl | C—H | C—Cl | C—H | H | CF3 | H | 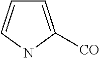 |
| 12-105 | C—Cl | C—H | C—Cl | C—H | H | CF3 | H | 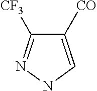 |
| 12-106 | C—Cl | C—H | C—Cl | C—H | H | CF3 | H | 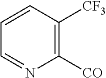 |
| 12-107 | C—Cl | C—H | C—Cl | C—H | H | CF3 | H | 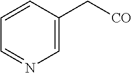 |
| 12-108 | C—Cl | C—H | C—Cl | C—H | H | CF3 | H | 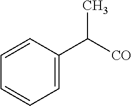 |
| 12-109 | C—Cl | C—H | C—Cl | C—H | H | CF3 | H | CH3NHCO |
| 12-110 | C—Cl | C—H | C—Cl | C—H | H | CF3 | H | CH3CH2NHCO |
| 12-111 | C—Cl | C—H | C—Cl | C—H | H | CF3 | H | CF3CH2NHCO |
| 12-112 | C—Cl | C—H | C—Cl | C—H | H | CF3 | H | n-PrNHCO |
| 12-113 | C—Cl | C—H | C—Cl | C—H | H | CF3 | H | iso-PrNHCO |
| 12-114 | C—Cl | C—H | C—Cl | C—H | H | CF3 | H | cyclo-PrNHCO |
| 12-115 | C—Cl | C—H | C—Cl | C—H | H | CF3 | H | n-BuNHCO |
| 12-116 | C—Cl | C—H | C—Cl | C—H | H | CF3 | H | tert-BuNHCO |
| 12-117 | C—Cl | C—H | C—Cl | C—H | H | CF3 | H | cyclo-BuNHCO |
| 12-118 | C—Cl | C—H | C—Cl | C—H | H | CF3 | H | cyclo-PenNHCO |
| 12-119 | C—Cl | C—H | C—Cl | C—H | H | CF3 | H | cyclo-HexNHCO |
| 12-120 | C—Cl | C—H | C—Cl | C—H | H | CF3 | H | CH2═CHCH2NHCO |
| 12-121 | C—Cl | C—H | C—Cl | C—H | H | CF3 | H | HCCCH2NHCO |
| 12-122 | C—Cl | C—H | C—Cl | C—H | H | CF3 | H | cyclo-PrCH2NHCO |
| 12-123 | C—Cl | C—H | C—Cl | C—H | H | CF3 | H | NCCH2NHCO |
| 12-124 | C—Cl | C—H | C—Cl | C—H | H | CF3 | H | CF3(CH3)CHNHCO |
| 12-125 | C—Cl | C—H | C—Cl | C—H | H | CF3 | H | CF3(CH3)2CNCO |
| 12-126 | C—Cl | C—H | C—Cl | C—H | H | CF3 | H | NCCH20C0 |
| 12-127 | C—Cl | C—H | C—Cl | C—H | H | CF3 | H | FCH2CH2NHCO |
| 12-128 | C—Cl | C—H | C—Cl | C—H | H | CF3 | H | CF3CH2CH2NHCO |
| 12-129 | C—Cl | C—H | C—Cl | C—H | H | CF3 | H | HCF2CF2CH2NHCO |
| 12-130 | C—Cl | C—H | C—Cl | C—H | H | CF3 | H | 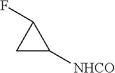 |
| 12-131 | C—Cl | C—H | C—Cl | C—H | H | CF3 | H | 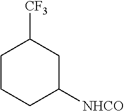 |
| 12-132 | C—Cl | C—H | C—Cl | C—H | H | CF3 | H | EtO2CCH2NHCO |
| 12-133 | C—Cl | C—H | C—Cl | C—H | H | CF3 | H | 2-CF3—PhNHCO |
| 12-134 | C—Cl | C—H | C—Cl | C—H | H | CF3 | H | 3-CF3—PhNHCO |
| 12-135 | C—Cl | C—H | C—Cl | C—H | H | CF3 | H | 4-CF3—PhNHCO |
| 12-136 | C—Cl | C—H | C—Cl | C—H | H | CF3 | H | 2-py-NHCO |
| 12-137 | C—Cl | C—H | C—Cl | C—H | H | CF3 | H | 3-py-NHCO |
| 12-138 | C—Cl | C—H | C—Cl | C—H | H | CF3 | H | 4-py-NHCO |
| 12-139 | C—Cl | C—H | C—Cl | C—H | H | CF3 | H | 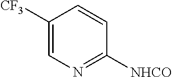 |
| 12-140 | C—Cl | C—H | C—Cl | C—H | H | CF3 | H | 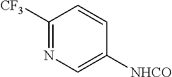 |
| 12-141 | C—Cl | C—H | C—Cl | C—H | H | CF3 | H | PhCH2HNCO |
| 12-142 | C—Cl | C—H | C—Cl | C—H | H | CF3 | H | 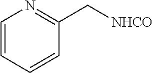 |
| 12-143 | C—Cl | C—H | C—Cl | C—H | H | CF3 | H | (CH3)2NCO |
| 12-144 | C—Cl | C—H | C—Cl | C—H | H | CF3 | H | pyrrolidineCO |
| 12-145 | C—Cl | C—H | C—Cl | C—H | H | CF3 | H | piperidineCO |
| 12-146 | C—Cl | C—H | C—Cl | C—H | H | CF3 | H | morpholineCO |
| 12-147 | C—Cl | C—H | C—Cl | C—H | H | CF3 | H | CH3OCONH |
| 12-148 | C—Cl | C—H | C—Cl | C—H | H | CF3 | H | CH3CH2OCO |
| 12-149 | C—Cl | C—H | C—Cl | C—H | H | CF3 | H | n-PrOCO |
| 12-150 | C—Cl | C—H | C—Cl | C—H | H | CF3 | H | cyclo-PrOCO |
| 12-151 | C—Cl | C—H | C—Cl | C—H | H | CF3 | H | CF3CH20C0 |
| 12-152 | C—Cl | C—H | C—Cl | C—H | H | CF3 | H | CH2═CHCH2OCO |
| 12-153 | C—Cl | C—H | C—Cl | C—H | H | CF3 | H | HCCCH2OCO |
| 12-154 | C—Cl | C—H | C—Cl | C—H | H | CF3 | H | PhOCO |
| 12-155 | C—Cl | C—H | C—Cl | C—H | H | CF3 | H | PhCH2OCO |
| 12-156 | C—Cl | C—H | C—Cl | C—H | H | CF3 | H | tert-BuOCO |
| 12-157 | C—Cl | C—H | C—Cl | C—H | H | CF3 | H | CH3SO2 |
| 12-158 | C—Cl | C—H | C—Cl | C—H | H | CF3 | H | CH3CH2SO2 |
| 12-159 | C—Cl | C—H | C—Cl | C—H | H | CF3 | H | CF3SO2 |
| 12-160 | C—Cl | C—H | C—Cl | C—H | H | CF3 | H | CF3CH2SO2 |
| 12-161 | C—Cl | C—H | C—Cl | C—H | H | CF3 | H | PhSO2 |
| 12-162 | C—Cl | C—H | C—Cl | C—H | H | CF3 | H | CH3NHCONHSO2 |
| 12-163 | C—Cl | C—H | C—Cl | C—H | H | CF3 | H | (CH3)2NCONHSO2 |
| 12-164 | C—Cl | C—H | C—Cl | C—H | H | CF3 | H | CH3CS |
| 12-165 | C—Cl | C—H | C—Cl | C—H | H | CF3 | H | CH3CH2CS |
| 12-166 | C—Cl | C—H | C—Cl | C—H | H | CF3 | H | cyclo-PrCS |
| 12-167 | C—Cl | C—H | C—Cl | C—H | H | CF3 | H | CF3CH2CS |
| 12-168 | C—Cl | C—H | C—Br | C—H | H | CF3 | H | CH3CO |
| 12-169 | C—Cl | C—H | C—Br | C—H | H | CF3 | H | CH3CH2CO |
| 12-170 | C—Cl | C—H | C—Br | C—H | H | CF3 | H | n-PrCO |
| 12-171 | C—Cl | C—H | C—Br | C—H | H | CF3 | H | cyclo-PrCO |
| 12-172 | C—Cl | C—H | C—Br | C—H | H | CF3 | H | cyclo-PrCH2CO |
| 12-173 | C—Cl | C—H | C—Br | C—H | H | CF3 | H | CF3CH2CO |
| 12-175 | C—Cl | C—H | C—Br | C—H | H | CF3 | H | CH3S(O)CH2CO |
| 12-176 | C—Cl | C—H | C—Br | C—H | H | CF3 | H | CH3SO2CH2CO |
| 12-177 | C—Cl | C—H | C—Br | C—H | H | CF3 | H | CH3CH2NHCO |
| 12-178 | C—Br | C—H | C—Br | C—H | H | CF3 | H | CH3CO |
| 12-179 | C—Br | C—H | C—Br | C—H | H | CF3 | H | CH3CH2CO |
| 12-180 | C—Br | C—H | C—Br | C—H | H | CF3 | H | n-PrCO |
| 12-181 | C—Br | C—H | C—Br | C—H | H | CF3 | H | cyclo-PrCO |
| 12-182 | C—Br | C—H | C—Br | C—H | H | CF3 | H | cyclo-PrCH2CO |
| 12-183 | C—Br | C—H | C—Br | C—H | H | CF3 | H | CF3CH2CO |
| 12-185 | C—Br | C—H | C—Br | C—H | H | CF3 | H | CH3S(O)CH2CO |
| 12-186 | C—Br | C—H | C—Br | C—H | H | CF3 | H | CH3SO2CH2CO |
| 12-187 | C—Br | C—H | C—Br | C—H | H | CF3 | H | CH3CH2NHCO |
| 12-188 | C—Cl | C—F | C—Cl | C—H | H | CF3 | H | CH3CO |
| 12-189 | C—Cl | C—F | C—Cl | C—H | H | CF3 | H | CH3CH2CO |
| 12-190 | C—Cl | C—F | C—Cl | C—H | H | CF3 | H | n-PrCO |
| 12-191 | C—Cl | C—F | C—Cl | C—H | H | CF3 | H | cyclo-PrCO |
| 12-192 | C—Cl | C—F | C—Cl | C—H | H | CF3 | H | cyclo-PrCH2C0 |
| 12-193 | C—Cl | C—F | C—Cl | C—H | H | CF3 | H | CF3CH2CO |
| 12-195 | C—Cl | C—F | C—Cl | C—H | H | CF3 | H | CH3S(O)CH2CO |
| 12-196 | C—Cl | C—F | C—Cl | C—H | H | CF3 | H | CH3SO2CH2CO |
| 12-197 | C—Cl | C—F | C—Cl | C—H | H | CF3 | H | CH3CH2NHCO |
| 12-198 | C—Cl | C—Cl | C—Cl | C—H | H | CF3 | H | CH3CO |
| 12-199 | C—Cl | C—Cl | C—Cl | C—H | H | CF3 | H | CH3CH2CO |
| 12-200 | C—Cl | C—Cl | C—Cl | C—H | H | CF3 | H | n-PrCO |
| 12-201 | C—Cl | C—Cl | C—Cl | C—H | H | CF3 | H | cyclo-PrCO |
| 12-202 | C—Cl | C—Cl | C—Cl | C—H | H | CF3 | H | cyclo-PrCH2C0 |
| 12-203 | C—Cl | C—Cl | C—Cl | C—H | H | CF3 | H | CF3CH2CO |
| 12-205 | C—Cl | C—Cl | C—Cl | C—H | H | CF3 | H | CH3CH2OCH2CH2-00 |
| 12-206 | C—Cl | C—Cl | C—Cl | C—H | H | CF3 | H | CH3SCH2C0 |
| 12-207 | C—Cl | C—Cl | C—Cl | C—H | H | CF3 | H | CH3S(O)CH2CO |
| 12-208 | C—Cl | C—Cl | C—Cl | C—H | H | CF3 | H | CH3SO2CH2CO |
| 12-209 | C—Cl | C—Cl | C—Cl | C—H | H | CF3 | H | CH3CH2NHCO |
| 12-210 | C—Cl | C—Br | C—Cl | C—H | H | CF3 | H | CH3CO |
| 12-211 | C—Cl | C—Br | C—Cl | C—H | H | CF3 | H | CH3CH2CO |
| 12-212 | C—Cl | C—Br | C—Cl | C—H | H | CF3 | H | n-PrCO |
| 12-213 | C—Cl | C—Br | C—Cl | C—H | H | CF3 | H | cyclo-PrCO |
| 12-214 | C—Cl | C—Br | C—Cl | C—H | H | CF3 | H | cyclo-PrCH2CO |
| 12-215 | C—Cl | C—Br | C—Cl | C—H | H | CF3 | H | CF3CH2CO |
| 12-217 | C—Cl | C—Br | C—Cl | C—H | H | CF3 | H | CH3S(O)CH2CO |
| 12-218 | C—Cl | C—Br | C—Cl | C—H | H | CF3 | H | CH3SO2CH2CO |
| 12-219 | C—Cl | C—Br | C—Cl | C—H | H | CF3 | H | CH3CH2NHCO |
| 12-220 | C—Cl | C—Cl | C—Br | C—H | H | CF3 | H | CH3CO |
| 12-221 | C—Cl | C—Cl | C—Br | C—H | H | CF3 | H | CH3CH2CO |
| 12-222 | C—Cl | C—Cl | C—Br | C—H | H | CF3 | H | n-PrCO |
| 12-223 | C—Cl | C—Cl | C—Br | C—H | H | CF3 | H | cyclo-PrCO |
| 12-224 | C—Cl | C—Cl | C—Br | C—H | H | CF3 | H | cyclo-PrCH2CO |
| 12-225 | C—Cl | C—Cl | C—Br | C—H | H | CF3 | H | CF3CH2CO |
| 12-227 | C—Cl | C—Cl | C—Br | C—H | H | CF3 | H | CH3S(O)CH2CO |
| 12-228 | C—Cl | C—Cl | C—Br | C—H | H | CF3 | H | CH3SO2CH2CO |
| 12-229 | C—Cl | C—Cl | C—Br | C—H | H | CF3 | H | CH3CH2NHCO |
| 12-230 | C—Cl | C—F | C—Cl | C—H | F | CF3 | H | CH3CO |
| 12-231 | C—Cl | C—F | C—Cl | C—H | F | CF3 | H | CH3CH2CO |
| 12-232 | C—Cl | C—F | C—Cl | C—H | F | CF3 | H | n-PrCO |
| 12-233 | C—Cl | C—F | C—Cl | C—H | F | CF3 | H | cyclo-PrCO |
| 12-234 | C—Cl | C—F | C—Cl | C—H | F | CF3 | H | cyclo-PrCH2CO |
| 12-235 | C—Cl | C—F | C—Cl | C—H | F | CF3 | H | CF3CH2CO |
| 12-237 | C—Cl | C—F | C—Cl | C—H | F | CF3 | H | CH3S(O)CH2CO |
| 12-238 | C—Cl | C—F | C—Cl | C—H | F | CF3 | H | CH3SO2CH2CO |
| 12-239 | C—Cl | C—F | C—Cl | C—H | F | CF3 | H | CH3CH2NHCO |
| 12-240 | C—CF3 | C—H | C—H | C—H | H | CF3 | H | CH3CO |
| 12-241 | C—CF3 | C—H | C—H | C—H | H | CF3 | H | CH3CH2CO |
| 12-242 | C—CF3 | C—H | C—H | C—H | H | CF3 | H | n-PrCO |
| 12-243 | C—CF3 | C—H | C—H | C—H | H | CF3 | H | cyclo-PrCO |
| 12-244 | C—CF3 | C—H | C—H | C—H | H | CF3 | H | cyclo-PrCH2CO |
| 12-245 | C—CF3 | C—H | C—H | C—H | H | CF3 | H | CF3CH2CO |
| 12-247 | C—CF3 | C—H | C—H | C—H | H | CF3 | H | CH3S(O)CH2CO |
| 12-248 | C—CF3 | C—H | C—H | C—H | H | CF3 | H | CH3SO2CH2CO |
| 12-249 | C—CF3 | C—H | C—H | C—H | H | CF3 | H | CH3CH2NHCO |
| 12-250 | C—CF3 | C—F | C—H | C—H | H | CF3 | H | CH3CO |
| 12-251 | C—CF3 | C—F | C—H | C—H | H | CF3 | H | CH3CH2CO |
| 12-252 | C—CF3 | C—F | C—H | C—H | H | CF3 | H | n-PrCO |
| 12-253 | C—CF3 | C—F | C—H | C—H | H | CF3 | H | cyclo-PrCO |
| 12-254 | C—CF3 | C—F | C—H | C—H | H | CF3 | H | cyclo-PrCH2CO |
| 12-255 | C—CF3 | C—F | C—H | C—H | H | CF3 | H | CF3CH2CO |
| 12-257 | C—CF3 | C—F | C—H | C—H | H | CF3 | H | CH3S(O)CH2CO |
| 12-258 | C—CF3 | C—F | C—H | C—H | H | CF3 | H | CH3SO2CH2CO |
| 12-259 | C—CF3 | C—F | C—H | C—H | H | CF3 | H | CH3CH2NHCO |
| 12-260 | C—CF3 | C—H | C—H | C—H | F | CF3 | H | CH3CO |
| 12-261 | C—CF3 | C—H | C—H | C—H | F | CF3 | H | CH3CH2CO |
| 12-262 | C—CF3 | C—H | C—H | C—H | F | CF3 | H | n-PrCO |
| 12-263 | C—CF3 | C—H | C—H | C—H | F | CF3 | H | cyclo-PrCO |
| 12-264 | C—CF3 | C—H | C—H | C—H | F | CF3 | H | cyclo-PrCH2CO |
| 12-265 | C—CF3 | C—H | C—H | C—H | F | CF3 | H | CF3CH2CO |
| 12-267 | C—CF3 | C—H | C—H | C—H | F | CF3 | H | CH3S(O)CH2CO |
| 12-268 | C—CF3 | C—H | C—H | C—H | F | CF3 | H | CH3SO2CH2CO |
| 12-269 | C—CF3 | C—H | C—H | C—H | F | CF3 | H | CH3CH2NHCO |
| 12-270 | C—H | C—H | C—CF3 | C—H | F | CF3 | H | CH3CO |
| 12-271 | C—H | C—H | C—CF3 | C—H | F | CF3 | H | CH3CH2CO |
| 12-272 | C—H | C—H | C—CF3 | C—H | F | CF3 | H | n-PrCO |
| 12-273 | C—H | C—H | C—CF3 | C—H | F | CF3 | H | cyclo-PrCO |
| 12-274 | C—H | C—H | C—CF3 | C—H | F | CF3 | H | cyclo-PrCH2CO |
| 12-275 | C—H | C—H | C—CF3 | C—H | F | CF3 | H | CF3CH2CO |
| 12-277 | C—H | C—H | C—CF3 | C—H | F | CF3 | H | CH3S(O)CH2CO |
| 12-278 | C—H | C—H | C—CF3 | C—H | F | CF3 | H | CH3SO2CH2CO |
| 12-279 | C—H | C—H | C—CF3 | C—H | F | CF3 | H | CH3CH2NHCO |
| 12-280 | C—Cl | C—H | C—CF3 | C—H | H | CF3 | H | CH3CO |
| 12-281 | C—Cl | C—H | C—CF3 | C—H | H | CF3 | H | CH3CH2CO |
| 12-282 | C—Cl | C—H | C—CF3 | C—H | H | CF3 | H | n-PrCO |
| 12-283 | C—Cl | C—H | C—CF3 | C—H | H | CF3 | H | cyclo-PrCO |
| 12-284 | C—Cl | C—H | C—CF3 | C—H | H | CF3 | H | cyclo-PrCH2CO |
| 12-285 | C—Cl | C—H | C—CF3 | C—H | H | CF3 | H | CF3CH2CO |
| 12-287 | C—Cl | C—H | C—CF3 | C—H | H | CF3 | H | CH3CH2OCH2CH2—CO |
| 12-288 | C—Cl | C—H | C—CF3 | C—H | H | CF3 | H | CH3SCH2CO |
| 12-289 | C—Cl | C—H | C—CF3 | C—H | H | CF3 | H | CH3S(O)CH2CO |
| 12-290 | C—Cl | C—H | C—CF3 | C—H | H | CF3 | H | CH3SO2CH2CO |
| 12-291 | C—Cl | C—H | C—CF3 | C—H | H | CF3 | H | CH3CH2NHCO |
| 12-292 | C—CF3 | C—H | C—CF3 | C—H | H | CF3 | H | CH3CO |
| 12-293 | C—CF3 | C—H | C—CF3 | C—H | H | CF3 | H | CH3CH2CO |
| 12-294 | C—CF3 | C—H | C—CF3 | C—H | H | CF3 | H | n-PrCO |
| 12-295 | C—CF3 | C—H | C—CF3 | C—H | H | CF3 | H | cyclo-PrCO |
| 12-296 | C—CF3 | C—H | C—CF3 | C—H | H | CF3 | H | cyclo-PrCH2CO |
| 12-297 | C—CF3 | C—H | C—CF3 | C—H | H | CF3 | H | CF3CH2CO |
| 12-299 | C—CF3 | C—H | C—CF3 | C—H | H | CF3 | H | CH3CH2OCH2CH2—CO |
| 12-300 | C—CF3 | C—H | C—CF3 | C—H | H | CF3 | H | CH3SCH2CO |
| 12-301 | C—CF3 | C—H | C—CF3 | C—H | H | CF3 | H | CH3S(O)CH2CO |
| 12-302 | C—CF3 | C—H | C—CF3 | C—H | H | CF3 | H | CH3SO2CH2CO |
| 12-303 | C—CF3 | C—H | C—CF3 | C—H | H | CF3 | H | CH3CH2NHCO |
| 12-304 | C—Cl | C-Cl | C—CF3 | C—H | H | CF3 | H | CH3CO |
| 12-305 | C—Cl | C-Cl | C—CF3 | C—H | H | CF3 | H | CH3CH2CO |
| 12-306 | C—Cl | C-Cl | C—CF3 | C—H | H | CF3 | H | n-PrCO |
| 12-307 | C—Cl | C-Cl | C—CF3 | C—H | H | CF3 | H | cyclo-PrCO |
| 12-308 | C—Cl | C-Cl | C—CF3 | C—H | H | CF3 | H | cyclo-PrCH2CO |
| 12-309 | C—Cl | C-Cl | C—CF3 | C—H | H | CF3 | H | CF3CH2CO |
| 12-311 | C—Cl | C-Cl | C—CF3 | C—H | H | CF3 | H | CH3S(O)CH2CO |
| 12-312 | C—Cl | C-Cl | C—CF3 | C—H | H | CF3 | H | CH3SO2CH2CO |
| 12-313 | C—Cl | C-Cl | C—CF3 | C—H | H | CF3 | H | CH3CH2NHCO |
| 12-314 | C—Cl | N | C—H | C—H | H | CF3 | H | CH3CO |
| 12-315 | C—Cl | N | C—H | C—H | H | CF3 | H | CH3CH2CO |
| 12-316 | C—Cl | N | C—H | C—H | H | CF3 | H | n-PrCO |
| 12-317 | C—Cl | N | C—H | C—H | H | CF3 | H | cyclo-PrCO |
| 12-318 | C—Cl | N | C—H | C—H | H | CF3 | H | cyclo-PrCH2CO |
| 12-319 | C—Cl | N | C—H | C—H | H | CF3 | H | CF3CH2CO |
| 12-321 | C—Cl | N | C—H | C—H | H | CF3 | H | CH3S(O)CH2CO |
| 12-322 | C—Cl | N | C—H | C—H | H | CF3 | H | CH3SO2CH2CO |
| 12-323 | C—Cl | N | C—H | C—H | H | CF3 | H | CH3CH2NHCO |
| 12-324 | C—Cl | N | C—Cl | C—H | H | CF3 | H | CH3CO |
| 12-325 | C—Cl | N | C—Cl | C—H | H | CF3 | H | CH3CH2CO |
| 12-326 | C—Cl | N | C—Cl | C—H | H | CF3 | H | n-PrCO |
| 12-327 | C—Cl | N | C—Cl | C—H | H | CF3 | H | cyclo-PrCO |
| 12-328 | C—Cl | N | C—Cl | C—H | H | CF3 | H | cyclo-PrCH2CO |
| 12-329 | C—Cl | N | C—Cl | C—H | H | CF3 | H | CF3CH2CO |
| 12-330 | C—Cl | N | C—Cl | C—H | H | CF3 | H | CH3SCH2CO |
| 12-331 | C—Cl | N | C—Cl | C—H | H | CF3 | H | CH3S(O)CH2CO |
| 12-332 | C—Cl | N | C—Cl | C—H | H | CF3 | H | CH3SO2CH2CO |
| 12-333 | C—Cl | N | C—Cl | C—H | H | CF3 | H | CH3CH2NHCO |
| 12-334 | C—CF3 | N | C—H | C—H | H | CF3 | H | CH3CO |
| 12-335 | C—CF3 | N | C—H | C—H | H | CF3 | H | CH3CH2CO |
| 12-336 | C—CF3 | N | C—H | C—H | H | CF3 | H | n-PrCO |
| 12-337 | C—CF3 | N | C—H | C—H | H | CF3 | H | cyclo-PrCO |
| 12-338 | C—CF3 | N | C—H | C—H | H | CF3 | H | cyclo-PrCH2CO |
| 12-339 | C—CF3 | N | C—H | C—H | H | CF3 | H | CF3CH2CO |
| 12-340 | C—CF3 | N | C—H | C—H | H | CF3 | H | CH3SCH2C0 |
| 12-341 | C—CF3 | N | C—H | C—H | H | CF3 | H | CH3S(O)CH2CO |
| 12-342 | C—CF3 | N | C—H | C—H | H | CF3 | H | CH3SO2CH2CO |
| 12-343 | C—CF3 | N | C—H | C—H | H | CF3 | H | CH3CH2NHCO |
| 12-344 | C—CF3 | N | C—CF3 | C—H | H | CF3 | H | CH3CO |
| 12-345 | C—CF3 | N | C—CF3 | C—H | H | CF3 | H | CH3CH2CO |
| 12-346 | C—CF3 | N | C—CF3 | C—H | H | CF3 | H | n-PrCO |
| 12-347 | C—CF3 | N | C—CF3 | C—H | H | CF3 | H | cyclo-PrCO |
| 12-348 | C—CF3 | N | C—CF3 | C—H | H | CF3 | H | cyclo-PrCH2CO |
| 12-349 | C—CF3 | N | C—CF3 | C—H | H | CF3 | H | CF3CH2CO |
| 12-350 | C—CF3 | N | C—CF3 | C—H | H | CF3 | H | CH3SCH2CO |
| 12-351 | C—CF3 | N | C—CF3 | C—H | H | CF3 | H | CH3S(O)CH2CO |
| 12-352 | C—CF3 | N | C—CF3 | C—H | H | CF3 | H | CH3SO2CH2CO |
| 12-353 | C—CF3 | N | C—CF3 | C—H | H | CF3 | H | CH3CH2NHCO |
| 12-354 | C—CF3 | C—H | C—H | N | H | CF3 | H | CH3CO |
| 12-355 | C—CF3 | C—H | C—H | N | H | CF3 | H | CH3CH2CO |
| 12-356 | C—CF3 | C—H | C—H | N | H | CF3 | H | n-PrCO |
| 12-357 | C—CF3 | C—H | C—H | N | H | CF3 | H | cyclo-PrCO |
| 12-358 | C—CF3 | C—H | C—H | N | H | CF3 | H | cyclo-PrCH2CO |
| 12-359 | C—CF3 | C—H | C—H | N | H | CF3 | H | CF3CH2CO |
| 12-360 | C—CF3 | C—H | C—H | N | H | CF3 | H | CH3SCH2CO |
| 12-361 | C—CF3 | C—H | C—H | N | H | CF3 | H | CH3S(O)CH2CO |
| 12-362 | C—CF3 | C—H | C—H | N | H | CF3 | H | CH3SO2CH2CO |
| 12-363 | C—CF3 | C—H | C—H | N | H | CF3 | H | CH3CH2NHCO |
| 12-364 | C—H | C—H | C—CF3 | N | H | CF3 | H | CH3CO |
| 12-365 | C—H | C—H | C—CF3 | N | H | CF3 | H | CH3CH2CO |
| 12-366 | C—H | C—H | C—CF3 | N | H | CF3 | H | n-PrCO |
| 12-367 | C—H | C—H | C—CF3 | N | H | CF3 | H | cyclo-PrCO |
| 12-368 | C—H | C—H | C—CF3 | N | H | CF3 | H | cyclo-PrCH2CO |
| 12-369 | C—H | C—H | C—CF3 | N | H | CF3 | H | CF3CH2CO |
| 12-370 | C—H | C—H | C—CF3 | N | H | CF3 | H | CH3SCH2CO |
| 12-371 | C—H | C—H | C—CF3 | N | H | CF3 | H | CH3S(O)CH2CO |
| 12-372 | C—H | C—H | C—CF3 | N | H | CF3 | H | CH3SO2CH2CO |
| 12-373 | C—H | C—H | C—CF3 | N | H | CF3 | H | CH3CH2NHCO |
| 12-374 | C—CF3 | C—H | C—Cl | N | H | CF3 | H | CH3CO |
| 12-375 | C—CF3 | C—H | C—Cl | N | H | CF3 | H | CH3CH2CO |
| 12-376 | C—CF3 | C—H | C—Cl | N | H | CF3 | H | n-PrCO |
| 12-377 | C—CF3 | C—H | C—Cl | N | H | CF3 | H | cyclo-PrCO |
| 12-378 | C—CF3 | C—H | C—Cl | N | H | CF3 | H | cyclo-PrCH2CO |
| 12-379 | C—CF3 | C—H | C—Cl | N | H | CF3 | H | CF3CH2CO |
| 12-380 | C—CF3 | C—H | C—Cl | N | H | CF3 | H | CH3SCH2CO |
| 12-381 | C—CF3 | C—H | C—Cl | N | H | CF3 | H | CH3S(O)CH2CO |
| 12-382 | C—CF3 | C—H | C—Cl | N | H | CF3 | H | CH3SO2CH2CO |
| 12-383 | C—CF3 | C—H | C—Cl | N | H | CF3 | H | CH3CH2NHCO |
| 12-384 | C—CF3 | C—H | C—Br | N | H | CF3 | H | CH3CO |
| 12-385 | C—CF3 | C—H | C—Br | N | H | CF3 | H | CH3CH2CO |
| 12-386 | C—CF3 | C—H | C—Br | N | H | CF3 | H | n-PrCO |
| 12-387 | C—CF3 | C—H | C—Br | N | H | CF3 | H | cyclo-PrCO |
| 12-388 | C—CF3 | C—H | C—Br | N | H | CF3 | H | cyclo-PrCH2CO |
| 12-389 | C—CF3 | C—H | C—Br | N | H | CF3 | H | CF3CH2CO |
| 12-390 | C—CF3 | C—H | C—Br | N | H | CF3 | H | CH3SCH2CO |
| 12-391 | C—CF3 | C—H | C—Br | N | H | CF3 | H | CH3S(O)CH2CO |
| 12-392 | C—CF3 | C—H | C—Br | N | H | CF3 | H | CH3SO2CH2CO |
| 12-393 | C—CF3 | C—H | C—Br | N | H | CF3 | H | CH3CH2NHCO |
| 12-394 | C—CF3 | C—H | C—CF3 | N | H | CF3 | H | CH3CO |
| 12-395 | C—CF3 | C—H | C—CF3 | N | H | CF3 | H | CH3CH2CO |
| 12-396 | C—CF3 | C—H | C—CF3 | N | H | CF3 | H | n-PrCO |
| 12-397 | C—CF3 | C—H | C—CF3 | N | H | CF3 | H | cyclo-PrCO |
| 12-398 | C—CF3 | C—H | C—CF3 | N | H | CF3 | H | cyclo-PrCH2CO |
| 12-399 | C—CF3 | C—H | C—CF3 | N | H | CF3 | H | CF3CH2CO |
| 12-400 | C—CF3 | C—H | C—CF3 | N | H | CF3 | H | CH3SCH2CO |
| 12-401 | C—CF3 | C—H | C—CF3 | N | H | CF3 | H | CH3S(O)CH2CO |
| 12-402 | C—CF3 | C—H | C—CF3 | N | H | CF3 | H | CH3SO2CH2CO |
| 12-403 | C—CF3 | C—H | C—CF3 | N | H | CF3 | H | CH3CH2NHCO |
| 12-404 | C—Cl | C—Cl | C—Cl | C—H | H | CF3 | H | H |
| 12-405 | C—Cl | C—Cl | C—Cl | C—H | H | CF3 | H | tert-BuOCO |
| 12-406 | C—CF3 | C—H | C—CF3 | C—H | H | CF3 | H | H |
| 12-407 | C—CF3 | C—H | C—CF3 | C—H | H | CF3 | H | tert-BuOCO |

TABLE 13

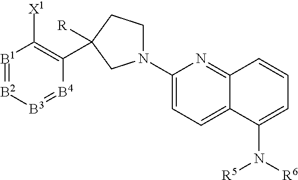

| No. | B¹ | B² | B³ | B⁴ | X¹ | R | R⁵ | R⁶ |
|---|---|---|---|---|---|---|---|---|
| 13-1 | C—Cl | C—H | C—H | C—H | H | CF3 | H | CH3CO |
| 13-2 | C—Cl | C—H | C—H | C—H | H | CF3 | H | CH3CH2CO |
| 13-3 | C—Cl | C—H | C—H | C—H | H | CF3 | H | n-PrCO |
| 13-4 | C—Cl | C—H | C—H | C—H | H | CF3 | H | cyclo-PrCO |
| 13-5 | C—Cl | C—H | C—H | C—H | H | CF3 | H | cyclo-PrCH2CO |
| 13-6 | C—Cl | C—H | C—H | C—H | H | CF3 | H | CF3CH2CO |
| 13-7 | C—Cl | C—H | C—H | C—H | H | CF3 | H | CH3SCH2CO |
| 13-8 | C—Cl | C—H | C—H | C—H | H | CF3 | H | CH3S(O)CH2CO |
| 13-9 | C—Cl | C—H | C—H | C—H | H | CF3 | H | CH3SO2CH2CO |
| 13-10 | C—Cl | C—H | C—H | C—H | H | CF3 | H | CH3CH2NHCO |
| 13-11 | C—Cl | C—H | C—H | C—H | F | CF3 | H | CH3CO |
| 13-12 | C—Cl | C—H | C—H | C—H | F | CF3 | H | CH3CH2CO |
| 13-13 | C—Cl | C—H | C—H | C—H | F | CF3 | H | n-PrCO |
| 13-14 | C—Cl | C—H | C—H | C—H | F | CF3 | H | cyclo-PrCO |
| 13-15 | C—Cl | C—H | C—H | C—H | F | CF3 | H | cyclo-PrCH2CO |
| 13-16 | C—Cl | C—H | C—H | C—H | F | CF3 | H | CF3CH2CO |
| 13-17 | C—Cl | C—H | C—H | C—H | F | CF3 | H | CH3SCH2CO |
| 13-18 | C—Cl | C—H | C—H | C—H | F | CF3 | H | CH3S(O)CH2CO |
| 13-19 | C—Cl | C—H | C—H | C—H | F | CF3 | H | CH3SO2CH2CO |
| 13-20 | C—Cl | C—H | C—H | C—H | F | CF3 | H | CH3CH2NHCO |
| 13-21 | C—H | C—H | C—Cl | C—H | F | CF3 | H | CH3CO |
| 13-22 | C—H | C—H | C—Cl | C—H | F | CF3 | H | CH3CH2CO |
| 13-23 | C—H | C—H | C—Cl | C—H | F | CF3 | H | n-PrCO |
| 13-24 | C—H | C—H | C—Cl | C—H | F | CF3 | H | cyclo-PrCO |
| 13-25 | C—H | C—H | C—Cl | C—H | F | CF3 | H | cyclo-PrCH2CO |
| 13-26 | C—H | C—H | C—Cl | C—H | F | CF3 | H | CF3CH2CO |
| 13-27 | C—H | C—H | C—Cl | C—H | F | CF3 | H | CH3SCH2CO |
| 13-28 | C—H | C—H | C—Cl | C—H | F | CF3 | H | CH3S(O)CH2CO |
| 13-29 | C—H | C—H | C—Cl | C—H | F | CF3 | H | CH3SO2CH2CO |
| 13-30 | C—H | C—H | C—Cl | C—H | F | CF3 | H | CH3CH2NHCO |
| 13-31 | C—Cl | C—H | C—Cl | C—H | H | CF3 | H | H |
| 13-32 | C—Cl | C—H | C—Cl | C—H | H | CF3 | H | HCO |
| 13-33 | C—Cl | C—H | C—Cl | C—H | H | CF3 | H | CH3CO |
| 13-34 | C—Cl | C—H | C—Cl | C—H | H | CF3 | H | CH3CH2CO |
| 13-35 | C—Cl | C—H | C—Cl | C—H | H | CF3 | H | n-PrCO |
| 13-36 | C—Cl | C—H | C—Cl | C—H | H | CF3 | H | iso-PrCO |
| 13-37 | C—Cl | C—H | C—Cl | C—H | H | CF3 | H | cyclo-PrCO |
| 13-38 | C—Cl | C—H | C—Cl | C—H | H | CF3 | H | n-BuCO |
| 13-39 | C—Cl | C—H | C—Cl | C—H | H | CF3 | H | tert-BuCO |
| 13-40 | C—Cl | C—H | C—Cl | C—H | H | CF3 | H | iso-BuCO |
| 13-41 | C—Cl | C—H | C—Cl | C—H | H | CF3 | H | cyclo-BuCO |
| 13-42 | C—Cl | C—H | C—Cl | C—H | H | CF3 | H | (CH3)3CCH2CO |
| 13-43 | C—Cl | C—H | C—Cl | C—H | H | CF3 | H | cyclo-PenCO |
| 13-44 | C—Cl | C—H | C—Cl | C—H | H | CF3 | H | cyclo-HexCO |
| 13-45 | C—Cl | C—H | C—Cl | C—H | H | CF3 | H | cyclo-PrCH2CO |
| 13-46 | C—Cl | C—H | C—Cl | C—H | H | CF3 | H | CF3CO |
| 13-47 | C—Cl | C—H | C—Cl | C—H | H | CF3 | H | CCl3CO |
| 13-48 | C—Cl | C—H | C—Cl | C—H | H | CF3 | H | ClCH2CO |
| 13-49 | C—Cl | C—H | C—Cl | C—H | H | CF3 | H | CF2ClCO |
| 13-50 | C—Cl | C—H | C—Cl | C—H | H | CF3 | H | ClCH2CH2CO |
| 13-51 | C—Cl | C—H | C—Cl | C—H | H | CF3 | H | HCF2CF2CO |
| 13-52 | C—Cl | C—H | C—Cl | C—H | H | CF3 | H | CF3CH2CO |
| 13-54 | C—Cl | C—H | C—Cl | C—H | H | CF3 | H | HCF2CO |
| 13-55 | C—Cl | C—H | C—Cl | C—H | H | CF3 | H | ClCH2CO |
| 13-56 | C—Cl | C—H | C—Cl | C—H | H | CF3 | H | 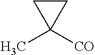 |
| 13-57 | C—Cl | C—H | C—Cl | C—H | H | CF3 | H | 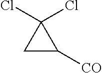 |
| 13-58 | C—Cl | C—H | C—Cl | C—H | H | CF3 | H | 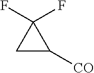 |
| 13-59 | C—Cl | C—H | C—Cl | C—H | H | CF3 | H | 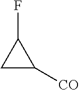 |
| 13-60 | C—Cl | C—H | C—Cl | C—H | H | CF3 | H | 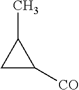 |
| 13-61 | C—Cl | C—H | C—Cl | C—H | H | CF3 | H | 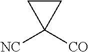 |
| 13-62 | C—Cl | C—H | C—Cl | C—H | H | CF3 | H | CH2═CH—CO |
| 13-63 | C—Cl | C—H | C—Cl | C—H | H | CF3 | H | CH3CH═CH—CO |
| 13-64 | C—Cl | C—H | C—Cl | C—H | H | CF3 | H | CH2═C(CH3)—CO |
| 13-65 | C—Cl | C—H | C—Cl | C—H | H | CF3 | H | CH3CH═C(CH3)—CO |
| 13-66 | C—Cl | C—H | C—Cl | C—H | H | CF3 | H | (CH3)2C═CH—CO |
| 13-67 | C—Cl | C—H | C—Cl | C—H | H | CF3 | H | CH2═C(Cl)—CO |
| 13-68 | C—Cl | C—H | C—Cl | C—H | H | CF3 | H | ClCH═C(CH3)—CO |
| 13-69 | C—Cl | C—H | C—Cl | C—H | H | CF3 | H | CH2═CHCH2—CO |
| 13-70 | C—Cl | C—H | C—Cl | C—H | H | CF3 | H | HCC—CO |
| 13-71 | C—Cl | C—H | C—Cl | C—H | H | CF3 | H | CH3CC—CO |
| 13-72 | C—Cl | C—H | C—Cl | C—H | H | CF3 | H | NCCH2—CO |
| 13-73 | C—Cl | C—H | C—Cl | C—H | H | CF3 | H | CH3OCH2—CO |
| 13-74 | C—Cl | C—H | C—Cl | C—H | H | CF3 | H | C2H5OCH2—CO |
| 13-75 | C—Cl | C—H | C—Cl | C—H | H | CF3 | H | CH3OCH2CH2—CO |
| 13-76 | C—Cl | C—H | C—Cl | C—H | H | CF3 | H | CH3CH2OCH2CH2—CO |
| 13-77 | C—Cl | C—H | C—Cl | C—H | H | CF3 | H | CH3SCH2CO |
| 13-78 | C—Cl | C—H | C—Cl | C—H | H | CF3 | H | CH3S(O)CH2CO |
| 13-79 | C—Cl | C—H | C—Cl | C—H | H | CF3 | H | CH3SO2CH2CO |
| 13-80 | C—Cl | C—H | C—Cl | C—H | H | CF3 | H | CH3O(CO)CH2—CO |
| 13-81 | C—Cl | C—H | C—Cl | C—H | H | CF3 | H | CH3(CO)OCH2—CO |
| 13-82 | C—Cl | C—H | C—Cl | C—H | H | CF3 | H | CH3O(CO)—CO |
| 13-83 | C—Cl | C—H | C—Cl | C—H | H | CF3 | H | CF3O(CO)—CO |
| 13-84 | C—Cl | C—H | C—Cl | C—H | H | CF3 | H | CH3(CO)CH2—CO |
| 13-85 | C—Cl | C—H | C—Cl | C—H | H | CF3 | H | (CH3)2NCH2—CO |
| 13-86 | C—Cl | C—H | C—Cl | C—H | H | CF3 | H | PhCO |
| 13-87 | C—Cl | C—H | C—Cl | C—H | H | CF3 | H | PhCH2CO |
| 13-88 | C—Cl | C—H | C—Cl | C—H | H | CF3 | H | 2-py-CO |
| 13-89 | C—Cl | C—H | C—Cl | C—H | H | CF3 | H | 3-py-CO |
| 13-90 | C—Cl | C—H | C—Cl | C—H | H | CF3 | H | 4-py-CO |
| 13-91 | C—Cl | C—H | C—Cl | C—H | H | CF3 | H | 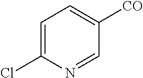 |
| 13-92 | C—Cl | C—H | C—Cl | C—H | H | CF3 | H | 2-F—PhCO |
| 13-93 | C—Cl | C—H | C—Cl | C—H | H | CF3 | H | 3-F—PhCO |
| 13-94 | C—Cl | C—H | C—Cl | C—H | H | CF3 | H | 4-F—PhCO |
| 13-95 | C—Cl | C—H | C—Cl | C—H | H | CF3 | H | 2-Cl—PhCO |
| 13-96 | C—Cl | C—H | C—Cl | C—H | H | CF3 | H | 3-Cl—PhCO |
| 13-97 | C—Cl | C—H | C—Cl | C—H | H | CF3 | H | 4-Cl—PhCO |
| 13-98 | C—Cl | C—H | C—Cl | C—H | H | CF3 | H | 2-Br—PhCO |
| 13-99 | C—Cl | C—H | C—Cl | C—H | H | CF3 | H | 2-CF3—PhCO |
| 13-100 | C—Cl | C—H | C—Cl | C—H | H | CF3 | H | 2-CH3—PhCO |
| 13-101 | C—Cl | C—H | C—Cl | C—H | H | CF3 | H | 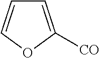 |
| 13-102 | C—Cl | C—H | C—Cl | C—H | H | CF3 | H | 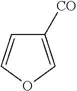 |
| 13-103 | C—Cl | C—H | C—Cl | C—H | H | CF3 | H | 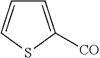 |
| 13-104 | C—Cl | C—H | C—Cl | C—H | H | CF3 | H | 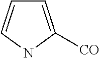 |
| 13-105 | C—Cl | C—H | C—Cl | C—H | H | CF3 | H | 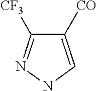 |
| 13-106 | C—Cl | C—H | C—Cl | C—H | H | CF3 | H | 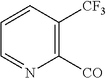 |
| 13-107 | C—Cl | C—H | C—Cl | C—H | H | CF3 | H | 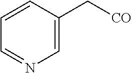 |
| 13-108 | C—Cl | C—H | C—Cl | C—H | H | CF3 | H | 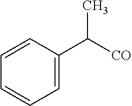 |
| 13-109 | C—Cl | C—H | C—Cl | C—H | H | CF3 | H | CH3NHCO |
| 13-110 | C—Cl | C—H | C—Cl | C—H | H | CF3 | H | CH3CH2NHCO |
| 13-111 | C—Cl | C—H | C—Cl | C—H | H | CF3 | H | CF3CH2NHCO |
| 13-112 | C—Cl | C—H | C—Cl | C—H | H | CF3 | H | n-PrNHCO |
| 13-113 | C—Cl | C—H | C—Cl | C—H | H | CF3 | H | iso-PrNHCO |
| 13-114 | C—Cl | C—H | C—Cl | C—H | H | CF3 | H | cyclo-PrNHCO |
| 13-115 | C—Cl | C—H | C—Cl | C—H | H | CF3 | H | n-BuNHCO |
| 13-116 | C—Cl | C—H | C—Cl | C—H | H | CF3 | H | tert-BuNHCO |
| 13-117 | C—Cl | C—H | C—Cl | C—H | H | CF3 | H | cyclo-BuNHCO |
| 13-118 | C—Cl | C—H | C—Cl | C—H | H | CF3 | H | cyclo-PenNHCO |
| 13-119 | C—Cl | C—H | C—Cl | C—H | H | CF3 | H | cyclo-HexNHCO |
| 13-120 | C—Cl | C—H | C—Cl | C—H | H | CF3 | H | CH2═CHCH2NHCO |
| 13-121 | C—Cl | C—H | C—Cl | C—H | H | CF3 | H | HCCCH2NHCO |
| 13-122 | C—Cl | C—H | C—Cl | C—H | H | CF3 | H | cyclo-PrCH2NHCO |
| 13-123 | C—Cl | C—H | C—Cl | C—H | H | CF3 | H | NCCH2NHCO |
| 13-124 | C—Cl | C—H | C—Cl | C—H | H | CF3 | H | CF3(CH3)CHNHCO |
| 13-125 | C—Cl | C—H | C—Cl | C—H | H | CF3 | H | CF3(CH3)2CNCO |
| 13-126 | C—Cl | C—H | C—Cl | C—H | H | CF3 | H | NCCH20C0 |
| 13-127 | C—Cl | C—H | C—Cl | C—H | H | CF3 | H | FCH2CH2NHCO |
| 13-128 | C—Cl | C—H | C—Cl | C—H | H | CF3 | H | CF3CH2CH2NHCO |
| 13-129 | C—Cl | C—H | C—Cl | C—H | H | CF3 | H | HCF2CF2CH2NHCO |
| 13-130 | C—Cl | C—H | C—Cl | C—H | H | CF3 | H | 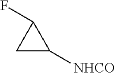 |
| 13-131 | C—Cl | C—H | C—Cl | C—H | H | CF3 | H | 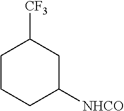 |
| 13-132 | C—Cl | C—H | C—Cl | C—H | H | CF3 | H | EtO2CCH2NHCO |
| 13-133 | C—Cl | C—H | C—Cl | C—H | H | CF3 | H | 2-CF3—PhNHCO |
| 13-134 | C—Cl | C—H | C—Cl | C—H | H | CF3 | H | 3-CF3—PhNHCO |
| 13-135 | C—Cl | C—H | C—Cl | C—H | H | CF3 | H | 4-CF3—PhNHCO |
| 13-136 | C—Cl | C—H | C—Cl | C—H | H | CF3 | H | 2-py-NHCO |
| 13-137 | C—Cl | C—H | C—Cl | C—H | H | CF3 | H | 3-py-NHCO |
| 13-138 | C—Cl | C—H | C—Cl | C—H | H | CF3 | H | 4-py-NHCO |
| 13-139 | C—Cl | C—H | C—Cl | C—H | H | CF3 | H | 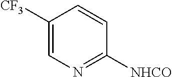 |
| 13-140 | C—Cl | C—H | C—Cl | C—H | H | CF3 | H | 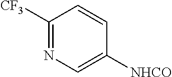 |
| 13-141 | C—Cl | C—H | C—Cl | C—H | H | CF3 | H | PhCH2HNCO |
| 13-142 | C—Cl | C—H | C—Cl | C—H | H | CF3 | H | 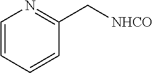 |
| 13-143 | C—Cl | C—H | C—Cl | C—H | H | CF3 | H | (CH3)2NCO |
| 13-144 | C—Cl | C—H | C—Cl | C—H | H | CF3 | H | pyrrolidineCO |
| 13-145 | C—Cl | C—H | C—Cl | C—H | H | CF3 | H | piperidineCO |
| 13-146 | C—Cl | C—H | C—Cl | C—H | H | CF3 | H | morpholineCO |
| 13-147 | C—Cl | C—H | C—Cl | C—H | H | CF3 | H | CH3OCONH |
| 13-148 | C—Cl | C—H | C—Cl | C—H | H | CF3 | H | CH3CH2OCO |
| 13-149 | C—Cl | C—H | C—Cl | C—H | H | CF3 | H | n-PrOCO |
| 13-150 | C—Cl | C—H | C—Cl | C—H | H | CF3 | H | cyclo-PrOCO |
| 13-151 | C—Cl | C—H | C—Cl | C—H | H | CF3 | H | CF3CH20C0 |
| 13-152 | C—Cl | C—H | C—Cl | C—H | H | CF3 | H | CH2═CHCH2OCO |
| 13-153 | C—Cl | C—H | C—Cl | C—H | H | CF3 | H | HCCCH2OCO |
| 13-154 | C—Cl | C—H | C—Cl | C—H | H | CF3 | H | PhOCO |
| 13-155 | C—Cl | C—H | C—Cl | C—H | H | CF3 | H | PhCH2OCO |
| 13-156 | C—Cl | C—H | C—Cl | C—H | H | CF3 | H | tert-BuOCO |
| 13-157 | C—Cl | C—H | C—Cl | C—H | H | CF3 | H | CH3SO2 |
| 13-158 | C—Cl | C—H | C—Cl | C—H | H | CF3 | H | CH3CH2SO2 |
| 13-159 | C—Cl | C—H | C—Cl | C—H | H | CF3 | H | CF3SO2 |
| 13-160 | C—Cl | C—H | C—Cl | C—H | H | CF3 | H | CF3CH2SO2 |
| 13-161 | C—Cl | C—H | C—Cl | C—H | H | CF3 | H | PhSO2 |
| 13-162 | C—Cl | C—H | C—Cl | C—H | H | CF3 | H | CH3NHCONHSO2 |
| 13-163 | C—Cl | C—H | C—Cl | C—H | H | CF3 | H | (CH3)2NCONHSO2 |
| 13-164 | C—Cl | C—H | C—Cl | C—H | H | CF3 | H | CH3CS |
| 13-165 | C—Cl | C—H | C—Cl | C—H | H | CF3 | H | CH3CH2CS |
| 13-166 | C—Cl | C—H | C—Cl | C—H | H | CF3 | H | cyclo-PrCS |
| 13-167 | C—Cl | C—H | C—Cl | C—H | H | CF3 | H | CF3CH2CS |
| 13-168 | C—Cl | C—H | C—Br | C—H | H | CF3 | H | CH3CO |
| 13-169 | C—Cl | C—H | C—Br | C—H | H | CF3 | H | CH3CH2CO |
| 13-170 | C—Cl | C—H | C—Br | C—H | H | CF3 | H | n-PrCO |
| 13-171 | C—Cl | C—H | C—Br | C—H | H | CF3 | H | cyclo-PrCO |
| 13-172 | C—Cl | C—H | C—Br | C—H | H | CF3 | H | cyclo-PrCH2CO |
| 13-173 | C—Cl | C—H | C—Br | C—H | H | CF3 | H | CF3CH2CO |
| 13-175 | C—Cl | C—H | C—Br | C—H | H | CF3 | H | CH3S(O)CH2CO |
| 13-176 | C—Cl | C—H | C—Br | C—H | H | CF3 | H | CH3SO2CH2CO |
| 13-177 | C—Cl | C—H | C—Br | C—H | H | CF3 | H | CH3CH2NHCO |
| 13-178 | C—Br | C—H | C—Br | C—H | H | CF3 | H | CH3CO |
| 13-179 | C—Br | C—H | C—Br | C—H | H | CF3 | H | CH3CH2CO |
| 13-180 | C—Br | C—H | C—Br | C—H | H | CF3 | H | n-PrCO |
| 13-181 | C—Br | C—H | C—Br | C—H | H | CF3 | H | cyclo-PrCO |
| 13-182 | C—Br | C—H | C—Br | C—H | H | CF3 | H | cyclo-PrCH2CO |
| 13-183 | C—Br | C—H | C—Br | C—H | H | CF3 | H | CF3CH2CO |
| 13-185 | C—Br | C—H | C—Br | C—H | H | CF3 | H | CH3S(O)CH2CO |
| 13-186 | C—Br | C—H | C—Br | C—H | H | CF3 | H | CH3SO2CH2CO |
| 13-187 | C—Br | C—H | C—Br | C—H | H | CF3 | H | CH3CH2NHCO |
| 13-188 | C—Cl | C—F | C—Cl | C—H | H | CF3 | H | CH3CO |
| 13-189 | C—Cl | C—F | C—Cl | C—H | H | CF3 | H | CH3CH2CO |
| 13-190 | C—Cl | C—F | C—Cl | C—H | H | CF3 | H | n-PrCO |
| 13-191 | C—Cl | C—F | C—Cl | C—H | H | CF3 | H | cyclo-PrCO |
| 13-192 | C—Cl | C—F | C—Cl | C—H | H | CF3 | H | cyclo-PrCH2C0 |
| 13-193 | C—Cl | C—F | C—Cl | C—H | H | CF3 | H | CF3CH2CO |
| 13-195 | C—Cl | C—F | C—Cl | C—H | H | CF3 | H | CH3S(O)CH2CO |
| 13-196 | C—Cl | C—F | C—Cl | C—H | H | CF3 | H | CH3SO2CH2CO |
| 13-197 | C—Cl | C—F | C—Cl | C—H | H | CF3 | H | CH3CH2NHCO |
| 13-198 | C—Cl | C—Cl | C—Cl | C—H | H | CF3 | H | CH3CO |
| 13-199 | C—Cl | C—Cl | C—Cl | C—H | H | CF3 | H | CH3CH2CO |
| 13-200 | C—Cl | C—Cl | C—Cl | C—H | H | CF3 | H | n-PrCO |
| 13-201 | C—Cl | C—Cl | C—Cl | C—H | H | CF3 | H | cyclo-PrCO |
| 13-202 | C—Cl | C—Cl | C—Cl | C—H | H | CF3 | H | cyclo-PrCH2C0 |
| 13-203 | C—Cl | C—Cl | C—Cl | C—H | H | CF3 | H | CF3CH2CO |
| 13-205 | C—Cl | C—Cl | C—Cl | C—H | H | CF3 | H | CH3CH2OCH2CH2-00 |
| 13-206 | C—Cl | C—Cl | C—Cl | C—H | H | CF3 | H | CH3SCH2C0 |
| 13-207 | C—Cl | C—Cl | C—Cl | C—H | H | CF3 | H | CH3S(O)CH2CO |
| 13-208 | C—Cl | C—Cl | C—Cl | C—H | H | CF3 | H | CH3SO2CH2CO |
| 13-209 | C—Cl | C—Cl | C—Cl | C—H | H | CF3 | H | CH3CH2NHCO |
| 13-210 | C—Cl | C—Br | C—Cl | C—H | H | CF3 | H | CH3CO |
| 13-211 | C—Cl | C—Br | C—Cl | C—H | H | CF3 | H | CH3CH2CO |
| 13-212 | C—Cl | C—Br | C—Cl | C—H | H | CF3 | H | n-PrCO |
| 13-213 | C—Cl | C—Br | C—Cl | C—H | H | CF3 | H | cyclo-PrCO |
| 13-214 | C—Cl | C—Br | C—Cl | C—H | H | CF3 | H | cyclo-PrCH2CO |
| 13-215 | C—Cl | C—Br | C—Cl | C—H | H | CF3 | H | CF3CH2CO |
| 13-217 | C—Cl | C—Br | C—Cl | C—H | H | CF3 | H | CH3S(O)CH2CO |
| 13-218 | C—Cl | C—Br | C—Cl | C—H | H | CF3 | H | CH3SO2CH2CO |
| 13-219 | C—Cl | C—Br | C—Cl | C—H | H | CF3 | H | CH3CH2NHCO |
| 13-220 | C—Cl | C—Cl | C—Br | C—H | H | CF3 | H | CH3CO |
| 13-221 | C—Cl | C—Cl | C—Br | C—H | H | CF3 | H | CH3CH2CO |
| 13-222 | C—Cl | C—Cl | C—Br | C—H | H | CF3 | H | n-PrCO |
| 13-223 | C—Cl | C—Cl | C—Br | C—H | H | CF3 | H | cyclo-PrCO |
| 13-224 | C—Cl | C—Cl | C—Br | C—H | H | CF3 | H | cyclo-PrCH2CO |
| 13-225 | C—Cl | C—Cl | C—Br | C—H | H | CF3 | H | CF3CH2CO |
| 13-227 | C—Cl | C—Cl | C—Br | C—H | H | CF3 | H | CH3S(O)CH2CO |
| 13-228 | C—Cl | C—Cl | C—Br | C—H | H | CF3 | H | CH3SO2CH2CO |
| 13-229 | C—Cl | C—Cl | C—Br | C—H | H | CF3 | H | CH3CH2NHCO |
| 13-230 | C—Cl | C—F | C—Cl | C—H | F | CF3 | H | CH3CO |
| 13-231 | C—Cl | C—F | C—Cl | C—H | F | CF3 | H | CH3CH2CO |
| 13-232 | C—Cl | C—F | C—Cl | C—H | F | CF3 | H | n-PrCO |
| 13-233 | C—Cl | C—F | C—Cl | C—H | F | CF3 | H | cyclo-PrCO |
| 13-234 | C—Cl | C—F | C—Cl | C—H | F | CF3 | H | cyclo-PrCH2CO |
| 13-235 | C—Cl | C—F | C—Cl | C—H | F | CF3 | H | CF3CH2CO |
| 13-237 | C—Cl | C—F | C—Cl | C—H | F | CF3 | H | CH3S(O)CH2CO |
| 13-238 | C—Cl | C—F | C—Cl | C—H | F | CF3 | H | CH3SO2CH2CO |
| 13-239 | C—Cl | C—F | C—Cl | C—H | F | CF3 | H | CH3CH2NHCO |
| 13-240 | C—CF3 | C—H | C—H | C—H | H | CF3 | H | CH3CO |
| 13-241 | C—CF3 | C—H | C—H | C—H | H | CF3 | H | CH3CH2CO |
| 13-242 | C—CF3 | C—H | C—H | C—H | H | CF3 | H | n-PrCO |
| 13-243 | C—CF3 | C—H | C—H | C—H | H | CF3 | H | cyclo-PrCO |
| 13-244 | C—CF3 | C—H | C—H | C—H | H | CF3 | H | cyclo-PrCH2CO |
| 13-245 | C—CF3 | C—H | C—H | C—H | H | CF3 | H | CF3CH2CO |
| 13-247 | C—CF3 | C—H | C—H | C—H | H | CF3 | H | CH3S(O)CH2CO |
| 13-248 | C—CF3 | C—H | C—H | C—H | H | CF3 | H | CH3SO2CH2CO |
| 13-249 | C—CF3 | C—H | C—H | C—H | H | CF3 | H | CH3CH2NHCO |
| 13-250 | C—CF3 | C—F | C—H | C—H | H | CF3 | H | CH3CO |
| 13-251 | C—CF3 | C—F | C—H | C—H | H | CF3 | H | CH3CH2CO |
| 13-252 | C—CF3 | C—F | C—H | C—H | H | CF3 | H | n-PrCO |
| 13-253 | C—CF3 | C—F | C—H | C—H | H | CF3 | H | cyclo-PrCO |
| 13-254 | C—CF3 | C—F | C—H | C—H | H | CF3 | H | cyclo-PrCH2CO |
| 13-255 | C—CF3 | C—F | C—H | C—H | H | CF3 | H | CF3CH2CO |
| 13-257 | C—CF3 | C—F | C—H | C—H | H | CF3 | H | CH3S(O)CH2CO |
| 13-258 | C—CF3 | C—F | C—H | C—H | H | CF3 | H | CH3SO2CH2CO |
| 13-259 | C—CF3 | C—F | C—H | C—H | H | CF3 | H | CH3CH2NHCO |
| 13-260 | C—CF3 | C—H | C—H | C—H | F | CF3 | H | CH3CO |
| 13-261 | C—CF3 | C—H | C—H | C—H | F | CF3 | H | CH3CH2CO |
| 13-262 | C—CF3 | C—H | C—H | C—H | F | CF3 | H | n-PrCO |
| 13-263 | C—CF3 | C—H | C—H | C—H | F | CF3 | H | cyclo-PrCO |
| 13-264 | C—CF3 | C—H | C—H | C—H | F | CF3 | H | cyclo-PrCH2CO |
| 13-265 | C—CF3 | C—H | C—H | C—H | F | CF3 | H | CF3CH2CO |
| 13-267 | C—CF3 | C—H | C—H | C—H | F | CF3 | H | CH3S(O)CH2CO |
| 13-268 | C—CF3 | C—H | C—H | C—H | F | CF3 | H | CH3SO2CH2CO |
| 13-269 | C—CF3 | C—H | C—H | C—H | F | CF3 | H | CH3CH2NHCO |
| 13-270 | C—H | C—H | C—CF3 | C—H | F | CF3 | H | CH3CO |
| 13-271 | C—H | C—H | C—CF3 | C—H | F | CF3 | H | CH3CH2CO |
| 13-272 | C—H | C—H | C—CF3 | C—H | F | CF3 | H | n-PrCO |
| 13-273 | C—H | C—H | C—CF3 | C—H | F | CF3 | H | cyclo-PrCO |
| 13-274 | C—H | C—H | C—CF3 | C—H | F | CF3 | H | cyclo-PrCH2CO |
| 13-275 | C—H | C—H | C—CF3 | C—H | F | CF3 | H | CF3CH2CO |
| 13-277 | C—H | C—H | C—CF3 | C—H | F | CF3 | H | CH3S(O)CH2CO |
| 13-278 | C—H | C—H | C—CF3 | C—H | F | CF3 | H | CH3SO2CH2CO |
| 13-279 | C—H | C—H | C—CF3 | C—H | F | CF3 | H | CH3CH2NHCO |
| 13-280 | C—Cl | C—H | C—CF3 | C—H | H | CF3 | H | CH3CO |
| 13-281 | C—Cl | C—H | C—CF3 | C—H | H | CF3 | H | CH3CH2CO |
| 13-282 | C—Cl | C—H | C—CF3 | C—H | H | CF3 | H | n-PrCO |
| 13-283 | C—Cl | C—H | C—CF3 | C—H | H | CF3 | H | cyclo-PrCO |
| 13-284 | C—Cl | C—H | C—CF3 | C—H | H | CF3 | H | cyclo-PrCH2CO |
| 13-285 | C—Cl | C—H | C—CF3 | C—H | H | CF3 | H | CF3CH2CO |
| 13-287 | C—Cl | C—H | C—CF3 | C—H | H | CF3 | H | CH3CH2OCH2CH2—CO |
| 13-288 | C—Cl | C—H | C—CF3 | C—H | H | CF3 | H | CH3SCH2CO |
| 13-289 | C—Cl | C—H | C—CF3 | C—H | H | CF3 | H | CH3S(O)CH2CO |
| 13-290 | C—Cl | C—H | C—CF3 | C—H | H | CF3 | H | CH3SO2CH2CO |
| 13-291 | C—Cl | C—H | C—CF3 | C—H | H | CF3 | H | CH3CH2NHCO |
| 13-292 | C—CF3 | C—H | C—CF3 | C—H | H | CF3 | H | CH3CO |
| 13-293 | C—CF3 | C—H | C—CF3 | C—H | H | CF3 | H | CH3CH2CO |
| 13-294 | C—CF3 | C—H | C—CF3 | C—H | H | CF3 | H | n-PrCO |
| 13-295 | C—CF3 | C—H | C—CF3 | C—H | H | CF3 | H | cyclo-PrCO |
| 13-296 | C—CF3 | C—H | C—CF3 | C—H | H | CF3 | H | cyclo-PrCH2CO |
| 13-297 | C—CF3 | C—H | C—CF3 | C—H | H | CF3 | H | CF3CH2CO |
| 13-299 | C—CF3 | C—H | C—CF3 | C—H | H | CF3 | H | CH3CH2OCH2CH2—CO |
| 13-300 | C—CF3 | C—H | C—CF3 | C—H | H | CF3 | H | CH3SCH2CO |
| 13-301 | C—CF3 | C—H | C—CF3 | C—H | H | CF3 | H | CH3S(O)CH2CO |
| 13-302 | C—CF3 | C—H | C—CF3 | C—H | H | CF3 | H | CH3SO2CH2CO |
| 13-303 | C—CF3 | C—H | C—CF3 | C—H | H | CF3 | H | CH3CH2NHCO |
| 13-304 | C—Cl | C-Cl | C—CF3 | C—H | H | CF3 | H | CH3CO |
| 13-305 | C—Cl | C-Cl | C—CF3 | C—H | H | CF3 | H | CH3CH2CO |
| 13-306 | C—Cl | C-Cl | C—CF3 | C—H | H | CF3 | H | n-PrCO |
| 13-307 | C—Cl | C-Cl | C—CF3 | C—H | H | CF3 | H | cyclo-PrCO |
| 13-308 | C—Cl | C-Cl | C—CF3 | C—H | H | CF3 | H | cyclo-PrCH2CO |
| 13-309 | C—Cl | C-Cl | C—CF3 | C—H | H | CF3 | H | CF3CH2CO |
| 13-311 | C—Cl | C-Cl | C—CF3 | C—H | H | CF3 | H | CH3S(O)CH2CO |
| 13-312 | C—Cl | C-Cl | C—CF3 | C—H | H | CF3 | H | CH3SO2CH2CO |
| 13-313 | C—Cl | C-Cl | C—CF3 | C—H | H | CF3 | H | CH3CH2NHCO |
| 13-314 | C—Cl | N | C—H | C—H | H | CF3 | H | CH3CO |
| 13-315 | C—Cl | N | C—H | C—H | H | CF3 | H | CH3CH2CO |
| 13-316 | C—Cl | N | C—H | C—H | H | CF3 | H | n-PrCO |
| 13-317 | C—Cl | N | C—H | C—H | H | CF3 | H | cyclo-PrCO |
| 13-318 | C—Cl | N | C—H | C—H | H | CF3 | H | cyclo-PrCH2CO |
| 13-319 | C—Cl | N | C—H | C—H | H | CF3 | H | CF3CH2CO |
| 13-321 | C—Cl | N | C—H | C—H | H | CF3 | H | CH3S(O)CH2CO |
| 13-322 | C—Cl | N | C—H | C—H | H | CF3 | H | CH3SO2CH2CO |
| 13-323 | C—Cl | N | C—H | C—H | H | CF3 | H | CH3CH2NHCO |
| 13-324 | C—Cl | N | C—Cl | C—H | H | CF3 | H | CH3CO |
| 13-325 | C—Cl | N | C—Cl | C—H | H | CF3 | H | CH3CH2CO |
| 13-326 | C—Cl | N | C—Cl | C—H | H | CF3 | H | n-PrCO |
| 13-327 | C—Cl | N | C—Cl | C—H | H | CF3 | H | cyclo-PrCO |
| 13-328 | C—Cl | N | C—Cl | C—H | H | CF3 | H | cyclo-PrCH2CO |
| 13-329 | C—Cl | N | C—Cl | C—H | H | CF3 | H | CF3CH2CO |
| 13-330 | C—Cl | N | C—Cl | C—H | H | CF3 | H | CH3SCH2CO |
| 13-331 | C—Cl | N | C—Cl | C—H | H | CF3 | H | CH3S(O)CH2CO |
| 13-332 | C—Cl | N | C—Cl | C—H | H | CF3 | H | CH3SO2CH2CO |
| 13-333 | C—Cl | N | C—Cl | C—H | H | CF3 | H | CH3CH2NHCO |
| 13-334 | C—CF3 | N | C—H | C—H | H | CF3 | H | CH3CO |
| 13-335 | C—CF3 | N | C—H | C—H | H | CF3 | H | CH3CH2CO |
| 13-336 | C—CF3 | N | C—H | C—H | H | CF3 | H | n-PrCO |
| 13-337 | C—CF3 | N | C—H | C—H | H | CF3 | H | cyclo-PrCO |
| 13-338 | C—CF3 | N | C—H | C—H | H | CF3 | H | cyclo-PrCH2CO |
| 13-339 | C—CF3 | N | C—H | C—H | H | CF3 | H | CF3CH2CO |
| 13-340 | C—CF3 | N | C—H | C—H | H | CF3 | H | CH3SCH2C0 |
| 13-341 | C—CF3 | N | C—H | C—H | H | CF3 | H | CH3S(O)CH2CO |
| 13-342 | C—CF3 | N | C—H | C—H | H | CF3 | H | CH3SO2CH2CO |
| 13-343 | C—CF3 | N | C—H | C—H | H | CF3 | H | CH3CH2NHCO |
| 13-344 | C—CF3 | N | C—CF3 | C—H | H | CF3 | H | CH3CO |
| 13-345 | C—CF3 | N | C—CF3 | C—H | H | CF3 | H | CH3CH2CO |
| 13-346 | C—CF3 | N | C—CF3 | C—H | H | CF3 | H | n-PrCO |
| 13-347 | C—CF3 | N | C—CF3 | C—H | H | CF3 | H | cyclo-PrCO |
| 13-348 | C—CF3 | N | C—CF3 | C—H | H | CF3 | H | cyclo-PrCH2CO |
| 13-349 | C—CF3 | N | C—CF3 | C—H | H | CF3 | H | CF3CH2CO |
| 13-350 | C—CF3 | N | C—CF3 | C—H | H | CF3 | H | CH3SCH2CO |
| 13-351 | C—CF3 | N | C—CF3 | C—H | H | CF3 | H | CH3S(O)CH2CO |
| 13-352 | C—CF3 | N | C—CF3 | C—H | H | CF3 | H | CH3SO2CH2CO |
| 13-353 | C—CF3 | N | C—CF3 | C—H | H | CF3 | H | CH3CH2NHCO |
| 13-354 | C—CF3 | C—H | C—H | N | H | CF3 | H | CH3CO |
| 13-355 | C—CF3 | C—H | C—H | N | H | CF3 | H | CH3CH2CO |
| 13-356 | C—CF3 | C—H | C—H | N | H | CF3 | H | n-PrCO |
| 13-357 | C—CF3 | C—H | C—H | N | H | CF3 | H | cyclo-PrCO |
| 13-358 | C—CF3 | C—H | C—H | N | H | CF3 | H | cyclo-PrCH2CO |
| 13-359 | C—CF3 | C—H | C—H | N | H | CF3 | H | CF3CH2CO |
| 13-360 | C—CF3 | C—H | C—H | N | H | CF3 | H | CH3SCH2CO |
| 13-361 | C—CF3 | C—H | C—H | N | H | CF3 | H | CH3S(O)CH2CO |
| 13-362 | C—CF3 | C—H | C—H | N | H | CF3 | H | CH3SO2CH2CO |
| 13-363 | C—CF3 | C—H | C—H | N | H | CF3 | H | CH3CH2NHCO |
| 13-364 | C—H | C—H | C—CF3 | N | H | CF3 | H | CH3CO |
| 13-365 | C—H | C—H | C—CF3 | N | H | CF3 | H | CH3CH2CO |
| 13-366 | C—H | C—H | C—CF3 | N | H | CF3 | H | n-PrCO |
| 13-367 | C—H | C—H | C—CF3 | N | H | CF3 | H | cyclo-PrCO |
| 13-368 | C—H | C—H | C—CF3 | N | H | CF3 | H | cyclo-PrCH2CO |
| 13-369 | C—H | C—H | C—CF3 | N | H | CF3 | H | CF3CH2CO |
| 13-370 | C—H | C—H | C—CF3 | N | H | CF3 | H | CH3SCH2CO |
| 13-371 | C—H | C—H | C—CF3 | N | H | CF3 | H | CH3S(O)CH2CO |
| 13-372 | C—H | C—H | C—CF3 | N | H | CF3 | H | CH3SO2CH2CO |
| 13-373 | C—H | C—H | C—CF3 | N | H | CF3 | H | CH3CH2NHCO |
| 13-374 | C—CF3 | C—H | C—Cl | N | H | CF3 | H | CH3CO |
| 13-375 | C—CF3 | C—H | C—Cl | N | H | CF3 | H | CH3CH2CO |
| 13-376 | C—CF3 | C—H | C—Cl | N | H | CF3 | H | n-PrCO |
| 13-377 | C—CF3 | C—H | C—Cl | N | H | CF3 | H | cyclo-PrCO |
| 13-378 | C—CF3 | C—H | C—Cl | N | H | CF3 | H | cyclo-PrCH2CO |
| 13-379 | C—CF3 | C—H | C—Cl | N | H | CF3 | H | CF3CH2CO |
| 13-380 | C—CF3 | C—H | C—Cl | N | H | CF3 | H | CH3SCH2CO |
| 13-381 | C—CF3 | C—H | C—Cl | N | H | CF3 | H | CH3S(O)CH2CO |
| 13-382 | C—CF3 | C—H | C—Cl | N | H | CF3 | H | CH3SO2CH2CO |
| 13-383 | C—CF3 | C—H | C—Cl | N | H | CF3 | H | CH3CH2NHCO |
| 13-384 | C—CF3 | C—H | C—Br | N | H | CF3 | H | CH3CO |
| 13-385 | C—CF3 | C—H | C—Br | N | H | CF3 | H | CH3CH2CO |
| 13-386 | C—CF3 | C—H | C—Br | N | H | CF3 | H | n-PrCO |
| 13-387 | C—CF3 | C—H | C—Br | N | H | CF3 | H | cyclo-PrCO |
| 13-388 | C—CF3 | C—H | C—Br | N | H | CF3 | H | cyclo-PrCH2CO |
| 13-389 | C—CF3 | C—H | C—Br | N | H | CF3 | H | CF3CH2CO |
| 13-390 | C—CF3 | C—H | C—Br | N | H | CF3 | H | CH3SCH2CO |
| 13-391 | C—CF3 | C—H | C—Br | N | H | CF3 | H | CH3S(O)CH2CO |
| 13-392 | C—CF3 | C—H | C—Br | N | H | CF3 | H | CH3SO2CH2CO |
| 13-393 | C—CF3 | C—H | C—Br | N | H | CF3 | H | CH3CH2NHCO |
| 13-394 | C—CF3 | C—H | C—CF3 | N | H | CF3 | H | CH3CO |
| 13-395 | C—CF3 | C—H | C—CF3 | N | H | CF3 | H | CH3CH2CO |
| 13-396 | C—CF3 | C—H | C—CF3 | N | H | CF3 | H | n-PrCO |
| 13-397 | C—CF3 | C—H | C—CF3 | N | H | CF3 | H | cyclo-PrCO |
| 13-398 | C—CF3 | C—H | C—CF3 | N | H | CF3 | H | cyclo-PrCH2CO |
| 13-399 | C—CF3 | C—H | C—CF3 | N | H | CF3 | H | CF3CH2CO |
| 13-400 | C—CF3 | C—H | C—CF3 | N | H | CF3 | H | CH3SCH2CO |
| 13-401 | C—CF3 | C—H | C—CF3 | N | H | CF3 | H | CH3S(O)CH2CO |
| 13-402 | C—CF3 | C—H | C—CF3 | N | H | CF3 | H | CH3SO2CH2CO |
| 13-403 | C—CF3 | C—H | C—CF3 | N | H | CF3 | H | CH3CH2NHCO |

TABLE 14

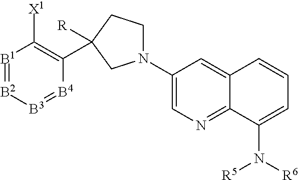

| No. | B¹ | B² | B³ | B⁴ | X¹ | R | R⁵ | R⁶ |
|---|---|---|---|---|---|---|---|---|
| 14-1 | C—Cl | C—H | C—H | C—H | H | CF3 | H | CH3CO |
| 14-2 | C—Cl | C—H | C—H | C—H | H | CF3 | H | CH3CH2CO |
| 14-3 | C—Cl | C—H | C—H | C—H | H | CF3 | H | n-PrCO |
| 14-4 | C—Cl | C—H | C—H | C—H | H | CF3 | H | cyclo-PrCO |
| 14-5 | C—Cl | C—H | C—H | C—H | H | CF3 | H | cyclo-PrCH2CO |
| 14-6 | C—Cl | C—H | C—H | C—H | H | CF3 | H | CF3CH2CO |
| 14-7 | C—Cl | C—H | C—H | C—H | H | CF3 | H | CH3SCH2CO |
| 14-8 | C—Cl | C—H | C—H | C—H | H | CF3 | H | CH3S(O)CH2CO |
| 14-9 | C—Cl | C—H | C—H | C—H | H | CF3 | H | CH3SO2CH2CO |
| 14-10 | C—Cl | C—H | C—H | C—H | H | CF3 | H | CH3CH2NHCO |
| 14-11 | C—Cl | C—H | C—H | C—H | F | CF3 | H | CH3CO |
| 14-12 | C—Cl | C—H | C—H | C—H | F | CF3 | H | CH3CH2CO |
| 14-13 | C—Cl | C—H | C—H | C—H | F | CF3 | H | n-PrCO |
| 14-14 | C—Cl | C—H | C—H | C—H | F | CF3 | H | cyclo-PrCO |
| 14-15 | C—Cl | C—H | C—H | C—H | F | CF3 | H | cyclo-PrCH2CO |
| 14-16 | C—Cl | C—H | C—H | C—H | F | CF3 | H | CF3CH2CO |
| 14-17 | C—Cl | C—H | C—H | C—H | F | CF3 | H | CH3SCH2CO |
| 14-18 | C—Cl | C—H | C—H | C—H | F | CF3 | H | CH3S(O)CH2CO |
| 14-19 | C—Cl | C—H | C—H | C—H | F | CF3 | H | CH3SO2CH2CO |
| 14-20 | C—Cl | C—H | C—H | C—H | F | CF3 | H | CH3CH2NHCO |
| 14-21 | C—H | C—H | C—Cl | C—H | F | CF3 | H | CH3CO |
| 14-22 | C—H | C—H | C—Cl | C—H | F | CF3 | H | CH3CH2CO |
| 14-23 | C—H | C—H | C—Cl | C—H | F | CF3 | H | n-PrCO |
| 14-24 | C—H | C—H | C—Cl | C—H | F | CF3 | H | cyclo-PrCO |
| 14-25 | C—H | C—H | C—Cl | C—H | F | CF3 | H | cyclo-PrCH2CO |
| 14-26 | C—H | C—H | C—Cl | C—H | F | CF3 | H | CF3CH2CO |
| 14-27 | C—H | C—H | C—Cl | C—H | F | CF3 | H | CH3SCH2CO |
| 14-28 | C—H | C—H | C—Cl | C—H | F | CF3 | H | CH3S(O)CH2CO |
| 14-29 | C—H | C—H | C—Cl | C—H | F | CF3 | H | CH3SO2CH2CO |
| 14-30 | C—H | C—H | C—Cl | C—H | F | CF3 | H | CH3CH2NHCO |
| 14-31 | C—Cl | C—H | C—Cl | C—H | H | CF3 | H | H |
| 14-32 | C—Cl | C—H | C—Cl | C—H | H | CF3 | H | HCO |
| 14-33 | C—Cl | C—H | C—Cl | C—H | H | CF3 | H | CH3CO |
| 14-34 | C—Cl | C—H | C—Cl | C—H | H | CF3 | H | CH3CH2CO |
| 14-35 | C—Cl | C—H | C—Cl | C—H | H | CF3 | H | n-PrCO |
| 14-36 | C—Cl | C—H | C—Cl | C—H | H | CF3 | H | iso-PrCO |
| 14-37 | C—Cl | C—H | C—Cl | C—H | H | CF3 | H | cyclo-PrCO |
| 14-38 | C—Cl | C—H | C—Cl | C—H | H | CF3 | H | n-BuCO |
| 14-39 | C—Cl | C—H | C—Cl | C—H | H | CF3 | H | tert-BuCO |
| 14-40 | C—Cl | C—H | C—Cl | C—H | H | CF3 | H | iso-BuCO |
| 14-41 | C—Cl | C—H | C—Cl | C—H | H | CF3 | H | cyclo-BuCO |
| 14-42 | C—Cl | C—H | C—Cl | C—H | H | CF3 | H | (CH3)3CCH2CO |
| 14-43 | C—Cl | C—H | C—Cl | C—H | H | CF3 | H | cyclo-PenCO |
| 14-44 | C—Cl | C—H | C—Cl | C—H | H | CF3 | H | cyclo-HexCO |
| 14-45 | C—Cl | C—H | C—Cl | C—H | H | CF3 | H | cyclo-PrCH2CO |
| 14-46 | C—Cl | C—H | C—Cl | C—H | H | CF3 | H | CF3CO |
| 14-47 | C—Cl | C—H | C—Cl | C—H | H | CF3 | H | CCl3CO |
| 14-48 | C—Cl | C—H | C—Cl | C—H | H | CF3 | H | ClCH2CO |
| 14-49 | C—Cl | C—H | C—Cl | C—H | H | CF3 | H | CF2ClCO |
| 14-50 | C—Cl | C—H | C—Cl | C—H | H | CF3 | H | ClCH2CH2CO |
| 14-51 | C—Cl | C—H | C—Cl | C—H | H | CF3 | H | HCF2CF2CO |
| 14-52 | C—Cl | C—H | C—Cl | C—H | H | CF3 | H | CF3CH2CO |
| 14-54 | C—Cl | C—H | C—Cl | C—H | H | CF3 | H | HCF2CO |
| 14-55 | C—Cl | C—H | C—Cl | C—H | H | CF3 | H | ClCH2CO |
| 14-56 | C—Cl | C—H | C—Cl | C—H | H | CF3 | H | 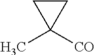 |
| 14-57 | C—Cl | C—H | C—Cl | C—H | H | CF3 | H | 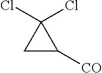 |
| 14-58 | C—Cl | C—H | C—Cl | C—H | H | CF3 | H | 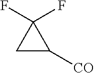 |
| 14-59 | C—Cl | C—H | C—Cl | C—H | H | CF3 | H | 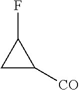 |
| 14-60 | C—Cl | C—H | C—Cl | C—H | H | CF3 | H | 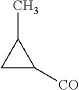 |
| 14-61 | C—Cl | C—H | C—Cl | C—H | H | CF3 | H | 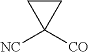 |
| 14-62 | C—Cl | C—H | C—Cl | C—H | H | CF3 | H | CH2═CH—CO |
| 14-63 | C—Cl | C—H | C—Cl | C—H | H | CF3 | H | CH3CH═CH—CO |
| 14-64 | C—Cl | C—H | C—Cl | C—H | H | CF3 | H | CH2═C(CH3)—CO |
| 14-65 | C—Cl | C—H | C—Cl | C—H | H | CF3 | H | CH3CH═C(CH3)—CO |
| 14-66 | C—Cl | C—H | C—Cl | C—H | H | CF3 | H | (CH3)2C═CH—CO |
| 14-67 | C—Cl | C—H | C—Cl | C—H | H | CF3 | H | CH2═C(Cl)—CO |
| 14-68 | C—Cl | C—H | C—Cl | C—H | H | CF3 | H | ClCH═C(CH3)—CO |
| 14-69 | C—Cl | C—H | C—Cl | C—H | H | CF3 | H | CH2═CHCH2—CO |
| 14-70 | C—Cl | C—H | C—Cl | C—H | H | CF3 | H | HCC—CO |
| 14-71 | C—Cl | C—H | C—Cl | C—H | H | CF3 | H | CH3CC—CO |
| 14-72 | C—Cl | C—H | C—Cl | C—H | H | CF3 | H | NCCH2—CO |
| 14-73 | C—Cl | C—H | C—Cl | C—H | H | CF3 | H | CH3OCH2—CO |
| 14-74 | C—Cl | C—H | C—Cl | C—H | H | CF3 | H | C2H5OCH2—CO |
| 14-75 | C—Cl | C—H | C—Cl | C—H | H | CF3 | H | CH3OCH2CH2—CO |
| 14-76 | C—Cl | C—H | C—Cl | C—H | H | CF3 | H | CH3CH2OCH2CH2—CO |
| 14-77 | C—Cl | C—H | C—Cl | C—H | H | CF3 | H | CH3SCH2CO |
| 14-78 | C—Cl | C—H | C—Cl | C—H | H | CF3 | H | CH3S(O)CH2CO |
| 14-79 | C—Cl | C—H | C—Cl | C—H | H | CF3 | H | CH3SO2CH2CO |
| 14-80 | C—Cl | C—H | C—Cl | C—H | H | CF3 | H | CH3O(CO)CH2—CO |
| 14-81 | C—Cl | C—H | C—Cl | C—H | H | CF3 | H | CH3(CO)OCH2—CO |
| 14-82 | C—Cl | C—H | C—Cl | C—H | H | CF3 | H | CH3O(CO)—CO |
| 14-83 | C—Cl | C—H | C—Cl | C—H | H | CF3 | H | CF3O(CO)—CO |
| 14-84 | C—Cl | C—H | C—Cl | C—H | H | CF3 | H | CH3(CO)CH2—CO |
| 14-85 | C—Cl | C—H | C—Cl | C—H | H | CF3 | H | (CH3)2NCH2—CO |
| 14-86 | C—Cl | C—H | C—Cl | C—H | H | CF3 | H | PhCO |
| 14-87 | C—Cl | C—H | C—Cl | C—H | H | CF3 | H | PhCH2CO |
| 14-88 | C—Cl | C—H | C—Cl | C—H | H | CF3 | H | 2-py-CO |
| 14-89 | C—Cl | C—H | C—Cl | C—H | H | CF3 | H | 3-py-CO |
| 14-90 | C—Cl | C—H | C—Cl | C—H | H | CF3 | H | 4-py-CO |
| 14-91 | C—Cl | C—H | C—Cl | C—H | H | CF3 | H | 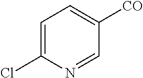 |
| 14-92 | C—Cl | C—H | C—Cl | C—H | H | CF3 | H | 2-F—PhCO |
| 14-93 | C—Cl | C—H | C—Cl | C—H | H | CF3 | H | 3-F—PhCO |
| 14-94 | C—Cl | C—H | C—Cl | C—H | H | CF3 | H | 4-F—PhCO |
| 14-95 | C—Cl | C—H | C—Cl | C—H | H | CF3 | H | 2-Cl—PhCO |
| 14-96 | C—Cl | C—H | C—Cl | C—H | H | CF3 | H | 3-Cl—PhCO |
| 14-97 | C—Cl | C—H | C—Cl | C—H | H | CF3 | H | 4-Cl—PhCO |
| 14-98 | C—Cl | C—H | C—Cl | C—H | H | CF3 | H | 2-Br—PhCO |
| 14-99 | C—Cl | C—H | C—Cl | C—H | H | CF3 | H | 2-CF3—PhCO |
| 14-100 | C—Cl | C—H | C—Cl | C—H | H | CF3 | H | 2-CH3—PhCO |
| 14-101 | C—Cl | C—H | C—Cl | C—H | H | CF3 | H | 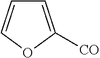 |
| 14-102 | C—Cl | C—H | C—Cl | C—H | H | CF3 | H | 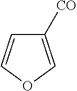 |
| 14-103 | C—Cl | C—H | C—Cl | C—H | H | CF3 | H | 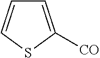 |
| 14-104 | C—Cl | C—H | C—Cl | C—H | H | CF3 | H | 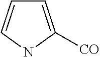 |
| 14-105 | C—Cl | C—H | C—Cl | C—H | H | CF3 | H | 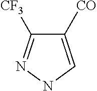 |
| 14-106 | C—Cl | C—H | C—Cl | C—H | H | CF3 | H | 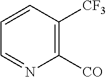 |
| 14-107 | C—Cl | C—H | C—Cl | C—H | H | CF3 | H | 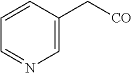 |
| 14-108 | C—Cl | C—H | C—Cl | C—H | H | CF3 | H | 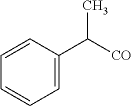 |
| 14-109 | C—Cl | C—H | C—Cl | C—H | H | CF3 | H | CH3NHCO |
| 14-110 | C—Cl | C—H | C—Cl | C—H | H | CF3 | H | CH3CH2NHCO |
| 14-111 | C—Cl | C—H | C—Cl | C—H | H | CF3 | H | CF3CH2NHCO |
| 14-112 | C—Cl | C—H | C—Cl | C—H | H | CF3 | H | n-PrNHCO |
| 14-113 | C—Cl | C—H | C—Cl | C—H | H | CF3 | H | iso-PrNHCO |
| 14-114 | C—Cl | C—H | C—Cl | C—H | H | CF3 | H | cyclo-PrNHCO |
| 14-115 | C—Cl | C—H | C—Cl | C—H | H | CF3 | H | n-BuNHCO |
| 14-116 | C—Cl | C—H | C—Cl | C—H | H | CF3 | H | tert-BuNHCO |
| 14-117 | C—Cl | C—H | C—Cl | C—H | H | CF3 | H | cyclo-BuNHCO |
| 14-118 | C—Cl | C—H | C—Cl | C—H | H | CF3 | H | cyclo-PenNHCO |
| 14-119 | C—Cl | C—H | C—Cl | C—H | H | CF3 | H | cyclo-HexNHCO |
| 14-120 | C—Cl | C—H | C—Cl | C—H | H | CF3 | H | CH2═CHCH2NHCO |
| 14-121 | C—Cl | C—H | C—Cl | C—H | H | CF3 | H | HCCCH2NHCO |
| 14-122 | C—Cl | C—H | C—Cl | C—H | H | CF3 | H | cyclo-PrCH2NHCO |
| 14-123 | C—Cl | C—H | C—Cl | C—H | H | CF3 | H | NCCH2NHCO |
| 14-124 | C—Cl | C—H | C—Cl | C—H | H | CF3 | H | CF3(CH3)CHNHCO |
| 14-125 | C—Cl | C—H | C—Cl | C—H | H | CF3 | H | CF3(CH3)2CNCO |
| 14-126 | C—Cl | C—H | C—Cl | C—H | H | CF3 | H | NCCH20C0 |
| 14-127 | C—Cl | C—H | C—Cl | C—H | H | CF3 | H | FCH2CH2NHCO |
| 14-128 | C—Cl | C—H | C—Cl | C—H | H | CF3 | H | CF3CH2CH2NHCO |
| 14-129 | C—Cl | C—H | C—Cl | C—H | H | CF3 | H | HCF2CF2CH2NHCO |
| 14-130 | C—Cl | C—H | C—Cl | C—H | H | CF3 | H | 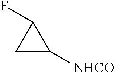 |
| 14-131 | C—Cl | C—H | C—Cl | C—H | H | CF3 | H | 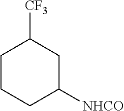 |
| 14-132 | C—Cl | C—H | C—Cl | C—H | H | CF3 | H | EtO2CCH2NHCO |
| 14-133 | C—Cl | C—H | C—Cl | C—H | H | CF3 | H | 2-CF3—PhNHCO |
| 14-134 | C—Cl | C—H | C—Cl | C—H | H | CF3 | H | 3-CF3—PhNHCO |
| 14-135 | C—Cl | C—H | C—Cl | C—H | H | CF3 | H | 4-CF3—PhNHCO |
| 14-136 | C—Cl | C—H | C—Cl | C—H | H | CF3 | H | 2-py-NHCO |
| 14-137 | C—Cl | C—H | C—Cl | C—H | H | CF3 | H | 3-py-NHCO |
| 14-138 | C—Cl | C—H | C—Cl | C—H | H | CF3 | H | 4-py-NHCO |
| 14-139 | C—Cl | C—H | C—Cl | C—H | H | CF3 | H | 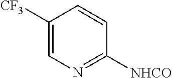 |
| 14-140 | C—Cl | C—H | C—Cl | C—H | H | CF3 | H | 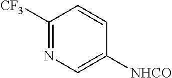 |
| 14-141 | C—Cl | C—H | C—Cl | C—H | H | CF3 | H | PhCH2HNCO |
| 14-142 | C—Cl | C—H | C—Cl | C—H | H | CF3 | H | 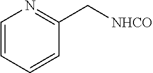 |
| 14-143 | C—Cl | C—H | C—Cl | C—H | H | CF3 | H | (CH3)2NCO |
| 14-144 | C—Cl | C—H | C—Cl | C—H | H | CF3 | H | pyrrolidineCO |
| 14-145 | C—Cl | C—H | C—Cl | C—H | H | CF3 | H | piperidineCO |
| 14-146 | C—Cl | C—H | C—Cl | C—H | H | CF3 | H | morpholineCO |
| 14-147 | C—Cl | C—H | C—Cl | C—H | H | CF3 | H | CH3OCONH |
| 14-148 | C—Cl | C—H | C—Cl | C—H | H | CF3 | H | CH3CH2OCO |
| 14-149 | C—Cl | C—H | C—Cl | C—H | H | CF3 | H | n-PrOCO |
| 14-150 | C—Cl | C—H | C—Cl | C—H | H | CF3 | H | cyclo-PrOCO |
| 14-151 | C—Cl | C—H | C—Cl | C—H | H | CF3 | H | CF3CH20C0 |
| 14-152 | C—Cl | C—H | C—Cl | C—H | H | CF3 | H | CH2═CHCH2OCO |
| 14-153 | C—Cl | C—H | C—Cl | C—H | H | CF3 | H | HCCCH2OCO |
| 14-154 | C—Cl | C—H | C—Cl | C—H | H | CF3 | H | PhOCO |
| 14-155 | C—Cl | C—H | C—Cl | C—H | H | CF3 | H | PhCH2OCO |
| 14-156 | C—Cl | C—H | C—Cl | C—H | H | CF3 | H | tert-BuOCO |
| 14-157 | C—Cl | C—H | C—Cl | C—H | H | CF3 | H | CH3SO2 |
| 14-158 | C—Cl | C—H | C—Cl | C—H | H | CF3 | H | CH3CH2SO2 |
| 14-159 | C—Cl | C—H | C—Cl | C—H | H | CF3 | H | CF3SO2 |
| 14-160 | C—Cl | C—H | C—Cl | C—H | H | CF3 | H | CF3CH2SO2 |
| 14-161 | C—Cl | C—H | C—Cl | C—H | H | CF3 | H | PhSO2 |
| 14-162 | C—Cl | C—H | C—Cl | C—H | H | CF3 | H | CH3NHCONHSO2 |
| 14-163 | C—Cl | C—H | C—Cl | C—H | H | CF3 | H | (CH3)2NCONHSO2 |
| 14-164 | C—Cl | C—H | C—Cl | C—H | H | CF3 | H | CH3CS |
| 14-165 | C—Cl | C—H | C—Cl | C—H | H | CF3 | H | CH3CH2CS |
| 14-166 | C—Cl | C—H | C—Cl | C—H | H | CF3 | H | cyclo-PrCS |
| 14-167 | C—Cl | C—H | C—Cl | C—H | H | CF3 | H | CF3CH2CS |
| 14-168 | C—Cl | C—H | C—Br | C—H | H | CF3 | H | CH3CO |
| 14-169 | C—Cl | C—H | C—Br | C—H | H | CF3 | H | CH3CH2CO |
| 14-170 | C—Cl | C—H | C—Br | C—H | H | CF3 | H | n-PrCO |
| 14-171 | C—Cl | C—H | C—Br | C—H | H | CF3 | H | cyclo-PrCO |
| 14-172 | C—Cl | C—H | C—Br | C—H | H | CF3 | H | cyclo-PrCH2CO |
| 14-173 | C—Cl | C—H | C—Br | C—H | H | CF3 | H | CF3CH2CO |
| 14-175 | C—Cl | C—H | C—Br | C—H | H | CF3 | H | CH3S(O)CH2CO |
| 14-176 | C—Cl | C—H | C—Br | C—H | H | CF3 | H | CH3SO2CH2CO |
| 14-177 | C—Cl | C—H | C—Br | C—H | H | CF3 | H | CH3CH2NHCO |
| 14-178 | C—Br | C—H | C—Br | C—H | H | CF3 | H | CH3CO |
| 14-179 | C—Br | C—H | C—Br | C—H | H | CF3 | H | CH3CH2CO |
| 14-180 | C—Br | C—H | C—Br | C—H | H | CF3 | H | n-PrCO |
| 14-181 | C—Br | C—H | C—Br | C—H | H | CF3 | H | cyclo-PrCO |
| 14-182 | C—Br | C—H | C—Br | C—H | H | CF3 | H | cyclo-PrCH2CO |
| 14-183 | C—Br | C—H | C—Br | C—H | H | CF3 | H | CF3CH2CO |
| 14-185 | C—Br | C—H | C—Br | C—H | H | CF3 | H | CH3S(O)CH2CO |
| 14-186 | C—Br | C—H | C—Br | C—H | H | CF3 | H | CH3SO2CH2CO |
| 14-187 | C—Br | C—H | C—Br | C—H | H | CF3 | H | CH3CH2NHCO |
| 14-188 | C—Cl | C—F | C—Cl | C—H | H | CF3 | H | CH3CO |
| 14-189 | C—Cl | C—F | C—Cl | C—H | H | CF3 | H | CH3CH2CO |
| 14-190 | C—Cl | C—F | C—Cl | C—H | H | CF3 | H | n-PrCO |
| 14-191 | C—Cl | C—F | C—Cl | C—H | H | CF3 | H | cyclo-PrCO |
| 14-192 | C—Cl | C—F | C—Cl | C—H | H | CF3 | H | cyclo-PrCH2C0 |
| 14-193 | C—Cl | C—F | C—Cl | C—H | H | CF3 | H | CF3CH2CO |
| 14-195 | C—Cl | C—F | C—Cl | C—H | H | CF3 | H | CH3S(O)CH2CO |
| 14-196 | C—Cl | C—F | C—Cl | C—H | H | CF3 | H | CH3SO2CH2CO |
| 14-197 | C—Cl | C—F | C—Cl | C—H | H | CF3 | H | CH3CH2NHCO |
| 14-198 | C—Cl | C—Cl | C—Cl | C—H | H | CF3 | H | CH3CO |
| 14-199 | C—Cl | C—Cl | C—Cl | C—H | H | CF3 | H | CH3CH2CO |
| 14-200 | C—Cl | C—Cl | C—Cl | C—H | H | CF3 | H | n-PrCO |
| 14-201 | C—Cl | C—Cl | C—Cl | C—H | H | CF3 | H | cyclo-PrCO |
| 14-202 | C—Cl | C—Cl | C—Cl | C—H | H | CF3 | H | cyclo-PrCH2C0 |
| 14-203 | C—Cl | C—Cl | C—Cl | C—H | H | CF3 | H | CF3CH2CO |
| 14-205 | C—Cl | C—Cl | C—Cl | C—H | H | CF3 | H | CH3CH2OCH2CH2-00 |
| 14-206 | C—Cl | C—Cl | C—Cl | C—H | H | CF3 | H | CH3SCH2C0 |
| 14-207 | C—Cl | C—Cl | C—Cl | C—H | H | CF3 | H | CH3S(O)CH2CO |
| 14-208 | C—Cl | C—Cl | C—Cl | C—H | H | CF3 | H | CH3SO2CH2CO |
| 14-209 | C—Cl | C—Cl | C—Cl | C—H | H | CF3 | H | CH3CH2NHCO |
| 14-210 | C—Cl | C—Br | C—Cl | C—H | H | CF3 | H | CH3CO |
| 14-211 | C—Cl | C—Br | C—Cl | C—H | H | CF3 | H | CH3CH2CO |
| 14-212 | C—Cl | C—Br | C—Cl | C—H | H | CF3 | H | n-PrCO |
| 14-213 | C—Cl | C—Br | C—Cl | C—H | H | CF3 | H | cyclo-PrCO |
| 14-214 | C—Cl | C—Br | C—Cl | C—H | H | CF3 | H | cyclo-PrCH2CO |
| 14-215 | C—Cl | C—Br | C—Cl | C—H | H | CF3 | H | CF3CH2CO |
| 14-217 | C—Cl | C—Br | C—Cl | C—H | H | CF3 | H | CH3S(O)CH2CO |
| 14-218 | C—Cl | C—Br | C—Cl | C—H | H | CF3 | H | CH3SO2CH2CO |
| 14-219 | C—Cl | C—Br | C—Cl | C—H | H | CF3 | H | CH3CH2NHCO |
| 14-220 | C—Cl | C—Cl | C—Br | C—H | H | CF3 | H | CH3CO |
| 14-221 | C—Cl | C—Cl | C—Br | C—H | H | CF3 | H | CH3CH2CO |
| 14-222 | C—Cl | C—Cl | C—Br | C—H | H | CF3 | H | n-PrCO |
| 14-223 | C—Cl | C—Cl | C—Br | C—H | H | CF3 | H | cyclo-PrCO |
| 14-224 | C—Cl | C—Cl | C—Br | C—H | H | CF3 | H | cyclo-PrCH2CO |
| 14-225 | C—Cl | C—Cl | C—Br | C—H | H | CF3 | H | CF3CH2CO |
| 14-227 | C—Cl | C—Cl | C—Br | C—H | H | CF3 | H | CH3S(O)CH2CO |
| 14-228 | C—Cl | C—Cl | C—Br | C—H | H | CF3 | H | CH3SO2CH2CO |
| 14-229 | C—Cl | C—Cl | C—Br | C—H | H | CF3 | H | CH3CH2NHCO |
| 14-230 | C—Cl | C—F | C—Cl | C—H | F | CF3 | H | CH3CO |
| 14-231 | C—Cl | C—F | C—Cl | C—H | F | CF3 | H | CH3CH2CO |
| 14-232 | C—Cl | C—F | C—Cl | C—H | F | CF3 | H | n-PrCO |
| 14-233 | C—Cl | C—F | C—Cl | C—H | F | CF3 | H | cyclo-PrCO |
| 14-234 | C—Cl | C—F | C—Cl | C—H | F | CF3 | H | cyclo-PrCH2CO |
| 14-235 | C—Cl | C—F | C—Cl | C—H | F | CF3 | H | CF3CH2CO |
| 14-237 | C—Cl | C—F | C—Cl | C—H | F | CF3 | H | CH3S(O)CH2CO |
| 14-238 | C—Cl | C—F | C—Cl | C—H | F | CF3 | H | CH3SO2CH2CO |
| 14-239 | C—Cl | C—F | C—Cl | C—H | F | CF3 | H | CH3CH2NHCO |
| 14-240 | C—CF3 | C—H | C—H | C—H | H | CF3 | H | CH3CO |
| 14-241 | C—CF3 | C—H | C—H | C—H | H | CF3 | H | CH3CH2CO |
| 14-242 | C—CF3 | C—H | C—H | C—H | H | CF3 | H | n-PrCO |
| 14-243 | C—CF3 | C—H | C—H | C—H | H | CF3 | H | cyclo-PrCO |
| 14-244 | C—CF3 | C—H | C—H | C—H | H | CF3 | H | cyclo-PrCH2CO |
| 14-245 | C—CF3 | C—H | C—H | C—H | H | CF3 | H | CF3CH2CO |
| 14-247 | C—CF3 | C—H | C—H | C—H | H | CF3 | H | CH3S(O)CH2CO |
| 14-248 | C—CF3 | C—H | C—H | C—H | H | CF3 | H | CH3SO2CH2CO |
| 14-249 | C—CF3 | C—H | C—H | C—H | H | CF3 | H | CH3CH2NHCO |
| 14-250 | C—CF3 | C—F | C—H | C—H | H | CF3 | H | CH3CO |
| 14-251 | C—CF3 | C—F | C—H | C—H | H | CF3 | H | CH3CH2CO |
| 14-252 | C—CF3 | C—F | C—H | C—H | H | CF3 | H | n-PrCO |
| 14-253 | C—CF3 | C—F | C—H | C—H | H | CF3 | H | cyclo-PrCO |
| 14-254 | C—CF3 | C—F | C—H | C—H | H | CF3 | H | cyclo-PrCH2CO |
| 14-255 | C—CF3 | C—F | C—H | C—H | H | CF3 | H | CF3CH2CO |
| 14-257 | C—CF3 | C—F | C—H | C—H | H | CF3 | H | CH3S(O)CH2CO |
| 14-258 | C—CF3 | C—F | C—H | C—H | H | CF3 | H | CH3SO2CH2CO |
| 14-259 | C—CF3 | C—F | C—H | C—H | H | CF3 | H | CH3CH2NHCO |
| 14-260 | C—CF3 | C—H | C—H | C—H | F | CF3 | H | CH3CO |
| 14-261 | C—CF3 | C—H | C—H | C—H | F | CF3 | H | CH3CH2CO |
| 14-262 | C—CF3 | C—H | C—H | C—H | F | CF3 | H | n-PrCO |
| 14-263 | C—CF3 | C—H | C—H | C—H | F | CF3 | H | cyclo-PrCO |
| 14-264 | C—CF3 | C—H | C—H | C—H | F | CF3 | H | cyclo-PrCH2CO |
| 14-265 | C—CF3 | C—H | C—H | C—H | F | CF3 | H | CF3CH2CO |
| 14-267 | C—CF3 | C—H | C—H | C—H | F | CF3 | H | CH3S(O)CH2CO |
| 14-268 | C—CF3 | C—H | C—H | C—H | F | CF3 | H | CH3SO2CH2CO |
| 14-269 | C—CF3 | C—H | C—H | C—H | F | CF3 | H | CH3CH2NHCO |
| 14-270 | C—H | C—H | C—CF3 | C—H | F | CF3 | H | CH3CO |
| 14-271 | C—H | C—H | C—CF3 | C—H | F | CF3 | H | CH3CH2CO |
| 14-272 | C—H | C—H | C—CF3 | C—H | F | CF3 | H | n-PrCO |
| 14-273 | C—H | C—H | C—CF3 | C—H | F | CF3 | H | cyclo-PrCO |
| 14-274 | C—H | C—H | C—CF3 | C—H | F | CF3 | H | cyclo-PrCH2CO |
| 14-275 | C—H | C—H | C—CF3 | C—H | F | CF3 | H | CF3CH2CO |
| 14-277 | C—H | C—H | C—CF3 | C—H | F | CF3 | H | CH3S(O)CH2CO |
| 14-278 | C—H | C—H | C—CF3 | C—H | F | CF3 | H | CH3SO2CH2CO |
| 14-279 | C—H | C—H | C—CF3 | C—H | F | CF3 | H | CH3CH2NHCO |
| 14-280 | C—Cl | C—H | C—CF3 | C—H | H | CF3 | H | CH3CO |
| 14-281 | C—Cl | C—H | C—CF3 | C—H | H | CF3 | H | CH3CH2CO |
| 14-282 | C—Cl | C—H | C—CF3 | C—H | H | CF3 | H | n-PrCO |
| 14-283 | C—Cl | C—H | C—CF3 | C—H | H | CF3 | H | cyclo-PrCO |
| 14-284 | C—Cl | C—H | C—CF3 | C—H | H | CF3 | H | cyclo-PrCH2CO |
| 14-285 | C—Cl | C—H | C—CF3 | C—H | H | CF3 | H | CF3CH2CO |
| 14-287 | C—Cl | C—H | C—CF3 | C—H | H | CF3 | H | CH3CH2OCH2CH2—CO |
| 14-288 | C—Cl | C—H | C—CF3 | C—H | H | CF3 | H | CH3SCH2CO |
| 14-289 | C—Cl | C—H | C—CF3 | C—H | H | CF3 | H | CH3S(O)CH2CO |
| 14-290 | C—Cl | C—H | C—CF3 | C—H | H | CF3 | H | CH3SO2CH2CO |
| 14-291 | C—Cl | C—H | C—CF3 | C—H | H | CF3 | H | CH3CH2NHCO |
| 14-292 | C—CF3 | C—H | C—CF3 | C—H | H | CF3 | H | CH3CO |
| 14-293 | C—CF3 | C—H | C—CF3 | C—H | H | CF3 | H | CH3CH2CO |
| 14-294 | C—CF3 | C—H | C—CF3 | C—H | H | CF3 | H | n-PrCO |
| 14-295 | C—CF3 | C—H | C—CF3 | C—H | H | CF3 | H | cyclo-PrCO |
| 14-296 | C—CF3 | C—H | C—CF3 | C—H | H | CF3 | H | cyclo-PrCH2CO |
| 14-297 | C—CF3 | C—H | C—CF3 | C—H | H | CF3 | H | CF3CH2CO |
| 14-299 | C—CF3 | C—H | C—CF3 | C—H | H | CF3 | H | CH3CH2OCH2CH2—CO |
| 14-300 | C—CF3 | C—H | C—CF3 | C—H | H | CF3 | H | CH3SCH2CO |
| 14-301 | C—CF3 | C—H | C—CF3 | C—H | H | CF3 | H | CH3S(O)CH2CO |
| 14-302 | C—CF3 | C—H | C—CF3 | C—H | H | CF3 | H | CH3SO2CH2CO |
| 14-303 | C—CF3 | C—H | C—CF3 | C—H | H | CF3 | H | CH3CH2NHCO |
| 14-304 | C—Cl | C-Cl | C—CF3 | C—H | H | CF3 | H | CH3CO |
| 14-305 | C—Cl | C-Cl | C—CF3 | C—H | H | CF3 | H | CH3CH2CO |
| 14-306 | C—Cl | C-Cl | C—CF3 | C—H | H | CF3 | H | n-PrCO |
| 14-307 | C—Cl | C-Cl | C—CF3 | C—H | H | CF3 | H | cyclo-PrCO |
| 14-308 | C—Cl | C-Cl | C—CF3 | C—H | H | CF3 | H | cyclo-PrCH2CO |
| 14-309 | C—Cl | C-Cl | C—CF3 | C—H | H | CF3 | H | CF3CH2CO |
| 14-311 | C—Cl | C-Cl | C—CF3 | C—H | H | CF3 | H | CH3S(O)CH2CO |
| 14-312 | C—Cl | C-Cl | C—CF3 | C—H | H | CF3 | H | CH3SO2CH2CO |
| 14-313 | C—Cl | C-Cl | C—CF3 | C—H | H | CF3 | H | CH3CH2NHCO |
| 14-314 | C—Cl | N | C—H | C—H | H | CF3 | H | CH3CO |
| 14-315 | C—Cl | N | C—H | C—H | H | CF3 | H | CH3CH2CO |
| 14-316 | C—Cl | N | C—H | C—H | H | CF3 | H | n-PrCO |
| 14-317 | C—Cl | N | C—H | C—H | H | CF3 | H | cyclo-PrCO |
| 14-318 | C—Cl | N | C—H | C—H | H | CF3 | H | cyclo-PrCH2CO |
| 14-319 | C—Cl | N | C—H | C—H | H | CF3 | H | CF3CH2CO |
| 14-321 | C—Cl | N | C—H | C—H | H | CF3 | H | CH3S(O)CH2CO |
| 14-322 | C—Cl | N | C—H | C—H | H | CF3 | H | CH3SO2CH2CO |
| 14-323 | C—Cl | N | C—H | C—H | H | CF3 | H | CH3CH2NHCO |
| 14-324 | C—Cl | N | C—Cl | C—H | H | CF3 | H | CH3CO |
| 14-325 | C—Cl | N | C—Cl | C—H | H | CF3 | H | CH3CH2CO |
| 14-326 | C—Cl | N | C—Cl | C—H | H | CF3 | H | n-PrCO |
| 14-327 | C—Cl | N | C—Cl | C—H | H | CF3 | H | cyclo-PrCO |
| 14-328 | C—Cl | N | C—Cl | C—H | H | CF3 | H | cyclo-PrCH2CO |
| 14-329 | C—Cl | N | C—Cl | C—H | H | CF3 | H | CF3CH2CO |
| 14-330 | C—Cl | N | C—Cl | C—H | H | CF3 | H | CH3SCH2CO |
| 14-331 | C—Cl | N | C—Cl | C—H | H | CF3 | H | CH3S(O)CH2CO |
| 14-332 | C—Cl | N | C—Cl | C—H | H | CF3 | H | CH3SO2CH2CO |
| 14-333 | C—Cl | N | C—Cl | C—H | H | CF3 | H | CH3CH2NHCO |
| 14-334 | C—CF3 | N | C—H | C—H | H | CF3 | H | CH3CO |
| 14-335 | C—CF3 | N | C—H | C—H | H | CF3 | H | CH3CH2CO |
| 14-336 | C—CF3 | N | C—H | C—H | H | CF3 | H | n-PrCO |
| 14-337 | C—CF3 | N | C—H | C—H | H | CF3 | H | cyclo-PrCO |
| 14-338 | C—CF3 | N | C—H | C—H | H | CF3 | H | cyclo-PrCH2CO |
| 14-339 | C—CF3 | N | C—H | C—H | H | CF3 | H | CF3CH2CO |
| 14-340 | C—CF3 | N | C—H | C—H | H | CF3 | H | CH3SCH2C0 |
| 14-341 | C—CF3 | N | C—H | C—H | H | CF3 | H | CH3S(O)CH2CO |
| 14-342 | C—CF3 | N | C—H | C—H | H | CF3 | H | CH3SO2CH2CO |
| 14-343 | C—CF3 | N | C—H | C—H | H | CF3 | H | CH3CH2NHCO |
| 14-344 | C—CF3 | N | C—CF3 | C—H | H | CF3 | H | CH3CO |
| 14-345 | C—CF3 | N | C—CF3 | C—H | H | CF3 | H | CH3CH2CO |
| 14-346 | C—CF3 | N | C—CF3 | C—H | H | CF3 | H | n-PrCO |
| 14-347 | C—CF3 | N | C—CF3 | C—H | H | CF3 | H | cyclo-PrCO |
| 14-348 | C—CF3 | N | C—CF3 | C—H | H | CF3 | H | cyclo-PrCH2CO |
| 14-349 | C—CF3 | N | C—CF3 | C—H | H | CF3 | H | CF3CH2CO |
| 14-350 | C—CF3 | N | C—CF3 | C—H | H | CF3 | H | CH3SCH2CO |
| 14-351 | C—CF3 | N | C—CF3 | C—H | H | CF3 | H | CH3S(O)CH2CO |
| 14-352 | C—CF3 | N | C—CF3 | C—H | H | CF3 | H | CH3SO2CH2CO |
| 14-353 | C—CF3 | N | C—CF3 | C—H | H | CF3 | H | CH3CH2NHCO |
| 14-354 | C—CF3 | C—H | C—H | N | H | CF3 | H | CH3CO |
| 14-355 | C—CF3 | C—H | C—H | N | H | CF3 | H | CH3CH2CO |
| 14-356 | C—CF3 | C—H | C—H | N | H | CF3 | H | n-PrCO |
| 14-357 | C—CF3 | C—H | C—H | N | H | CF3 | H | cyclo-PrCO |
| 14-358 | C—CF3 | C—H | C—H | N | H | CF3 | H | cyclo-PrCH2CO |
| 14-359 | C—CF3 | C—H | C—H | N | H | CF3 | H | CF3CH2CO |
| 14-360 | C—CF3 | C—H | C—H | N | H | CF3 | H | CH3SCH2CO |
| 14-361 | C—CF3 | C—H | C—H | N | H | CF3 | H | CH3S(O)CH2CO |
| 14-362 | C—CF3 | C—H | C—H | N | H | CF3 | H | CH3SO2CH2CO |
| 14-363 | C—CF3 | C—H | C—H | N | H | CF3 | H | CH3CH2NHCO |
| 14-364 | C—H | C—H | C—CF3 | N | H | CF3 | H | CH3CO |
| 14-365 | C—H | C—H | C—CF3 | N | H | CF3 | H | CH3CH2CO |
| 14-366 | C—H | C—H | C—CF3 | N | H | CF3 | H | n-PrCO |
| 14-367 | C—H | C—H | C—CF3 | N | H | CF3 | H | cyclo-PrCO |
| 14-368 | C—H | C—H | C—CF3 | N | H | CF3 | H | cyclo-PrCH2CO |
| 14-369 | C—H | C—H | C—CF3 | N | H | CF3 | H | CF3CH2CO |
| 14-370 | C—H | C—H | C—CF3 | N | H | CF3 | H | CH3SCH2CO |
| 14-371 | C—H | C—H | C—CF3 | N | H | CF3 | H | CH3S(O)CH2CO |
| 14-372 | C—H | C—H | C—CF3 | N | H | CF3 | H | CH3SO2CH2CO |
| 14-373 | C—H | C—H | C—CF3 | N | H | CF3 | H | CH3CH2NHCO |
| 14-374 | C—CF3 | C—H | C—Cl | N | H | CF3 | H | CH3CO |
| 14-375 | C—CF3 | C—H | C—Cl | N | H | CF3 | H | CH3CH2CO |
| 14-376 | C—CF3 | C—H | C—Cl | N | H | CF3 | H | n-PrCO |
| 14-377 | C—CF3 | C—H | C—Cl | N | H | CF3 | H | cyclo-PrCO |
| 14-378 | C—CF3 | C—H | C—Cl | N | H | CF3 | H | cyclo-PrCH2CO |
| 14-379 | C—CF3 | C—H | C—Cl | N | H | CF3 | H | CF3CH2CO |
| 14-380 | C—CF3 | C—H | C—Cl | N | H | CF3 | H | CH3SCH2CO |
| 14-381 | C—CF3 | C—H | C—Cl | N | H | CF3 | H | CH3S(O)CH2CO |
| 14-382 | C—CF3 | C—H | C—Cl | N | H | CF3 | H | CH3SO2CH2CO |
| 14-383 | C—CF3 | C—H | C—Cl | N | H | CF3 | H | CH3CH2NHCO |
| 14-384 | C—CF3 | C—H | C—Br | N | H | CF3 | H | CH3CO |
| 14-385 | C—CF3 | C—H | C—Br | N | H | CF3 | H | CH3CH2CO |
| 14-386 | C—CF3 | C—H | C—Br | N | H | CF3 | H | n-PrCO |
| 14-387 | C—CF3 | C—H | C—Br | N | H | CF3 | H | cyclo-PrCO |
| 14-388 | C—CF3 | C—H | C—Br | N | H | CF3 | H | cyclo-PrCH2CO |
| 14-389 | C—CF3 | C—H | C—Br | N | H | CF3 | H | CF3CH2CO |
| 14-390 | C—CF3 | C—H | C—Br | N | H | CF3 | H | CH3SCH2CO |
| 14-391 | C—CF3 | C—H | C—Br | N | H | CF3 | H | CH3S(O)CH2CO |
| 14-392 | C—CF3 | C—H | C—Br | N | H | CF3 | H | CH3SO2CH2CO |
| 14-393 | C—CF3 | C—H | C—Br | N | H | CF3 | H | CH3CH2NHCO |
| 14-394 | C—CF3 | C—H | C—CF3 | N | H | CF3 | H | CH3CO |
| 14-395 | C—CF3 | C—H | C—CF3 | N | H | CF3 | H | CH3CH2CO |
| 14-396 | C—CF3 | C—H | C—CF3 | N | H | CF3 | H | n-PrCO |
| 14-397 | C—CF3 | C—H | C—CF3 | N | H | CF3 | H | cyclo-PrCO |
| 14-398 | C—CF3 | C—H | C—CF3 | N | H | CF3 | H | cyclo-PrCH2CO |
| 14-399 | C—CF3 | C—H | C—CF3 | N | H | CF3 | H | CF3CH2CO |
| 14-400 | C—CF3 | C—H | C—CF3 | N | H | CF3 | H | CH3SCH2CO |
| 14-401 | C—CF3 | C—H | C—CF3 | N | H | CF3 | H | CH3S(O)CH2CO |
| 14-402 | C—CF3 | C—H | C—CF3 | N | H | CF3 | H | CH3SO2CH2CO |
| 14-403 | C—CF3 | C—H | C—CF3 | N | H | CF3 | H | CH3CH2NHCO |

TABLE 15

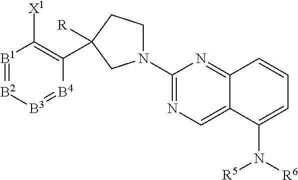

| No. | B¹ | B² | B³ | B⁴ | X¹ | R | R⁵ | R⁶ |
|---|---|---|---|---|---|---|---|---|
| 15-1 | C—Cl | C—H | C—H | C—H | H | CF3 | H | CH3CO |
| 15-2 | C—Cl | C—H | C—H | C—H | H | CF3 | H | CH3CH2CO |
| 15-3 | C—Cl | C—H | C—H | C—H | H | CF3 | H | n-PrCO |
| 15-4 | C—Cl | C—H | C—H | C—H | H | CF3 | H | cyclo-PrCO |
| 15-5 | C—Cl | C—H | C—H | C—H | H | CF3 | H | cyclo-PrCH2CO |
| 15-6 | C—Cl | C—H | C—H | C—H | H | CF3 | H | CF3CH2CO |
| 15-7 | C—Cl | C—H | C—H | C—H | H | CF3 | H | CH3SCH2CO |
| 15-8 | C—Cl | C—H | C—H | C—H | H | CF3 | H | CH3S(O)CH2CO |
| 15-9 | C—Cl | C—H | C—H | C—H | H | CF3 | H | CH3SO2CH2CO |
| 15-10 | C—Cl | C—H | C—H | C—H | H | CF3 | H | CH3CH2NHCO |
| 15-11 | C—Cl | C—H | C—H | C—H | F | CF3 | H | CH3CO |
| 15-12 | C—Cl | C—H | C—H | C—H | F | CF3 | H | CH3CH2CO |
| 15-13 | C—Cl | C—H | C—H | C—H | F | CF3 | H | n-PrCO |
| 15-14 | C—Cl | C—H | C—H | C—H | F | CF3 | H | cyclo-PrCO |
| 15-15 | C—Cl | C—H | C—H | C—H | F | CF3 | H | cyclo-PrCH2CO |
| 15-16 | C—Cl | C—H | C—H | C—H | F | CF3 | H | CF3CH2CO |
| 15-17 | C—Cl | C—H | C—H | C—H | F | CF3 | H | CH3SCH2CO |
| 15-18 | C—Cl | C—H | C—H | C—H | F | CF3 | H | CH3S(O)CH2CO |
| 15-19 | C—Cl | C—H | C—H | C—H | F | CF3 | H | CH3SO2CH2CO |
| 15-20 | C—Cl | C—H | C—H | C—H | F | CF3 | H | CH3CH2NHCO |
| 15-21 | C—H | C—H | C—Cl | C—H | F | CF3 | H | CH3CO |
| 15-22 | C—H | C—H | C—Cl | C—H | F | CF3 | H | CH3CH2CO |
| 15-23 | C—H | C—H | C—Cl | C—H | F | CF3 | H | n-PrCO |
| 15-24 | C—H | C—H | C—Cl | C—H | F | CF3 | H | cyclo-PrCO |
| 15-25 | C—H | C—H | C—Cl | C—H | F | CF3 | H | cyclo-PrCH2CO |
| 15-26 | C—H | C—H | C—Cl | C—H | F | CF3 | H | CF3CH2CO |
| 15-27 | C—H | C—H | C—Cl | C—H | F | CF3 | H | CH3SCH2CO |
| 15-28 | C—H | C—H | C—Cl | C—H | F | CF3 | H | CH3S(O)CH2CO |
| 15-29 | C—H | C—H | C—Cl | C—H | F | CF3 | H | CH3SO2CH2CO |
| 15-30 | C—H | C—H | C—Cl | C—H | F | CF3 | H | CH3CH2NHCO |
| 15-31 | C—Cl | C—H | C—Cl | C—H | H | CF3 | H | H |
| 15-32 | C—Cl | C—H | C—Cl | C—H | H | CF3 | H | HCO |
| 15-33 | C—Cl | C—H | C—Cl | C—H | H | CF3 | H | CH3CO |
| 15-34 | C—Cl | C—H | C—Cl | C—H | H | CF3 | H | CH3CH2CO |
| 15-35 | C—Cl | C—H | C—Cl | C—H | H | CF3 | H | n-PrCO |
| 15-36 | C—Cl | C—H | C—Cl | C—H | H | CF3 | H | iso-PrCO |
| 15-37 | C—Cl | C—H | C—Cl | C—H | H | CF3 | H | cyclo-PrCO |
| 15-38 | C—Cl | C—H | C—Cl | C—H | H | CF3 | H | n-BuCO |
| 15-39 | C—Cl | C—H | C—Cl | C—H | H | CF3 | H | tert-BuCO |
| 15-40 | C—Cl | C—H | C—Cl | C—H | H | CF3 | H | iso-BuCO |
| 15-41 | C—Cl | C—H | C—Cl | C—H | H | CF3 | H | cyclo-BuCO |
| 15-42 | C—Cl | C—H | C—Cl | C—H | H | CF3 | H | (CH3)3CCH2CO |
| 15-43 | C—Cl | C—H | C—Cl | C—H | H | CF3 | H | cyclo-PenCO |
| 15-44 | C—Cl | C—H | C—Cl | C—H | H | CF3 | H | cyclo-HexCO |
| 15-45 | C—Cl | C—H | C—Cl | C—H | H | CF3 | H | cyclo-PrCH2CO |
| 15-46 | C—Cl | C—H | C—Cl | C—H | H | CF3 | H | CF3CO |
| 15-47 | C—Cl | C—H | C—Cl | C—H | H | CF3 | H | CCl3CO |
| 15-48 | C—Cl | C—H | C—Cl | C—H | H | CF3 | H | ClCH2CO |
| 15-49 | C—Cl | C—H | C—Cl | C—H | H | CF3 | H | CF2ClCO |
| 15-50 | C—Cl | C—H | C—Cl | C—H | H | CF3 | H | ClCH2CH2CO |
| 15-51 | C—Cl | C—H | C—Cl | C—H | H | CF3 | H | HCF2CF2CO |
| 15-52 | C—Cl | C—H | C—Cl | C—H | H | CF3 | H | CF3CH2CO |
| 15-54 | C—Cl | C—H | C—Cl | C—H | H | CF3 | H | HCF2CO |
| 15-55 | C—Cl | C—H | C—Cl | C—H | H | CF3 | H | ClCH2CO |
| 15-56 | C—Cl | C—H | C—Cl | C—H | H | CF3 | H | 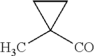 |
| 15-57 | C—Cl | C—H | C—Cl | C—H | H | CF3 | H | 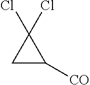 |
| 15-58 | C—Cl | C—H | C—Cl | C—H | H | CF3 | H | 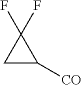 |
| 15-59 | C—Cl | C—H | C—Cl | C—H | H | CF3 | H | 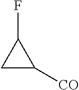 |
| 15-60 | C—Cl | C—H | C—Cl | C—H | H | CF3 | H | 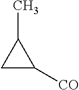 |
| 15-61 | C—Cl | C—H | C—Cl | C—H | H | CF3 | H | 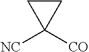 |
| 15-62 | C—Cl | C—H | C—Cl | C—H | H | CF3 | H | CH2═CH—CO |
| 15-63 | C—Cl | C—H | C—Cl | C—H | H | CF3 | H | CH3CH═CH—CO |
| 15-64 | C—Cl | C—H | C—Cl | C—H | H | CF3 | H | CH2═C(CH3)—CO |
| 15-65 | C—Cl | C—H | C—Cl | C—H | H | CF3 | H | CH3CH═C(CH3)—CO |
| 15-66 | C—Cl | C—H | C—Cl | C—H | H | CF3 | H | (CH3)2C═CH—CO |
| 15-67 | C—Cl | C—H | C—Cl | C—H | H | CF3 | H | CH2═C(Cl)—CO |
| 15-68 | C—Cl | C—H | C—Cl | C—H | H | CF3 | H | ClCH═C(CH3)—CO |
| 15-69 | C—Cl | C—H | C—Cl | C—H | H | CF3 | H | CH2═CHCH2—CO |
| 15-70 | C—Cl | C—H | C—Cl | C—H | H | CF3 | H | HCC—CO |
| 15-71 | C—Cl | C—H | C—Cl | C—H | H | CF3 | H | CH3CC—CO |
| 15-72 | C—Cl | C—H | C—Cl | C—H | H | CF3 | H | NCCH2—CO |
| 15-73 | C—Cl | C—H | C—Cl | C—H | H | CF3 | H | CH3OCH2—CO |
| 15-74 | C—Cl | C—H | C—Cl | C—H | H | CF3 | H | C2H5OCH2—CO |
| 15-75 | C—Cl | C—H | C—Cl | C—H | H | CF3 | 11 | CH3OCH2CH2—CO |
| 15-76 | C—Cl | C—H | C—Cl | C—H | H | CF3 | H | CH3CH2OCH2CH2—CO |
| 15-77 | C—Cl | C—H | C—Cl | C—H | H | CF3 | H | CH3SCH2CO |
| 15-78 | C—Cl | C—H | C—Cl | C—H | H | CF3 | H | CH3S(O)CH2CO |
| 15-79 | C—Cl | C—H | C—Cl | C—H | H | CF3 | H | CH3SO2CH2CO |
| 15-80 | C—Cl | C—H | C—Cl | C—H | H | CF3 | H | CH3O(CO)CH2—CO |
| 15-81 | C—Cl | C—H | C—Cl | C—H | H | CF3 | H | CH3(CO)OCH2—CO |
| 15-82 | C—Cl | C—H | C—Cl | C—H | H | CF3 | H | CH3O(CO)—CO |
| 15-83 | C—Cl | C—H | C—Cl | C—H | H | CF3 | H | CF3O(CO)—CO |
| 15-84 | C—Cl | C—H | C—Cl | C—H | H | CF3 | H | CH3(CO)CH2—CO |
| 15-85 | C—Cl | C—H | C—Cl | C—H | H | CF3 | H | (CH3)2NCH2—CO |
| 15-86 | C—Cl | C—H | C—Cl | C—H | H | CF3 | H | PhCO |
| 15-87 | C—Cl | C—H | C—Cl | C—H | H | CF3 | H | PhCH2CO |
| 15-88 | C—Cl | C—H | C—Cl | C—H | H | CF3 | H | 2-py-CO |
| 15-89 | C—Cl | C—H | C—Cl | C—H | H | CF3 | H | 3-py-CO |
| 15-90 | C—Cl | C—H | C—Cl | C—H | H | CF3 | H | 4-py-CO |
| 15-91 | C—Cl | C—H | C—Cl | C—H | H | CF3 | H | 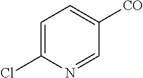 |
| 15-92 | C—Cl | C—H | C—Cl | C—H | H | CF3 | H | 2-F-PhCO |
| 15-93 | C—Cl | C—H | C—Cl | C—H | H | CF3 | H | 3-F-PhCO |
| 15-94 | C—Cl | C—H | C—Cl | C—H | H | CF3 | H | 4-F-PhCO |
| 15-95 | C—Cl | C—H | C—Cl | C—H | H | CF3 | H | 2-Cl-PhCO |
| 15-96 | C—Cl | C—H | C—Cl | C—H | H | CF3 | H | 3-Cl-PhCO |
| 15-97 | C—Cl | C—H | C—Cl | C—H | H | CF3 | H | 4-Cl-PhCO |
| 15-98 | C—Cl | C—H | C—Cl | C—H | H | CF3 | H | 2-Br-PhCO |
| 15-99 | C—Cl | C—H | C—Cl | C—H | H | CF3 | H | 2-CF3-PhCO |
| 15-100 | C—Cl | C—H | C—Cl | C—H | H | CF3 | H | 2-CH3-PhCO |
| 15-101 | C—Cl | C—H | C—Cl | C—H | H | CF3 | H | 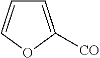 |
| 15-102 | C—Cl | C—H | C—Cl | C—H | H | CF3 | H | 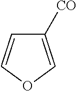 |
| 15-103 | C—Cl | C—H | C—Cl | C—H | H | CF3 | H | 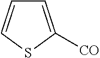 |
| 15-104 | C—Cl | C—H | C—Cl | C—H | H | CF3 | H | 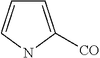 |
| 15-105 | C—Cl | C—H | C—Cl | C—H | H | CF3 | H | 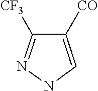 |
| 15-106 | C—Cl | C—H | C—Cl | C—H | H | CF3 | H | 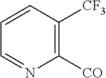 |
| 15-107 | C—Cl | C—H | C—Cl | C—H | H | CF3 | H | 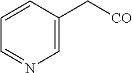 |
| 15-108 | C—Cl | C—H | C—Cl | C—H | H | CF3 | H | 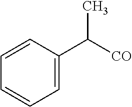 |
| 15-109 | C—Cl | C—H | C—Cl | C—H | H | CF3 | H | CH3NHCO |
| 15-110 | C—Cl | C—H | C—Cl | C—H | H | CF3 | H | CH3CH2NHCO |
| 15-111 | C—Cl | C—H | C—Cl | C—H | H | CF3 | H | CF3CH2NHCO |
| 15-112 | C—Cl | C—H | C—Cl | C—H | H | CF3 | H | n-PrNHCO |
| 15-113 | C—Cl | C—H | C—Cl | C—H | H | CF3 | H | iso-PrNHCO |
| 15-114 | C—Cl | C—H | C—Cl | C—H | H | CF3 | H | cyclo-PrNHCO |
| 15-115 | C—Cl | C—H | C—Cl | C—H | H | CF3 | H | n-BuNHCO |
| 15-116 | C—Cl | C—H | C—Cl | C—H | H | CF3 | H | tert-BuNHCO |
| 15-117 | C—Cl | C—H | C—Cl | C—H | H | CF3 | H | cyclo-BuNHCO |
| 15-118 | C—Cl | C—H | C—Cl | C—H | H | CF3 | H | cyclo-PenNHCO |
| 15-119 | C—Cl | C—H | C—Cl | C—H | H | CF3 | H | cyclo-HexNHCO |
| 15-120 | C—Cl | C—H | C—Cl | C—H | H | CF3 | H | CH2═CHCH2NHCO |
| 15-121 | C—Cl | C—H | C—Cl | C—H | H | CF3 | H | HCCCH2NHCO |
| 15-122 | C—Cl | C—H | C—Cl | C—H | H | CF3 | H | cyclo-PrCH2NHCO |
| 15-123 | C—Cl | C—H | C—Cl | C—H | H | CF3 | H | NCCH2NHCO |
| 15-124 | C—Cl | C—H | C—Cl | C—H | H | CF3 | H | CF3(CH3)CHNHCO |
| 15-125 | C—Cl | C—H | C—Cl | C—H | H | CF3 | H | CF3(CH3)2CNCO |
| 15-126 | C—Cl | C—H | C—Cl | C—H | H | CF3 | H | NCCH2OCO |
| 15-127 | C—Cl | C—H | C—Cl | C—H | H | CF3 | H | FCH2CH2NHCO |
| 15-128 | C—Cl | C—H | C—Cl | C—H | H | CF3 | H | CF3CH2CH2NHCO |
| 15-129 | C—Cl | C—H | C—Cl | C—H | H | CF3 | H | HCF2CF2CH2NHCO |
| 15-130 | C—Cl | C—H | C—Cl | C—H | H | CF3 | H | 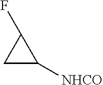 |
| 15-131 | C—Cl | C—H | C—Cl | C—H | H | CF3 | H | 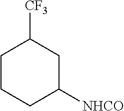 |
| 15-132 | C—Cl | C—H | C—Cl | C—H | H | CF3 | H | EtO2CCH2NHCO |
| 15-133 | C—Cl | C—H | C—Cl | C—H | H | CF3 | H | 2-CF3-PhNHCO |
| 15-134 | C—Cl | C—H | C—Cl | C—H | H | CF3 | H | 3-CF3-PhNHCO |
| 15-135 | C—Cl | C—H | C—Cl | C—H | H | CF3 | H | 4-CF3-PhNHCO |
| 15-136 | C—Cl | C—H | C—Cl | C—H | H | CF3 | H | 2-py-NHCO |
| 15-137 | C—Cl | C—H | C—Cl | C—H | H | CF3 | H | 3-py-NHCO |
| 15-138 | C—Cl | C—H | C—Cl | C—H | H | CF3 | H | 4-py-NHCO |
| 15-139 | C—Cl | C—H | C—Cl | C—H | H | CF3 | H | 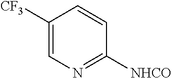 |
| 15-140 | C—Cl | C—H | C—Cl | C—H | H | CF3 | H | 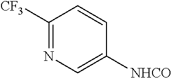 |
| 15-141 | C—Cl | C—H | C—Cl | C—H | H | CF3 | H | PhCH2HNCO |
| 15-142 | C—Cl | C—H | C—Cl | C—H | H | CF3 | H | 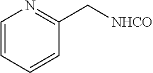 |
| 15-143 | C—Cl | C—H | C—Cl | C—H | H | CF3 | H | (CH3)2NCO |
| 15-144 | C—Cl | C—H | C—Cl | C—H | H | CF3 | H | pyrrolidineCO |
| 15-145 | C—Cl | C—H | C—Cl | C—H | H | CF3 | H | piperidineCO |
| 15-146 | C—Cl | C—H | C—Cl | C—H | H | CF3 | H | morpholineCO |
| 15-147 | C—Cl | C—H | C—Cl | C—H | H | CF3 | H | CH3OCONH |
| 15-148 | C—Cl | C—H | C—Cl | C—H | H | CF3 | H | CH3CH2OCO |
| 15-149 | C—Cl | C—H | C—Cl | C—H | H | CF3 | H | n-PrOCO |
| 15-150 | C—Cl | C—H | C—Cl | C—H | H | CF3 | H | cyclo-PrOCO |
| 15-151 | C—Cl | C—H | C—Cl | C—H | H | CF3 | H | CF3CH2OCO |
| 15-152 | C—Cl | C—H | C—Cl | C—H | H | CF3 | H | CH2═CHCH2OCO |
| 15-153 | C—Cl | C—H | C—Cl | C—H | H | CF3 | H | HCCCH2OCO |
| 15-154 | C—Cl | C—H | C—Cl | C—H | H | CF3 | H | PhOCO |
| 15-155 | C—Cl | C—H | C—Cl | C—H | H | CF3 | H | PhCH2OCO |
| 15-156 | C—Cl | C—H | C—Cl | C—H | H | CF3 | H | tert-BuOCO |
| 15-157 | C—Cl | C—H | C—Cl | C—H | H | CF3 | H | CH3SO2 |
| 15-158 | C—Cl | C—H | C—Cl | C—H | H | CF3 | H | CH3CH2SO2 |
| 15-159 | C—Cl | C—H | C—Cl | C—H | H | CF3 | H | CF3SO2 |
| 15-160 | C—Cl | C—H | C—Cl | C—H | H | CF3 | H | CF3CH2SO2 |
| 15-161 | C—Cl | C—H | C—Cl | C—H | H | CF3 | H | PhSO2 |
| 15-162 | C—Cl | C—H | C—Cl | C—H | H | CF3 | H | CH3NHCONHSO2 |
| 15-163 | C—Cl | C—H | C—Cl | C—H | H | CF3 | H | (CH3)2NCONHSO2 |
| 15-164 | C—Cl | C—H | C—Cl | C—H | H | CF3 | H | CH3CS |
| 15-165 | C—Cl | C—H | C—Cl | C—H | H | CF3 | H | CH3CH2CS |
| 15-166 | C—Cl | C—H | C—Cl | C—H | H | CF3 | H | cyclo-PrCS |
| 15-167 | C—Cl | C—H | C—Cl | C—H | H | CF3 | H | CF3CH2CS |
| 15-168 | C—Cl | C—H | C—Br | C—H | H | CF3 | H | CH3CO |
| 15-169 | C—Cl | C—H | C—Br | C—H | H | CF3 | H | CH3CH2CO |
| 15-170 | C—Cl | C—H | C—Br | C—H | H | CF3 | H | n-PrCO |
| 15-171 | C—Cl | C—H | C—Br | C—H | H | CF3 | H | cyclo-PrCO |
| 15-172 | C—Cl | C—H | C—Br | C—H | H | CF3 | H | cyclo-PrCH2CO |
| 15-173 | C—Cl | C—H | C—Br | C—H | H | CF3 | H | CF3CH2CO |
| 15-175 | C—Cl | C—H | C—Br | C—H | H | CF3 | H | CH3S(O)CH2CO |
| 15-176 | C—Cl | C—H | C—Br | C—H | H | CF3 | H | CH3SO2CH2CO |
| 15-177 | C—Cl | C—H | C—Br | C—H | H | CF3 | H | CH3CH2NHCO |
| 15-178 | C—Br | C—H | C—Br | C—H | H | CF3 | H | CH3CO |
| 15-179 | C—Br | C—H | C—Br | C—H | H | CF3 | H | CH3CH2CO |
| 15-180 | C—Br | C—H | C—Br | C—H | H | CF3 | H | n-PrCO |
| 15-181 | C—Br | C—H | C—Br | C—H | H | CF3 | H | cyclo-PrCO |
| 15-182 | C—Br | C—H | C—Br | C—H | H | CF3 | H | cyclo-PrCH2CO |
| 15-183 | C—Br | C—H | C—Br | C—H | H | CF3 | H | CF3CH2CO |
| 15-185 | C—Br | C—H | C—Br | C—H | H | CF3 | H | CH3S(O)CH2CO |
| 15-186 | C—Br | C—H | C—Br | C—H | H | CF3 | H | CH3SO2CH2CO |
| 15-187 | C—Br | C—H | C—Br | C—H | H | CF3 | H | CH3CH2NHCO |
| 15-188 | C—Cl | C—F | C—Cl | C—H | H | CF3 | H | CH3CO |
| 15-189 | C—Cl | C—F | C—Cl | C—H | H | CF3 | H | CH3CH2CO |
| 15-190 | C—Cl | C—F | C—Cl | C—H | H | CF3 | H | n-PrCO |
| 15-191 | C—Cl | C—F | C—Cl | C—H | H | CF3 | H | cyclo-PrCO |
| 15-192 | C—Cl | C—F | C—Cl | C—H | H | CF3 | H | cyclo-PrCH2CO |
| 15-193 | C—Cl | C—F | C—Cl | C—H | H | CF3 | H | CF3CH2CO |
| 15-195 | C—Cl | C—F | C—Cl | C—H | H | CF3 | H | CH3S(O)CH2CO |
| 15-196 | C—Cl | C—F | C—Cl | C—H | H | CF3 | H | CH3SO2CH2CO |
| 15-197 | C—Cl | C—F | C—Cl | C—H | H | CF3 | H | CH3CH2NHCO |
| 15-198 | C—Cl | C—Cl | C—Cl | C—H | H | CF3 | H | CH3CO |
| 15-199 | C—Cl | C—Cl | C—Cl | C—H | H | CF3 | H | CH3CH2CO |
| 15-200 | C—Cl | C—Cl | C—Cl | C—H | H | CF3 | H | n-PrCO |
| 15-201 | C—Cl | C—Cl | C—Cl | C—H | H | CF3 | H | cyclo-PrCO |
| 15-202 | C—Cl | C—Cl | C—Cl | C—H | H | CF3 | H | cyclo-PrCH2CO |
| 15-203 | C—Cl | C—Cl | C—Cl | C—H | H | CF3 | H | CF3CH2CO |
| 15-205 | C—Cl | C—Cl | C—Cl | C—H | H | CF3 | H | CH3CH2OCH2CH2—CO |
| 15-206 | C—Cl | C—Cl | C—Cl | C—H | H | CF3 | H | CH3SCH2CO |
| 15-207 | C—Cl | C—Cl | C—Cl | C—H | H | CF3 | H | CH3S(O)CH2CO |
| 15-208 | C—Cl | C—Cl | C—Cl | C—H | H | CF3 | H | CH3SO2CH2CO |
| 15-209 | C—Cl | C—Cl | C—Cl | C—H | H | CF3 | H | CH3CH2NHCO |
| 15-210 | C—Cl | C—Br | C—Cl | C—H | H | CF3 | H | CH3CO |
| 15-211 | C—Cl | C—Br | C—Cl | C—H | H | CF3 | H | CH3CH2CO |
| 15-212 | C—Cl | C—Br | C—Cl | C—H | H | CF3 | H | n-PrCO |
| 15-213 | C—Cl | C—Br | C—Cl | C—H | H | CF3 | H | cyclo-PrCO |
| 15-214 | C—Cl | C—Br | C—Cl | C—H | H | CF3 | H | cyclo-PrCH2CO |
| 15-215 | C—Cl | C—Br | C—Cl | C—H | H | CF3 | H | CF3CH2CO |
| 15-217 | C—Cl | C—Br | C—Cl | C—H | H | CF3 | H | CH3S(O)CH2CO |
| 15-218 | C—Cl | C—Br | C—Cl | C—H | H | CF3 | H | CH3SO2CH2CO |
| 15-219 | C—Cl | C—Br | C—Cl | C—H | H | CF3 | H | CH3CH2NHCO |
| 15-220 | C—Cl | C—Cl | C—Br | C—H | H | CF3 | H | CH3CO |
| 15-221 | C—Cl | C—Cl | C—Br | C—H | H | CF3 | H | CH3CH2CO |
| 15-222 | C—Cl | C—Cl | C—Br | C—H | H | CF3 | H | n-PrCO |
| 15-223 | C—Cl | C—Cl | C—Br | C—H | H | CF3 | H | cyclo-PrCO |
| 15-224 | C—Cl | C—Cl | C—Br | C—H | H | CF3 | H | cyclo-PrCH2CO |
| 15-225 | C—Cl | C—Cl | C—Br | C—H | H | CF3 | H | CF3CH2CO |
| 15-227 | C—Cl | C—Cl | C—Br | C—H | H | CF3 | H | CH3S(O)CH2CO |
| 15-228 | C—Cl | C—Cl | C—Br | C—H | H | CF3 | H | CH3SO2CH2CO |
| 15-229 | C—Cl | C—Cl | C—Br | C—H | H | CF3 | H | CH3CH2NHCO |
| 15-230 | C—Cl | C—F | C—Cl | C—H | F | CF3 | H | CH3CO |
| 15-231 | C—Cl | C—F | C—Cl | C—H | F | CF3 | H | CH3CH2CO |
| 15-232 | C—Cl | C—F | C—Cl | C—H | F | CF3 | H | n-PrCO |
| 15-233 | C—Cl | C—F | C—Cl | C—H | F | CF3 | H | cyclo-PrCO |
| 15-234 | C—Cl | C—F | C—Cl | C—H | F | CF3 | H | cyclo-PrCH2CO |
| 15-235 | C—Cl | C—F | C—Cl | C—H | F | CF3 | H | CF3CH2CO |
| 15-237 | C—Cl | C—F | C—Cl | C—H | F | CF3 | H | CH3S(O)CH2CO |
| 15-238 | C—Cl | C—F | C—Cl | C—H | F | CF3 | H | CH3SO2CH2CO |
| 15-239 | C—Cl | C—F | C—Cl | C—H | F | CF3 | H | CH3CH2NHCO |
| 15-240 | C—CF3 | C—H | C—H | C—H | H | CF3 | H | CH3CO |
| 15-241 | C—CF3 | C—H | C—H | C—H | H | CF3 | H | CH3CH2CO |
| 15-242 | C—CF3 | C—H | C—H | C—H | H | CF3 | H | n-PrCO |
| 15-243 | C—CF3 | C—H | C—H | C—H | H | CF3 | H | cyclo-PrCO |
| 15-244 | C—CF3 | C—H | C—H | C—H | H | CF3 | H | cyclo-PrCH2CO |
| 15-245 | C—CF3 | C—H | C—H | C—H | H | CF3 | H | CF3CH2CO |
| 15-247 | C—CF3 | C—H | C—H | C—H | H | CF3 | H | CH3S(O)CH2CO |
| 15-248 | C—CF3 | C—H | C—H | C—H | H | CF3 | H | CH3SO2CH2CO |
| 15-249 | C—CF3 | C—H | C—H | C—H | H | CF3 | H | CH3CH2NHCO |
| 15-250 | C—CF3 | C—F | C—H | C—H | H | CF3 | H | CH3CO |
| 15-251 | C—CF3 | C—F | C—H | C—H | H | CF3 | H | CH3CH2CO |
| 15-252 | C—CF3 | C—F | C—H | C—H | H | CF3 | H | n-PrCO |
| 15-253 | C—CF3 | C—F | C—H | C—H | H | CF3 | H | cyclo-PrCO |
| 15-254 | C—CF3 | C—F | C—H | C—H | H | CF3 | H | cyclo-PrCH2CO |
| 15-255 | C—CF3 | C—F | C—H | C—H | H | CF3 | H | CF3CH2CO |
| 15-257 | C—CF3 | C—F | C—H | C—H | H | CF3 | H | CH3S(O)CH2CO |
| 15-258 | C—CF3 | C—F | C—H | C—H | H | CF3 | H | CH3SO2CH2CO |
| 15-259 | C—CF3 | C—F | C—H | C—H | H | CF3 | H | CH3CH2NHCO |
| 15-260 | C—CF3 | C—H | C—H | C—H | F | CF3 | H | CH3CO |
| 15-261 | C—CF3 | C—H | C—H | C—H | F | CF3 | H | CH3CH2CO |
| 15-262 | C—CF3 | C—H | C—H | C—H | F | CF3 | H | n-PrCO |
| 15-263 | C—CF3 | C—H | C—H | C—H | F | CF3 | H | cyclo-PrCO |
| 15-264 | C—CF3 | C—H | C—H | C—H | F | CF3 | H | cyclo-PrCH2CO |
| 15-265 | C—CF3 | C—H | C—H | C—H | F | CF3 | H | CF3CH2CO |
| 15-267 | C—CF3 | C—H | C—H | C—H | F | CF3 | H | CH3S(O)CH2CO |
| 15-268 | C—CF3 | C—H | C—H | C—H | F | CF3 | H | CH3SO2CH2CO |
| 15-269 | C—CF3 | C—H | C—H | C—H | F | CF3 | H | CH3CH2NHCO |
| 15-270 | C—H | C—H | C—CF3 | C—H | F | CF3 | H | CH3CO |
| 15-271 | C—H | C—H | C—CF3 | C—H | F | CF3 | H | CH3CH2CO |
| 15-272 | C—H | C—H | C—CF3 | C—H | F | CF3 | H | n-PrCO |
| 15-273 | C—H | C—H | C—CF3 | C—H | F | CF3 | H | cyclo-PrCO |
| 15-274 | C—H | C—H | C—CF3 | C—H | F | CF3 | H | cyclo-PrCH2CO |
| 15-275 | C—H | C—H | C—CF3 | C—H | F | CF3 | H | CF3CH2CO |
| 15-277 | C—H | C—H | C—CF3 | C—H | F | CF3 | H | CH3S(O)CH2CO |
| 15-278 | C—H | C—H | C—CF3 | C—H | F | CF3 | H | CH3SO2CH2CO |
| 15-279 | C—H | C—H | C—CF3 | C—H | F | CF3 | H | CH3CH2NHCO |
| 15-280 | C—Cl | C—H | C—CF3 | C—H | H | CF3 | H | CH3CO |
| 15-281 | C—Cl | C—H | C—CF3 | C—H | H | CF3 | H | CH3CH2CO |
| 15-282 | C—Cl | C—H | C—CF3 | C—H | H | CF3 | H | n-PrCO |
| 15-283 | C—Cl | C—H | C—CF3 | C—H | H | CF3 | H | cyclo-PrCO |
| 15-284 | C—Cl | C—H | C—CF3 | C—H | H | CF3 | H | cyclo-PrCH2CO |
| 15-285 | C—Cl | C—H | C—CF3 | C—H | H | CF3 | H | CF3CH2CO |
| 15-287 | C—Cl | C—H | C—CF3 | C—H | H | CF3 | H | CH3CH2OCH2CH2—CO |
| 15-288 | C—Cl | C—H | C—CF3 | C—H | H | CF3 | H | CH3SCH2CO |
| 15-289 | C—Cl | C—H | C—CF3 | C—H | H | CF3 | H | CH3S(O)CH2CO |
| 15-290 | C—Cl | C—H | C—CF3 | C—H | H | CF3 | H | CH3SO2CH2CO |
| 15-291 | C—Cl | C—H | C—CF3 | C—H | H | CF3 | H | CH3CH2NHCO |
| 15-292 | C—CF3 | C—H | C—CF3 | C—H | H | CF3 | H | CH3CO |
| 15-293 | C—CF3 | C—H | C—CF3 | C—H | H | CF3 | H | CH3CH2CO |
| 15-294 | C—CF3 | C—H | C—CF3 | C—H | H | CF3 | H | n-PrCO |
| 15-295 | C—CF3 | C—H | C—CF3 | C—H | H | CF3 | H | cyclo-PrCO |
| 15-296 | C—CF3 | C—H | C—CF3 | C—H | H | CF3 | H | cyclo-PrCH2CO |
| 15-297 | C—CF3 | C—H | C—CF3 | C—H | H | CF3 | H | CF3CH2CO |
| 15-299 | C—CF3 | C—H | C—CF3 | C—H | H | CF3 | H | CH3CH2OCH2CH2—CO |
| 15-300 | C—CF3 | C—H | C—CF3 | C—H | H | CF3 | H | CH3SCH2CO |
| 15-301 | C—CF3 | C—H | C—CF3 | C—H | H | CF3 | H | CH3S(O)CH2CO |
| 15-302 | C—CF3 | C—H | C—CF3 | C—H | H | CF3 | H | CH3SO2CH2CO |
| 15-303 | C—CF3 | C—H | C—CF3 | C—H | H | CF3 | H | CH3CH2NHCO |
| 15-304 | C—Cl | C—Cl | C—CF3 | C—H | H | CF3 | H | CH3CO |
| 15-305 | C—Cl | C—Cl | C—CF3 | C—H | H | CF3 | H | CH3CH2CO |
| 15-306 | C—Cl | C—Cl | C—CF3 | C—H | H | CF3 | H | n-PrCO |
| 15-307 | C—Cl | C—Cl | C—CF3 | C—H | H | CF3 | H | cyclo-PrCO |
| 15-308 | C—Cl | C—Cl | C—CF3 | C—H | H | CF3 | H | cyclo-PrCH2CO |
| 15-309 | C—Cl | C—Cl | C—CF3 | C—H | H | CF3 | H | CF3CH2CO |
| 15-311 | C—Cl | C—Cl | C—CF3 | C—H | H | CF3 | H | CH3S(O)CH2CO |
| 15-312 | C—Cl | C—Cl | C—CF3 | C—H | H | CF3 | H | CH3SO2CH2CO |
| 15-313 | C—Cl | C—Cl | C—CF3 | C—H | H | CF3 | H | CH3CH2NHCO |
| 15-314 | C—Cl | N | C—H | C—H | H | CF3 | H | CH3CO |
| 15-315 | C—Cl | N | C—H | C—H | H | CF3 | H | CH3CH2CO |
| 15-316 | C—Cl | N | C—H | C—H | H | CF3 | H | n-PrCO |
| 15-317 | C—Cl | N | C—H | C—H | H | CF3 | H | cyclo-PrCO |
| 15-318 | C—Cl | N | C—H | C—H | H | CF3 | H | cyclo-PrCH2CO |
| 15-319 | C—Cl | N | C—H | C—H | H | CF3 | H | CF3CH2CO |
| 15-321 | C—Cl | N | C—H | C—H | H | CF3 | H | CH3S(O)CH2CO |
| 15-322 | C—Cl | N | C—H | C—H | H | CF3 | H | CH3SO2CH2CO |
| 15-323 | C—Cl | N | C—H | C—H | H | CF3 | H | CH3CH2NHCO |
| 15-324 | C—Cl | N | C—Cl | C—H | H | CF3 | H | CH3CO |
| 15-325 | C—Cl | N | C—Cl | C—H | H | CF3 | H | CH3CH2CO |
| 15-326 | C—Cl | N | C—Cl | C—H | H | CF3 | H | n-PrCO |
| 15-327 | C—Cl | N | C—Cl | C—H | H | CF3 | H | cyclo-PrCO |
| 15-328 | C—Cl | N | C—Cl | C—H | H | CF3 | H | cyclo-PrCH2CO |
| 15-329 | C—Cl | N | C—Cl | C—H | H | CF3 | H | CF3CH2CO |
| 15-330 | C—Cl | N | C—Cl | C—H | H | CF3 | H | CH3SCH2CO |
| 15-331 | C—Cl | N | C—Cl | C—H | H | CF3 | H | CH3S(O)CH2CO |
| 15-332 | C—Cl | N | C—Cl | C—H | H | CF3 | H | CH3SO2CH2CO |
| 15-333 | C—Cl | N | C—Cl | C—H | H | CF3 | H | CH3CH2NHCO |
| 15-334 | C—CF3 | N | C—H | C—H | H | CF3 | H | CH3CO |
| 15-335 | C—CF3 | N | C—H | C—H | H | CF3 | H | CH3CH2CO |
| 15-336 | C—CF3 | N | C—H | C—H | H | CF3 | H | n-PrCO |
| 15-337 | C—CF3 | N | C—H | C—H | H | CF3 | H | cyclo-PrCO |
| 15-338 | C—CF3 | N | C—H | C—H | H | CF3 | H | cyclo-PrCH2CO |
| 15-339 | C—CF3 | N | C—H | C—H | H | CF3 | H | CF3CH2CO |
| 15-340 | C—CF3 | N | C—H | C—H | H | CF3 | H | CH3SCH2CO |
| 15-341 | C—CF3 | N | C—H | C—H | H | CF3 | H | CH3S(O)CH2CO |
| 15-342 | C—CF3 | N | C—H | C—H | H | CF3 | H | CH3SO2CH2CO |
| 15-343 | C—CF3 | N | C—H | C—H | H | CF3 | H | CH3CH2NHCO |
| 15-344 | C—CF3 | N | C—CF3 | C—H | H | CF3 | H | CH3CO |
| 15-345 | C—CF3 | N | C—CF3 | C—H | H | CF3 | H | CH3CH2CO |
| 15-346 | C—CF3 | N | C—CF3 | C—H | H | CF3 | H | n-PrCO |
| 15-347 | C—CF3 | N | C—CF3 | C—H | H | CF3 | H | cyclo-PrCO |
| 15-348 | C—CF3 | N | C—CF3 | C—H | H | CF3 | H | cyclo-PrCH2CO |
| 15-349 | C—CF3 | N | C—CF3 | C—H | H | CF3 | H | CF3CH2CO |
| 15-350 | C—CF3 | N | C—CF3 | C—H | H | CF3 | H | CH3SCH2CO |
| 15-351 | C—CF3 | N | C—CF3 | C—H | H | CF3 | H | CH3S(O)CH2CO |
| 15-352 | C—CF3 | N | C—CF3 | C—H | H | CF3 | H | CH3SO2CH2CO |
| 15-353 | C—CF3 | N | C—CF3 | C—H | H | CF3 | H | CH3CH2NHCO |
| 15-354 | C—CF3 | C—H | C—H | N | H | CF3 | H | CH3CO |
| 15-355 | C—CF3 | C—H | C—H | N | H | CF3 | H | CH3CH2CO |
| 15-356 | C—CF3 | C—H | C—H | N | H | CF3 | H | n-PrCO |
| 15-357 | C—CF3 | C—H | C—H | N | H | CF3 | H | cyclo-PrCO |
| 15-358 | C—CF3 | C—H | C—H | N | H | CF3 | H | cyclo-PrCH2CO |
| 15-359 | C—CF3 | C—H | C—H | N | H | CF3 | H | CF3CH2CO |
| 15-360 | C—CF3 | C—H | C—H | N | H | CF3 | H | CH3SCH2CO |
| 15-361 | C—CF3 | C—H | C—H | N | H | CF3 | H | CH3S(O)CH2CO |
| 15-362 | C—CF3 | C—H | C—H | N | H | CF3 | H | CH3SO2CH2CO |
| 15-363 | C—CF3 | C—H | C—H | N | H | CF3 | H | CH3CH2NHCO |
| 15-364 | C—H | C—H | C—CF3 | N | H | CF3 | H | CH3CO |
| 15-365 | C—H | C—H | C—CF3 | N | H | CF3 | H | CH3CH2CO |
| 15-366 | C—H | C—H | C—CF3 | N | H | CF3 | H | n-PrCO |
| 15-367 | C—H | C—H | C—CF3 | N | H | CF3 | H | cyclo-PrCO |
| 15-368 | C—H | C—H | C—CF3 | N | H | CF3 | H | cyclo-PrCH2CO |
| 15-369 | C—H | C—H | C—CF3 | N | H | CF3 | H | CF3CH2CO |
| 15-370 | C—H | C—H | C—CF3 | N | H | CF3 | H | CH3SCH2CO |
| 15-371 | C—H | C—H | C—CF3 | N | H | CF3 | H | CH3S(O)CH2CO |
| 15-372 | C—H | C—H | C—CF3 | N | H | CF3 | H | CH3SO2CH2CO |
| 15-373 | C—H | C—H | C—CF3 | N | H | CF3 | H | CH3CH2NHCO |
| 15-374 | C—CF3 | C—H | C—Cl | N | H | CF3 | H | CH3CO |
| 15-375 | C—CF3 | C—H | C—Cl | N | H | CF3 | H | CH3CH2CO |
| 15-376 | C—CF3 | C—H | C—Cl | N | H | CF3 | H | n-PrCO |
| 15-377 | C—CF3 | C—H | C—Cl | N | H | CF3 | H | cyclo-PrCO |
| 15-378 | C—CF3 | C—H | C—Cl | N | H | CF3 | H | cyclo-PrCH2CO |
| 15-379 | C—CF3 | C—H | C—Cl | N | H | CF3 | H | CF3CH2CO |
| 15-380 | C—CF3 | C—H | C—Cl | N | H | CF3 | H | CH3SCH2CO |
| 15-381 | C—CF3 | C—H | C—Cl | N | H | CF3 | H | CH3S(O)CH2CO |
| 15-382 | C—CF3 | C—H | C—Cl | N | H | CF3 | H | CH3SO2CH2CO |
| 15-383 | C—CF3 | C—H | C—Cl | N | H | CF3 | H | CH3CH2NHCO |
| 15-384 | C—CF3 | C—H | C—Br | N | H | CF3 | H | CH3CO |
| 15-385 | C—CF3 | C—H | C—Br | N | H | CF3 | H | CH3CH2CO |
| 15-386 | C—CF3 | C—H | C—Br | N | H | CF3 | H | n-PrCO |
| 15-387 | C—CF3 | C—H | C—Br | N | H | CF3 | H | cyclo-PrCO |
| 15-388 | C—CF3 | C—H | C—Br | N | H | CF3 | H | cyclo-PrCH2CO |
| 15-389 | C—CF3 | C—H | C—Br | N | H | CF3 | H | CF3CH2CO |
| 15-390 | C—CF3 | C—H | C—Br | N | H | CF3 | H | CH3SCH2CO |
| 15-391 | C—CF3 | C—H | C—Br | N | H | CF3 | H | CH3S(O)CH2CO |
| 15-392 | C—CF3 | C—H | C—Br | N | H | CF3 | H | CH3SO2CH2CO |
| 15-393 | C—CF3 | C—H | C—Br | N | H | CF3 | H | CH3CH2NHCO |
| 15-394 | C—CF3 | C—H | C—CF3 | N | H | CF3 | H | CH3CO |
| 15-395 | C—CF3 | C—H | C—CF3 | N | H | CF3 | H | CH3CH2CO |
| 15-396 | C—CF3 | C—H | C—CF3 | N | H | CF3 | H | n-PrCO |
| 15-397 | C—CF3 | C—H | C—CF3 | N | H | CF3 | H | cyclo-PrCO |
| 15-398 | C—CF3 | C—H | C—CF3 | N | H | CF3 | H | cyclo-PrCH2CO |
| 15-399 | C—CF3 | C—H | C—CF3 | N | H | CF3 | H | CF3CH2CO |
| 15-400 | C—CF3 | C—H | C—CF3 | N | H | CF3 | H | CH3SCH2CO |
| 15-401 | C—CF3 | C—H | C—CF3 | N | H | CF3 | H | CH3S(O)CH2CO |
| 15-402 | C—CF3 | C—H | C—CF3 | N | H | CF3 | H | CH3SO2CH2CO |
| 15-403 | C—CF3 | C—H | C—CF3 | N | H | CF3 | H | CH3CH2NHCO |

TABLE 16

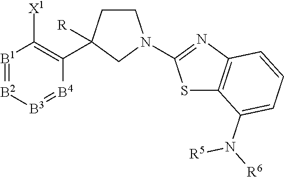

| No. | B¹ | B² | B³ | B⁴ | X¹ | R | R⁵ | R⁶ |
|---|---|---|---|---|---|---|---|---|
| 16-1 | C—Cl | C—H | C—H | C—H | H | CF3 | H | CH3CO |
| 16-2 | C—Cl | C—H | C—H | C—H | H | CF3 | H | CH3CH2CO |
| 16-3 | C—Cl | C—H | C—H | C—H | H | CF3 | H | n-PrCO |
| 16-4 | C—Cl | C—H | C—H | C—H | H | CF3 | H | cyclo-PrCO |
| 16-5 | C—Cl | C—H | C—H | C—H | H | CF3 | H | cyclo-PrCH2CO |
| 16-6 | C—Cl | C—H | C—H | C—H | H | CF3 | H | CF3CH2CO |
| 16-7 | C—Cl | C—H | C—H | C—H | H | CF3 | H | CH3SCH2CO |
| 16-8 | C—Cl | C—H | C—H | C—H | H | CF3 | H | CH3S(O)CH2CO |
| 16-9 | C—Cl | C—H | C—H | C—H | H | CF3 | H | CH3SO2CH2CO |
| 16-10 | C—Cl | C—H | C—H | C—H | H | CF3 | H | CH3CH2NHCO |
| 16-11 | C—Cl | C—H | C—H | C—H | F | CF3 | H | CH3CO |
| 16-12 | C—Cl | C—H | C—H | C—H | F | CF3 | H | CH3CH2CO |
| 16-13 | C—Cl | C—H | C—H | C—H | F | CF3 | H | n-PrCO |
| 16-14 | C—Cl | C—H | C—H | C—H | F | CF3 | H | cyclo-PrCO |
| 16-15 | C—Cl | C—H | C—H | C—H | F | CF3 | H | cyclo-PrCH2CO |
| 16-16 | C—Cl | C—H | C—H | C—H | F | CF3 | H | CF3CH2CO |
| 16-17 | C—Cl | C—H | C—H | C—H | F | CF3 | H | CH3SCH2CO |
| 16-18 | C—Cl | C—H | C—H | C—H | F | CF3 | H | CH3S(O)CH2CO |
| 16-19 | C—Cl | C—H | C—H | C—H | F | CF3 | H | CH3SO2CH2CO |
| 16-20 | C—Cl | C—H | C—H | C—H | F | CF3 | H | CH3CH2NHCO |
| 16-21 | C—H | C—H | C—Cl | C—H | F | CF3 | H | CH3CO |
| 16-22 | C—H | C—H | C—Cl | C—H | F | CF3 | H | CH3CH2CO |
| 16-23 | C—H | C—H | C—Cl | C—H | F | CF3 | H | n-PrCO |
| 16-24 | C—H | C—H | C—Cl | C—H | F | CF3 | H | cyclo-PrCO |
| 16-25 | C—H | C—H | C—Cl | C—H | F | CF3 | H | cyclo-PrCH2CO |
| 16-26 | C—H | C—H | C—Cl | C—H | F | CF3 | H | CF3CH2CO |
| 16-27 | C—H | C—H | C—Cl | C—H | F | CF3 | H | CH3SCH2CO |
| 16-28 | C—H | C—H | C—Cl | C—H | F | CF3 | H | CH3S(O)CH2CO |
| 16-29 | C—H | C—H | C—Cl | C—H | F | CF3 | H | CH3SO2CH2CO |
| 16-30 | C—H | C—H | C—Cl | C—H | F | CF3 | H | CH3CH2NHCO |
| 16-31 | C—Cl | C—H | C—Cl | C—H | H | CF3 | H | H |
| 16-32 | C—Cl | C—H | C—Cl | C—H | H | CF3 | H | HCO |
| 16-33 | C—Cl | C—H | C—Cl | C—H | H | CF3 | H | CH3CO |
| 16-34 | C—Cl | C—H | C—Cl | C—H | H | CF3 | H | CH3CH2CO |
| 16-35 | C—Cl | C—H | C—Cl | C—H | H | CF3 | H | n-PrCO |
| 16-36 | C—Cl | C—H | C—Cl | C—H | H | CF3 | H | iso-PrCO |
| 16-37 | C—Cl | C—H | C—Cl | C—H | H | CF3 | H | cyclo-PrCO |
| 16-38 | C—Cl | C—H | C—Cl | C—H | H | CF3 | H | n-BuCO |
| 16-39 | C—Cl | C—H | C—Cl | C—H | H | CF3 | H | tert-BuCO |
| 16-40 | C—Cl | C—H | C—Cl | C—H | H | CF3 | H | iso-BuCO |
| 16-41 | C—Cl | C—H | C—Cl | C—H | H | CF3 | H | cyclo-BuCO |
| 16-42 | C—Cl | C—H | C—Cl | C—H | H | CF3 | H | (CH3)3CCH2CO |
| 16-43 | C—Cl | C—H | C—Cl | C—H | H | CF3 | H | cyclo-PenCO |
| 16-44 | C—Cl | C—H | C—Cl | C—H | H | CF3 | H | cyclo-HexCO |
| 16-45 | C—Cl | C—H | C—Cl | C—H | H | CF3 | H | cyclo-PrCH2CO |
| 16-46 | C—Cl | C—H | C—Cl | C—H | H | CF3 | H | CF3CO |
| 16-47 | C—Cl | C—H | C—Cl | C—H | H | CF3 | H | CCl3CO |
| 16-48 | C—Cl | C—H | C—Cl | C—H | H | CF3 | H | ClCH2CO |
| 16-49 | C—Cl | C—H | C—Cl | C—H | H | CF3 | H | CF2ClCO |
| 16-50 | C—Cl | C—H | C—Cl | C—H | H | CF3 | H | ClCH2CH2CO |
| 16-51 | C—Cl | C—H | C—Cl | C—H | H | CF3 | H | HCF2CF2CO |
| 16-52 | C—Cl | C—H | C—Cl | C—H | H | CF3 | H | CF3CH2CO |
| 16-54 | C—Cl | C—H | C—Cl | C—H | H | CF3 | H | HCF2CO |
| 16-55 | C—Cl | C—H | C—Cl | C—H | H | CF3 | H | ClCH2CO |
| 16-56 | C—Cl | C—H | C—Cl | C—H | H | CF3 | H | 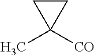 |
| 16-57 | C—Cl | C—H | C—Cl | C—H | H | CF3 | H | 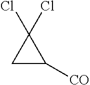 |
| 16-58 | C—Cl | C—H | C—Cl | C—H | H | CF3 | H | 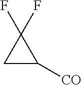 |
| 16-59 | C—Cl | C—H | C—Cl | C—H | H | CF3 | H | 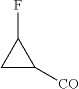 |
| 16-60 | C—Cl | C—H | C—Cl | C—H | H | CF3 | H | 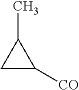 |
| 16-61 | C—Cl | C—H | C—Cl | C—H | H | CF3 | H | 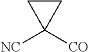 |
| 16-62 | C—Cl | C—H | C—Cl | C—H | H | CF3 | H | CH2═CH—CO |
| 16-63 | C—Cl | C—H | C—Cl | C—H | H | CF3 | H | CH3CH═CH—CO |
| 16-64 | C—Cl | C—H | C—Cl | C—H | H | CF3 | H | CH2═C(CH3)—CO |
| 16-65 | C—Cl | C—H | C—Cl | C—H | H | CF3 | H | CH3CH═C(CH3)—CO |
| 16-66 | C—Cl | C—H | C—Cl | C—H | H | CF3 | H | (CH3)2C═CH—CO |
| 16-67 | C—Cl | C—H | C—Cl | C—H | H | CF3 | H | CH2═C(Cl)—CO |
| 16-68 | C—Cl | C—H | C—Cl | C—H | H | CF3 | H | ClCH═C(CH3)—CO |
| 16-69 | C—Cl | C—H | C—Cl | C—H | H | CF3 | H | CH2═CHCH2—CO |
| 16-70 | C—Cl | C—H | C—Cl | C—H | H | CF3 | H | HCC—CO |
| 16-71 | C—Cl | C—H | C—Cl | C—H | H | CF3 | H | CH3CC—CO |
| 16-72 | C—Cl | C—H | C—Cl | C—H | H | CF3 | H | NCCH2—CO |
| 16-73 | C—Cl | C—H | C—Cl | C—H | H | CF3 | H | CH3OCH2—CO |
| 16-74 | C—Cl | C—H | C—Cl | C—H | H | CF3 | H | C2H5OCH2—CO |
| 16-75 | C—Cl | C—H | C—Cl | C—H | H | CF3 | H | CH3OCH2CH2—CO |
| 16-76 | C—Cl | C—H | C—Cl | C—H | H | CF3 | H | CH3CH2OCH2CH2—CO |
| 16-77 | C—Cl | C—H | C—Cl | C—H | H | CF3 | H | CH3SCH2CO |
| 16-78 | C—Cl | C—H | C—Cl | C—H | H | CF3 | H | CH3S(O)CH2CO |
| 16-79 | C—Cl | C—H | C—Cl | C—H | H | CF3 | H | CH3SO2CH2CO |
| 16-80 | C—Cl | C—H | C—Cl | C—H | H | CF3 | H | CH3O(CO)CH2—CO |
| 16-81 | C—Cl | C—H | C—Cl | C—H | H | CF3 | H | CH3(CO)OCH2—CO |
| 16-82 | C—Cl | C—H | C—Cl | C—H | H | CF3 | H | CH3O(CO)—CO |
| 16-83 | C—Cl | C—H | C—Cl | C—H | H | CF3 | H | CF3O(CO)—CO |
| 16-84 | C—Cl | C—H | C—Cl | C—H | H | CF3 | H | CH3(CO)CH2—CO |
| 16-85 | C—Cl | C—H | C—Cl | C—H | H | CF3 | H | (CH3)2NCH2—CO |
| 16-86 | C—Cl | C—H | C—Cl | C—H | H | CF3 | H | PhCO |
| 16-87 | C—Cl | C—H | C—Cl | C—H | H | CF3 | H | PhCH2CO |
| 16-88 | C—Cl | C—H | C—Cl | C—H | H | CF3 | H | 2-py-CO |
| 16-89 | C—Cl | C—H | C—Cl | C—H | H | CF3 | H | 3-py-CO |
| 16-90 | C—Cl | C—H | C—Cl | C—H | H | CF3 | H | 4-py-CO |
| 16-91 | C—Cl | C—H | C—Cl | C—H | H | CF3 | H | 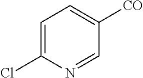 |
| 16-92 | C—Cl | C—H | C—Cl | C—H | H | CF3 | H | 2-F-PhCO |
| 16-93 | C—Cl | C—H | C—Cl | C—H | H | CF3 | H | 3-F-PhCO |
| 16-94 | C—Cl | C—H | C—Cl | C—H | H | CF3 | H | 4-F-PhCO |
| 16-95 | C—Cl | C—H | C—Cl | C—H | H | CF3 | H | 2-Cl-PhCO |
| 16-96 | C—Cl | C—H | C—Cl | C—H | H | CF3 | H | 3-Cl-PhCO |
| 16-97 | C—Cl | C—H | C—Cl | C—H | H | CF3 | H | 4-Cl-PhCO |
| 16-98 | C—Cl | C—H | C—Cl | C—H | H | CF3 | H | 2-Br-PhCO |
| 16-99 | C—Cl | C—H | C—Cl | C—H | H | CF3 | H | 2-CF3-PhCO |
| 16-100 | C—Cl | C—H | C—Cl | C—H | H | CF3 | H | 2-CH3-PhCO |
| 16-101 | C—Cl | C—H | C—Cl | C—H | H | CF3 | H | 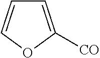 |
| 16-102 | C—Cl | C—H | C—Cl | C—H | H | CF3 | H | 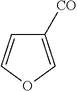 |
| 16-103 | C—Cl | C—H | C—Cl | C—H | H | CF3 | H | 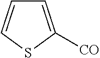 |
| 16-104 | C—Cl | C—H | C—Cl | C—H | H | CF3 | H | 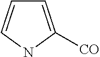 |
| 16-105 | C—Cl | C—H | C—Cl | C—H | H | CF3 | H | 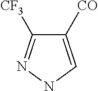 |
| 16-106 | C—Cl | C—H | C—Cl | C—H | H | CF3 | H | 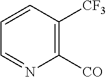 |
| 16-107 | C—Cl | C—H | C—Cl | C—H | H | CF3 | H | 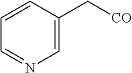 |
| 16-108 | C—Cl | C—H | C—Cl | C—H | H | CF3 | H | 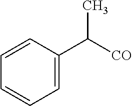 |
| 16-109 | C—Cl | C—H | C—Cl | C—H | H | CF3 | H | CH3NHCO |
| 16-110 | C—Cl | C—H | C—Cl | C—H | H | CF3 | H | CH3CH2NHCO |
| 16-111 | C—Cl | C—H | C—Cl | C—H | H | CF3 | H | CF3CH2NHCO |
| 16-112 | C—Cl | C—H | C—Cl | C—H | H | CF3 | H | n-PrNHCO |
| 16-113 | C—Cl | C—H | C—Cl | C—H | H | CF3 | H | iso-PrNHCO |
| 16-114 | C—Cl | C—H | C—Cl | C—H | H | CF3 | H | cyclo-PrNHCO |
| 16-115 | C—Cl | C—H | C—Cl | C—H | H | CF3 | H | n-BuNHCO |
| 16-116 | C—Cl | C—H | C—Cl | C—H | H | CF3 | H | tert-BuNHCO |
| 16-117 | C—Cl | C—H | C—Cl | C—H | H | CF3 | H | cyclo-BuNHCO |
| 16-118 | C—Cl | C—H | C—Cl | C—H | H | CF3 | H | cyclo-PenNHCO |
| 16-119 | C—Cl | C—H | C—Cl | C—H | H | CF3 | H | cyclo-HexNHCO |
| 16-120 | C—Cl | C—H | C—Cl | C—H | H | CF3 | H | CH2═CHCH2NHCO |
| 16-121 | C—Cl | C—H | C—Cl | C—H | H | CF3 | H | HCCCH2NHCO |
| 16-122 | C—Cl | C—H | C—Cl | C—H | H | CF3 | H | cyclo-PrCH2NHCO |
| 16-123 | C—Cl | C—H | C—Cl | C—H | H | CF3 | H | NCCH2NHCO |
| 16-124 | C—Cl | C—H | C—Cl | C—H | H | CF3 | H | CF3(CH3)CHNHCO |
| 16-125 | C—Cl | C—H | C—Cl | C—H | H | CF3 | H | CF3(CH3)2CNCO |
| 16-126 | C—Cl | C—H | C—Cl | C—H | H | CF3 | H | NCCH2OCO |
| 16-127 | C—Cl | C—H | C—Cl | C—H | H | CF3 | H | FCH2CH2NHCO |
| 16-128 | C—Cl | C—H | C—Cl | C—H | H | CF3 | H | CF3CH2CH2NHCO |
| 16-129 | C—Cl | C—H | C—Cl | C—H | H | CF3 | H | HCF2CF2CH2NHCO |
| 16-130 | C—Cl | C—H | C—Cl | C—H | H | CF3 | H | 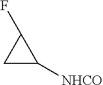 |
| 16-131 | C—Cl | C—H | C—Cl | C—H | H | CF3 | H | 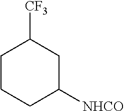 |
| 16-132 | C—Cl | C—H | C—Cl | C—H | H | CF3 | H | EtO2CCH2NHCO |
| 16-133 | C—Cl | C—H | C—Cl | C—H | H | CF3 | H | 2-CF3-PhNHCO |
| 16-134 | C—Cl | C—H | C—Cl | C—H | H | CF3 | H | 3-CF3-PhNHCO |
| 16-135 | C—Cl | C—H | C—Cl | C—H | H | CF3 | H | 4-CF3-PhNHCO |
| 16-136 | C—Cl | C—H | C—Cl | C—H | H | CF3 | H | 2-py-NHCO |
| 16-137 | C—Cl | C—H | C—Cl | C—H | H | CF3 | H | 3-py-NHCO |
| 16-138 | C—Cl | C—H | C—Cl | C—H | H | CF3 | H | 4-py-NHCO |
| 16-139 | C—Cl | C—H | C—Cl | C—H | H | CF3 | H | 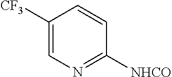 |
| 16-140 | C—Cl | C—H | C—Cl | C—H | H | CF3 | H | 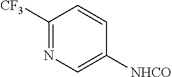 |
| 16-141 | C—Cl | C—H | C—Cl | C—H | H | CF3 | H | PhCH2HNCO |
| 16-142 | C—Cl | C—H | C—Cl | C—H | H | CF3 | H | 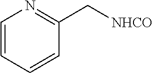 |
| 16-143 | C—Cl | C—H | C—Cl | C—H | H | CF3 | H | (CH3)2NCO |
| 16-144 | C—Cl | C—H | C—Cl | C—H | H | CF3 | H | pyrrolidineCO |
| 16-145 | C—Cl | C—H | C—Cl | C—H | H | CF3 | H | piperidineCO |
| 16-146 | C—Cl | C—H | C—Cl | C—H | H | CF3 | H | morpholineCO |
| 16-147 | C—Cl | C—H | C—Cl | C—H | H | CF3 | H | CH3OCONH |
| 16-148 | C—Cl | C—H | C—Cl | C—H | H | CF3 | H | CH3CH2OCO |
| 16-149 | C—Cl | C—H | C—Cl | C—H | H | CF3 | H | n-PrOCO |
| 16-150 | C—Cl | C—H | C—Cl | C—H | H | CF3 | H | cyclo-PrOCO |
| 16-151 | C—Cl | C—H | C—Cl | C—H | H | CF3 | H | CF3CH2OCO |
| 16-152 | C—Cl | C—H | C—Cl | C—H | H | CF3 | H | CH2═CHCH2OCO |
| 16-153 | C—Cl | C—H | C—Cl | C—H | H | CF3 | H | HCCCH2OCO |
| 16-154 | C—Cl | C—H | C—Cl | C—H | H | CF3 | H | PhOCO |
| 16-155 | C—Cl | C—H | C—Cl | C—H | H | CF3 | H | PhCH2OCO |
| 16-156 | C—Cl | C—H | C—Cl | C—H | H | CF3 | H | tert-BuOCO |
| 16-157 | C—Cl | C—H | C—Cl | C—H | H | CF3 | H | CH3SO2 |
| 16-158 | C—Cl | C—H | C—Cl | C—H | H | CF3 | H | CH3CH2SO2 |
| 16-159 | C—Cl | C—H | C—Cl | C—H | H | CF3 | H | CF3SO2 |
| 16-160 | C—Cl | C—H | C—Cl | C—H | H | CF3 | H | CF3CH2SO2 |
| 16-161 | C—Cl | C—H | C—Cl | C—H | H | CF3 | H | PhSO2 |
| 16-162 | C—Cl | C—H | C—Cl | C—H | H | CF3 | H | CH3NHCONHSO2 |
| 16-163 | C—Cl | C—H | C—Cl | C—H | H | CF3 | H | (CH3)2NCONHSO2 |
| 16-164 | C—Cl | C—H | C—Cl | C—H | H | CF3 | H | CH3CS |
| 16-165 | C—Cl | C—H | C—Cl | C—H | H | CF3 | H | CH3CH2CS |
| 16-166 | C—Cl | C—H | C—Cl | C—H | H | CF3 | H | cyclo-PrCS |
| 16-167 | C—Cl | C—H | C—Cl | C—H | H | CF3 | H | CF3CH2CS |
| 16-168 | C—Cl | C—H | C—Br | C—H | H | CF3 | H | CH3CO |
| 16-169 | C—Cl | C—H | C—Br | C—H | H | CF3 | H | CH3CH2CO |
| 16-170 | C—Cl | C—H | C—Br | C—H | H | CF3 | H | n-PrCO |
| 16-171 | C—Cl | C—H | C—Br | C—H | H | CF3 | H | cyclo-PrCO |
| 16-172 | C—Cl | C—H | C—Br | C—H | H | CF3 | H | cyclo-PrCH2CO |
| 16-173 | C—Cl | C—H | C—Br | C—H | H | CF3 | H | CF3CH2CO |
| 16-175 | C—Cl | C—H | C—Br | C—H | H | CF3 | H | CH3S(O)CH2CO |
| 16-176 | C—Cl | C—H | C—Br | C—H | H | CF3 | H | CH3SO2CH2CO |
| 16-177 | C—Cl | C—H | C—Br | C—H | H | CF3 | H | CH3CH2NHCO |
| 16-178 | C—Br | C—H | C—Br | C—H | H | CF3 | H | CH3CO |
| 16-179 | C—Br | C—H | C—Br | C—H | H | CF3 | H | CH3CH2CO |
| 16-180 | C—Br | C—H | C—Br | C—H | H | CF3 | H | n-PrCO |
| 16-181 | C—Br | C—H | C—Br | C—H | H | CF3 | H | cyclo-PrCO |
| 16-182 | C—Br | C—H | C—Br | C—H | H | CF3 | H | cyclo-PrCH2CO |
| 16-183 | C—Br | C—H | C—Br | C—H | H | CF3 | H | CF3CH2CO |
| 16-185 | C—Br | C—H | C—Br | C—H | H | CF3 | H | CH3S(O)CH2CO |
| 16-186 | C—Br | C—H | C—Br | C—H | H | CF3 | H | CH3SO2CH2CO |
| 16-187 | C—Br | C—H | C—Br | C—H | H | CF3 | H | CH3CH2NHCO |
| 16-188 | C—Cl | C—F | C—Cl | C—H | H | CF3 | H | CH3CO |
| 16-189 | C—Cl | C—F | C—Cl | C—H | H | CF3 | H | CH3CH2CO |
| 16-190 | C—Cl | C—F | C—Cl | C—H | H | CF3 | H | n-PrCO |
| 16-191 | C—Cl | C—F | C—Cl | C—H | H | CF3 | H | cyclo-PrCO |
| 16-192 | C—Cl | C—F | C—Cl | C—H | H | CF3 | H | cyclo-PrCH2CO |
| 16-193 | C—Cl | C—F | C—Cl | C—H | H | CF3 | H | CF3CH2CO |
| 16-195 | C—Cl | C—F | C—Cl | C—H | H | CF3 | H | CH3S(O)CH2CO |
| 16-196 | C—Cl | C—F | C—Cl | C—H | H | CF3 | H | CH3SO2CH2CO |
| 16-197 | C—Cl | C—F | C—Cl | C—H | H | CF3 | H | CH3CH2NHCO |
| 16-198 | C—Cl | C—Cl | C—Cl | C—H | H | CF3 | H | CH3CO |
| 16-199 | C—Cl | C—Cl | C—Cl | C—H | H | CF3 | H | CH3CH2CO |
| 16-200 | C—Cl | C—Cl | C—Cl | C—H | H | CF3 | H | n-PrCO |
| 16-201 | C—Cl | C—Cl | C—Cl | C—H | H | CF3 | H | cyclo-PrCO |
| 16-202 | C—Cl | C—Cl | C—Cl | C—H | H | CF3 | H | cyclo-PrCH2CO |
| 16-203 | C—Cl | C—Cl | C—Cl | C—H | H | CF3 | H | CF3CH2CO |
| 16-205 | C—Cl | C—Cl | C—Cl | C—H | H | CF3 | H | CH3CH2OCH2CH2—CO |
| 16-206 | C—Cl | C—Cl | C—Cl | C—H | H | CF3 | H | CH3SCH2CO |
| 16-207 | C—Cl | C—Cl | C—Cl | C—H | H | CF3 | H | CH3S(O)CH2CO |
| 16-208 | C—Cl | C—Cl | C—Cl | C—H | H | CF3 | H | CH3SO2CH2CO |
| 16-209 | C—Cl | C—Cl | C—Cl | C—H | H | CF3 | H | CH3CH2NHCO |
| 16-210 | C—Cl | C—Br | C—Cl | C—H | H | CF3 | H | CH3CO |
| 16-211 | C—Cl | C—Br | C—Cl | C—H | H | CF3 | H | CH3CH2CO |
| 16-212 | C—Cl | C—Br | C—Cl | C—H | H | CF3 | H | n-PrCO |
| 16-213 | C—Cl | C—Br | C—Cl | C—H | H | CF3 | H | cyclo-PrCO |
| 16-214 | C—Cl | C—Br | C—Cl | C—H | H | CF3 | H | cyclo-PrCH2CO |
| 16-215 | C—Cl | C—Br | C—Cl | C—H | H | CF3 | H | CF3CH2CO |
| 16-217 | C—Cl | C—Br | C—Cl | C—H | H | CF3 | H | CH3S(O)CH2CO |
| 16-218 | C—Cl | C—Br | C—Cl | C—H | H | CF3 | H | CH3SO2CH2CO |
| 16-219 | C—Cl | C—Br | C—Cl | C—H | H | CF3 | H | CH3CH2NHCO |
| 16-220 | C—Cl | C—Cl | C—Br | C—H | H | CF3 | H | CH3CO |
| 16-221 | C—Cl | C—Cl | C—Br | C—H | H | CF3 | H | CH3CH2CO |
| 16-222 | C—Cl | C—Cl | C—Br | C—H | H | CF3 | H | n-PrCO |
| 16-223 | C—Cl | C—Cl | C—Br | C—H | H | CF3 | H | cyclo-PrCO |
| 16-224 | C—Cl | C—Cl | C—Br | C—11 | H | CF3 | H | cyclo-PrCH2CO |
| 16-225 | C—Cl | C—Cl | C—Br | C—H | H | CF3 | H | CF3CH2CO |
| 16-227 | C—Cl | C—Cl | C—Br | C—H | H | CF3 | H | CH3S(O)CH2CO |
| 16-228 | C—Cl | C—Cl | C—Br | C—H | H | CF3 | H | CH3SO2CH2CO |
| 16-229 | C—Cl | C—Cl | C—Br | C—H | H | CF3 | H | CH3CH2NHCO |
| 16-230 | C—Cl | C—F | C—Cl | C—H | F | CF3 | H | CH3CO |
| 16-231 | C—Cl | C—F | C—Cl | C—H | F | CF3 | H | CH3CH2CO |
| 16-232 | C—Cl | C—F | C—Cl | C—H | F | CF3 | H | n-PrCO |
| 16-233 | C—Cl | C—F | C—Cl | C—H | F | CF3 | H | cyclo-PrCO |
| 16-234 | C—Cl | C—F | C—Cl | C—H | F | CF3 | H | cyclo-PrCH2CO |
| 16-235 | C—Cl | C—F | C—Cl | C—H | F | CF3 | H | CF3CH2CO |
| 16-237 | C—Cl | C—F | C—Cl | C—H | F | CF3 | H | CH3S(O)CH2CO |
| 16-238 | C—Cl | C—F | C—Cl | C—H | F | CF3 | H | CH3SO2CH2CO |
| 16-239 | C—Cl | C—F | C—Cl | C—H | F | CF3 | H | CH3CH2NHCO |
| 16-240 | C—CF3 | C—H | C—H | C—H | H | CF3 | H | CH3CO |
| 16-241 | C—CF3 | C—H | C—H | C—H | H | CF3 | H | CH3CH2CO |
| 16-242 | C—CF3 | C—H | C—H | C—H | H | CF3 | H | n-PrCO |
| 16-243 | C—CF3 | C—H | C—H | C—H | H | CF3 | H | cyclo-PrCO |
| 16-244 | C—CF3 | C—H | C—H | C—H | H | CF3 | H | cyclo-PrCH2CO |
| 16-245 | C—CF3 | C—H | C—H | C—H | H | CF3 | H | CF3CH2CO |
| 16-247 | C—CF3 | C—H | C—H | C—H | H | CF3 | H | CH3S(O)CH2CO |
| 16-248 | C—CF3 | C—H | C—H | C—H | H | CF3 | H | CH3SO2CH2CO |
| 16-249 | C—CF3 | C—H | C—H | C—H | H | CF3 | H | CH3CH2NHCO |
| 16-250 | C—CF3 | C—F | C—H | C—H | H | CF3 | H | CH3CO |
| 16-251 | C—CF3 | C—F | C—H | C—H | H | CF3 | H | CH3CH2CO |
| 16-252 | C—CF3 | C—F | C—H | C—H | H | CF3 | H | n-PrCO |
| 16-253 | C—CF3 | C—F | C—H | C—H | H | CF3 | H | cyclo-PrCO |
| 16-254 | C—CF3 | C—F | C—H | C—H | H | CF3 | H | cyclo-PrCH2CO |
| 16-255 | C—CF3 | C—F | C—H | C—H | H | CF3 | H | CF3CH2CO |
| 16-257 | C—CF3 | C—F | C—H | C—H | H | CF3 | H | CH3S(O)CH2CO |
| 16-258 | C—CF3 | C—F | C—H | C—H | H | CF3 | H | CH3SO2CH2CO |
| 16-259 | C—CF3 | C—F | C—H | C—H | H | CF3 | H | CH3CH2NHCO |
| 16-260 | C—CF3 | C—H | C—H | C—H | F | CF3 | H | CH3CO |
| 16-261 | C—CF3 | C—H | C—H | C—H | F | CF3 | H | CH3CH2CO |
| 16-262 | C—CF3 | C—H | C—H | C—H | F | CF3 | H | n-PrCO |
| 16-263 | C—CF3 | C—H | C—H | C—H | F | CF3 | H | cyclo-PrCO |
| 16-264 | C—CF3 | C—H | C—H | C—H | F | CF3 | H | cyclo-PrCH2CO |
| 16-265 | C—CF3 | C—H | C—H | C—H | F | CF3 | H | CF3CH2CO |
| 16-267 | C—CF3 | C—H | C—H | C—H | F | CF3 | H | CH3S(O)CH2CO |
| 16-268 | C—CF3 | C—H | C—H | C—H | F | CF3 | H | CH3SO2CH2CO |
| 16-269 | C—CF3 | C—H | C—H | C—H | F | CF3 | H | CH3CH2NHCO |
| 16-270 | C—H | C—H | C—CF3 | C—H | F | CF3 | H | CH3CO |
| 16-271 | C—H | C—H | C—CF3 | C—H | F | CF3 | H | CH3CH2CO |
| 16-272 | C—H | C—H | C—CF3 | C—H | F | CF3 | H | n-PrCO |
| 16-273 | C—H | C—H | C—CF3 | C—H | F | CF3 | H | cyclo-PrCO |
| 16-274 | C—H | C—H | C—CF3 | C—H | F | CF3 | H | cyclo-PrCH2CO |
| 16-275 | C—H | C—H | C—CF3 | C—H | F | CF3 | H | CF3CH2CO |
| 16-277 | C—H | C—H | C—CF3 | C—H | F | CF3 | H | CH3S(O)CH2CO |
| 16-278 | C—H | C—H | C—CF3 | C—H | F | CF3 | H | CH3SO2CH2CO |
| 16-279 | C—H | C—H | C—CF3 | C—H | F | CF3 | H | CH3CH2NHCO |
| 16-280 | C—Cl | C—H | C—CF3 | C—H | H | CF3 | H | CH3CO |
| 16-281 | C—Cl | C—H | C—CF3 | C—H | H | CF3 | H | CH3CH2CO |
| 16-282 | C—Cl | C—H | C—CF3 | C—H | H | CF3 | H | n-PrCO |
| 16-283 | C—Cl | C—H | C—CF3 | C—H | H | CF3 | H | cyclo-PrCO |
| 16-284 | C—Cl | C—H | C—CF3 | C—H | H | CF3 | H | cyclo-PrCH2CO |
| 16-285 | C—Cl | C—H | C—CF3 | C—H | H | CF3 | H | CF3CH2CO |
| 16-287 | C—Cl | C—H | C—CF3 | C—H | H | CF3 | H | CH3CH2OCH2CH2—CO |
| 16-288 | C—Cl | C—H | C—CF3 | C—H | H | CF3 | H | CH3SCH2CO |
| 16-289 | C—Cl | C—H | C—CF3 | C—H | H | CF3 | H | CH3S(O)CH2CO |
| 16-290 | C—Cl | C—H | C—CF3 | C—H | H | CF3 | H | CH3SO2CH2CO |
| 16-291 | C—Cl | C—H | C—CF3 | C—H | H | CF3 | H | CH3CH2NHCO |
| 16-292 | C—CF3 | C—H | C—CF3 | C—H | H | CF3 | H | CH3CO |
| 16-293 | C—CF3 | C—H | C—CF3 | C—H | H | CF3 | H | CH3CH2CO |
| 16-294 | C—CF3 | C—H | C—CF3 | C—H | H | CF3 | H | n-PrCO |
| 16-295 | C—CF3 | C—H | C—CF3 | C—H | H | CF3 | H | cyclo-PrCO |
| 16-296 | C—CF3 | C—H | C—CF3 | C—H | H | CF3 | H | cyclo-PrCH2CO |
| 16-297 | C—CF3 | C—H | C—CF3 | C—H | H | CF3 | H | CF3CH2CO |
| 16-299 | C—CF3 | C—H | C—CF3 | C—H | H | CF3 | H | CH3CH2OCH2CH2—CO |
| 16-300 | C—CF3 | C—H | C—CF3 | C—H | H | CF3 | H | CH3SCH2CO |
| 16-301 | C—CF3 | C—H | C—CF3 | C—H | H | CF3 | H | CH3S(O)CH2CO |
| 16-302 | C—CF3 | C—H | C—CF3 | C—H | H | CF3 | H | CH3SO2CH2CO |
| 16-303 | C—CF3 | C—H | C—CF3 | C—H | H | CF3 | H | CH3CH2NHCO |
| 16-304 | C—Cl | C—Cl | C—CF3 | C—H | H | CF3 | H | CH3CO |
| 16-305 | C—Cl | C—Cl | C—CF3 | C—H | H | CF3 | H | CH3CH2CO |
| 16-306 | C—Cl | C—Cl | C—CF3 | C—H | H | CF3 | H | n-PrCO |
| 16-307 | C—Cl | C—Cl | C—CF3 | C—H | H | CF3 | H | cyclo-PrCO |
| 16-308 | C—Cl | C—Cl | C—CF3 | C—H | H | CF3 | H | cyclo-PrCH2CO |
| 16-309 | C—Cl | C—Cl | C—CF3 | C—H | H | CF3 | H | CF3CH2CO |
| 16-311 | C—Cl | C—Cl | C—CF3 | C—H | H | CF3 | H | CH3S(O)CH2CO |
| 16-312 | C—Cl | C—Cl | C—CF3 | C—H | H | CF3 | H | CH3SO2CH2CO |
| 16-313 | C—Cl | C—Cl | C—CF3 | C—H | H | CF3 | H | CH3CH2NHCO |
| 16-314 | C—Cl | N | C—H | C—H | H | CF3 | H | CH3CO |
| 16-315 | C—Cl | N | C—H | C—H | H | CF3 | H | CH3CH2CO |
| 16-316 | C—Cl | N | C—H | C—H | H | CF3 | H | n-PrCO |
| 16-317 | C—Cl | N | C—H | C—H | H | CF3 | H | cyclo-PrCO |
| 16-318 | C—Cl | N | C—H | C—H | H | CF3 | H | cyclo-PrCH2CO |
| 16-319 | C—Cl | N | C—H | C—H | H | CF3 | H | CF3CH2CO |
| 16-321 | C—Cl | N | C—H | C—H | H | CF3 | H | CH3S(O)CH2CO |
| 16-322 | C—Cl | N | C—H | C—H | H | CF3 | H | CH3SO2CH2CO |
| 16-323 | C—Cl | N | C—H | C—H | H | CF3 | H | CH3CH2NHCO |
| 16-324 | C—Cl | N | C—Cl | C—H | H | CF3 | H | CH3CO |
| 16-325 | C—Cl | N | C—Cl | C—H | H | CF3 | H | CH3CH2CO |
| 16-326 | C—Cl | N | C—Cl | C—H | H | CF3 | H | n-PrCO |
| 16-327 | C—Cl | N | C—Cl | C—H | H | CF3 | H | cyclo-PrCO |
| 16-328 | C—Cl | N | C—Cl | C—H | H | CF3 | H | cyclo-PrCH2CO |
| 16-329 | C—Cl | N | C—Cl | C—H | H | CF3 | H | CF3CH2CO |
| 16-330 | C—Cl | N | C—Cl | C—H | H | CF3 | H | CH3SCH2CO |
| 16-331 | C—Cl | N | C—Cl | C—H | H | CF3 | H | CH3S(O)CH2CO |
| 16-332 | C—Cl | N | C—Cl | C—H | H | CF3 | H | CH3SO2CH2CO |
| 16-333 | C—Cl | N | C—Cl | C—H | H | CF3 | H | CH3CH2NHCO |
| 16-334 | C—CF3 | N | C—H | C—H | H | CF3 | H | CH3CO |
| 16-335 | C—CF3 | N | C—H | C—H | H | CF3 | H | CH3CH2CO |
| 16-336 | C—CF3 | N | C—H | C—H | H | CF3 | H | n-PrCO |
| 16-337 | C—CF3 | N | C—H | C—H | H | CF3 | H | cyclo-PrCO |
| 16-338 | C—CF3 | N | C—H | C—H | H | CF3 | H | cyclo-PrCH2CO |
| 16-339 | C—CF3 | N | C—H | C—H | H | CF3 | H | CF3CH2CO |
| 16-340 | C—CF3 | N | C—H | C—H | H | CF3 | H | CH3SCH2CO |
| 16-341 | C—CF3 | N | C—H | C—H | H | CF3 | H | CH3S(O)CH2CO |
| 16-342 | C—CF3 | N | C—H | C—H | H | CF3 | H | CH3SO2CH2CO |
| 16-343 | C—CF3 | N | C—H | C—H | H | CF3 | H | CH3CH2NHCO |
| 16-344 | C—CF3 | N | C—CF3 | C—H | H | CF3 | H | CH3CO |
| 16-345 | C—CF3 | N | C—CF3 | C—H | H | CF3 | H | CH3CH2CO |
| 16-346 | C—CF3 | N | C—CF3 | C—H | H | CF3 | H | n-PrCO |
| 16-347 | C—CF3 | N | C—CF3 | C—H | H | CF3 | H | cyclo-PrCO |
| 16-348 | C—CF3 | N | C—CF3 | C—H | H | CF3 | H | cyclo-PrCH2CO |
| 16-349 | C—CF3 | N | C—CF3 | C—H | H | CF3 | H | CF3CH2CO |
| 16-350 | C—CF3 | N | C—CF3 | C—H | H | CF3 | H | CH3SCH2CO |
| 16-351 | C—CF3 | N | C—CF3 | C—H | H | CF3 | H | CH3S(O)CH2CO |
| 16-352 | C—CF3 | N | C—CF3 | C—H | H | CF3 | H | CH3SO2CH2CO |
| 16-353 | C—CF3 | N | C—CF3 | C—H | H | CF3 | H | CH3CH2NHCO |
| 16-354 | C—CF3 | C—H | C—H | N | H | CF3 | H | CH3CO |
| 16-355 | C—CF3 | C—H | C—H | N | H | CF3 | H | CH3CH2CO |
| 16-356 | C—CF3 | C—H | C—H | N | H | CF3 | H | n-PrCO |
| 16-357 | C—CF3 | C—H | C—H | N | H | CF3 | H | cyclo-PrCO |
| 16-358 | C—CF3 | C—H | C—H | N | H | CF3 | H | cyclo-PrCH2CO |
| 16-359 | C—CF3 | C—H | C—H | N | H | CF3 | H | CF3CH2CO |
| 16-360 | C—CF3 | C—H | C—H | N | H | CF3 | H | CH3SCH2CO |
| 16-361 | C—CF3 | C—H | C—H | N | H | CF3 | H | CH3S(O)CH2CO |
| 16-362 | C—CF3 | C—H | C—H | N | H | CF3 | H | CH3SO2CH2CO |
| 16-363 | C—CF3 | C—H | C—H | N | H | CF3 | H | CH3CH2NHCO |
| 16-364 | C—H | C—H | C—CF3 | N | H | CF3 | H | CH3CO |
| 16-365 | C—H | C—H | C—CF3 | N | H | CF3 | H | CH3CH2CO |
| 16-366 | C—H | C—H | C—CF3 | N | H | CF3 | H | n-PrCO |
| 16-367 | C—H | C—H | C—CF3 | N | H | CF3 | H | cyclo-PrCO |
| 16-368 | C—H | C—H | C—CF3 | N | H | CF3 | H | cyclo-PrCH2CO |
| 16-369 | C—H | C—H | C—CF3 | N | H | CF3 | H | CF3CH2CO |
| 16-370 | C—H | C—H | C—CF3 | N | H | CF3 | H | CH3SCH2CO |
| 16-371 | C—H | C—H | C—CF3 | N | H | CF3 | H | CH3S(O)CH2CO |
| 16-372 | C—H | C—H | C—CF3 | N | H | CF3 | H | CH3SO2CH2CO |
| 16-373 | C—H | C—H | C—CF3 | N | H | CF3 | H | CH3CH2NHCO |
| 16-374 | C—CF3 | C—H | C—Cl | N | H | CF3 | H | CH3CO |
| 16-375 | C—CF3 | C—H | C—Cl | N | H | CF3 | H | CH3CH2CO |
| 16-376 | C—CF3 | C—H | C—Cl | N | H | CF3 | H | n-PrCO |
| 16-377 | C—CF3 | C—H | C—Cl | N | H | CF3 | H | cyclo-PrCO |
| 16-378 | C—CF3 | C—H | C—Cl | N | H | CF3 | H | cyclo-PrCH2CO |
| 16-379 | C—CF3 | C—H | C—Cl | N | H | CF3 | H | CF3CH2CO |
| 16-380 | C—CF3 | C—H | C—Cl | N | H | CF3 | H | CH3SCH2CO |
| 16-381 | C—CF3 | C—H | C—Cl | N | H | CF3 | H | CH3S(O)CH2CO |
| 16-382 | C—CF3 | C—H | C—Cl | N | H | CF3 | H | CH3SO2CH2CO |
| 16-383 | C—CF3 | C—H | C—Cl | N | H | CF3 | H | CH3CH2NHCO |
| 16-384 | C—CF3 | C—H | C—Br | N | H | CF3 | H | CH3CO |
| 16-385 | C—CF3 | C—H | C—Br | N | H | CF3 | H | CH3CH2CO |
| 16-386 | C—CF3 | C—H | C—Br | N | H | CF3 | H | n-PrCO |
| 16-387 | C—CF3 | C—H | C—Br | N | H | CF3 | H | cyclo-PrCO |
| 16-388 | C—CF3 | C—H | C—Br | N | H | CF3 | H | cyclo-PrCH2CO |
| 16-389 | C—CF3 | C—H | C—Br | N | H | CF3 | H | CF3CH2CO |
| 16-390 | C—CF3 | C—H | C—Br | N | H | CF3 | H | CH3SCH2CO |
| 16-391 | C—CF3 | C—H | C—Br | N | H | CF3 | H | CH3S(O)CH2CO |
| 16-392 | C—CF3 | C—H | C—Br | N | H | CF3 | H | CH3SO2CH2CO |
| 16-393 | C—CF3 | C—H | C—Br | N | H | CF3 | H | CH3CH2NHCO |
| 16-394 | C—CF3 | C—H | C—CF3 | N | H | CF3 | H | CH3CO |
| 16-395 | C—CF3 | C—H | C—CF3 | N | H | CF3 | H | CH3CH2CO |
| 16-396 | C—CF3 | C—H | C—CF3 | N | H | CF3 | H | n-PrCO |
| 16-397 | C—CF3 | C—H | C—CF3 | N | H | CF3 | H | cyclo-PrCO |
| 16-398 | C—CF3 | C—H | C—CF3 | N | H | CF3 | H | cyclo-PrCH2CO |
| 16-399 | C—CF3 | C—H | C—CF3 | N | H | CF3 | H | CF3CH2CO |
| 16-400 | C—CF3 | C—H | C—CF3 | N | H | CF3 | H | CH3SCH2CO |
| 16-401 | C—CF3 | C—H | C—CF3 | N | H | CF3 | H | CH3S(O)CH2CO |
| 16-402 | C—CF3 | C—H | C—CF3 | N | H | CF3 | H | CH3SO2CH2CO |
| 16-403 | C—CF3 | C—H | C—CF3 | N | H | CF3 | H | CH3CH2NHCO |

TABLE 17

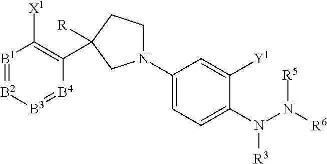

| No. | B¹ | B² | B³ | B⁴ | X¹ | R | Y¹ | R³ | R⁵ | R⁶ |
|---|---|---|---|---|---|---|---|---|---|---|
| 17-1 | C—Cl | C—H | C—Cl | C—H | H | CF3 | C—CF3 | H | H | H |
| 17-2 | C—Cl | C—H | C—Cl | C—H | H | CF3 | C—CF3 | H | H | tert-BuOCO |
| 17-3 | C—Cl | C—H | C—Cl | C—H | H | CF3 | C—CF3 | H | H | CH3CO |
| 17-4 | C—Cl | C—H | C—Cl | C—H | H | CF3 | C—CF3 | H | H | CH3CH2CO |
| 17-5 | C—Cl | C—H | C—Cl | C—H | H | CF3 | C—CF3 | H | H | nPrCO |
| 17-6 | C—Cl | C—H | C—Cl | C—H | H | CF3 | C—CF3 | H | H | cyclo-PrCO |
| 17-7 | C—Cl | C—H | C—Cl | C—H | H | CF3 | C—CF3 | H | H | cyclo-PrCH2CO |
| 17-8 | C—Cl | C—H | C—Cl | C—H | H | CF3 | C—CF3 | H | H | CF3CH2CO |
| 17-9 | C—Cl | C—H | C—Cl | C—H | H | CF3 | C—CF3 | H | H | CH3SCH2CO |
| 17-10 | C—Cl | C—H | C—Cl | C—H | H | CF3 | C—CF3 | H | H | CH3S(O)CH2CO |
| 17-11 | C—Cl | C—H | C—Cl | C—H | H | CF3 | C—CF3 | H | H | CH3SO2CH2CO |
| 17-12 | C—Cl | C—H | C—Cl | C—H | H | CF3 | C—CF3 | H | H | CH3CH2NHCO |
| 17-13 | C—Cl | C—H | C—Cl | C—H | H | CF3 | C—CF3 | H | H | H |
| 17-14 | C—Cl | C—H | C—Cl | C—H | H | CF3 | C—CF3 | H | H | tert-BuOCO |
| 17-15 | C—Cl | C—H | C—Cl | C—H | H | CF3 | C—CF3 | H | H | CH3CO |
| 17-16 | C—Cl | C—H | C—Cl | C—H | H | CF3 | C—CF3 | H | H | CH3CH2CO |
| 17-17 | C—Cl | C—H | C—Cl | C—H | H | CF3 | C—CF3 | H | H | nPrCO |
| 17-18 | C—Cl | C—H | C—Cl | C—H | H | CF3 | C—CF3 | H | H | cyclo-PrCO |
| 17-19 | C—Cl | C—H | C—Cl | C—H | H | CF3 | C—CF3 | H | H | cyclo-PrCH2CO |
| 17-20 | C—Cl | C—H | C—Cl | C—H | H | CF3 | C—CF3 | H | H | CF3CH2CO |
| 17-21 | C—Cl | C—H | C—Cl | C—H | H | CF3 | C—CF3 | H | H | CH3SCH2CO |
| 17-22 | C—Cl | C—H | C—Cl | C—H | H | CF3 | C—CF3 | H | H | CH3S(O)CH2CO |
| 17-23 | C—Cl | C—H | C—Cl | C—H | H | CF3 | C—CF3 | H | H | CH3SO2CH2CO |
| 17-24 | C—Cl | C—H | C—Cl | C—H | H | CF3 | C—CF3 | H | H | CH3CH2NHCO |
| 17-25 | C—Cl | C—H | C—CF3 | C—H | H | CF3 | C—CF3 | H | H | H |
| 17-26 | C—Cl | C—H | C—CF3 | C—H | H | CF3 | C—CF3 | H | H | tert-BuOCO |
| 17-27 | C—Cl | C—H | C—CF3 | C—H | H | CF3 | C—CF3 | H | H | CH3CO |
| 17-28 | C—Cl | C—H | C—CF3 | C—H | H | CF3 | C—CF3 | H | H | CH3CH2CO |
| 17-29 | C—Cl | C—H | C—CF3 | C—H | H | CF3 | C—CF3 | H | H | nPrCO |
| 17-30 | C—Cl | C—H | C—CF3 | C—H | H | CF3 | C—CF3 | H | H | cyclo-PrCO |
| 17-31 | C—Cl | C—H | C—CF3 | C—H | H | CF3 | C—CF3 | H | H | cyclo-PrCH2CO |
| 17-32 | C—Cl | C—H | C—CF3 | C—H | H | CF3 | C—CF3 | H | H | CF3CH2CO |
| 17-33 | C—Cl | C—H | C—CF3 | C—H | H | CF3 | C—CF3 | H | H | CH3SCH2CO |
| 17-34 | C—Cl | C—H | C—CF3 | C—H | H | CF3 | C—CF3 | H | H | CH3S(O)CH2CO |
| 17-35 | C—Cl | C—H | C—CF3 | C—H | H | CF3 | C—CF3 | H | H | CH3SO2CH2CO |
| 17-36 | C—Cl | C—H | C—CF3 | C—H | H | CF3 | C—CF3 | H | H | CH3CH2NHCO |
| 17-37 | C—CF3 | C—H | C—CF3 | C—H | H | CF3 | C—CF3 | H | H | H |
| 17-38 | C—CF3 | C—H | C—CF3 | C—H | H | CF3 | C—CF3 | H | H | tert-BuOCO |
| 17-39 | C—CF3 | C—H | C—CF3 | C—H | H | CF3 | C—CF3 | H | H | CH3CO |
| 17-40 | C—CF3 | C—H | C—CF3 | C—H | H | CF3 | C—CF3 | H | H | CH3CH2CO |
| 17-41 | C—CF3 | C—H | C—CF3 | C—H | H | CF3 | C—CF3 | H | H | nPrCO |
| 17-42 | C—CF3 | C—H | C—CF3 | C—H | H | CF3 | C—CF3 | H | H | cyclo-PrCO |
| 17-43 | C—CF3 | C—H | C—CF3 | C—H | H | CF3 | C—CF3 | H | H | cyclo-PrCH2CO |
| 17-44 | C—CF3 | C—H | C—CF3 | C—H | H | CF3 | C—CF3 | H | H | CF3CH2CO |
| 17-45 | C—CF3 | C—H | C—CF3 | C—H | H | CF3 | C—CF3 | H | H | CH3SCH2CO |
| 17-46 | C—CF3 | C—H | C—CF3 | C—H | H | CF3 | C—CF3 | H | H | CH3S(O)CH2CO |
| 17-47 | C—CF3 | C—H | C—CF3 | C—H | H | CF3 | C—CF3 | H | H | CH3SO2CH2CO |
| 17-48 | C—CF3 | C—H | C—CF3 | C—H | H | CF3 | C—CF3 | H | H | CH3CH2NHCO |

TABLE 18

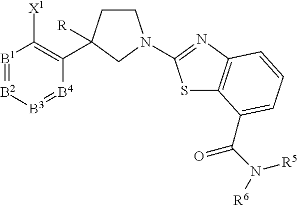

| No. | B¹ | B² | B³ | B⁴ | X¹ | R | R⁵ | R⁶ |
|---|---|---|---|---|---|---|---|---|
| 18-1 | C—Cl | C—H | C—Cl | C—H | H | CF3 | CH3 | H |
| 18-2 | C—Cl | C—H | C—Cl | C—H | H | CF3 | CH3CH2 | H |
| 18-3 | C—Cl | C—H | C—Cl | C—H | H | CF3 | CF3CH2 | H |
| 18-4 | C—Cl | C—H | C—Cl | C—H | H | CF3 | CH3OCH2 | H |
| 18-5 | C—Cl | C—H | C—Cl | C—H | H | CF3 | CH3OCH2CH2 | H |
| 18-6 | C—Cl | C—H | C—Cl | C—H | H | CF3 | CH3SCH2CH2 | H |
| 18-7 | C—Cl | C—H | C—Cl | C—H | H | CF3 | CH3S(O)CH2CH2 | H |
| 18-8 | C—Cl | C—H | C—Cl | C—H | H | CF3 | CH3SO2CH2CH2 | H |
| 18-9 | C—Cl | C—H | C—Cl | C—H | H | CF3 | 2-PyridylCH2 | H |
| 18-10 | C—Cl | C—Cl | C—Cl | C—H | H | CF3 | CH3 | H |
| 18-11 | C—Cl | C—Cl | C—Cl | C—H | H | CF3 | CH3CH2 | H |
| 18-12 | C—Cl | C—Cl | C—Cl | C—H | H | CF3 | CF3CH2 | H |
| 18-13 | C—Cl | C—Cl | C—Cl | C—H | H | CF3 | CH3OCH2 | H |
| 18-14 | C—Cl | C—Cl | C—Cl | C—H | H | CF3 | CH3OCH2CH2 | H |
| 18-15 | C—Cl | C—Cl | C—Cl | C—H | H | CF3 | CH3SCH2CH2 | H |
| 18-16 | C—Cl | C—Cl | C—Cl | C—H | H | CF3 | CH3S(O)CH2CH2 | H |
| 18-17 | C—Cl | C—Cl | C—Cl | C—H | H | CF3 | CH3SO2CH2CH2 | H |
| 18-18 | C—Cl | C—Cl | C—Cl | C—H | H | CF3 | 2-PyridylCH2 | H |
| 18-19 | C—CF3 | C—H | C—H | C—H | H | CF3 | CH3 | H |
| 18-20 | C—CF3 | C—H | C—H | C—H | H | CF3 | CH3CH2 | H |
| 18-21 | C—CF3 | C—H | C—H | C—H | H | CF3 | CF3CH2 | H |
| 18-22 | C—CF3 | C—H | C—H | C—H | H | CF3 | CH3OCH2 | H |
| 18-23 | C—CF3 | C—H | C—H | C—H | H | CF3 | CH3OCH2CH2 | H |
| 18-24 | C—CF3 | C—H | C—H | C—H | H | CF3 | CH3SCH2CH2 | H |
| 18-25 | C—CF3 | C—H | C—H | C—H | H | CF3 | CH3S(O)CH2CH2 | H |
| 18-26 | C—CF3 | C—H | C—H | C—H | H | CF3 | CH3SO2CH2CH2 | H |
| 18-27 | C—CF3 | C—H | C—H | C—H | H | CF3 | 2-PyridylCH2 | H |
| 18-28 | C—CF3 | C—H | C—CF3 | C—H | H | CF3 | CH3 | H |
| 18-29 | C—CF3 | C—H | C—CF3 | C—H | H | CF3 | CH3CH2 | H |
| 18-30 | C—CF3 | C—H | C—CF3 | C—H | H | CF3 | CF3CH2 | H |
| 18-31 | C—CF3 | C—H | C—CF3 | C—H | H | CF3 | CH3OCH2 | H |
| 18-32 | C—CF3 | C—H | C—CF3 | C—H | H | CF3 | CH3OCH2CH2 | H |
| 18-33 | C—CF3 | C—H | C—CF3 | C—H | H | CF3 | CH3SCH2CH2 | H |
| 18-34 | C—CF3 | C—H | C—CF3 | C—H | H | CF3 | CH3S(O)CH2CH2 | H |
| 18-35 | C—CF3 | C—H | C—CF3 | C—H | H | CF3 | CH3SO2CH2CH2 | H |
| 18-36 | C—CF3 | C—H | C—CF3 | C—H | H | CF3 | 2-PyridylCH2 | H |

TABLE 19

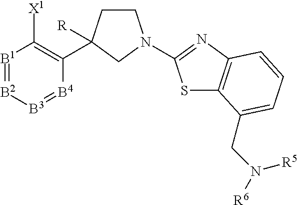

| No. | B¹ | B² | B³ | B⁴ | X¹ | R | R⁵ | R⁶ |
|---|---|---|---|---|---|---|---|---|
| 19-1 | C—Cl | C—H | C—Cl | C—H | H | CF3 | H | H |
| 19-2 | C—Cl | C—H | C—Cl | C—H | H | CF3 | H | CH3CO |
| 19-3 | C—Cl | C—H | C—Cl | C—H | H | CF3 | H | CH3CH2CO |
| 19-4 | C—Cl | C—H | C—Cl | C—H | H | CF3 | H | n-PrCO |
| 19-5 | C—Cl | C—H | C—Cl | C—H | H | CF3 | H | cyclo-PrCO |
| 19-6 | C—Cl | C—H | C—Cl | C—H | H | CF3 | H | cyclo-PrCH2CO |
| 19-7 | C—Cl | C—H | C—Cl | C—H | H | CF3 | H | CF3CH2CO |
| 19-8 | C—Cl | C—H | C—Cl | C—H | H | CF3 | H | CH3SCH2CO |
| 19-9 | C—Cl | C—H | C—Cl | C—H | H | CF3 | H | CH3S(O)CH2CO |
| 19-10 | C—Cl | C—H | C—Cl | C—H | H | CF3 | H | CH3SO2CH2CO |

TABLE 20

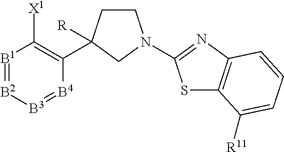

| No. | B¹ | B² | B³ | B⁴ | X¹ | R | R¹¹ |
|---|---|---|---|---|---|---|---|
| 20-1 | C—Cl | C—H | C—Cl | C—H | H | CF3 | CO2CH3 |
| 20-2 | C—Cl | C—H | C—Cl | C—H | H | CF3 | CO2H |
| 20-3 | C—Cl | C—H | C—Cl | C—H | H | CF3 | CH2OH |

TABLE 21

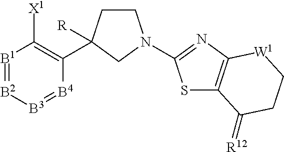

| No. | B¹ | B² | B³ | B⁴ | X¹ | R | W¹ | R¹² |
|---|---|---|---|---|---|---|---|---|
| 21-1 | C—Cl | C—H | C—Cl | C—H | H | CF3 | 1 | O |
| 21-2 | C—Cl | C—H | C—Cl | C—H | H | CF3 | 1 | HO—N |
| 21-3 | C—Cl | C—H | C—Cl | C—H | H | CF3 | 1 | HO—N |
| 21-4 | C—Cl | C—Cl | C—Cl | C—H | H | CF3 | 1 | O |
| 21-5 | C—Cl | C—Cl | C—Cl | C—H | H | CF3 | 1 | HO—N |
| 21-6 | C—CF3 | C—H | C—CF3 | C—H | H | CF3 | 1 | O |
| 21-7 | C—CF3 | C—H | C—CF3 | C—H | H | CF3 | 1 | HO—N |

| No. | 1H-NMR |
|---|---|
| 11-437 | 1H-NMR (CDCl3) δ: 1.74 (3H, t), 1.60-1.70 (1H, m), 1.80-1.88 (1H, m), |
| | 2.00-2.07 (1H, m), 2.21 (2H, q), 2.52-2.67 (2H, m), 2.80-2.92 (1H, m), |
| | 3.53-3.60 (2H, m), 3.91 (1H, d), 4.31 (1H, d), 5.04-5.15 (1H, m), |
| | 5.60-5.70 (1H, m), 7.25-7.39 (3H, m) |
| 12-33 | 1H-NMR (CDCl3) δ: 2.31 (3H, s), 2.60-2.63 (1H, m), 2.87-2.91 (1H, m), |
| | 3.59-3.65 (2H, m), 3.89 (1H, d), 4.16 (1H, d), 6.84 (1H, s), 7.02-7.07 |
| | (1H, m), 7.22-7.39 (6H, m), 7.53-7.56 (1H, m), 7.67-7.73 (1H, m). |
| 12-34 | 1H-NMR (CDCl3) δ: 1.11-1.16 (3H, m), 2.33-2.58 (3H, m), 2.85-2.90 |
| | (1H, m), 3.61-3.64 (2H, m), 3.89 (1H, d), 4.16 (1H, d), 6.83 (1H, s), |
| | 7.00-7.03 (1H, m), 7.33-7.73 (8H, m). |
| 12-37 | 1H-NMR (CDCl3) δ: 1.14-1.26 (4H, m), 1.54-1.57 (1H, m), 2.58-2.62 |
| | (1H, m), 2.88-2.91 (1H, m), 3.65-3.68 (2H, m), 3.90 (1H, d), 4.18 |
| | (1H, d), 6.85 (1H, s), 7.04-7.07 (1H, m), 7.36-7.50 (8H, m). |
| 12-156 | 1H-NMR (CDCl3) δ: 1.58 (9H, s), 2.56-2.60 (1H, m), 2.85-2.91 (1H, m), |
| | 3.64-3.67 (2H, m), 3.90 (1H, d), 4.16 (1H, d), 6.74 (1H, br s), 6.83 |
| | (1H, d), 7.01 (1H, d), 7.15-7.61 (6H, m), 7.79 (1H, d). |
| 12-199 | 1H-NMR (acetone-d6) δ: 1.30-1.32 (3H, m), 2.57-2.60 (2H, m), 2.68-2.77 |
| | (1H, m), 2.97-3.05 (1H, m), 3.67-3.71 (2H, m), 4.01 (1H, d), 4.31 |
| | (1H, d), 7.02 (1H, s), 7.19-7.22 (1H, m), 7.42-7.47 (2H, m), 7.62-7.65 |
| | (1H, m), 7.80 (2H, s), 7.96 (1H, d), 8.30 (1H, |
| 12-201 | 1H-NMR (acetone-d6) δ: 0.84-0.89 (4H, m), 1.19-1.28 (1H, m), 2.81-2.84 |
| | (1H, m), 3.04-3.06 (1H, m), 3.66-3.69 (2H, m), 4.09 (1H, d), |
| | 4.36 (1H, d), 6.96-6.98 (1H, m), 7.15-7.32 (2H, m), 7.47 (1H, d), |
| | 7.65 (1H, br s), 7.85 (2H, s), 8.04 (1H, d), 9.24 (1H, b |
| 12-293 | 1H-NMR (acetone-d6) δ: 1.30-1.34 (3H, m), 2.58-2.61 (2H, m), 2.82-2.85 |
| | (1H, m), 3.15-3.18 (1H, m), 3.69-3.77 (2H, m), 4.11 (1H, d), |
| | 4.46 (1H, d), 7.07 (1H, s), 7.24-7.27 (1H, m), 7.45-7.48 |
| | (2H, m), 7.61-7.64 (1H, m), 7.98 (1H, d), 8.21-8.27 (4H, m). |
| 12-295 | 1H-NMR (acetone-d6) δ: 0.84-0.89 (4H, m), 1.26-1.30 (1H, m), 2.90-2.94 |
| | (1H, m), 3.18-3.22 (1H, m), 3.71-3.74 (2H, m), 4.18 (1H, d), 4.54 (1H, d), |
| | 7.01 (1H, d), 7.17-7.32 (2H, m), 7.47 (1H, d), 7.62 (1H, br s), |
| | 8.04 (1H, d), 8.14 (1H, s), 8.28 (2H, s), 9. |
| 12-405 | 1H-NMR (CDCl3) δ: 1.53 (9H, s), 2.56-2.60 (1H, m), 2.86-2.88 (1H, m), |
| | 3.64-3.67 (2H, m), 3.91 (1H, d), 4.15 (1H, d), 6.73 (1H, br s), 6.83 |
| | (1H, d), 7.00 (1H, d), 7.33-7.43 (4H, m), 7.55-7.59 (1H, m), 7.77 (1H, d). |
| 12-407 | 1H-NMR (CDCl3) δ: 1.55 (9H, s), 2.63-2.68 (1H, m), 2.98-3.03 (1H, m), |
| | 3.67-3.70 (2H, m), 3.96 (1H, d), 4.29 (1H, d), 6.73 (1H, br s), 6.87 |
| | (1H, d), 7.03 (1H, d), 7.36 (1H, t), 7.48 (1H, d), 7.59 (1H, d), 7.79 |
| | (1H, d), 7.91 (3H, d). |
| 13-34 | 1H-NMR (CDCl3) δ: 1.31-1.34 (3H, m), 2.24-2.63 (3H, m), 2.78-2.90 |
| | (1H, m), 3.75-3.84 (2H, m), 4.11 (1H, d), 4.58 (1H, d), 6.75 (1H, d), |
| | 7.20-7.94 (8H, m). |
| 13-199 | 1H-NMR (CDCl3) δ: 1.27-1.32 (3H, m), 2.52-2.72 (3H, m), 2.89-2.91 |
| | (1H, m), 3.74-3.83 (2H, m), 4.12 (1H, d), 4.57 (1H, d), 6.74 (1H, d), |
| | 7.34-7.91 (7H, m). |
| 13-293 | 1H-NMR (CDCl3) δ: 1.32-1.35 (3H, m), 2.52-2.70 (3H, m), 3.04-3.08 |
| | (1H, m), 3.77-3.88 (2H, m), 4.17 (1H, d), 4.74 (1H, d), 6.77 (1H, d), |
| | 7.35-7.53 (2H, m), 7.69-7.75 (2H, m), 7.91-7.92 (4H, m). |
| 16-31 | 1H-NMR (CDCl3) δ: 2.54-2.70 (1H, m), 2.90-2.94 (1H, m), 3.73-3.80 (3H, |
| | m), 4.06 (1H, d), 4.55 (1H, d), 6.49-6.52 (1H, m), 7.23-7.35 (5H, m). |
| 16-34 | 1H-NMR (CDCl3) δ: 1.24-1.32 (3H, m), 2.49-2.58 (3H, m), 2.89-2.97 (1H, |
| | m), 3.70-3.79 (2H, m), 4.06 (1H, d), 4.50 (1H, d), 7.31 (7H, m). |
| 16-37 | 1H-NMR (CDCl3) δ: 0.88-0.99 (2H, m), 1.03-1.19 (2H, m), 1.59-1.63 |
| | (1H, m), 2.56-2.64 (1H, m), 2.89-2.97 (1H, m), 3.70-3.83 (2H, m), |
| | 4.06 (1H, d), 4.51 (1H, d), 7.14-7.54 (6H, m). |
| 16-45 | 1H-NMR (CDCl3) δ: 0.35-0.36 (2H, m), 0.74-0.76 (2H, m), 1.10-1.15 (1H, |
| | m), 2.39 (2H, d), 2.54-2.65 (1H, m), 2.91-2.95 (1H, m), 3.71-3.80 (2H, m), |
| | 4.07 (1H, d), 4.50 (1H, d), 7.26-7.74 (7H, m). |
| 16-52 | 1H-NMR (CDCl3) δ: 2.60-2.64 (1H, m), 2.93-2.96 (1H, m), 3.30 (2H, q), |
| | 3.74-3.80 (2H, m), 4.07 (1H, d), 4.52 (1H, d), 7.15-7.54 (6H, m). |
| 16-77 | 1H-NMR (CDCl3) δ: 2.24 (3H, s), 2.58-2.64 (1H, m), 2.92-2.96 (1H, m), |
| | 3.42 (2H, s), 3.73-3.84 (2H, m), 4.08 (1H, d), 4.51 (1H, d), 7.26-7.53 |
| | (6H, m), 8.79 (1H, br s). |
| 16-156 | 1H-NMR (CDCl3) δ: 1.55 (9H, s), 2.56-2.66 (1H, m), 2.91-2.96 (1H, m), |
| | 3.73-3.79 (2H, m), 4.09 (1H, d), 4.52 (1H, d), 6.32 (1H, br s), |
| | 7.26-7.43 (6H, m). |
| 18-2 | 1H-NMR (CDCl3) δ: 1.29 (3H, t, ), 2.56-2.62 (1H, m), 2.90-2.95 (1H, m), |
| | 3.45-3.64 (2H, m), 3.73-3.85 (2H, m), 4.09 (1H, dz), 4.54 (1H, d), 6.45 |
| | (1H, br s), 7.19-7.54 (5H, m), 7.67-7.75 (1H, m). |
| 18-3 | 1H-NMR (CDCl3) δ: 2.63-2.66 (1H, m), 2.94-2.99 (1H, m), 3.79-3.82 (2H, |
| | m), 4.09-4.25 (3H, m), 4.56 (1H, d), 6.58 (1H, br s), 7.26-7.45 (5H, m), |
| | 7.78-7.81 (1H, m). |
| 18-9 | 1H-NMR (CDCl3) δ: 2.57-2.62 (1H, m), 2.91-2.95 (1H, m), 3.74-3.83 (2H, |
| | m), 4.11 (1H, d), 4.55 (1H, d), 4.79-4.81 (2H, m), 7.21-7.52 (7H, m), |
| | 7.66-7.83 (2H, m), 7.96 (1H, br s), 8.57-8.59 (1H, m). |
| 19-3 | 1H-NMR (CDCl3) δ: 1.21 (3H, t), 2.25-2.38 (2H, m), 2.56-2.67 (1H, m), |
| | 2.92-2.96 (1H, m), 3.71-3.83 (2H, m), 4.08 (1H, d), 4.52-4.59 |
| | (3H, m), 5.70 (1H, br s), 7.01 (1H, d), 7.26-7.34 (4H, m), 7.57 (1H, d). |
| 19-7 | 1H-NMR (CDCl3) δ: 2.58-2.63 (1H, m), 2.92-2.96 (1H, m), 3.08-3.22 (2H, |
| | m), 3.68-3.78 (2H, m), 4.04 (1H, d), 4.49-4.64 (3H, m), 6.13 (1H, br s), |
| | 6.99 (1H, d), 7.29-7.31 (4H, m), 7.57 (1H, d). |
| 20-1 | 1H-NMR (CDCl3) δ: 2.47-2.63 (1H, m), 2.84-2.89 (1H, m), 3.56-3.76 (2H, |
| | m), 3.91-3.99 (4H, m), 4.43-4.47 (1H, m), 7.07-7.17 (2H, m), 7.29-7.43 |
| | (2H, m), 7.70-7.80 (2H, m). |
| 21-1 | 1H-NMR (CDCl3) δ: 2.11-2.19 (2H, m), 2.51-2.67 (3H, m), 2.84-2.99 (3H, |
| | m), 3.63-3.70 (2H, m), 4.01 (1H, d), 4.48 (1H, d), 7.25-7.41 (3H, m) |
| 21-2 | 1H-NMR (CDCl3) δ: 2.00-2.19 (2H, m), 2.51-2.67 (3H, m), 2.84-2.98 (3H, |
| | m), 3.62-3.72 (2H, m), 4.03 (1H, d), 4.46 (1H, d), 7.22-7.40 (3H, m) |
| 21-3 | 1H-NMR (CDCl3) δ: 1.92-2.04 (2H, m), 2.53-2.95 (6H, m), 3.65-3.71 (2H, |
| | m), 3.94-3.98 (4H, m), 4.41 (1H, d), 7.26-7.40 (3H, m) |

### PREPARATION EXAMPLE 1

### Granules

To a mixture containing 10 parts of the compound of the present invention, 30 parts of bentonite (montmorillonite), 58 parts of talc and 2 parts of lignin sulfonate was added 25 parts of water, and the mixture was well kneaded and granulated with 10 to 40 meshes by an extruding granulator and dried at 40 to 50° C. to obtain granules.

### PREPARATION EXAMPLE 2

### Granules

95 parts of clay mineral granules having particle diameter distribution within the range of 0.2 to 2 mm were put into a rotary mixer, and then wetted evenly by spraying of 5 parts of the compound of the present invention together with a liquid diluent under rotating condition and dried at 40 to 50° C. to obtain granules.

### PREPARATION EXAMPLE 3

### Emulsion

30 parts of the compound of the present invention, 55 parts of xylene, 8 parts of polyoxyethylene alkyl phenyl ether and 7 parts of calcium alkylbenzenesulfonate were mixed together to obtain the emulsion.

### PREPARATION EXAMPLE 4

### Wettable Agent

15 parts of the compound of the present invention, 80 parts of a mixture of white carbon (hydrated amorphous silicon oxide fine powder) and powdered clay (1:5), formalin condensate of 2 parts of sodium alkylbenzenesulfonate and 3 parts of sodium alkylnaphthalenesulfonate were mixed together and the mixture was crushed to obtain a wettable agent.

### PREPARATION EXAMPLE 5

### Wettable Granules

20 parts of the active compound of the present invention, 30 parts of lignin sodium sulfonate, 15 parts of bentonite and 35 parts of calcined diatomaceous earth powder were well mixed, and after addition of water, the mixture was then extruded with a screen of 0.3 mm and dried to obtain wettable granules.

### BIOLOGICAL EXAMPLES

Unless not mentioned otherwise, the test solutions were prepared as follows:

Containing as solvent: Dimethylformamide, 3 parts by weight; and as emulsifier: Polyoxyethylene alkyl phenyl ether, 1 part by weight

To prepare the test solution, 1 part by weight of an active compound is mixed with the above-mentioned amount of solvent containing the above-mentioned amount of emulsifier, and the mixture is diluted with water to the desired concentration.

Test Against Tobacco Cutworm (Spodoptera litura) Larvae

Leaves of sweet potato were immersed in the test solution at the appropriate concentration, and the leaves were dried in air. The leaves were then placed in a petri dish having a diameter of 9 cm, and ten Spodoptera litura at third instar larvae were released therein. The petri dishes were placed in a temperature-controlled chamber at 25° C. After 2 days and 4 days more sweet potato leaves were added. After 7 days, the number of dead larvae was counted to calculate the insecticidal activity. An insecticidal activity of 100% means that all larvae were killed, whereas an insecticidal activity of 0% means that no larva was killed. In the current test, the results of two petri dishes for each treatment were averaged.

In this test the following compounds showed an insecticidal activity of 100% at an active compound concentration of 100 ppm.

12-33, 12-34, 12-37, 12-199, 12-201, 12-293, 12-295, 12-407, 13-199, 13-293
Test Against Cucurbit Leaf Beetle (Aulacophora femoralis)

Leaves of cucumber were immersed in the test solution at the appropriate concentration, and the leaves were dried in air. The leaves were then put in a plastic cup containing sterilized black soil and five Aulacophora femoralis at second instar larvae were released in the cup. The cups were placed in a temperature-controlled chamber at 25° C. After 7 days, the number of dead larvae was counted, and thus the insecticidal activity was calculated. An insecticidal activity of 100% means that all larvae were killed, whereas an insecticidal activity of 0% means that no larva was killed.

In this test the following compounds showed an insecticidal activity of 100% at an active compound concentration of 100 ppm.

12-34, 12-37, 12-201
Boophilus microplus —Test (Injection)
Solvent: dimethyl sulfoxide

To produce a suitable preparation of active compound, 10 mg of active compound are dissolved in 0.5 ml solvent, and the concentrate is diluted with solvent to the desired concentration. Five adult engorged female ticks (Boophilus microplus) are injected with 1 μl compound solution into the abdomen. Ticks are transferred into replica plates and incubated in a climate chamber for a period of time. Egg deposition of fertile eggs is monitored.

After 2 days mortality in % is determined. 100% means that all eggs are infertile; 0% means that all eggs are fertile.

In this test for example, the following compounds from the preparation examples showed good activity of 100% at application rate of 20 μg/animal: 12-201

Ctenocephalides felis—Test (CTECFE)
Solvent: dimethyl sulfoxide

To produce a suitable preparation of active compound, 10 mg of active compound are dissolved in 0.5 ml solvent, and the concentrate is diluted with cattle blood to the desired concentration. Approximately 10 to 15 adult unfed (Ctenocepahlides felis) are placed in flea chambers. The blood chamber, sealed with parafilm on the bottom, are filled with cattle blood supplied with compound solution and placed on top of the flea chamber, so that the fleas are able to suck the blood. The blood chamber is heated to 37° C. whereas the flea chamber is kept at room temperature. After 2 days mortality in % is determined. 100% means that all the fleas have been killed; 0% means that none of the fleas have been killed.

In this test for example, the following compounds

from the preparation examples showed good activity of 100% at application rate of 100 ppm: 12-201
Lucilia cuprina (48 h)
Species: Lucilia cuprina 1^{st} instar larvae (age 24 hrs)
Solvent: dimethyl sulfoxide

10 mg active compound are dissolve in 0.5 ml Dimethylsulfoxid. Serial dilutions are made to obtain the desired rates. Approximately 20-30 Lucilia cuprina 1^{st} instar larvae are transferred into a test tube containing 1 cm³ of minced horse meat and 0.5 ml aqueous dilution of test compound. After 48 hrs percentage of larval mortality are recorded. 100% efficacy=all larvae are killed, % efficacy=normally developed larvae after 48 hrs.

In this test for example, the following compounds from the preparation examples showed good activity of 100% at application rate of 100 ppm: 12-201

1. Arylpyrrolidine compounds of formula (I) 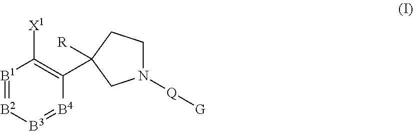
wherein
R represents optionally substituted C₁₋₁₂-alkyl or C₁₋₁₂-haloalkyl;
Q stands for a group selected among the groups Q-1 to Q-12 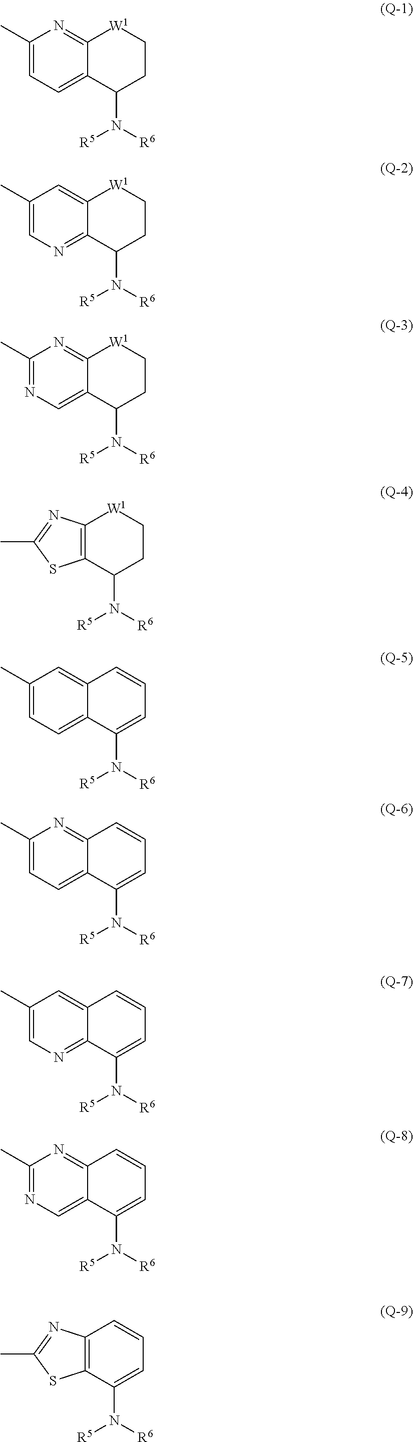 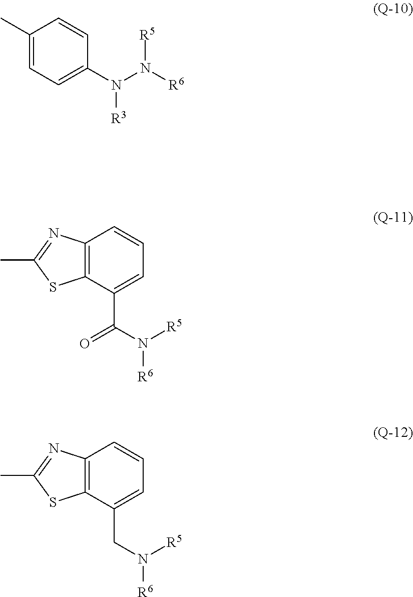
in which
W¹ stands for a single bond or methylene (i.e. a group —CH₂—), and which group may be optionally substituted by at least one substituent Y¹, whereas the substituent is preferably connected to a carbon atom:
B¹, B², B³ and B⁴ each independently represents C—X² or nitrogen, and if either B¹ and B², or B² and B³, or B³ and B⁴ stand for C—X², then the substituents X² may together with the carbon atoms to which they are bound form a 5- or 6-membered saturated or unsaturated hydrocarbon ring or heterocyclic ring;
X¹, X² each independently represents hydrogen, halogen, nitro, cyano, hydroxy, mercapto, amino, alkyl, haloalkyl, alkoxy, haloalkoxy, alkylsulfanyl, alkylsulfinyl, alkylsulfonyl, haloalkylsulfanyl, haloalkylsulfinyl, haloalkylsulfonyl, acylamino, alkoxycarbonylamino, haloalkoxycarbonylamino, alkoxyimino, haloalkoxyimino, alkylsulfonylamino, or pentafluorosulfur;
Y¹ each independently represents hydrogen, halogen, nitro, cyano, hydroxy, mercapto, amino, alkyl, haloalkyl, cycloalkyl, cyclohaloalkyl, alkenyl, alkynyl, alkoxy, haloalkoxy, alkylsulfanyl, alkylsulfinyl, alkylsulfonyl, haloalkylsulfanyl, haloalkylsulfinyl, haloalkylsulfonyl, alkylsulfonyloxy, haloalkylsulfonyloxy, alkylaminosulfonyl, haloalkylaminosulfonyl, dialkylaminosulfonyl, di(haloalkyl)aminosulfonyl, alkylamino, dialkylamino, acylamino, alkoxycarbonylamino, haloalkoxycarbonylamino, alkylsulfonylamino, haloalkylsulfonylamino, trialkylsilyl, alkoxyimino, haloalkoxyimino, alkoxyiminoalkyl, haloalkoxyiminoalkyl, alkylsulfinylimino, alkylsulfinyliminoalkyl, alkylsulfinyliminoalkylcarbonyl, alkylsulfoxyimino, alkylsulfoxyiminoalkyl, alkoxycarbonyl, alkylcarbonyl, aminocarbonyl, alkylaminocarbonyl, aminothiocarbonyl, alkylaminothiocarbonyl, dialkylaminocarbonyl, or dialkylaminothiocarbonyl;
R³ represents hydrogen, cyano, alkyl, alkenyl, alkynyl, cycloalkyl or alkoxycarbonyl;
R⁵ represents hydrogen, amino, hydroxy, cyano, alkyl, haloalkyl, cycloalkyl, alkenyl, alkynyl, alkoxy, aminocarbonylalkyl, iminoalkyl, alkylcarbonyl, alkylcarbonylamino, alkylimino, aryl, aralkyl, a heterocycle, a heterocyclic ring substituted alkyl, R⁷—C(═O)— or R′—C(═S)—;
R⁶ represents hydrogen, cyano, carbonyl, thiocarbonyl, alkylcarbonyl, alkylthiocarbonyl, haloalkylcarbonyl, haloalkylthiocarbonyl, alkylaminocarbonyl, alkylaminothiocarbonyl, dialkylaminocarbonyl, dialkylaminothiocarbonyl, alkoxyaminocarbonyl, alkoxyaminothiocarbonyl, alkoxycarbonyl, alkoxythiocarbonyl, alkylsulfanylcarbonyl, alkylsulfanylthiocarbonyl, alkylsulfonyl, haloalkylsulfonyl, cycloalkylcarbonyl, alkenylcarbonyl, alkynylcarbonyl, cycloalkylalkylcarbonyl, alkylsulfanylalkylcarbonyl, alkylsulfinylalkylcarbonyl, alkylsulfonylalkylcarbonyl, alkylcarbonylalkylcarbonyl, cycloalkylaminocarbonyl, alkenylaminocarbonyl, alkynylaminocarbonyl, alkylaminosulfonyl, dialkylaminosulfonyl, alkoxyalkylcarbonyl, alkylcarbonylaminoalkylcarbonyl, haloalkylcarbonylaminoalkylcarbonyl, alkylsulfanylalkylcarbonylaminoalkylcarbonyl, alkylsulfinylalkylcarbonylaminoalkylcarbonyl, alkylsulfonylalkylcarbonylaminoalkylcarbonyl, aralkylcarbonyl, heterocyclic substituted alkylcarbonyl, R⁷—C(═O)— or R⁷—C(═S)—, or
R⁵ and R⁶ may form a 3- to 6-membered heterocyclic ring, together with the nitrogen atom to which they are bound, and said ring may be substituted with keto, thioketo or a nitroimino group; or
R⁷ represents an optionally substituted phenyl or an optionally substituted heterocycle.

2. Compounds according to claim 1, wherein
R represents C₁₋₆ alkyl, or C₁₋₆ haloalkyl;
X¹, X² each independently represents hydrogen, halogen, nitro, cyano, hydroxy, mercapto, amino, C₁₋₆alkyl, C₁₋₆haloalkyl particularly CF₃, CF₂H, CFH₂, CH₂CF₃, CF₂CF₃, C₁₋₆alkoxy, C₁₋₆haloalkoxy, C₁₋₆alkylsulfanyl, C₁₋₆alkylsulfinyl, C₁₋₆alkylsulfonyl, C₁₋₆haloalkylsulfanyl, C₁₋₆haloalkylsulfinyl, C₁₋₆haloalkylsulfonyl, (total carbon atom number) C₂₋₇acylamino, (total carbon atom number) C₂₋₇alkoxycarbonylamino, (total carbon atom number) C₂₋₇haloalkoxycarbonylamino, C₁₋₆alkoxyimino, C₁₋₆haloalkoxyimino, C₁₋₆alkylsulfonylamino or pentafluorosulfur;
Y¹ represents hydrogen, halogen, nitro, cyano, hydroxy, mercapto, amino, C₁₋₆alkyl, C₁₋₆haloalkyl, C₃₋₇cycloalkyl, C₃₋₇cycloC₁₋₆haloalkyl, C₂₋₆alkenyl, C₂₋₆alkynyl, C₁₋₆alkoxy, C₁₋₆haloalkoxy, C₁₋₆alkylsulfanyl, C₁₋₆alkylsulfinyl, C₁₋₆alkylsulfonyl, C₁₋₆haloalkylsulfanyl, C₁₋₆haloalkylsulfinyl, C₁₋₆haloalkylsulfonyl, C₁₋₆alkylsulfonyloxy, C₁₋₆haloalkylsulfonyloxy, C₁₋₆alkylaminosulfonyl, C₁₋₆haloalkylaminosulfonyl, (total carbon atom number) C₂₋₁₂dialkylaminosulfonyl, (total carbon atom number) C₂₋₁₂di(haloalkyl)aminosulfonyl, C₁₋₆alkylamino, (total carbon atom number) C₂₋₁₂dialkylamino, (total carbon atom number) C₂₋₇acylamino, (total carbon atom number) C₂₋₇alkoxycarbonylamino, (total carbon atom number) C₂₋₇haloalkoxycarbonylamino, C₁₋₆alkylsulfonylamino, C₁₋₆haloalkylsulfonylamino, (total carbon atom number) C₃₋₁₈trialkylsilyl, C₁₋₆alkoxyimino, C₁₋₆haloalkoxyimino, (total carbon atom number) C₂₋₁₂alkoxyiminoalkyl, (total carbon atom number) C₂₋₁₂haloalkoxyiminoalkyl, C₁₋₆alkylsulfinylimino, (total carbon atom number) C₂₋₁₂alkylsulfinyliminoalkyl, (total carbon atom number) C₃₋₁₃alkylsulfinyliminoalkylcarbonyl, C₁₋₆alkylsulfoxyimino, (total carbon atom number) C₂₋₇alkylsulfoxyiminoalkyl, (total carbon atom number) C₂₋₇alkoxycarbonyl, (total carbon atom number) C₂₋₇alkylcarbonyl, aminocarbonyl, (total carbon atom number) C₂₋₇alkylaminocarbonyl, aminothiocarbonyl, (total carbon atom number) C₂₋₇alkylaminothiocarbonyl, (total carbon atom number) C₃₋₁₃dialkylaminocarbonyl or (total carbon atom number) C₃₋₁₃dialkylaminothiocarbonyl;
R³ represents hydrogen, cyano, C₁₋₆alkyl, C₂₋₆alkenyl, C₂₋₆alkynyl, C₃₋₇cycloalkyl or (total carbon atom number) C₂₋₇alkoxycarbonyl;
R⁵ represents hydrogen, amino, hydroxy, cyano, C₁₋₆alkyl, C₁₋₆haloalkyl, C₃₋₇cycloalkyl, C₂₋₆alkenyl, C₂₋₆ alkynyl, C₁₋₆alkoxy, (total carbon atom number) C₂₋₇aminocarbonylalkyl, C₁₋₆iminoalkyl, (total carbon atom number) C₂₋₇alkylcarbonyl, (total carbon atom number) C₂₋₇alkylcarbonylamino, (total carbon atom number) C₂₋₇alkylimino, (total carbon atom number) C₆₋₁₂aryl, (total carbon atom number) C₇₋₁₆aralkyl, a heterocyclic group, heterocyclic ring substituted C₁₋₆alkyl, R⁷—C(═O)— or R⁷—C(═S)—;
R⁶ represents hydrogen, cyano, carbonyl, thiocarbonyl, (total carbon atom number) C₂₋₇alkylcarbonyl, (total carbon atom number) C₂₋₇alkylthiocarbonyl, (total carbon atom number) C₂₋₇haloalkylcarbonyl, (total carbon atom number) C₂₋₇haloalkylthiocarbonyl, (total carbon atom number) C₂₋₇alkylaminocarbonyl, (total carbon atom number) C₂₋₇ alkylaminothiocarbonyl, (total carbon atom number) C₃₋₁₃dialkylaminocarbonyl, (total carbon atom number) C₃₋₁₃dialkylaminothiocarbonyl, (total carbon atom number) C₂₋₇ alkoxyaminocarbonyl, (total carbon atom number) C₂₋₇alkoxyaminothiocarbonyl, (total carbon atom number) C₂₋₇alkoxycarbonyl, (total carbon atom number) C₂₋₇alkoxythiocarbonyl, (total carbon atom number) C₂₋₇alkylsulfanylcarbonyl, (total carbon atom number) C₂₋₇alkylsulfanylthiocarbonyl, C₁₋₆alkylsulfonyl, C₁₋₆haloalkylsulfonyl, (total carbon atom number) C₄₋₈ cycloalkylcarbonyl, (total carbon atom number) C₃₋₇alkenylcarbonyl, (total carbon atom number) C₃₋₇alkynylcarbonyl, (total carbon atom number) C₅₋₁₄cycloalkylalkylcarbonyl, (total carbon atom number) C₃₋₁₃alkylsulfanylalkylcarbonyl, (total carbon atom number) C₃₋₁₃alkylsulfinylalkylcarbonyl, (total carbon atom number) C₃₋₁₃alkylsulfonylalkylcarbonyl, (total carbon atom number) C₄₋₁₄alkylcarbonylalkylcarbonyl, (total carbon atom number) C₄₋₈cycloalkylaminocarbonyl, (total carbon atom number) C₃₋₇alkenylaminocarbonyl, (total carbon atom number) C₃₋₇alkynylaminocarbonyl, C₁₋₆alkylaminosulfonyl, (total carbon atom number) C₂₋₁₂dialkylaminosulfonyl, (total carbon atom number) C₃₋₁₃alkoxyalkylcarbonyl, (total carbon atom number) C₄₋₁₄alkylcarbonylaminoalkylcarbonyl, (total carbon atom number) C₄₋₁₄haloalkylcarbonylaminoalkylcarbonyl, (total carbon atom number) C₅₋₂₀alkylsulfanylalkylcarbonylaminoalkylcarbonyl, (total carbon atom number) C₅₋₂₀alkylsulfinylalkylcarbonylaminoalkylcarbonyl, (total carbon atom number) C₅₋₂₀alkylsulfonylalkylcarbonylaminoalkylcarbonyl, (total carbon atom number) C₈₋₁₇aralkylcarbonyl, heterocyclic substituted (total carbon atom number) C₂₋₇alkylcarbonyl, R⁷—C(═O)— or R⁷—C(═S)—.

3. Compounds according to claim 1 or 2, wherein B¹, B² and B³ each independently represents C—X² and B⁴ represents nitrogen.

4. Compounds according to claim 1 or 2, wherein B¹, B³ and B⁴ each independently represents C—X² and B² represents nitrogen.

5. A pesticide containing as an active ingredient one or more compounds selected from the compounds according to any one of claims 1 to 4.

6. Method for controlling plant damaging animal pests characterized in that an active ingredient which is selected from one or more compounds according to any one of claims 1 to 4 is applied to said pests and/or their habitat.

7. Use of an active ingredient which is selected from one or more compounds according to any one of claims 1 to 4 for treating seed of conventional or transgenic plants.

8. Pharmaceutical containing as an active ingredient one or more compounds selected from the compounds according to any one of claims 1 to 4.

9. Use of an active ingredient which is selected from one or more compounds according to claim any one of claims 1 to 4 for the preparation of pharmaceutical compositions for controlling parasites in or on animals.
